SEQUENCE LISTING (5 sequences)

SEQ ID NO: 1 - DNA, 26 bp, Artificial Sequence
  Primer
tatataggatccatgagcgacgtggc

SEQ ID NO: 2 - DNA, 29 bp, Artificial Sequence
  Primer
aaatttctcgagtcaggccgtgctgctgg

SEQ ID NO: 3 - DNA, 25 bp, Artificial Sequence
  AKT1 Mutagenic Primer
acctggcggccacgctacttcctcc

SEQ ID NO: 4 - DNA, 23 bp, Artificial Sequence
  Selection Primer
ctcgagcatgcaactagagggcc

SEQ ID NO: 5 - protein, 14 aa, Artificial Sequence
  Biotinylated synthetic peptide
ARKRERAYSFGHHA

(19) United States
(12) Patent Application Publication
(10) Pub. No.: US 2008/0318947 A1
(43) Pub. Date: Dec. 25, 2008

(54) Inhibitors of Akt Activity

(76) Inventors: Dirk A. Heerding, Collegeville, PA (US); Meagan B. Rouse, Collegeville, PA (US); Mark Andrew Seefeld, Collegeville, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 11/911,892

(22) PCT Filed: Apr. 20, 2006
(86) PCT No.: PCT/US06/14807

§ 371 Date: Jun. 30, 2008

Related U.S. Application Data
(60) Provisional application No. 60/673,120, filed on Apr. 20, 2005.

(51) Int. Cl. A61K 31/5377; C07D 471/04; A61P 35/00; A61K 31/437

(52) U.S. Cl. 514/234.2; 546/118; 514/303; 544/127

(57) ABSTRACT

Invented are novel 1H-imidazo[4,5-c]pyridin-2-yl compounds, the use of such compounds as inhibitors of protein kinase B activity and in the treatment of cancer and arthritis.

### FIELD OF THE INVENTION

This invention relates to novel 1H-imidazo[4,5-c]pyridin-2-yl compounds, the use of such compounds as inhibitors of protein kinase B (hereinafter PKB/Akt, PKB or Akt) activity and in the treatment of cancer and arthritis.

### BACKGROUND OF THE INVENTION

The present invention relates to 1H-imidazo[4,5-c]pyridin-2-yl containing compounds that are inhibitors of the activity of one or more of the isoforms of the serine/threonine kinase, Akt (also known as protein kinase B). The present invention also relates to pharmaceutical compositions comprising such compounds and methods of using the instant compounds in the treatment of cancer and arthritis (Liu et al. Current Opin. Pharmacology 3:317-22 (2003)).

Apoptosis (programmed cell death) plays essential roles in embryonic development and pathogenesis of various diseases, such as degenerative neuronal diseases, cardiovascular diseases and cancer. Recent work has led to the identification of various pro- and anti-apoptotic gene products that are involved in the regulation or execution of programmed cell death. Expression of anti-apoptotic genes, such as Bcl2 or Bcl-x_{L}, inhibits apoptotic cell death induced by various stimuli. On the other hand, expression of pro-apoptotic genes, such as Bax or Bad, leads to programmed cell death (Adams et al. Science, 281:1322-1326 (1998)). The execution of programmed cell death is mediated by caspase-1 related proteinases, including caspase-3, caspase-7, caspase-8 and caspase-9 etc (Thornberry et al. Science, 281:1312-1316 (1998)).

The phosphatidylinositol 3′-OH kinase (PI3K)/Akt/PKB pathway appears important for regulating cell survival/cell death (Kulik et al. Mol. Cell. Biol. 17:1595-1606 (1997); Franke et al, Cell, 88:435-437 (1997); Kauffmann-Zeh et al. Nature 385:544-548 (1997) Hemmings Science, 275:628-630 (1997); Dudek et al., Science, 275:661-665 (1997)). Survival factors, such as platelet derived growth factor (PDGF), nerve growth factor (NGF) and insulin-like growth factor-1 (IGF-1), promote cell survival under various conditions by inducing the activity of PI3K (Kulik et al. 1997, Hemmings 1997). Activated PI3K leads to the production of phosphatidylinositol (3,4,5)-triphosphate (PtdIns (3,4,5)-P3), which in turn binds to, and promotes the activation of, the serine/threonine kinase Akt, which contains a pleckstrin homology (PH)-domain (Franke et al Cell, 81:727-736 (1995); Hemmings Science, 277:534 (1997); Downward, Curr. Opin. Cell Biol. 10:262-267 (1998), Alessi et al., EMBO J. 15: 6541-6551 (1996)). Specific inhibitors of PI3K or dominant negative Akt/PKB mutants abolish survival-promoting activities of these growth factors or cytokines. It has been previously disclosed that inhibitors of PI3K (LY294002 or wortmannin) blocked the activation of Akt/PKB by upstream kinases. In addition, introduction of constitutively active PI3K or Akt/PKB mutants promotes cell survival under conditions in which cells normally undergo apoptotic cell death (Kulik et al. 1997, Dudek et al. 1997).

Analysis of Akt levels in human tumors showed that Akt2 is overexpressed in a significant number of ovarian (J. Q. Cheung et al. Proc. Natl. Acad. Sci. U.S.A. 89:9267-9271 (1992)) and pancreatic cancers (J. Q. Cheung et al. Proc. Natl. Acad. Sci. U.S.A. 93:3636-3641 (1996)). Similarly, Akt3 was found to be overexpressed in breast and prostate cancer cell lines (Nakatani et al. J. Biol. Chem. 274:21528-21532 (1999). It was demonstrated that Akt-2 was over-expressed in 12% of ovarian carcinomas and that amplification of Akt was especially frequent in 50% of undifferentiated tumors, suggestion that Akt may also be associated with tumor aggressiveness (Bellacosa, et al., Int. J. Cancer, 64, pp. 280-285, 1995). Increased Akt1 kinase activity has been reported in breast, ovarian and prostate cancers (Sun et al. Am. J. Pathol. 159:431-7 (2001)).

The tumor suppressor PTEN, a protein and lipid phosphatase that specifically removes the 3′ phosphate of PtdIns(3,4,5)—P3, is a negative regulator of the PI3K/Akt pathway (Li et al. Science 275:1943-1947 (1997), Stambolic et al. Cell 95:29-39 (1998), Sun et al. Proc. Natl. Acad. Sci. U.S.A. 96:6199-6204 (1999)). Germline mutations of PTEN are responsible for human cancer syndromes such as Cowden disease (Liaw et al. Nature Genetics 16:64-67 (1997)). PTEN is deleted in a large percentage of human tumors and tumor cell lines without functional PTEN show elevated levels of activated Akt (Li et al. supra, Guldberg et al. Cancer Research 57:3660-3663 (1997), Risinger et al. Cancer Research 57:4736-4738 (1997)).

These observations demonstrate that the PI3K/Akt pathway plays important roles for regulating cell survival or apoptosis in tumorigenesis.

Three members of the Akt/PKB subfamily of second-messenger regulated serine/threonine protein kinases have been identified and termed Akt1/PKBα, Akt2/PKBβ, and Akt3/PKBγ respectively. The isoforms are homologous, particularly in regions encoding the catalytic domains. Akt/PKBs are activated by phosphorylation events occurring in response to PI3K signaling. PI3K phosphorylates membrane inositol phospholipids, generating the second messengers phosphatidyl-inositol 3,4,5-trisphosphate and phosphatidylinositol 3,4-bisphosphate, which have been shown to bind to the PH domain of Akt/PKB. The current model of Akt/PKB activation proposes recruitment of the enzyme to the membrane by 3′-phosphorylated phosphoinositides, where phosphorylation of the regulatory sites of Akt/PKB by the upstream kinases occurs (B. A. Hemmings, Science 275:628-630 (1997); B. A. Hemmings, Science 276:534 (1997); J. Downward, Science 279:673-674 (1998)).

Phosphorylation of Akt1/PKBα occurs on two regulatory sites, Thr^{3O8} in the catalytic domain activation loop and on Ser⁴⁷³ near the carboxy terminus (D. R. Alessi et al. EMBO J. 15:6541-6551 (1996) and R. Meier et al. J. Biol. Chem. 272:30491-30497 (1997)). Equivalent regulatory phosphorylation sites occur in Akt2/PKBβ and Akt3/PKBγ. The upstream kinase, which phosphorylates Akt/PKB at the activation loop site has been cloned and termed 3′-phosphoinositide dependent protein kinase 1 (PDK1). PDK1 phosphorylates not only Akt/PKB, but also p70 ribosomal S6 kinase, p90RSK, serum and glucocorticoid-regulated kinase (SGK), and protein kinase C. The upstream kinase phosphorylating the regulatory site of Akt/PKB near the carboxy terminus has not been identified yet, but recent reports imply a role for the integrin-linked kinase (ILK-1), a serine/threonine protein kinase, or autophosphorylation.

Inhibition of Akt activation and activity can be achieved by inhibiting PI3K with inhibitors such as LY294002 and wortmannin. However, PI3K inhibition has the potential to indiscriminately affect not just all three Akt isozymes but also other PH domain-containing signaling molecules that are dependent on PdtIns(3,4,5)-P3, such as the Tec family of tyrosine kinases. Furthermore, it has been disclosed that Akt can be activated by growth signals that are independent of PI3K.

Alternatively, Akt activity can be inhibited by blocking the activity of the upstream kinase PDK1. The compound UCN-01 is a reported inhibitor of PDK1. Biochem. J. 375(2):255 (2003). Again, inhibition of PDK1 would result in inhibition of multiple protein kinases whose activities depend on PDK1, such as atypical PKC isoforms, SGK, and S6 kinases (Williams et al. Curr. Biol. 10:439-448 (2000).

Small molecule inhibitors of Akt are useful in the treatment of tumors, especially those with activated Akt (e.g. PTEN null tumors and tumors with ras mutations). PTEN is a critical negative regulator of Akt and its function is lost in many cancers, including breast and prostate carcinomas, glioblastomas, and several cancer syndromes including Bannayan-Zonana syndrome (Maehama, T. et al. Annual Review of Biochemistry, 70: 247 (2001)), Cowden disease (Parsons, R.; Simpson, L. Methods in Molecular Biology (Totowa, N.J., United States), 222 (Tumor Suppressor Genes, Volume 1): 147 (2003)), and Lhermitte-Duclos disease (Backman, S. et al. Current Opinion in Neurobiology, 12(5): 516 (2002)). Akt3 is up-regulated in estrogen receptor-deficient breast cancers and androgen-independent prostate cancer cell lines and Akt2 is over-expressed in pancreatic and ovarian carcinomas. Akt1 is amplified in gastric cancers (Staal, Proc. Natl. Acad. Sci. USA 84: 5034-7 (1987) and upregulated in breast cancers (Stal et al. Breast Cancer Res. 5: R37-R44 (2003)). Therefore a small molecule Akt inhibitor is expected to be useful for the treatment of these types of cancer as well as other types of cancer. Akt inhibitors are also useful in combination with further chemotherapeutic agents.

It is an object of the instant invention to provide novel compounds that are inhibitors of Akt/PKB.

It is also an object of the present invention to provide pharmaceutical compositions that comprise a pharmaceutical carrier and compounds useful in the methods of the invention.

It is also an object of the present invention to provide a method for treating cancer that comprises administering such inhibitors of Akt/PKB activity.

It is also an object of the present invention to provide a method for treating arthritis that comprises administering such inhibitors of Akt/PKB activity.

### SUMMARY OF THE INVENTION

This invention relates to novel compounds of Formula (I):

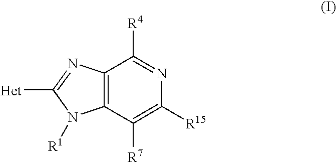

wherein:
Het is selected from the group consisting of:

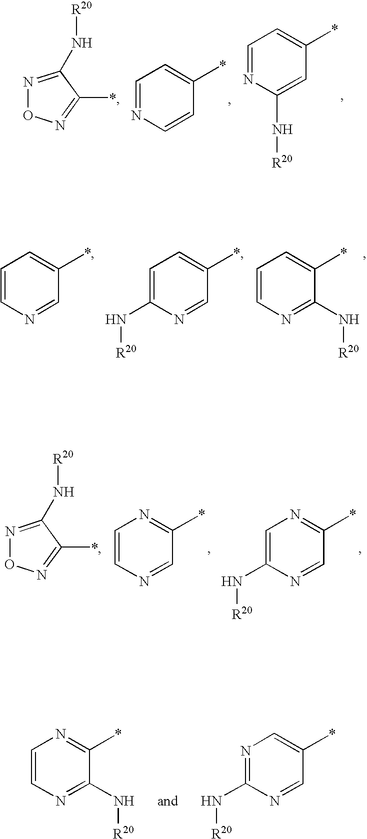

R²⁰ is selected from hydrogen, alkyl, alkyl substituted with one or more substituents selected from the group consisting of: hydroxy, alkoxy, amino, N-acylamino, cyclopropyl and halogen, cycloalkyl, cycloalkyl substituted with one or more substituents selected from the group consisting of: hydroxy, alkoxy, amino, N-acylamino and halogen, cycloalkyl containing from 1 to 4 heteroatoms, cycloalkyl containing from 1 to 4 heteroatoms substituted with one or more substituents selected from the group consisting of: hydroxy, alkoxy, amino, N-acylamino and halogen, C₁-C₁₂aryl and C₁-C₁₂aryl substituted with one or more substituents selected from the group consisting of: hydroxy, alkoxy, amino, N-acylamino and halogen;
R¹ is selected from hydrogen, alkyl, alkyl substituted with one or more substituents selected from the group consisting of: hydroxy, alkoxy, amino, N-acylamino, cyclopropyl and halogen, cycloalkyl, cycloalkyl substituted with one or more substituents selected from the group consisting of: hydroxy, alkoxy, amino, N-acylamino and halogen, cycloalkyl containing from 1 to 4 heteroatoms, cycloalkyl containing from 1 to 4 heteroatoms substituted with one or more substituents selected from the group consisting of: hydroxy, alkoxy, amino, N-acylamino and halogen, C₁-C₁₂aryl and C₁-C₁₂aryl substituted with one or more substituents selected from the group consisting of: hydroxy, alkoxy, amino, N-acylamino and halogen;
R⁴ is selected from hydrogen, halogen, alkyl, substituted alkyl, alkoxy, substituted alkoxy, acetamide, cyano, urea, substituted urea, aryl, substituted aryl, aryloxy, substituted aryloxy, oxo, hydroxy, acyloxy, amino, N-acylamino, substituted N-acylamino, cycloalkyl, substituted cycloalkyl, cycloalkyl containing from 1 to 4 heteroatoms and substituted cycloalkyl containing from 1 to 4 heteroatoms;
R¹⁵ is selected from halogen, alkyl, substituted alkyl, alkoxy, substituted alkoxy, acetamide, cyano, urea, substituted urea, aryl, substituted aryl, aryloxy, substituted aryloxy, oxo, hydroxy, acyloxy, amino, N-acylamino, substituted N-acylamino, cycloalkyl, substituted cycloalkyl, cycloalkyl containing from 1 to 4 heteroatoms, substituted cycloalkyl containing from 1 to 4 heteroatoms, cycloalkyloxy, substituted cycloalkyloxy, cycloalkyloxy containing from 1 to 4 heteroatoms and substituted cycloalkyloxy containing from 1 to 4 heteroatoms; and when R²⁰ is other than hydrogen, R¹⁵ can additionally be hydrogen;
R⁷ is selected from hydrogen, halogen, alkyl, substituted alkyl, alkoxy, substituted alkoxy, acetamide, cyano, urea, substituted urea, aryl, substituted aryl, aryloxy, substituted aryloxy, oxo, hydroxy, acyloxy, amino, N-acylamino, substituted N-acylamino, cycloalkyl, substituted cycloalkyl, cycloalkyl containing from 1 to 4 heteroatoms and substituted cycloalkyl containing from 1 to 4 heteroatoms;
   or R¹⁵ and R⁷ taken together represent a 5 to 6 member saturated ring containing up to one heteroatom selected from oxygen and nitrogen, where the ring is optionally substituted with one or more substituents selected from amino, methylamino and dimethylamino;
      and/or pharmaceutically acceptable salts, hydrates, solvates and pro-drugs thereof.

This invention relates to a method of treating cancer, which comprises administering to a subject in need thereof an effective amount of an Akt/PKB inhibiting compound of Formula (I).

This invention relates to a method of treating arthritis, which comprises administering to a subject in need thereof an effective amount of an Akt/PKB inhibiting compound of Formula (I).

The present invention also relates to the discovery that the compounds of Formula (I) are active as inhibitors of Akt/PKB.

In a further aspect of the invention there is provided novel processes and novel intermediates useful in preparing the presently invented Akt/PKB inhibiting compounds.

Included in the present invention are pharmaceutical compositions that comprise a pharmaceutical carrier and compounds useful in the methods of the invention.

Also included in the present invention are methods of co-administering the presently invented Akt/PKB inhibiting compounds with further active ingredients.

### DETAILED DESCRIPTION OF THE INVENTION

This invention relates to compounds of Formula (I) as described above.

The presently invented compounds of Formula (I) inhibit Akt/PKB activity. In particular, the compounds disclosed herein inhibit each of the three Akt/PKB isoforms.

Included in the presently invented compounds of Formula (I) are compounds of Formula (Ia):

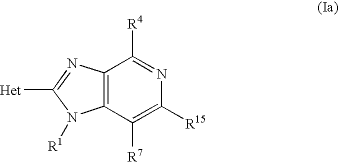

wherein:
Het is selected from the group consisting of:

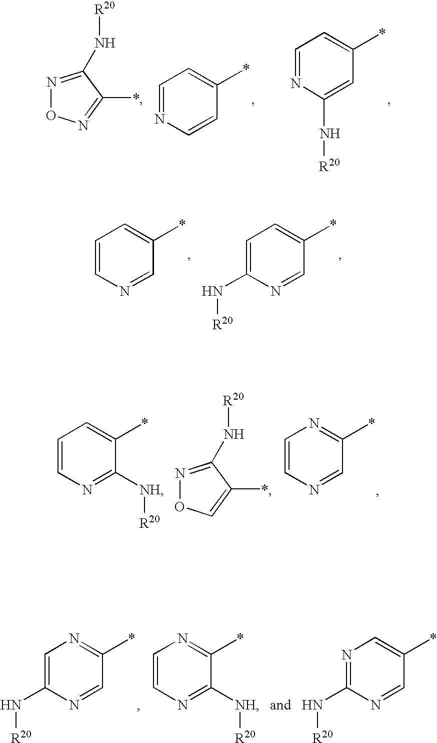

R²⁰ is selected from hydrogen, alkyl, alkyl substituted with one or more substituents selected from the group consisting of: hydroxy, alkoxy, amino, N-acylamino, cyclopropyl and halogen, cycloalkyl, cycloalkyl substituted with one or more substituents selected from the group consisting of: hydroxy, alkoxy, amino, N-acylamino and halogen, cycloalkyl containing from 1 to 4 heteroatoms, cycloalkyl containing from 1 to 4 heteroatoms substituted with one or more substituents selected from the group consisting of: hydroxy, alkoxy, amino, N-acylamino and halogen, C₁-C₁₂aryl and C₁-C₁₂aryl substituted with one or more substituents selected from the group consisting of: hydroxy, alkoxy, amino, N-acylamino and halogen;
R¹ is selected from hydrogen, alkyl, alkyl substituted with one or more substituents selected from the group consisting of: hydroxy, alkoxy, amino, N-acylamino, cyclopropyl and halogen, cycloalkyl, cycloalkyl substituted with one or more substituents selected from the group consisting of: hydroxy, alkoxy, amino, N-acylamino and halogen, cycloalkyl containing from 1 to 4 heteroatoms, cycloalkyl containing from 1 to 4 heteroatoms substituted with one or more substituents selected from the group consisting of: hydroxy, alkoxy, amino, N-acylamino and halogen, C₁-C₁₂aryl and C₁-C₁₂aryl substituted with one or more substituents selected from the group consisting of: hydroxy, alkoxy, amino, N-acylamino and halogen;
R⁴ is selected from hydrogen, halogen, alkyl, substituted alkyl, alkoxy, substituted alkoxy, acetamide, cyano, urea, substituted urea, aryl, substituted aryl, aryloxy, substituted aryloxy, oxo, hydroxy, acyloxy, amino, N-acylamino, substituted N-acylamino, cycloalkyl, substituted cycloalkyl, cycloalkyl containing from 1 to 4 heteroatoms and substituted cycloalkyl containing from 1 to 4 heteroatoms;
R¹⁵ is selected from halogen, alkyl, substituted alkyl, alkoxy, substituted alkoxy, acetamide, cyano, urea, substituted urea, aryl, substituted aryl, aryloxy, substituted aryloxy, oxo, hydroxy, acyloxy, amino, N-acylamino, substituted N-acylamino, cycloalkyl, substituted cycloalkyl, cycloalkyl containing from 1 to 4 heteroatoms, substituted cycloalkyl containing from 1 to 4 heteroatoms, cycloalkyloxy, substituted cycloalkyloxy, cycloalkyloxy containing from 1 to 4 heteroatoms and substituted cycloalkyloxy containing from 1 to 4 heteroatoms;
R⁷ is selected from hydrogen, halogen, alkyl, substituted alkyl, alkoxy, substituted alkoxy, acetamide, cyano, urea, substituted urea, aryl, substituted aryl, aryloxy, substituted aryloxy, oxo, hydroxy, acyloxy, amino, N-acylamino, substituted N-acylamino, cycloalkyl, substituted cycloalkyl, cycloalkyl containing from 1 to 4 heteroatoms and substituted cycloalkyl containing from 1 to 4 heteroatoms;
   or R¹⁵ and R⁷ taken together represent a 5 to 6 member saturated ring containing up to one heteroatom selected from oxygen and nitrogen, where the ring is optionally substituted with one or more substituents selected from amino, methylamino and dimethylamino;
      and/or pharmaceutically acceptable salts, hydrates, solvates and pro-drugs thereof.

Included among the presently invented compounds of Formula (I) are those having Formula (II):

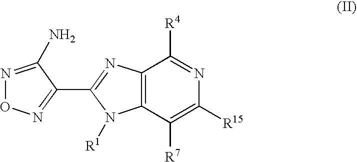

wherein:
R¹ is selected from hydrogen, alkyl, alkyl substituted with one or more substituents selected from the group consisting of: hydroxy, alkoxy, amino, N-acylamino, cyclopropyl and halogen, cycloalkyl, cycloalkyl substituted with one or more substituents selected from the group consisting of: hydroxy, alkoxy, amino, N-acylamino and halogen, cycloalkyl containing from 1 to 4 heteroatoms, cycloalkyl containing from 1 to 4 heteroatoms substituted with one or more substituents selected from the group consisting of: hydroxy, alkoxy, amino, N-acylamino and halogen, C₁-C₁₂aryl and C₁-C₁₂aryl substituted with one or more substituents selected from the group consisting of: hydroxy, alkoxy, amino, N-acylamino and halogen;
R⁴ is selected from hydrogen, halogen, alkyl, substituted alkyl, alkoxy, substituted alkoxy, acetamide, cyano, urea, substituted urea, aryl, substituted aryl, aryloxy, substituted aryloxy, oxo, hydroxy, acyloxy, amino, N-acylamino, substituted N-acylamino, cycloalkyl, substituted cycloalkyl, cycloalkyl containing from 1 to 4 heteroatoms and substituted cycloalkyl containing from 1 to 4 heteroatoms;
R¹⁵ is selected from halogen, alkyl, substituted alkyl, alkoxy, substituted alkoxy, acetamide, cyano, urea, substituted urea, aryl, substituted aryl, aryloxy, substituted aryloxy, oxo, hydroxy, acyloxy, amino, N-acylamino, substituted N-acylamino, cycloalkyl, substituted cycloalkyl, cycloalkyl containing from 1 to 4 heteroatoms, substituted cycloalkyl containing from 1 to 4 heteroatoms, cycloalkyloxy, substituted cycloalkyloxy, cycloalkyloxy containing from 1 to 4 heteroatoms and substituted cycloalkyloxy containing from 1 to 4 heteroatoms;
R⁷ is selected from hydrogen, halogen, alkyl, substituted alkyl, alkoxy, substituted alkoxy, acetamide, cyano, urea, substituted urea, aryl, substituted aryl, aryloxy, substituted aryloxy, oxo, hydroxy, acyloxy, amino, N-acylamino, substituted N-acylamino, cycloalkyl, substituted cycloalkyl, cycloalkyl containing from 1 to 4 heteroatoms and substituted cycloalkyl containing from 1 to 4 heteroatoms;
   or R¹⁵ and R⁷ taken together represent a 5 to 6 member saturated ring containing up to one heteroatom selected from oxygen and nitrogen, where the ring is optionally substituted with one or more substituents selected from amino, methylamino and dimethylamino;
      and/or pharmaceutically acceptable salts, hydrates, solvates and pro-drugs thereof.

Included among the presently invented compounds of Formula (I) are those in which R⁷ and R²⁰ are hydrogen.

Included among the presently invented compounds of Formula (II) are those in which R⁷ is hydrogen.

Included among the presently invented compounds are compounds of Formula (I) in which:
R²⁰ is selected from: alkyl, alkyl substituted with one or more substituents selected from the group consisting of: hydroxy, alkoxy, amino, N-acylamino, cyclopropyl and halogen, cycloalkyl containing from 1 to 3 heteroatoms and C₁-C₁₂aryl;
R¹ is selected from: alkyl, alkyl, substituted with one or more substituents selected from the group consisting of: hydroxy, alkoxy, amino, N-acylamino, cyclopropyl and halogen, cycloalkyl containing from 1 to 3 heteroatoms and C₁-C₁₂aryl;
R⁴ is selected from hydrogen, halogen, alkyl, substituted alkyl, alkoxy, substituted alkoxy, cycloalkyl, cycloalkyl containing from 1 to 3 heteroatoms, C₁-C₁₂aryl and C₁-C₁₂aryl substituted with one or more substituents selected from the group consisting of: alkyl, substituted alkyl, aryloxy, hydroxy, alkoxy, acyloxy, amino, N-acylamino, nitro, cyano and halogen;
R¹⁵ is selected from hydrogen, halogen, alkyl, substituted alkyl, alkoxy, substituted alkoxy, cycloalkyl, cycloalkyl containing from 1 to 3 heteroatoms, substituted cycloalkyl, substituted cycloalkyl containing from 1 to 3 heteroatoms, cycloalkyloxy, cycloalkyloxy containing from 1 to 3 heteroatoms, substituted cycloalkyloxy, substituted cycloalkyloxy containing from 1 to 3 heteroatoms, aryloxy, substituted arlyoxy, C₁-C₁₂aryl and C₁-C₁₂aryl substituted with one or more substituents selected from the group consisting of: alkyl, substituted alkyl, aryloxy, hydroxy, alkoxy, acyloxy, amino, N-acylamino, substituted N-acylamino, hydroxyalkyl, aminoalkoxy, aminoalkyl, nitro, nitrile, cyano and halogen; and
R⁷ is hydrogen;
   and/or pharmaceutically acceptable salts, hydrates, solvates and pro-drugs thereof.

Included among the presently invented compounds of Formula (II) are those in which:
R¹ is selected from: alkyl, alkyl substituted with one or more substituents selected from the group consisting of: hydroxy, alkoxy, amino, N-acylamino, cyclopropyl and halogen, cycloalkyl containing from 1 to 3 heteroatoms and C₁-C₁₂aryl;
R⁴ is selected from hydrogen, halogen, alkyl, substituted alkyl, alkoxy, substituted alkoxy, cycloalkyl, cycloalkyl containing from 1 to 3 heteroatoms, C₁-C₁₂aryl and C₁-C₁₂aryl substituted with one or more substituents selected from the group consisting of: alkyl, substituted alkyl, aryloxy, hydroxy, alkoxy, acyloxy, amino, N-acylamino, nitro, cyano and halogen;
R¹⁵ is selected from halogen, alkyl, substituted alkyl, alkoxy, substituted alkoxy, cycloalkyl, cycloalkyl containing from 1 to 3 heteroatoms, substituted cycloalkyl, substituted cycloalkyl containing from 1 to 3 heteroatoms, cycloalkyloxy, cycloalkyloxy containing from 1 to 3 heteroatoms, substituted cycloalkyloxy, substituted cycloalkyloxy containing from 1 to 3 heteroatoms, aryloxy, substituted aryloxy, C₁-C₁₂aryl and C₁-C₁₂aryl substituted with one or more substituents selected from the group consisting of: alkyl, substituted alkyl, aryloxy, hydroxy, alkoxy, acyloxy, amino, N-acylamino, substituted N-acylamino, hydroxyalkyl, aminoalkoxy, aminoalkyl, nitro, nitrile, cyano and halogen; and
R⁷ is hydrogen;
   and/or pharmaceutically acceptable salts, hydrates, solvates and pro-drugs thereof.

Included among the presently invented compounds of Formula (I) are those in which:
R²⁰ is selected hydrogen;
R¹ is selected from: alkyl, alkyl substituted with one or more substituents selected from the group consisting of: hydroxy, alkoxy, amino, N-acylamino, cyclopropyl and halogen;
R⁴ is selected from hydrogen, halogen, alkyl, substituted alkyl, alkoxy, substituted alkoxy, cycloalkyl, cycloalkyl containing from 1 to 3 heteroatoms, C₁-C₁₂aryl and C₁-C₁₂aryl substituted with one or more substituents selected from the group consisting of: alkyl, substituted alkyl, aryloxy, hydroxy, alkoxy, acyloxy, amino, N-acylamino, nitro, cyano and halogen;
R¹⁵ is selected from halogen, alkyl, substituted alkyl, oxo, cycloalkyl, alkoxy, substituted alkoxy, cycloalkyl containing from 1 to 3 heteroatoms, substituted cycloalkyl, substituted cycloalkyl containing from 1 to 3 heteroatoms, cycloalkyloxy, cycloalkyloxy containing from 1 to 3 heteroatoms, substituted cycloalkyloxy, substituted cycloalkyloxy containing from 1 to 3 heteroatoms, C₁-C₁₂aryl, C₁-C₁₂aryloxy, C₁-C₁₂aryloxy substituted with one or more substituents selected from the group consisting of: alkyl, substituted alkyl, aryloxy, hydroxy, alkoxy, acyloxy, amino, N-acylamino, substituted N-acylamino, hydroxyalkyl, aminoalkoxy, aminoalkyl, nitro, nitrile, cyano and halogen and C₁-C₁₂aryl substituted with one or more substituents selected from the group consisting of: alkyl, substituted alkyl, aryloxy, hydroxy, alkoxy, acyloxy, amino, N-acylamino, substituted N-acylamino, hydroxyalkyl, aminoalkoxy, aminoalkyl, nitro, nitrile, cyano and halogen; and
R⁷ is hydrogen; and/or pharmaceutically acceptable salts, hydrates, solvates and pro-drugs thereof.

Included among the presently invented compounds of Formula (II) are those in which:
R¹ is selected from: alkyl, alkyl substituted with one or more substituents selected from the group consisting of: hydroxy, alkoxy, amino, N-acylamino, cyclopropyl and halogen;
R⁴ is selected from hydrogen, halogen, alkyl, substituted alkyl, alkoxy, substituted alkoxy, cycloalkyl, cycloalkyl containing from 1 to 3 heteroatoms, C₁-C₁₂aryl and C₁-C₁₂aryl substituted with one or more substituents selected from the group consisting of: alkyl, substituted alkyl, aryloxy, hydroxy, alkoxy, acyloxy, amino, N-acylamino, nitro, cyano and halogen;
R¹⁵ is selected from halogen, alkyl, substituted alkyl, oxo, cycloalkyl, alkoxy, substituted alkoxy, cycloalkyl containing from 1 to 3 heteroatoms, substituted cycloalkyl, substituted cycloalkyl containing from 1 to 3 heteroatoms, cycloalkyloxy, cycloalkyloxy containing from 1 to 3 heteroatoms, substituted cycloalkyloxy, substituted cycloalkyloxy containing from 1 to 3 heteroatoms, C₁-C₁₂aryl, C₁-C₁₂aryloxy, C₁-C₁₂aryloxy substituted with one or more substituents selected from the group consisting of: alkyl, substituted alkyl, aryloxy, hydroxy, alkoxy, acyloxy, amino, N-acylamino, substituted N-acylamino, hydroxyalkyl, aminoalkoxy, aminoalkyl, nitro, nitrile, cyano and halogen and C₁-C₁₂aryl substituted with one or more substituents selected from the group consisting of: alkyl, substituted alkyl, aryloxy, hydroxy, alkoxy, acyloxy, amino, N-acylamino, substituted N-acylamino, hydroxyalkyl, aminoalkoxy, aminoalkyl, nitro, nitrile, cyano and halogen; and
R⁷ is hydrogen;
   and/or pharmaceutically acceptable salts, hydrates, solvates and pro-drugs thereof.

Included among the presently invented compounds of Formula (I) are those in which:
R²⁰ is hydrogen;
R¹ is from: alkyl;
R⁴ is selected from alkyl and alkyl substituted with one or more substituents selected from the group consisting of: hydroxy, alkoxy, amino, N-acylamino, cyclopropyl and halogen;
R¹⁵ is selected from halogen, alkyl, substituted alkyl, oxo, cycloalkyl, alkoxy, substituted alkoxy, cycloalkyl containing from 1 to 3 heteroatoms, substituted cycloalkyl, substituted cycloalkyl containing from 1 to 3 heteroatoms, cycloalkyloxy, cycloalkyloxy containing from 1 to 3 heteroatoms, substituted cycloalkyloxy, substituted cycloalkyloxy containing from 1 to 3 heteroatoms, C₁-C₁₂aryl, C₁-C₁₂aryloxy, C₁-C₁₂aryloxy substituted with from one to three substituents selected from the group consisting of: alkyl, hydroxy, alkoxy, acyloxy, amino, N-acylamino, substituted N-acylamino, hydroxyalkyl, aminoalkoxy, aminoalkyl, nitro, nitrile, cyano and halogen, and C₁-C₁₂aryl substituted with from one to three substituents selected from the group consisting of: alkyl, hydroxy, alkoxy, acyloxy, amino, N-acylamino, substituted N-acylamino, hydroxyalkyl, aminoalkoxy, aminoalkyl, nitro, nitrile, cyano and halogen, and
R⁷ is hydrogen;
   and/or pharmaceutically acceptable salts, hydrates, solvates and pro-drugs thereof.

Included among the presently invented compounds of Formula (II) are those in which:
R¹ is selected from: alkyl, alkyl substituted with one or more substituents selected from the group consisting of: hydroxy, alkoxy, amino, N-acylamino, cyclopropyl and halogen;
R⁴ is selected from hydrogen, halogen, alkyl, substituted alkyl, alkoxy, substituted alkoxy, cycloalkyl, cycloalkyl containing from 1 to 3 heteroatoms, C₁-C₁₂aryl and C₁-C₁₂aryl substituted with one or more substituents selected from the group consisting of: alkyl, substituted alkyl, aryloxy, hydroxy, alkoxy, acyloxy, amino, N-acylamino, nitro, cyano and halogen;
R¹⁵ is selected from halogen, alkyl, substituted alkyl, oxo, cycloalkyl, alkoxy, substituted alkoxy, cycloalkyl containing from 1 to 3 heteroatoms, substituted cycloalkyl, substituted cycloalkyl containing from 1 to 3 heteroatoms, cycloalkyloxy, cycloalkyloxy containing from 1 to 3 heteroatoms, substituted cycloalkyloxy, substituted cycloalkyloxy containing from 1 to 3 heteroatoms, C₁-C₁₂aryl, C₁-C₁₂aryloxy, C₁-C₁₂aryloxy substituted with from one to three substituents selected from the group consisting of: alkyl, hydroxy, alkoxy, acyloxy, amino, N-acylamino, substituted N-acylamino, hydroxyalkyl, aminoalkoxy, aminoalkyl, nitro, nitrile, cyano and halogen, and C₁-C₁₂aryl substituted with from one to three substituents selected from the group consisting of: alkyl, hydroxy, alkoxy, acyloxy, amino, N-acylamino, substituted N-acylamino, hydroxyalkyl, aminoalkoxy, aminoalkyl, nitro, nitrile, cyano and halogen, and
R⁷ is hydrogen;
   and/or pharmaceutically acceptable salts, hydrates, solvates and pro-drugs thereof.

Included among the presently invented compounds of Formula (I) are those in which:
R²⁰ is hydrogen;
R¹ is from: alkyl;
R⁴ is selected from alkyl and alkyl substituted with from one to three substituents selected from the group consisting of: hydroxy, alkoxy, amino, N-acylamino, cyclopropyl and halogen;
R¹⁵ is selected from halogen, alkyl, substituted alkyl, oxo, cycloalkyl, alkoxy, substituted alkoxy, cycloalkyl containing from 1 to 3 heteroatoms, substituted cycloalkyl, substituted cycloalkyl containing from 1 to 3 heteroatoms, cycloalkyloxy, cycloalkyloxy containing from 1 to 3 heteroatoms, substituted cycloalkyloxy, substituted cycloalkyloxy containing from 1 to 3 heteroatoms, C₁-C₁₂aryl, C₁-C₁₂aryloxy, C₁-C₁₂aryloxy substituted with from one to three substituents selected from the group consisting of: alkyl, hydroxy, alkoxy, acyloxy, amino, N-acylamino, substituted N-acylamino, hydroxyalkyl, aminoalkoxy, aminoalkyl, nitro, nitrile, cyano and halogen, and C₁-C₁₂aryl substituted with from one to three substituents selected from the group consisting of: alkyl, hydroxy, alkoxy, acyloxy, amino, N-acylamino, substituted N-acylamino, hydroxyalkyl, aminoalkoxy, aminoalkyl, nitro, nitrile, cyano and halogen, and
R⁷ is hydrogen,
   and/or pharmaceutically acceptable salts, hydrates, solvates and pro-drugs thereof.

Included among the presently invented compounds of Formula (II) are those in which:
R¹ is selected from: alkyl, alkyl substituted with from one to three substituents selected from the group consisting of: hydroxy, alkoxy, amino, N-acylamino, cyclopropyl and halogen;
R⁴ is selected from hydrogen, halogen, alkyl, substituted alkyl, alkoxy, substituted alkoxy, cycloalkyl, cycloalkyl containing from 1 to 3 heteroatoms, C₁-C₁₂aryl and C₁-C₁₂aryl substituted with from one to three substituents selected from the group consisting of: alkyl, substituted alkyl, aryloxy, hydroxy, alkoxy, acyloxy, amino, N-acylamino, nitro, cyano and halogen;
R¹⁵ is selected from alkyl, substituted alkyl, oxo, cycloalkyl, alkoxy, substituted alkoxy, cycloalkyl containing from 1 to 3 heteroatoms, substituted cycloalkyl, substituted cycloalkyl containing from 1 to 3 heteroatoms, cycloalkyloxy, cycloalkyloxy containing from 1 to 3 heteroatoms, substituted cycloalkyloxy, substituted cycloalkyloxy containing from 1 to 3 heteroatoms, C₁-C₁₂aryl, C₁-C₁₂aryloxy, C₁-C₁₂aryloxy substituted with from one to three substituents selected from the group consisting of: alkyl, hydroxy, alkoxy, acyloxy, amino, N-acylamino, substituted N-acylamino, hydroxyalkyl, aminoalkoxy, aminoalkyl, nitro, nitrile, cyano and halogen, and C₁-C₁₂aryl substituted with from one to three substituents selected from the group consisting of: alkyl, hydroxy, alkoxy, acyloxy, amino, N-acylamino, substituted N-acylamino, hydroxyalkyl, aminoalkoxy, aminoalkyl, nitro, nitrile, cyano and halogen, and
R⁷ is hydrogen;
   and/or pharmaceutically acceptable salts, hydrates, solvates and pro-drugs thereof.

Included among the presently invented compounds of Formula (II) are those in which:
R²⁰ is hydrogen;
R¹ is from: alkyl;
R⁴ is selected from alkyl and alkyl substituted with from one to three substituents selected from the group consisting of: hydroxy, alkoxy, amino, N-acylamino, cyclopropyl and halogen;
R¹⁵ is substituted alkoxy; and
R⁷ is hydrogen;
   and/or pharmaceutically acceptable salts, hydrates, solvates and pro-drugs thereof.

Included among the presently invented compounds of Formula (II) are those in which:
R¹ is selected from: alkyl;
R⁴ is selected from alkyl and alkyl substituted with from one to three substituents selected from the group consisting of: hydroxy, alkoxy, amino, N-acylamino, cyclopropyl and halogen;
R¹⁵ is substituted alkoxy; and
R⁷ is hydrogen;
   and/or pharmaceutically acceptable salts, hydrates, solvates and pro-drugs thereof.

Included among the novel compounds useful in the present invention are:
4,4′-[2-(4-amino-1,2,5-oxadiazol-3-yl)-1-ethyl-1H-imidazo[4,5-c]pyridine-4,6-diyl]bis(2-methyl-3-butyn-2-ol);
4-[2-(4-amino-1,2,5-oxadiazol-3-yl)-6-(3-aminophenyl)-1-ethyl-1H-imidazo[4,5-c]pyridin-4-yl]-2-methyl-3-butyn-2-ol;
4-[2-(4-amino-1,2,5-oxadiazol-3-yl)-6-(2-aminophenyl)-1-ethyl-1H-imidazo[4,5-c]pyridin-4-yl]-2-methyl-3-butyn-2-ol;
4-[6-[3-(aminomethyl)phenyl]-2-(4-amino-1,2,5-oxadiazol-3-yl)-1-ethyl-1H-imidazo[4,5-c]pyridin-4-yl]-2-methyl-3-butyn-2-ol;
2-[2-(4-amino-1,2,5-oxadiazol-3-yl)-1-ethyl-4-(3-hydroxy-3-methyl-1-butyn-1-yl)-1H-imidazo[4,5-c]pyridin-6-yl]benzonitrile;
4-{2-(4-amino-1,2,5-oxadiazol-3-yl)-1-ethyl-6-[2-(hydroxymethyl)phenyl]-1H-imidazo[4,5-c]pyridin-4-yl}-2-methyl-3-butyn-2-ol;
N-{4-[2-(4-amino-1,2,5-oxadiazol-3-yl)-1-ethyl-4-(3-hydroxy-3-methyl-1-butyn-1-yl)-1H-imidazo[4,5-c]pyridin-6-yl]phenyl}acetamide;
4-[2-(4-amino-1,2,5-oxadiazol-3-yl)-1-ethyl-6-(1H-indol-5-yl)-1H-imidazo[4,5-c]pyridin-4-yl]-2-methyl-3-butyn-2-ol;
N-{3-[2-(4-amino-1,2,5-oxadiazol-3-yl)-1-ethyl-4-(3-hydroxy-3-methyl-1-butyn-1-yl)-1H-imidazo[4,5-c]pyridin-6-yl]phenyl}acetamide;
4-[6-[3-(aminomethyl)phenyl]-1-ethyl-4-(1H-pyrrol-3-yl)-1H-imidazo[4,5-c]pyridin-2-yl]-1,2,5-oxadiazol-3-amine;
N¹-{3-[2-(4-amino-1,2,5-oxadiazol-3-yl)-1-ethyl-4-(3-hydroxy-3-methyl-1-butyn-1-yl)-1H-imidazo[4,5-c]pyridin-6-yl]phenyl}glycinamide;
4-[2-(4-amino-1,2,5-oxadiazol-3-yl)-6-(4-aminophenyl)-1-ethyl-1H-imidazo[4,5-c]pyridin-4-yl]-2-methyl-3-butyn-2-ol;
3-[2-(4-amino-1,2,5-oxadiazol-3-yl)-1-ethyl-4-(3-hydroxy-3-methyl-1-butyn-1-yl)-1H-imidazo[4,5-c]pyridin-6-yl]phenol;
4-(2-(4-amino-1,2,5-oxadiazol-3-yl)-6-{3-[(3-aminopropyl)oxy]phenyl}-1-ethyl-1H-imidazo[4,5-c]pyridin-4-yl)-2-methyl-3-butyn-2-ol;
4-[6-{3-[(2-aminoethyl)oxy]phenyl}-2-(4-amino-1,2,5-oxadiazol-3-yl)-1-ethyl-1H-imidazo[4,5-c]pyridin-4-yl]-2-methyl-3-butyn-2-ol;
2-[2-(4-amino-1,2,5-oxadiazol-3-yl)-1-ethyl-4-(3-hydroxy-3-methyl-1-butyn-1-yl)-1H-imidazo[4,5-c]pyridin-6-yl]phenol;
4-[6-[(4-aminobutyl)oxy]-2-(4-amino-1,2,5-oxadiazol-3-yl)-1-ethyl-1H-imidazo[4,5-c]pyridin-4-yl]-2-methyl-3-butyn-2-ol;
4-{2-(4-amino-1,2,5-oxadiazol-3-yl)-6-[(3-aminopropyl)oxy]-1-ethyl-1H-imidazo[4,5-c]pyridin-4-yl}-2-methyl-3-butyn-2-ol;
4-{2-(4-amino-1,2,5-oxadiazol-3-yl)-6-[(5-aminopentyl)oxy]-1-ethyl-1H-imidazo[4,5-c]pyridin-4-yl}-2-methyl-3-butyn-2-ol;
4-(2-(4-amino-1,2,5-oxadiazol-3-yl)-1-ethyl-6-{[2-(4-piperidinyl)ethyl]oxy}-1H-imidazo[4,5-c]pyridin-4-yl)-2-methyl-3-butyn-2-ol;
4-(2-(4-amino-1,2,5-oxadiazol-3-yl)-1-ethyl-6-{[2-(3-pyrrolidinyl)ethyl]oxy}-1H-imidazo[4,5-c]pyridin-4-yl)-2-methyl-3-butyn-2-ol;
4-[2-(4-amino-1,2,5-oxadiazol-3-yl)-1-ethyl-6-(methyloxy)-1H-imidazo[4,5-c]pyridin-4-yl]-2-methyl-3-butyn-2-ol;
4-{2-(4-amino-1,2,5-oxadiazol-3-yl)-1-ethyl-6-[(2-morpholinylmethyl)oxy]-1H-imidazo[4,5-c]pyridin-4-yl}-2-methyl-3-butyn-2-ol;
4-[2-(4-amino-1,2,5-oxadiazol-3-yl)-1-ethyl-6-(3-pyrrolidinyloxy)-1H-imidazo[4,5-c]pyridin-4-yl]-2-methyl-3-butyn-2-ol;
4-{2-(4-amino-1,2,5-oxadiazol-3-yl)-1-ethyl-6-[(3S)-3-pyrrolidinyloxy]-1H-imidazo[4,5-c]pyridin-4-yl}-2-methyl-3-butyn-2-ol;
4-{2-(4-amino-1,2,5-oxadiazol-3-yl)-1-ethyl-6-[(3R)-3-pyrrolidinyloxy]-1H-imidazo[4,5-c]pyridin-4-yl}-2-methyl-3-butyn-2-ol;
4-(2-(4-amino-1,2,5-oxadiazol-3-yl)-6-{[(2R)-2-amino-3-(3-thienyl)propyl]oxy}-1-ethyl-1H-imidazo[4,5-c]pyridin-4-yl)-2-methyl-3-butyn-2-ol;
4-[6-{[(2S)-2-amino-3-(1H-indol-3-yl)propyl]oxy}-2-(4-amino-1,2,5-oxadiazol-3-yl)-1-ethyl-1H-imidazo[4,5-c]pyridin-4-yl]-2-methyl-3-butyn-2-ol;
4-[6-{[(1R,2S)-2-aminocyclopentyl]oxy}-2-(4-amino-1,2,5-oxadiazol-3-yl)-1-ethyl-1H-imidazo[4,5-c]pyridin-4-yl]-2-methyl-3-butyn-2-ol;
4-(2-(4-amino-1,2,5-oxadiazol-3-yl)-1-ethyl-6-{[2-(methylamino)ethyl]oxy}-1H-imidazo[4,5-c]pyridin-4-yl)-2-methyl-3-butyn-2-ol;
4-[6-{[(1S,2R)-2-aminocyclopentyl]oxy}-2-(4-amino-1,2,5-oxadiazol-3-yl)-1-ethyl-1H-imidazo[4,5-c]pyridin-4-yl]-2-methyl-3-butyn-2-ol;
4-{2-(4-amino-1,2,5-oxadiazol-3-yl)-1-ethyl-6-[(phenylmethyl)oxy]-1H-imidazo[4,5-c]pyridin-4-yl}-2-methyl-3-butyn-2-ol;
4-{2-(4-amino-1,2,5-oxadiazol-3-yl)-1-ethyl-6-[(4-piperidinylmethyl)oxy]-1H-imidazo[4,5-c]pyridine-4-yl}-2-methyl-3-butyn-2-ol;
4-(2-(4-amino-1,2,5-oxadiazol-3-yl)-1-ethyl-6-{[2-(3-piperidinyl)ethyl]oxy}-1H-imidazo[4,5-c]pyridin-4-yl)-2-methyl-3-butyn-2-ol;
4-{2-(4-amino-1,2,5-oxadiazol-3-yl)-1-ethyl-6-[(3-piperidinylmethyl)oxy]-1H-imidazo[4,5-c]pyridin-4-yl}-2-methyl-3-butyn-2-ol;
4-[6-{[(2R)-2-amino-3-(1H-indol-3-yl)propyl]oxy}-2-(4-amino-1,2,5-oxadiazol-3-yl)-1-ethyl-1H-imidazo[4,5-c]pyridin-4-yl]-2-methyl-3-butyn-2-ol;
4-(2-(4-amino-1,2,5-oxadiazol-3-yl)-1-ethyl-6-{[(2R)-2-pyrrolidinylmethyl]oxy}-1H-imidazo[4,5-c]pyridin-4-yl)-2-methyl-3-butyn-2-ol;
4-(2-(4-amino-1,2,5-oxadiazol-3-yl)-1-ethyl-6-{[(2S)-2-pyrrolidinylmethyl]oxy}-1H-imidazo[4,5-c]pyridin-4-yl)-2-methyl-3-butyn-2-ol;
4-{2-(4-amino-1,2,5-oxadiazol-3-yl)-1-ethyl-6-[(1H-indol-3-ylmethyl)oxy]-1H-imidazo[4,5-c]pyridin-4-yl}-2-methyl-3-butyn-2-ol;
4-[6-[(4-amino-2-methylbutyl)oxy]-2-(4-amino-1,2,5-oxadiazol-3-yl)-1-ethyl-1H-imidazo[4,5-c]pyridin-4-yl]-2-methyl-3-butyn-2-ol;
4-(2-(4-amino-1,2,5-oxadiazol-3-yl)-6-{[(2S)-2-amino-2-phenylethyl]oxy}-1-ethyl-1H-imidazo[4,5-c]pyridin-4-yl)-2-methyl-3-butyn-2-ol;
4-(2-(4-amino-1,2,5-oxadiazol-3-yl)-6-{[(2R)-2-amino-2-phenylethyl]oxy}-1-ethyl-1H-imidazo[4,5-c]pyridin-4-yl)-2-methyl-3-butyn-2-ol;
4-(2-(4-amino-1,2,5-oxadiazol-3-yl)-6-{[(2R)-2-amino-3-phenylpropyl]oxy}-1-ethyl-1H-imidazo[4,5-c]pyridin-4-yl)-2-methyl-3-butyn-2-ol;
4-(2-(4-amino-1,2,5-oxadiazol-3-yl)-6-{[(2S)-2-amino-3-phenylpropyl]oxy}-1-ethyl-1H-imidazo[4,5-c]pyridin-4-yl)-2-methyl-3-butyn-2-ol;
4-[6-{[(2S)-2-amino-3-methylbutyl]oxy}-2-(4-amino-1,2,5-oxadiazol-3-yl)-1-ethyl-1H-imidazo[4,5-c]pyridin-4-yl]-2-methyl-3-butyn-2-ol;
4-[6-[(2-aminoethyl)oxy]-2-(4-amino-1,2,5-oxadiazol-3-yl)-1-ethyl-1H-imidazo[4,5-c]pyridin-4-yl]-3-butyn-2-ol;
3-[6-[(2-aminoethyl)oxy]-2-(4-amino-1,2,5-oxadiazol-3-yl)-1-ethyl-1H-imidazo[4,5-c]pyridin-4-yl]-2-propyn-1-ol;
4-(2-(4-amino-1,2,5-oxadiazol-3-yl)-6-{[(2R)-2-amino-3-phenylpropyl]oxy}-1-ethyl-1H-imidazo[4,5-c]pyridin-4-yl)-2-methyl-3-butyn-2-ol;
4-(2-(4-amino-1,2,5-oxadiazol-3-yl)-6-{[(2S)-2-azetidinylmethyl]oxy}-1-ethyl-1H-imidazo[4,5-c]pyridin-4-yl)-2-methyl-3-butyn-2-ol;
4-[6-{[(1R,2S)-2-aminocyclohexyl]oxy}-2-(4-amino-1,2,5-oxadiazol-3-yl)-1-ethyl-1H-imidazo[4,5-c]pyridin-4-yl]-2-methyl-3-butyn-2-ol;
4-[6-{[(1S,2R)-2-aminocyclohexyl]oxy}-2-(4-amino-1,2,5-oxadiazol-3-yl)-1-ethyl-1H-imidazo[4,5-c]pyridin-4-yl]-2-methyl-3-butyn-2-ol;
(racemic)4-[6-{[(1S,2S)-2-aminocyclohexyl]oxy}-2-(4-amino-1,2,5-oxadiazol-3-yl)-1-ethyl-1H-imidazo[4,5-c]pyridin-4-yl]-2-methyl-3-butyn-2-ol;
4-(2-(4-amino-1,2,5-oxadiazol-3-yl)-1-ethyl-6-{[2-(2-morpholinyl)ethyl]oxy}-1H-imidazo[4,5-c]pyridin-4-yl)-2-methyl-3-butyn-2-ol;
4-[2-(4-amino-1,2,5-oxadiazol-3-yl)-1-ethyl-6-({3-[(2S)-2-pyrrolidinyl]propyl}oxy-1H-imidazo[4,5-c]pyridin-4-yl]-2-methyl-3-butyn-2-ol;
2-(4-amino-1,2,5-oxadiazol-3-yl)-1-ethyl-4-(3-hydroxy-3-methyl-1-butyn-1-yl)-1,5-dihydro-6H-imidazo[4,5-c]pyridin-6-one;
4-(2-(4-amino-1,2,5-oxadiazol-3-yl)-6-{[(2S)-2-aminopropyl]oxy}-1-ethyl-1H-imidazo[4,5-c]pyridin-4-yl)-2-methyl-3-butyn-2-ol;
4-[6-{[(2R)-2-amino-3-methylbutyl]oxy}-2-(4-amino-1,2,5-oxadiazol-3-yl)-1-ethyl-1H-imidazo[4,5-c]pyridin-4-yl]-2-methyl-3-butyn-2-ol;
4-[6-{[(2R)-2-amino-4-methylpentyl]oxy}-2-(4-amino-1,2,5-oxadiazol-3-yl)-1-ethyl-1H-imidazo[4,5-c]pyridin-4-yl]-2-methyl-3-butyn-2-ol;
4-[6-{[(2S)-2-amino-4-methylpentyl]oxy}-2-(4-amino-1,2,5-oxadiazol-3-yl)-1-ethyl-1H-imidazo[4,5-c]pyridin-4-yl]-2-methyl-3-butyn-2-ol;
4-(2-(4-amino-1,2,5-oxadiazol-3-yl)-6-{[(2R)-2-aminopropyl]oxy}-1-ethyl-1H-imidazo[4,5-c]pyridin-4-yl)-2-methyl-3-butyn-2-ol;
4-[6-{[(2S)-2-amino-3-cyclohexylpropyl]oxy}-2-(4-amino-1,2,5-oxadiazol-3-yl)-1-ethyl-1H-imidazo[4,5-c]pyridin-4-yl]-2-methyl-3-butyn-2-ol;
4-(2-(4-amino-1,2,5-oxadiazol-3-yl)-6-{[(2S)-2-amino-4-phenylbutyl]oxy}-1-ethyl-1H-imidazo[4,5-c]pyridin-4-yl)-2-methyl-3-butyn-2-ol;
4-[6-[(2-aminoethyl)oxy]-1-ethyl-4-(3-furanyl)-1H-imidazo[4,5-c]pyridin-2-yl]-1,2,5-oxadiazol-3-amine;
4-[6-{[(2S)-2-amino-3-(1H-imidazol-4-yl)propyl]oxy}-2-(4-amino-1,2,5-oxadiazol-3-yl)-1-ethyl-1H-imidazo[4,5-c]pyridin-4-yl]-2-methyl-3-butyn-2-ol;
(5S)-5-({[2-(4-amino-1,2,5-oxadiazol-3-yl)-1-ethyl-4-(3-hydroxy-3-methyl-1-butyn-1-yl)-1H-imidazo[4,5-c]pyridin-6-yl]oxy}methyl)-2-pyrrolidinone;
(5R)-5-({[2-(4-amino-1,2,5-oxadiazol-3-yl)-1-ethyl-4-(3-hydroxy-3-methyl-1-butyn-1-yl)-1H-imidazo[4,5-c]pyridin-6-yl]oxy}methyl)-2-pyrrolidinone;
4-(2-(4-amino-1,2,5-oxadiazol-3-yl)-1-ethyl-6-{[2-(1-pyrrolidinyl)ethyl]oxy}-1H-imidazo[4,5-c]pyridin-4-yl)-2-methyl-3-butyn-2-ol;
4-(2-(4-amino-1,2,5-oxadiazol-3-yl)-1-ethyl-6-{[2-({[4-(methyloxy)phenyl]methyl}amino)ethyl]oxy}-1H-imidazo[4,5-c]pyridin-4-yl)-2-methyl-3-butyn-2-ol;
4-(2-(4-amino-1,2,5-oxadiazol-3-yl)-1-ethyl-6-{[2-({[4-(trifluoromethyl)phenyl]methyl}amino)ethyl]oxy}-1H-imidazo[4,5-c]pyridin-4-yl)-2-methyl-3-butyn-2-ol;
4-[2-(4-amino-1,2,5-oxadiazol-3-yl)-1-ethyl-6-(4-piperidinyloxy)-1H-imidazo[4,5-c]pyridin-4-yl]-2-methyl-3-butyn-2-ol;
4-(2-(4-amino-1,2,5-oxadiazol-3-yl)-1-ethyl-6-{[2-(4-morpholinyl)ethyl]oxy}-1-H-imidazo[4,5-c]pyridin-4-yl)-2-methyl-3-butyn-2-ol;
4-(2-(4-amino-1,2,5-oxadiazol-3-yl)-1-ethyl-6-{[2-(phenylamino)ethyl]oxy}-1H-imidazo[4,5-c]pyridin-4-yl)-2-methyl-3-butyn-2-ol;
4-[6-{[(2S)-2-amino-3-(1-methyl-1H-indol-3-yl)propyl]oxy}-2-(4-amino-1,2,5-oxadiazol-3-yl)-1-ethyl-1H-imidazo[4,5-c]pyridin-4-yl]-2-methyl-3-butyn-2-ol;
4-[2-(4-amino-1,2,5-oxadiazol-3-yl)-6-({(2S)-2-amino-3-[(phenylmethyl)thio]propyl}oxy)-1-ethyl-1H-imidazo[4,5-c]pyridin-4-yl]-2-methyl-3-butyn-2-ol;
4-(2-(4-amino-1,2,5-oxadiazol-3-yl)-6-{[(2R)-2-amino-3-(3-pyridinyl)propyl]oxy}-1-ethyl-1H-imidazo[4,5-c]pyridin-4-yl)-2-methyl-3-butyn-2-ol;
4-(2-(4-amino-1,2,5-oxadiazol-3-yl)-6-{[(2R)-2-amino-4-phenylbutyl]oxy}-1-ethyl-1H-imidazo[4,5-c]pyridin-4-yl)-2-methyl-3-butyn-2-ol;
4-{2-(4-amino-1,2,5-oxadiazol-3-yl)-6-[(2-amino-1-phenylethyl)oxy]-1-ethyl-1H-imidazo[4,5-c]pyridin-4-yl}-2-methyl-3-butyn-2-ol;
4-[6-(aminomethyl)-2-(4-amino-1,2,5-oxadiazol-3-yl)-1-ethyl-1H-imidazo[4,5-c]pyridin-4-yl]-2-methyl-3-butyn-2-ol;
4-{2-(4-amino-1,2,5-oxadiazol-3-yl)-1-ethyl-6-[(methylamino)methyl]-1H-imidazo[4,5-c]pyridin-4-yl}-2-methyl-3-butyn-2-ol;
4-(2-(4-amino-1,2,5-oxadiazol-3-yl)-1-ethyl-6-{[(phenylmethyl)amino]methyl}-1H-imidazo[4,5-c]pyridin-4-yl)-2-methyl-3-butyn-2-ol;
4-[2-(4-amino-1,2,5-oxadiazol-3-yl)-6-(3-aminopropyl)-1-ethyl-1H-imidazo[4,5-c]pyridin-4-yl]-2-methyl-3-butyn-2-ol;
4-[6-(2-aminoethyl)-2-(4-amino-1,2,5-oxadiazol-3-yl)-1-ethyl-1H-imidazo[4,5-c]pyridin-4-yl]-2-methyl-3-butyn-2-ol;
4-[2-(4-amino-1,2,5-oxadiazol-3-yl)-1-ethyl-6-(4-morpholinylmethyl)-1H-imidazo[4,5-c]pyridin-4-yl]-2-methyl-3-butyn-2-ol;
4-(2-(4-amino-1,2,5-oxadiazol-3-yl)-1-ethyl-6-{[methyl(phenylmethyl)amino]methyl}-1H-imidazo[4,5-c]pyridin-4-yl)-2-methyl-3-butyn-2-ol;
4-(2-(4-amino-1,2,5-oxadiazol-3-yl)-1-ethyl-6-{[methyl(2-phenylethyl)amino]methyl}-1H-imidazo[4,5-c]pyridin-4-yl)-2-methyl-3-butyn-2-ol;
4-(2-(4-amino-1,2,5-oxadiazol-3-yl)-6-{[(1R)-2-amino-1-phenylethyl]oxy}-1-ethyl-1H-imidazo[4,5-c]pyridin-4-yl)-2-methyl-3-butyn-2-ol;
4-(2-(4-amino-1,2,5-oxadiazol-3-yl)-1-ethyl-6-{[2-(methylamino)-1-phenylethyl]oxy}-1H-imidazo[4,5-c]pyridin-4-yl)-2-methyl-3-butyn-2-ol;
4-[6-[(2-amino-1-cyclohexylethyl)oxy]-2-(4-amino-1,2,5-oxadiazol-3-yl)-1-ethyl-1H-imidazo[4,5-c]pyridin-4-yl]-2-methyl-3-butyn-2-ol;
4-(2-(4-amino-1,2,5-oxadiazol-3-yl)-6-{[2-amino-(tetrahydro-2H-pyran-4-yl)ethyl]oxy}-1-ethyl-1H-imidazo[4,5-c]pyridin-4-yl)-2-methyl-3-butyn-2-ol;
4-(2-(4-amino-1,2,5-oxadiazol-3-yl)-6-{[2-amino-1-(3-pyridinyl)ethyl]oxy}-1-ethyl-1H-imidazo[4,5-c]pyridin-4-yl)-2-methyl-3-butyn-2-ol;
4-[6-[(2-amino-1-cyclopropylethyl)oxy]-2-(4-amino-1,2,5-oxadiazol-3-yl)-1-ethyl-1H-imidazo[4,5-c]pyridin-4-yl]-2-methyl-3-butyn-2-ol;
(rac)-4-[6-{[2-amino-1-(1,3-benzodioxol-4-yl)ethyl]oxy}-2-(4-amino-1,2,5-oxadiazol-3-yl)-1-ethyl-1H-imidazo[4,5-c]pyridin-4-yl]-2-methyl-3-butyn-2-ol;
(ent-1)-4-[6-{[2-amino-1-(1,3-benzodioxol-4-yl)ethyl]oxy}-2-(4-amino-1,2,5-oxadiazol-3-yl)-1-ethyl-1H-imidazo[4,5-c]pyridin-4-yl]-2-methyl-3-butyn-2-ol;
(ent-2)-4-[6-{[2-amino-1-(1,3-benzodioxol-4-yl)ethyl]oxy}-2-(4-amino-1,2,5-oxadiazol-3-yl)-1-ethyl-1H-imidazo[4,5-c]pyridin-4-yl]-2-methyl-3-butyn-2-ol;
4-(2-(4-amino-1,2,5-oxadiazol-3-yl)-6-{[2-(dimethylamino)-1-phenylethyl]oxy}-1-ethyl-1H-imidazo[4,5-c]pyridin-4-yl)-2-methyl-3-butyn-2-ol;
4-[6-{[(cis)-1-amino-2,3-dihydro-1H-inden-2-yl]oxy}-2-(4-amino-1,2,5-oxadiazol-3-yl)-1-ethyl-1H-imidazo[4,5-c]pyridin-4-yl]-2-methyl-3-butyn-2-ol;
4-(2-(4-amino-1,2,5-oxadiazol-3-yl)-1-ethyl-6-{[(S)-(2R)-2-morpholinyl(phenyl)methyl]oxy}-1H-imidazo[4,5-c]pyridin-4-yl)-2-methyl-3-butyn-2-ol;
4-(2-(4-amino-1,2,5-oxadiazol-3-yl)-1-ethyl-6-{[(R)-(2S)-2-morpholinyl(phenyl)methyl]oxy}-1H-imidazo[4,5-c]pyridin-4-yl)-2-methyl-3-butyn-2-ol;
4-[2-(4-amino-1,2,5-oxadiazol-3-yl)-1-ethyl-6-(1-pyrrolidinylmethyl)-1H-imidazo[4,5-c]pyridin-4-yl]-2-methyl-3-butyn-2-ol;
4-{2-(4-amino-1,2,5-oxadiazol-3-yl)-6-[(dimethylamino)methyl]-1-ethyl-1H-imidazo[4,5-c]pyridin-4-yl}-2-methyl-3-butyn-2-ol;
4-[2-(4-amino-1,2,5-oxadiazol-3-yl)-1-ethyl-6-(1-piperidinylmethyl)-1H-imidazo[4,5-c]pyridin-4-yl]-2-methyl-3-butyn-2-ol;
N-{[2-(4-amino-1,2,5-oxadiazol-3-yl)-1-ethyl-4-(3-hydroxy-3-methyl-1-butyn-1-yl)-1H-imidazo[4,5-c]pyridin-6-yl]methyl}-N-methylacetamide;
4-(2-(4-amino-1,2,5-oxadiazol-3-yl)-6-{[(2R)-2-amino-3-(4-pyridinyl)propyl]oxy}-1-ethyl-1H-imidazo[4,5-c]pyridin-4-yl)-2-methyl-3-butyn-2-ol;
4-(2-(4-amino-1,2,5-oxadiazol-3-yl)-6-{[(1S)-2-amino-1-phenylethyl]oxy}-1-ethyl-1H-imidazo[4,5-c]pyridin-4-yl)-2-methyl-3-butyn-2-ol;
4-[6-[(2-amino-1-methylethyl)oxy]-2-(4-amino-1,2,5-oxadiazol-3-yl)-1-ethyl-1H-imidazo[4,5-c]pyridin-4-yl]-2-methyl-3-butyn-2-ol;
4-(2-(4-amino-1,2,5-oxadiazol-3-yl)-6-{[2-amino-1-(phenylmethyl)ethyl]oxy}-1-ethyl-1H-imidazo[4,5-c]pyridin-4-yl)-2-methyl-3-butyn-2-ol;
4-{2-(4-amino-1,2,5-oxadiazol-3-yl)-6-[(3-amino-1-phenylpropyl)oxy]-1-ethyl-1H-imidazo[4,5-c]pyridin-4-yl}-2-methyl-3-butyn-2-ol;
4-(2-(4-amino-1,2,5-oxadiazol-3-yl)-6-{[(1S)-3-amino-1-phenylpropyl]oxy}-1-ethyl-1H-imidazo[4,5-c]pyridin-4-yl)-2-methyl-3-butyn-2-ol;
4-(2-(4-amino-1,2,5-oxadiazol-3-yl)-6-{[(1R)-3-amino-1-phenylpropyl]oxy}-1-ethyl-1H-imidazo[4,5-c]pyridin-4-yl)-2-methyl-3-butyn-2-ol;
4-[6-[(3-amino-1-cyclohexylpropyl)oxy]-2-(4-amino-1,2,5-oxadiazol-3-yl)-1-ethyl-1H-imidazo[4,5-c]pyridin-4-yl]-2-methyl-3-butyn-2-ol;
4-(2-(4-amino-1,2,5-oxadiazol-3-yl)-6-{[2-amino-1-(4-pyridinyl)ethyl]oxy}-1-ethyl-1H-imidazo[4,5-c]pyridin-4-yl)-2-methyl-3-butyn-2-ol;
4-(2-(4-amino-1,2,5-oxadiazol-3-yl)-6-{[2-amino-1-(2-pyridinyl)ethyl]oxy}-1-ethyl-1H-imidazo[4,5-c]pyridin-4-yl)-2-methyl-3-butyn-2-ol;
4-[6-{[1-(aminomethyl)-3-phenylpropyl]oxy}-2-(4-amino-1,2,5-oxadiazol-3-yl)-1-ethyl-1H-imidazo[4,5-c]pyridin-4-yl]-2-methyl-3-butyn-2-ol;
4-{2-(4-amino-1,2,5-oxadiazol-3-yl)-6-[(4-amino-1-phenylbutyl)oxy]-1-ethyl-1H-imidazo[4,5-c]pyridin-4-yl}-2-methyl-3-butyn-2-ol;
(rac)-4-(2-(4-amino-1,2,5-oxadiazol-3-yl)-6-{[(1R,2S)-1-amino-1,2,3,4-tetrahydro-2-naphthalenyl]oxy}-1-ethyl-1H-imidazo[4,5-c]pyridin-4-yl)-2-methyl-3-butyn-2-ol;
4-[2-(4-amino-1,2,5-oxadiazol-3-yl)-1-ethyl-6-({(R)-phenyl[(2S)-2-pyrrolidinyl]methyl}oxy)-1H-imidazo[4,5-c]pyridin-4-yl]-2-methyl-3-butyn-2-ol;
4-[2-(4-amino-1,2,5-oxadiazol-3-yl)-1-ethyl-6-({(S)-phenyl[(2R)-2-pyrrolidinyl]methyl}oxy)-1H-imidazo[4,5-c]pyridin-4-yl]-2-methyl-3-butyn-2-ol;
4-(2-(4-amino-1,2,5-oxadiazol-3-yl)-6-{[2-amino-1-(4-piperidinyl)ethyl]oxy}-1-ethyl-1H-imidazo[4,5-c]pyridin-4-yl)-2-methyl-3-butyn-2-ol;
4-[6-{[2-amino-1-(1-methyl-4-piperidinyl)ethyl]oxy}-2-(4-amino-1,2,5-oxadiazol-3-yl)-1-ethyl-1H-imidazo[4,5-c]pyridin-4-yl]-2-methyl-3-butyn-2-ol;
4-(2-(4-amino-1,2,5-oxadiazol-3-yl)-6-{[3-amino-1-(4-piperidinyl)propyl]oxy}-1-ethyl-1H-imidazo[4,5-c]pyridin-4-yl)-2-methyl-3-butyn-2-ol;
4-[6-{[3-amino-1-(1-methyl-4-piperidinyl)propyl]oxy}-2-(4-amino-1,2,5-oxadiazol-3-yl)-1-ethyl-1H-imidazo[4,5-c]pyridin-4-yl]-2-methyl-3-butyn-2-ol;
4-[6-{[3-amino-1-(1-methyl-4-piperidinyl)propyl]oxy}-2-(4-amino-1,2,5-oxadiazol-3-yl)-1-ethyl-1H-imidazo[4,5-c]pyridin-4-yl]-2-methyl-3-butyn-2-ol;
(ent-2)-4-[6-[(2-amino-1-cyclohexylethyl)oxy]-2-(4-amino-1,2,5-oxadiazol-3-yl)-1-ethyl-1H-imidazo[4,5-c]pyridin-4-yl]-2-methyl-3-butyn-2-ol;
(ent-1)-4-[6-[(2-amino-1-cyclohexylethyl)oxy]-2-(4-amino-1,2,5-oxadiazol-3-yl)-1-ethyl-1H-imidazo[4,5-c]pyridin-4-yl]-2-methyl-1-butyn-2-ol;
1-[6-[(2-aminoethyl)oxy]-2-(4-amino-1,2,5-oxadiazol-3-yl)-1-ethyl-1H-imidazo[4,5-c]pyridin-4-yl]-3-methyl-1-pentyn-3-ol;
4-(2-(4-amino-1,2,5-oxadiazol-3-yl)-6-{[(1R,2S)-2-amino-1-phenylpropyl]oxy}-1-ethyl-1H-imidazo[4,5-c]pyridin-4-yl)-2-methyl-3-butyn-2-ol;
4-(2-(4-amino-1,2,5-oxadiazol-3-yl)-1-ethyl-6-{[3-(methylamino)-1-phenylpropyl]oxy}-1H-imidazo[4,5-c]pyridin-4-yl)-2-methyl-3-butyn-2-ol;
4-(2-(4-amino-1,2,5-oxadiazol-3-yl)-6-{[3-(dimethylamino)-1-phenylpropyl]oxy}-1-ethyl-1H-imidazo[4,5-c]pyridin-4-yl)-2-methyl-3-butyn-2-ol;
4-[6-{[3-amino-1-(4-chlorophenyl)propyl]oxy}-2-(4-amino-1,2,5-oxadiazol-3-yl)-1-ethyl-1H-imidazo[4,5-c]pyridin-4-yl]-2-methyl-3-butyn-2-ol;
4-[6-{[3-amino-1-(3-chlorophenyl)propyl]oxy}-2-(4-amino-1,2,5-oxadiazol-3-yl)-1-ethyl-1H-imidazo[4,5-c]pyridin-4-yl]-2-methyl-3-butyn-2-ol;
4-[6-{[3-amino-1-(2-chlorophenyl)propyl]oxy}-2-(4-amino-1,2,5-oxadiazol-3-yl)-1-ethyl-1H-imidazo[4,5-c]pyridin-4-yl]-2-methyl-3-butyn-2-ol;
4-[6-({2-amino-1-[3-(methyloxy)phenyl]ethyl}oxy)-2-(4-amino-1,2,5-oxadiazol-3-yl)-1-ethyl-1H-imidazo[4,5-c]pyridin-4-yl]-2-methyl-3-butyn-2-ol;
4-[6-{[(1S,2S)-2-amino-3-(methyloxy)-1-phenylpropyl]oxy}-2-(4-amino-1,2,5-oxadiazol-3-yl)-1-ethyl-1H-imidazo[4,5-c]pyridin-4-yl]-2-methyl-3-butyn-2-ol;
4-[6-({3-amino-1-[2-fluoro-3-(methyloxy)phenyl]propyl}oxy)-2-(4-amino-1,2,5-oxadiazol-3-yl)-1-ethyl-1H-imidazo[4,5-c]pyridin-4-yl]-2-methyl-3-butyn-2-ol;
4-[6-({3-amino-1-[3-(methyloxy)phenyl]propyl}oxy)-2-(4-amino-1,2,5-oxadiazol-3-yl)-1-ethyl-1H-imidazo[4,5-c]pyridin-4-yl]-2-methyl-3-butyn-2-ol;
4-[6-({2-amino-1-[2-fluoro-3-(methyloxy)phenyl]ethyl}oxy)-2-(4-amino-1,2,5-oxadiazol-3-yl)-1-ethyl-1H-imidazo[4,5-c]pyridin-4-yl]-2-methyl-3-butyn-2-ol;
4-[6-{[3-amino-1-(2-fluorophenyl)propyl]oxy}-2-(4-amino-1,2,5-oxadiazol-3-yl)-1-ethyl-1H-imidazo[4,5-c]pyridin-4-yl]-2-methyl-3-butyn-2-ol;
4-[6-{[(2R)-2-amino-3-(4-fluorophenyl)propyl]oxy}-2-(4-amino-1,2,5-oxadiazol-3-yl)-1-ethyl-1H-imidazo[4,5-c]pyridin-4-yl]-2-methyl-3-butyn-2-ol;
4-[6-{[(2R)-2-amino-3-(2-fluorophenyl)propyl]oxy}-2-(4-amino-1,2,5-oxadiazol-3-yl)-1-ethyl-1H-imidazo[4,5-c]pyridin-4-yl]-2-methyl-3-butyn-2-ol;
4-[2-(4-amino-1,2,5-oxadiazol-3-yl)-6-({(2R)-2-amino-3-[2-(trifluoromethyl)phenyl]propyl}oxy)-1-ethyl-1H-imidazo[4,5-c]pyridin-4-yl]-2-methyl-3-butyn-2-ol;
4-[6-{[(2R)-2-amino-3-(4-chlorophenyl)propyl]oxy}-2-(4-amino-1,2,5-oxadiazol-3-yl)-1-ethyl-1H-imidazo[4,5-c]pyridin-4-yl]-2-methyl-3-butyn-2-ol;
4-[6-{[(2R)-2-amino-3-(3-chlorophenyl)propyl]oxy}-2-(4-amino-1,2,5-oxadiazol-3-yl)-1-ethyl-1H-imidazo[4,5-c]pyridin-4-yl]-2-methyl-3-butyn-2-ol;
4-[6-{[(2R)-2-amino-3-(2-chlorophenyl)propyl]oxy}-2-(4-amino-1,2,5-oxadiazol-3-yl)-1-ethyl-1H-imidazo[4,5-c]pyridin-4-yl]-2-methyl-3-butyn-2-ol;
4-[2-(4-amino-1,2,5-oxadiazol-3-yl)-6-({(2R)-2-amino-3-[3-(trifluoromethyl)phenyl]propyl}oxy)-1-ethyl-1H-imidazo[4,5-c]pyridin-4-yl]-2-methyl-3-butyn-2-ol;
4-[6-{[(2R)-2-amino-3-(3-fluorophenyl)propyl]oxy}-2-(4-amino-1,2,5-oxadiazol-3-yl)-1-ethyl-1H-imidazo[4,5-c]pyridin-4-yl]-2-methyl-3-butyn-2-ol;
4-[2-(4-amino-1,2,5-oxadiazol-3-yl)-6-({(2R)-2-amino-3-[4-(trifluoromethyl)phenyl]propyl}oxy)-1-ethyl-1H-imidazo[4,5-c]pyridin-4-yl]-2-methyl-3-butyn-2-ol;
4-[6-{[(2R)-2-amino-3-(1-benzothien-2-yl)propyl]oxy}-2-(4-amino-1,2,5-oxadiazol-3-yl)-1-ethyl-1H-imidazo[4,5-c]pyridin-4-yl]-2-methyl-3-butyn-2-ol;
4-[6-{[(2R)-2-amino-3-cyclohexylpropyl]oxy}-2-(4-amino-1,2,5-oxadiazol-3-yl)-1-ethyl-1H-imidazo[4,5-c]pyridin-4-yl]-2-methyl-3-butyn-2-ol;
4-{2-(4-amino-1,2,5-oxadiazol-3-yl)-6-[(3-amino-3-phenylpropyl)oxy]-1-ethyl-1H-imidazo[4,5-c]pyridin-4-yl}-2-methyl-3-butyn-2-ol;
4-{2-(4-amino-1,2,5-oxadiazol-3-yl)-6-[(3-amino-2-phenylpropyl)oxy]-1-ethyl-1H-imidazo[4,5-c]pyridin-4-yl}-2-methyl-3-butyn-2-ol;
4-[6-{[2-amino-1-(3-chlorophenyl)ethyl]oxy}-2-(4-amino-1,2,5-oxadiazol-3-yl)-1-ethyl-1H-imidazo[4,5-c]pyridin-4-yl]-2-methyl-3-butyn-2-ol;
4-[6-{[2-amino-1-(2-chlorophenyl)ethyl]oxy}-2-(4-amino-1,2,5-oxadiazol-3-yl)-1-ethyl-1H-imidazo[4,5-c]pyridin-4-yl]-2-methyl-3-butyn-2-ol;
4-[6-{[2-amino-1-(4-chlorophenyl)ethyl]oxy}-2-(4-amino-1,2,5-oxadiazol-3-yl)-1-ethyl-1H-imidazo[4,5-c]pyridin-4-yl]-2-methyl-3-butyn-2-ol;
4-[6-{[3-amino-1-(3-fluorophenyl)propyl]oxy}-2-(4-amino-1,2,5-oxadiazol-3-yl)-1-ethyl-1H-imidazo[4,5-c]pyridin-4-yl]-2-methyl-3-butyn-2-ol;
4-[6-{[3-amino-1-(4-fluorophenyl)propyl]oxy}-2-(4-amino-1,2,5-oxadiazol-3-yl)-1-ethyl-1H-imidazo[4,5-c]pyridin-4-yl]-2-methyl-3-butyn-2-ol;
4-[6-{[2-amino-1-(2-fluorophenyl)ethyl]oxy}-2-(4-amino-1,2,5-oxadiazol-3-yl)-1-ethyl-1H-imidazo[4,5-c]pyridin-4-yl]-2-methyl-3-butyn-2-ol;
4-[6-{[2-amino-1-(3-fluorophenyl)ethyl]oxy}-2-(4-amino-1,2,5-oxadiazol-3-yl)-1-ethyl-1H-imidazo[4,5-c]pyridin-4-yl]-2-methyl-3-butyn-2-ol;
4-[6-{[2-amino-1-(4-fluorophenyl)ethyl]oxy}-2-(4-amino-1,2,5-oxadiazol-3-yl)-1-ethyl-1H-imidazo[4,5-c]pyridin-4-yl]-2-methyl-3-butyn-2-ol;
4-[6-({3-amino-1-[4-fluoro-3-(methyloxy)phenyl]propyl}oxy)-2-(4-amino-1,2,5-oxadiazol-3-yl)-1-ethyl-1H-imidazo[4,5-c]pyridin-4-yl]-2-methyl-3-butyn-2-ol;
4-[6-({2-amino-1-[4-fluoro-3-(methyloxy)phenyl]ethyl}oxy)-2-(4-amino-1,2,5-oxadiazol-3-yl)-1-ethyl-1H-imidazo[4,5-c]pyridin-4-yl]-2-methyl-3-butyn-2-ol;
4-[6-{[(2R)-2-Amino-3-(2-furanyl)propyl]oxy}-2-(4-amino-1,2,5-oxadiazol-3-yl)-1-ethyl-1H-imidazo[4,5-c]pyridin-4-yl]-2-methyl-3-butyn-2-ol;
3-(3-Amino-1-{[2-(4-amino-1,2,5-oxadiazol-3-yl)-1-ethyl-4-(3-hydroxy-3-methyl-1-butyn-1-yl)-1H-imidazo[4,5-c]pyridin-6-yl]oxy}propyl)phenol;
4-((2R)-2-Amino-3-{[2-(4-amino-1,2,5-oxadiazol-3-yl)-1-ethyl-4-(3-hydroxy-3-methyl-1-butyn-1-yl)-1H-imidazo[4,5-c]pyridin-6-yl]oxy}propyl)phenol;
4-((2S)-2-Amino-3-{[2-(4-amino-1,2,5-oxadiazol-3-yl)-1-ethyl-4-(3-hydroxy-3-methyl-1-butyn-1-yl)-1H-imidazo[4,5-c]pyridin-6-yl]oxy}propyl)phenol;
4-(2-(4-Amino-1,2,5-oxadiazol-3-yl)-1-ethyl-6-{[(3R)-1,2,3,4-tetrahydro-3-isoquinolinylmethyl]-oxy}-1H-imidazo[4,5-c]pyridin-4-yl)-2-methyl-3-butyn-2-ol;
4-[6-({(2R)-2-Amino-3-[3-(methyloxy)phenyl]propyl}oxy)-2-(4-amino-1,2,5-oxadiazol-3-yl)-1-ethyl-1H-imidazo[4,5-c]pyridin-4-yl]-2-methyl-3-butyn-2-ol;
3-(3-Amino-1-{[2-(4-amino-1,2,5-oxadiazol-3-yl)-1-ethyl-4-(3-hydroxy-3-methyl-1-butyn-1-yl)-1H-imidazo[4,5-c]pyridin-6-yl]oxy}propyl)phenol;
4-[2-(4-amino-1,2,5-oxadiazol-3-yl)-1-ethyl-6-({2-[(3R)-1,2,3,4-tetrahydro-3-isoquinolinyl]ethyl}oxy)-1H-imidazo[4,5-c]pyridin-4-yl]-2-methyl-3-butyn-2-ol;
4-(2-(4-amino-1,2,5-oxadiazol-3-yl)-6-{[(2S)-2-amino-3-(3-pyridinyl)propyl]oxy}-1-ethyl-1H-imidazo[4,5-c]pyridin-4-yl)-2-methyl-3-butyn-2-ol;
4-(2-(4-amino-1,2,5-oxadiazol-3-yl)-6-{[(2S)-2-amino-3-(4-pyridinyl)propyl]oxy}-1-ethyl-1H-imidazo[4,5-c]pyridin-4-yl)-2-methyl-3-butyn-2-ol;
4-[6-({(2R)-2-amino-3-[4-(methyloxy)phenyl]propyl}oxy)-2-(4-amino-1,2,5-oxadiazol-3-yl)-1-ethyl-1H-imidazo[4,5-c]pyridin-4-yl]-2-methyl-3-butyn-2-ol;
4-[6-{[(2S)-2-amino-3-(2-furanyl)propyl]oxy}-2-(4-amino-1,2,5-oxadiazol-3-yl)-1-ethyl-1H-imidazo[4,5-c]pyridin-4-yl]-2-methyl-3-butyn-2-ol;
4-[6-({(2R)-2-amino-3-[2-(methyloxy)phenyl]propyl}oxy)-2-(4-amino-1,2,5-oxadiazol-3-yl)-1-ethyl-1H-imidazo[4,5-c]pyridin-4-yl]-2-methyl-3-butyn-2-ol;
4-[6-[(3-amino-3-cyclohexylpropyl)oxy]-2-(4-amino-1,2,5-oxadiazol-3-yl)-1-ethyl-1H-imidazo[4,5-c]pyridin-4-yl]-2-methyl-3-butyn-2-ol;
4-(2-(4-amino-1,2,5-oxadiazol-3-yl)-6-{[3-amino-3-(tetrahydro-2H-pyran-4-yl)propyl]oxy}-1-ethyl-1H-imidazo[4,5-c]pyridin-4-yl)-2-methyl-3-butyn-2-ol;
4-(2-(4-amino-1,2,5-oxadiazol-3-yl)-6-{[3-amino-3-(tetrahydro-2H-pyran-4-yl)propyl]oxy}-1-ethyl-1H-imidazo[4,5-c]pyridin-4-yl)-2-methyl-3-butyn-2-ol;
4-[2-(4-amino-1,2,5-oxadiazol-3-yl)-1-ethyl-6-({2-[(3R)-1,2,3,4-tetrahydro-3-isoquinolinyl]ethyl}oxy)-1H-imidazo[4,5-c]pyridin-4-yl]-2-methyl-3-butyn-2-ol;
4-(2-(4-amino-1,2,5-oxadiazol-3-yl)-1-ethyl-6-{[(3S)-1,2,3,4-tetrahydro-3-isoquinolinylmethyl]oxy}-1H-imidazo[4,5-c]pyridin-4-yl)-2-methyl-3-butyn-2-ol;
4-[6-{[2-(aminomethyl)-4-phenylbutyl]oxy}-2-(4-amino-1,2,5-oxadiazol-3-yl)-1-ethyl-1H-imidazo[4,5-c]pyridin-4-yl]-2-methyl-3-butyn-2-ol;
4-[6-[(3-amino-1-{[3-(methyloxy)phenyl]methyl}propyl)oxy]-2-(4-amino-1,2,5-oxadiazol-3-yl)-1-ethyl-1H-imidazo[4,5-c]pyridin-4-yl]-2-methyl-3-butyn-2-ol;
4-(2-(4-amino-1,2,5-oxadiazol-3-yl)-6-{[3-amino-1-(3-thienylmethyl)propyl]oxy}-1-ethyl-1H-imidazo[4,5-c]pyridin-4-yl)-2-methyl-3-butyn-2-ol;
4-[6-[(3-amino-1-{[3,4-bis(methyloxy)phenyl]methyl}propyl)oxy]-2-(4-amino-1,2,5-oxadiazol-3-yl)-1-ethyl-1H-imidazo[4,5-c]pyridin-4-yl]-2-methyl-3-butyn-2-ol; and
4-{2-(4-amino-1,2,5-oxadiazol-3-yl)-6-[(3-aminoethyl)oxy]-1-ethyl-1H-imidazo[4,5-c]pyridin-4-yl}-2-methyl-3-butyn-2-ol;
   and/or pharmaceutically acceptable salts, hydrates, solvates and pro-drugs thereof.

Included among the novel compounds useful in the present invention are:
4-(2-(4-amino-1,2,5-oxadiazol-3-yl)-6-{[(2R)-2-amino-3-phenylpropyl]oxy}-1-ethyl-1H-imidazo[4,5-c]pyridin-4-yl)-2-methyl-3-butyn-2-ol;
4-{2-(4-amino-1,2,5-oxadiazol-3-yl)-6-[(2-amino-1-phenylethyl)oxy]-1-ethyl-1H-imidazo[4,5-c]pyridin-4-yl}-2-methyl-3-butyn-2-ol;
4-(2-(4-amino-1,2,5-oxadiazol-3-yl)-6-{[(1R)-2-amino-1-phenylethyl]oxy}-1-ethyl-1H-imidazo[4,5-c]pyridin-4-yl)-2-methyl-3-butyn-2-ol;
(rac)-4-[6-{[2-amino-1-(1,3-benzodioxol-4-yl)ethyl]oxy}-2-(4-amino-1,2,5-oxadiazol-3-yl)-1-ethyl-1H-imidazo[4,5-c]pyridin-4-yl]-2-methyl-3-butyn-2-ol;
(ent-1)-4-[6-{[2-amino-1-(1,3-benzodioxol-4-yl)ethyl]oxy}-2-(4-amino-1,2,5-oxadiazol-3-yl)-1-ethyl-1H-imidazo[4,5-c]pyridin-4-yl]-2-methyl-3-butyn-2-ol;
(ent-2)-4-[6-{[2-amino-1-(1,3-benzodioxol-4-yl)ethyl]oxy}-2-(4-amino-1,2,5-oxadiazol-3-yl)-1-ethyl-1H-imidazo[4,5-c]pyridin-4-yl]-2-methyl-3-butyn-2-ol;
4-(2-(4-amino-1,2,5-oxadiazol-3-yl)-6-{[2-amino-1-(phenylmethyl)ethyl]oxy}-1-ethyl-1H-imidazo[4,5-c]pyridin-4-yl)-2-methyl-3-butyn-2-ol;
4-(2-(4-amino-1,2,5-oxadiazol-3-yl)-6-{[(1S)-3-amino-1-phenylpropyl]oxy}-1-ethyl-1H-imidazo[4,5-c]pyridin-4-yl)-2-methyl-3-butyn-2-ol;
4-[2-(4-amino-1,2,5-oxadiazol-3-yl)-1-ethyl-6-({(S)-phenyl[(2R)-2-pyrrolidinyl]methyl}oxy)-1H-imidazo[4,5-c]pyridin-4-yl]-2-methyl-3-butyn-2-ol;
4-[6-({2-amino-1-[3-(methyloxy)phenyl]ethyl}oxy)-2-(4-amino-1,2,5-oxadiazol-3-yl)-1-ethyl-1H-imidazo[4,5-c]pyridin-4-yl]-2-methyl-3-butyn-2-ol;
4-[6-{[(2R)-2-amino-3-(2-chlorophenyl)propyl]oxy}-2-(4-amino-1,2,5-oxadiazol-3-yl)-1-ethyl-1H-imidazo[4,5-c]pyridin-4-yl]-2-methyl-3-butyn-2-ol;
4-[6-{[2-amino-1-(3-chlorophenyl)ethyl]oxy}-2-(4-amino-1,2,5-oxadiazol-3-yl)-1-ethyl-1H-imidazo[4,5-c]pyridin-4-yl]-2-methyl-3-butyn-2-ol;
4-[6-{[2-amino-1-(2-chlorophenyl)ethyl]oxy}-2-(4-amino-1,2,5-oxadiazol-3-yl)-1-ethyl-1H-imidazo[4,5-c]pyridin-4-yl]-2-methyl-3-butyn-2-ol;
(ent-2)-4-[6-[(2-amino-1-cyclohexylethyl)oxy]-2-(4-amino-1,2,5-oxadiazol-3-yl)-1-ethyl-1H-imidazo[4,5-c]pyridin-4-yl]-2-methyl-3-butyn-2-ol; and
(ent-1)-4-[6-[(2-amino-1-cyclohexylethyl)oxy]-2-(4-amino-1,2,5-oxadiazol-3-yl)-1-ethyl-1H-imidazo[4,5-c]pyridin-4-yl]-2-methyl-3-butyn-2-ol;
   and/or pharmaceutically acceptable salts, hydrates, solvates and pro-drugs thereof.

Compounds of Formula (I) are included in the pharmaceutical compositions of the invention and used in the methods of the invention.

Certain of the compounds described herein may contain one or more chiral atoms, or may otherwise be capable of existing as two enantiomers. Accordingly, the compounds of this invention include mixtures of enantiomers as well as purified enantiomers or enantiomerically enriched mixtures. Also, it is understood that all tautomers and mixtures of tautomers are included within the scope of the compounds of formula I or II.

By the term “aryl” as used herein, unless otherwise defined, is meant a cyclic or polycyclic aromatic ring containing from 1 to 14 carbon atoms and optionally containing from one to five heteroatoms, provided that when the number of carbon atoms is 1 the aromatic ring contains at least four heteroatoms, when the number of carbon atoms is 2 the aromatic ring contains at least three heteroatoms, when the number of carbons is 3 the aromatic ring contains at least two heteroatoms and when the number of carbon atoms is 4 the aromatic ring contains at least one heteroatom.

By the term “C₁-C₁₂aryl” as used herein, unless otherwise defined, is meant phenyl, naphthalene, tetrahydronaphthalene, 3,4-methylenedioxyphenyl, pyridine, biphenyl, quinoline, isoquinoline, tetrahydroquinoline, tetrahydroisoquinoline, pyrimidine, quinazoline, thiophene, furan, pyrrole, pyrazole, imidazole, indole, indole 3-yl, dihydroindole, indene, dihydroindene, pyrazine, 1,3-dihydro-2H-benzimidazol, benzothiohpene and tetrazole.

As an alternative, the term “C₁-C₁₂aryl” as used herein, can be selected from the group consisting of: phenyl, naphthalene, 3,4-methylenedioxyphenyl, pyridine, biphenyl, quinoline, pyrimidine, quinazoline, thiophene, furan, pyrrole, pyrazole, imidazole, indole, indene, pyrazine, 1,3-dihydro-2H-benzimidazol, benzothiohpene and tetrazole.

The term “substituted” as used herein, unless otherwise defined, is meant that the subject chemical moiety has one or more substituents selected from the group consisting of: —CO₂R²⁰, aryl,

aryl substituted with one or more substituents selected from alkyl, hydroxyl, alkoxy, amino, trifluoromethyl, N-acylamino and halogen,
cycloalkyl substituted with one or more substituents selected from alkyl, hydroxyl, alkoxy, amino, N-acylamino and halogen,
cycloalkyl containing from 1 to 4 heteroatoms substituted with one or more substituents selected from alkyl, hydroxyl, alkoxy, amino, N-acylamino and halogen,
cycloalkyl, cycloalkyl containing from 1 to 4 heteroatoms, —C(O)NHS(O)₂R²⁰, —NHS(O)₂R²⁰, hydroxyalkyl, alkoxy, aryloxy, —C(O)NR²¹R²², acyloxy, alkyl, amino, phenylamino, alkylamino, nitrile, acetamide, urea, alkylurea, benzoate, sulfonamide, benzoateurea, aminoalkyl, aminoalkoxy, alkylaminoalkoxy, alkylaminoalkoxy substituted with from one to three substituents selected from methoxyphenyl, trifluoromethylphenyl, aryl and alkyl,
alkylamino, alkylamino substituted with from one to three substituents selected from methoxyphenyl, trifluoromethylphenyl, C₁-C₁₂aryl and alkyl,
aminoalkyl substituted with from one to three substituents selected from methoxyphenyl, trifluoromethylphenyl, C₁-C₁₂aryl and alkyl, dialkylaminoalkoxy, alkoxyalkylamide, triphenylalkyl, (phenylc₁-C₄alkyl)thio, (phenylC₁-C₄alkyl)thioalkyl, C₁-C₁₂arylalkylurea, dialkylamino, N-acylamino, alkylN-acylamino, aminoalkylN-acylamino, hydroxy, —(CH₂)_{g}C(O)OR²³, —S(O)ₙR²³, nitro, tetrazole, cyano, oxo, halogen and trifluoromethyl, where g is 0-6, R²³ is hydrogen or alkyl, R²⁰ is selected form hydrogen, C₁-C₄alkyl, aryl and trifluoromethyl, and R²¹ and R²² are independently selected form hydrogen, C₁-C₄alkyl, aryl and trifluoromethyl, and n is 0-2.

As an alternative, the term “substituted” as used herein, can mean that the subject chemical moiety has one or more substituents selected from the group consisting of: —CO₂R²⁰, aryl,

aryl substituted with one or more substituents selected from alkyl, hydroxyl, alkoxy, amino, N-acylamino and halogen,
cycloalkyl substituted with one or more substituents selected from alkyl, hydroxyl, alkoxy, amino, N-acylamino and halogen,
cycloalkyl containing from 1 to 4 heteroatoms substituted with one or more substituents selected from alkyl, hydroxyl, alkoxy, amino, N-acylamino and halogen, cycloalkyl, cycloalkyl containing from 1 to 4 heteroatoms, —C(O)NHS(O)₂R²⁰, —NHS(O)₂R²⁰, hydroxyalkyl, alkoxy, —C(O)NR²¹R²², acyloxy, alkyl, amino, alkylamino, nitrile, acetamide, urea, alkylurea, benzoate, sulfonamide, benzoateurea, aminoalkyl, aminoalkoxy, alkylaminoalkoxy, dialkylaminoalkoxy, alkoxyalkylamide, alkoxyC₁-C₁₂aryl, triphenylalkyl, C₁-C₁₂arylalkylurea, C₁-C₁₂aryl, haloC₁-C₁₂aryl, dialkylamino, N-acylamino, aminoalkylN-acylamino, hydroxy, —(CH₂)_{g}C(O)OR²³, —S(O)ₙR²³, nitro, tetrazole, cyano, oxo, halogen and trifluoromethyl, where g is 0-6, R²³ is hydrogen or alkyl, R²⁰ is selected form hydrogen, C₁-C₄alkyl, aryl and trifluoromethyl, and R²¹ and R²² are independently selected form hydrogen, C₁-C₄alkyl, aryl and trifluoromethyl, and n is 0-2.

As an alternative, the term “substituted” as used herein, can mean that the subject chemical moiety has from one to four substituents selected from the group consisting of: amino, alkylamino, dialkylamino, aryl, aryl substituted with from one to four substituents selected from alkyl, hydroxyl, alkoxy, amino, trifluoromethyl, N-acylamino and halogen, cycloalkyl containing from 1 to 4 heteroatoms, cycloalkyl containing from 1 to 4 heteroatoms substituted with from one to four substituents selected from alkyl, hydroxyl, alkoxy, amino, N-acylamino and halogen, cycloalkyl, and cycloalkyl substituted with from one to four substituents selected from alkyl, hydroxyl, alkoxy, amino, N-acylamino and halogen.

As an alternative, the term “substituted” as used herein, can mean that the subject chemical moiety has from one to four substituents selected from the group consisting of: amino, alkylamino, dialkylamino, aryl, aryl substituted with from one to four substituents selected from alkyl, hydroxyl, alkoxy, amino, N-acylamino and halogen, cycloalkyl containing from 1 to 4 heteroatoms, cycloalkyl containing from 1 to 4 heteroatoms substituted with from one to four substituents selected from alkyl, hydroxyl, alkoxy, amino, N-acylamino and halogen, cycloalkyl, and cycloalkyl substituted with from one to four substituents selected from alkyl, hydroxyl, alkoxy, amino, N-acylamino and halogen.

When referring to the term “substituted” as used herein, suitably, aryl is a C₁-C₁₂aryl, and suitably, substituted aryl is a substituted C₁-C₁₂aryl.

When referring to the term “substituted” as used herein, suitably, the subject chemical moiety is substituted with from one to four substituents.

By the term “alkoxy” as used herein is meant —Oalkyl where alkyl is as described herein including —OCH₃ and —OC(CH₃)₂CH₃.

The term “cycloalkyl” as used herein unless otherwise defined, is meant a nonaromatic, unsaturated or saturated, cyclic or polycyclic C₃-C₁₂.

Examples of cycloalkyl and substituted cycloalkyl substituents as used herein include: cyclohexyl, aminocyclohexyl, cyclobutyl, aminocyclobutyl, 4-hydroxy-cyclohexyl, 2-ethylcyclohexyl, propyl 4-methoxycyclohexyl, 4-methoxycyclohexyl, 4-carboxycyclohexyl, cyclopropyl, aminocyclopentyl, cyclopentyl.

The term “cycloalkyl containing from 1 to 4 heteroatoms” and the term “cycloalkyl containing from 1 to 3 heteroatoms” as used herein unless otherwise defined, is meant a nonaromatic, unsaturated or saturated, cyclic or polycyclic ring containing from 1 to 12 carbons and containing from one to four heteroatoms or from one to three heteroatoms (respectively), provided that when the number of carbon atoms is 1 the aromatic ring contains at least four heteroatoms (applicable only where “cycloalkyl containing from 1 to 4 heteroatoms” is indicated), when the number of carbon atoms is 2 the aromatic ring contains at least three heteroatoms, when the number of carbon atoms is 3 the nonaromatic ring contains at least two heteroatoms and when the number of carbon atoms is 4 the nonaromatic ring contains at least one heteroatom.

Examples of cycloalkyl containing from 1 to 4 heteroatoms, cycloalkyl containing from 1 to 3 heteroatoms, substituted cycloalkyl containing from 1 to 4 heteroatoms and substituted cycloalkyl containing from 1 to 3 heteroatoms as used herein include: piperidyl, piperidine, pyrrolidine, 3-methylaminopyrrolidine, piperazinly, tetrazole, hexahydrodiazepine, azetidinyl, pyran, tetrahydropyran, and morpholine.

By the term “acyloxy” as used herein is meant —OC(O)alkyl where alkyl is as described herein. Examples of acyloxy substituents as used herein include: —OC(O)CH₃, —OC(O)CH(CH₃)₂ and —OC(O)(CH₂)₃CH₃.

By the term “N-acylamino” as used herein is meant —N(H)C(O)alkyl, where alkyl is as described herein. Examples of N-acylamino substituents as used herein include: —N(H)C(O)CH₃, —N(H)C(O)CH(CH₃)₂ and —N(H)C(O)(CH₂)₃CH₃.

By the term “aryloxy” as used herein is meant —Oaryl where aryl is phenyl, naphthyl, 3,4-methylenedioxyphenyl, pyridyl or biphenyl optionally substituted with one or more substituents selected from the group consisting of: alkyl, hydroxyalkyl, alkoxy, trifluoromethyl, acyloxy, amino, N-acylamino, hydroxy, —(CH₂)_{g}C(O)OR²⁵, —S(O)ₙR²⁵, nitro, cyano, halogen and protected —OH, where g is 0-6, R²⁵ is hydrogen or alkyl, and n is 0-2. Examples of aryloxy substituents as used herein include: phenoxy, 4-fluorophenyloxy and biphenyloxy.

By the term “heteroatom” as used herein is meant oxygen, nitrogen or sulfur.

By the term “halogen” as used herein is meant a substituent selected from bromide, iodide, chloride and fluoride.

By the term “alkyl” and derivatives thereof and in all carbon chains as used herein, including alkyl chains defined by the term “—(CH₂)ₙ”, “—(CH₂)ₘ” and the like, is meant a linear or branched, saturated or unsaturated hydrocarbon chain, and unless otherwise defined, the carbon chain will contain from 1 to 12 carbon atoms. Examples of alkyl and substituted alkyl substituents as used herein include: —CH₃, —CH₂—CH₃, —CH₂—CH₂—CH₃, —CH(CH₃)₂, —CH₂—CH₂—C(CH₃)₃, —CH₂—CF₃, —C≡C—C(CH₃)₃, —C≡C—CH₂—OH, cyclopropylmethyl, —CH₂—C(CH₃)₂—CH₂—NH₂, —C≡C—C₆H₅, —C≡C—C(CH₃)₂—OH, —CH₂—CH(OH)—CH(OH)—CH(OH)—CH(OH)—CH₂—OH, piperidinylmethyl, methoxyphenylethyl, —C(CH₃)₃, —(CH₂)₃—CH₃, —CH₂—CH(CH₃)₂, —CH(CH₃)—CH₂—CH₃, —CH═CH₂, and —C≡C—CH₃.

By the term “treating” and derivatives thereof as used herein, is meant prophylatic and therapeutic therapy.

As used herein, the term “effective amount” and derivatives thereof means that amount of a drug or pharmaceutical agent that will elicit the biological or medical response of a tissue, system, animal or human that is being sought, for instance, by a researcher or clinician. Furthermore, the term “therapeutically effective amount” and derivatives thereof means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

Compounds of Formula (I) are included in the pharmaceutical compositions of the invention and used in the methods of the invention. Where a —COOH or —OH group is present, pharmaceutically acceptable esters can be employed, for example methyl, ethyl, pivaloyloxymethyl, and the like for —COOH, and acetate maleate and the like for —OH, and those esters known in the art for modifying solubility or hydrolysis characterstics, for use as sustained release or prodrug formulations.

The novel compounds of Formulas I and II are prepared as shown in Schemes I to IX below, or by analogous methods, wherein the ‘Het’ and ‘R’ substituents are as defined in Formulas I and II respectively and provided that the ‘Het’ and ‘R’ substituents do not include any such substituents that render inoperative the processes of Schemes I to IX. Suitably, the novel compound of Formula I wherein the Het group is other than amino-oxadiazol can be prepared by methods analogous to those described in International Application No. PCT/US2004/024340, having an International filing date of Jul. 28, 2004, and having International Publication No. WO 2005/011700, having an International Publication date of Feb. 10, 2005. All of the starting materials are commercially available or are readily made from commercially available starting materials by those of skill in the art.

### General Schemes

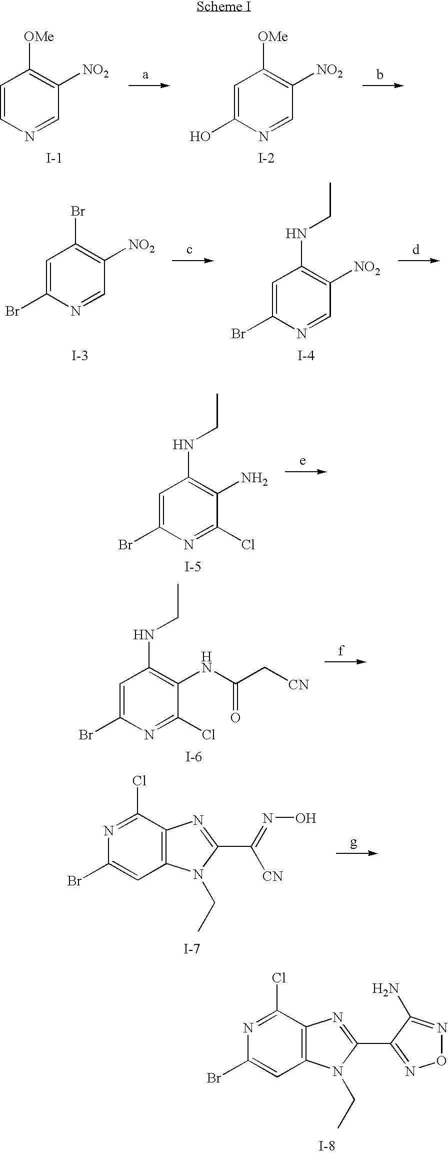

Reagents: (a) NH₃, t-BuOK, t-BuOOH, THF; (b) POBr₃, CH₃CN; (c) EtNH₂, THF; (d) SnCl₂, HCl; (e) cyanoacetic acid, EDCl, NMM, DMF; (f) HOAc, 100° C.; then NaNO₂; (h) NH₂OH, Et₃N, dioxane.

Compounds of Formula (I) can be prepared in a manner analogous to those shown in Scheme 1. Hydroxylation of 3-nitro-4-methoxy pyridine (I-1) using t-BuOOH and t-BuOK in NH₃ gives 2-hydroxy-4-(methyloxy)-5-nitropyridine (I-2) according to the procedure of Makosza, et. al. J. Org. Chem. 1998, 63, 4199. Both the 4-methoxy and the 2-hydroxyl substituents are converted to bromides using POBr₃ in a polar aprotic solvent such as CH₃CN at reflux to give compound (I-3). The 4-bromo group is then displaced by a primary amine such as ethyl amine in a polar solvent such as ethanol to give compounds such as I-4. In the case of liquid amines, the reaction can be carried out in the absence of solvent. The reduction of the nitro group with concomitant introduction of the chloro group is achieved using tin (II) chloride according to the method described by Kelley et al. J. Med. Chem. 1995, 38(20), 4131-34. The 6-bromo-2-chloro diaminopyridine (I-5) is condensed with an appropriate acid such as cyanoacetic acid using an appropriate coupling reagent such as EDCl in a polar aprotic solvent such as DMF. The resulting amide (I-6) will undergo cyclodehydration in refluxing acetic acid and when followed by treatment in situ with NaNO₂ will afford a hydroxylamine such as (I-7). Reaction of (I-7) with hydroxylamine gives a bis-oxime that cyclodehydrates in the presence of an appropriate base such as triethylamine to give an aminofurazan such as (I-8).

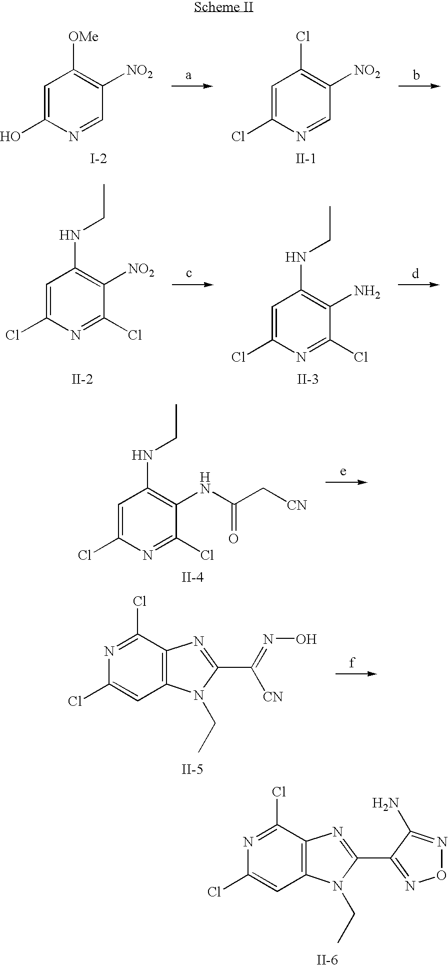

Reagents: (a) POCl₃, CH₃CN; (b) EtNH₂, THF; (c) SnCl₂, HCl; (d) cyanoacetic acid, EDCl, NMM, DMF; (e) HOAc, 100° C.; then NaNO₂; (f) NH₂OH, Et₃N, dioxane.

Displacement of both the 4-methoxy and the 2-hydroxyl substituents of pyridone (I-2) to bromides using POBr₃ in a polar aprotic solvent such as CH₃CN at reflux gives compound (II-1). The 4-chloro group is then displaced by a primary amine such as ethyl amine in a polar solvent such as ethanol to give compounds such as II-2. In the case of liquid amines, the reaction can be carried out in the absence of solvent. The reduction of the nitro group with concomitant introduction of the chloro group is achieved using tin (II) chloride according to the method described by Kelley et al. J. Med. Chem. 1995, 38(20), 4131-34. The dichloro diaminopyridine (II-3) is condensed with an appropriate acid such as cyanoacetic acid using an appropriate coupling reagent such as EDCl in a polar aprotic solvent such as DMF. The resulting amide (II-4) will undergo cyclodehydration in refluxing acetic acid and when followed by treatment in situ with NaNO₂ will afford a hydroxylamine such as (II-5). Reaction of (II-5) with hydroxylamine gives a bis-oxime that cyclodehydrates in the presence of an appropriate base such as triethylamine to give an aminofurazan such as II-6.

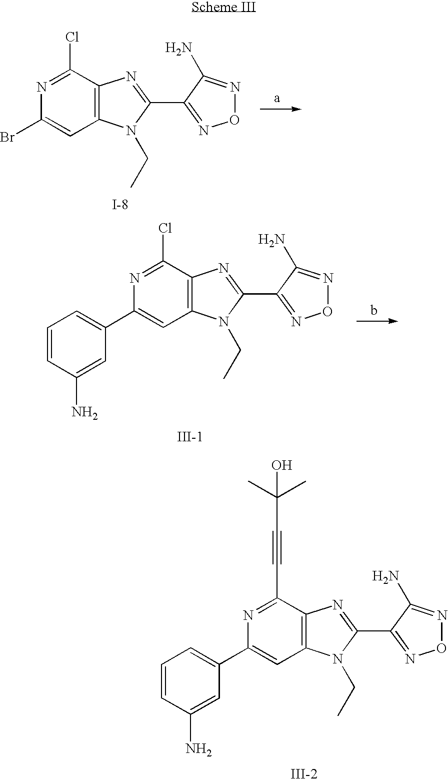

Reagents: (a) 3-aminophenylboronic acid, K₂CO₃, Pd(PPh₃)₄, dioxane, H₂O, 80 C; (b) 2-hydroxy-2-methyl-3-butyne, PdCl₂(PPh₃)₂, CuI, Et₃N, DMF, 100 C.

Reaction of bromide (I-8) with an aryl boronic acid such as 3-aminophenyl boronic acid in the presence of a catalyst, preferably tetrakistriphenylphosphino palladium and a base such as potassium carbonate or triethylamine in a suitable solvent mixture such as dioxane and water gives the corresponding aryl compound such as (III-1). Treatment of an appropriate aryl halide such as (III-1) with a catalyst such as dichlorobistriphenylphosphine palladium and a terminal alkyne in the presence of a suitable base such as triethylamine in an appropriate solvent such as dimethylformamide gives an aryl alkyne such as (III-2).

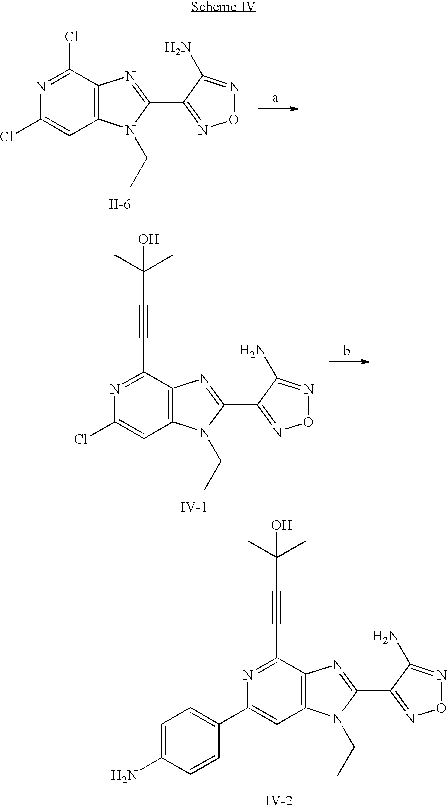

Reagents: (a) 2-hydroxy-2-methyl-3-butyne, PdCl₂(PPh₃)₂, CuI, Et₃N, DMF, 100 C; (b) 4-aminophenylboronic acid, K₂CO₃, Pd(PPh₃)₄, dioxane, H₂O, 80 C.

Treatment of an appropriate aryl halide such as (II-6) with an appropriate catalyst such as dichlorobistriphenylphosphine palladium and a terminal alkyne in the presence of a suitable base such as triethylamine in an appropriate solvent such as dimethylformamide gives the corresponding aryl alkyne such as (IV-1). Subsequent reaction with an aryl boronic acid such as 4-phenyl boronic acid in the presence of a catalyst, preferably tetrakistriphenylphosphino palladium and a base such as potassium carbonate or triethylamine in a suitable solvent mixture such as dioxane and water gives the corresponding aryl compound such as (IV-2).

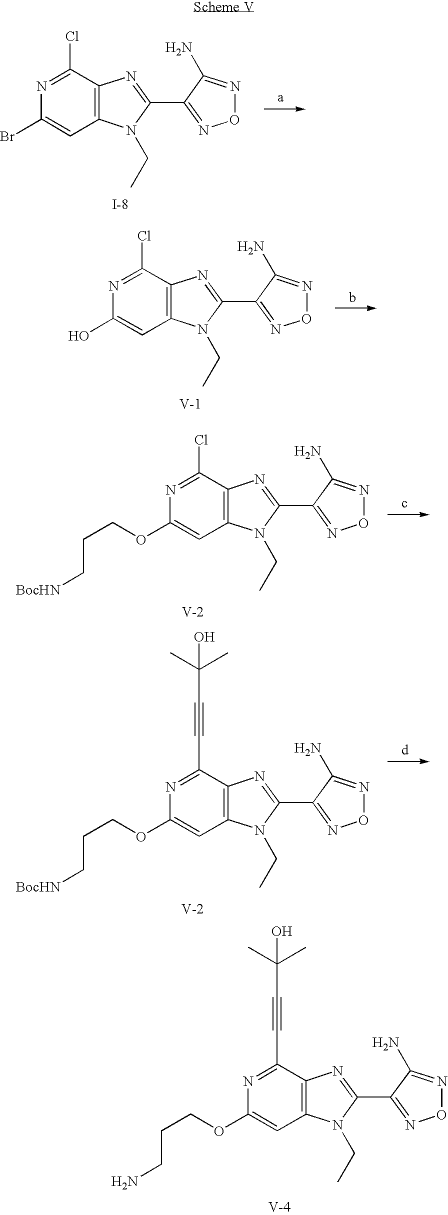

Reagents: (a) B(OMe)₃, n-BuLi, THF, −100° C.; H₂O₂, 2M NaOH; (b) PPh₃, DEAD, 1,1-dimethylethyl (3-hydroxypropyl)carbamate, THF, RT; (c) 2-hydroxy-2-methyl-3-butyne, PdCl₂(PPh₃)₂, CuI, Et₃N, DMF 100° C.; (d) 4M HCl in dioxane, RT.

The hydroxyl group is introduced by generating an aryl anion via halogen-metal exchange using a suitable base such as n-butyl lithium, reacting the anion with an appropriate boron electrophile such as trimethyl borate and oxidizing the resulting boronate with an appropriate oxidizing agent such as hydrogen peroxide in aqueous base to give imidazopyridinols such as (V-1). Etherification of the imidazopyridinol is carried out with an appropriate alcohol such as 1,1-dimethylethyl (3-hydroxypropyl)carbamate using the methods described by Mitsunobu, Synthesis 1981, 1 to give ethers such as (V-2). Treatment of an appropriate aryl halide such as (V-2) with an appropriate catalyst such as dichlorodiphenylphosphine palladium(II) and a terminal alkyne in the presence of a suitable base such as triethylamine in an appropriate solvent such as DMF gives the corresponding aryl alkyne such as (V-3). Removal of the Boc protecting group is achieved using α-protic acid such as trifluoroacetic acid or HCl in a polar solvent such as methanol giving compounds such as (V-4). Many different protecting groups are available to one skilled in the art and can be used here as long as they do not interfere with the processes listed herein.

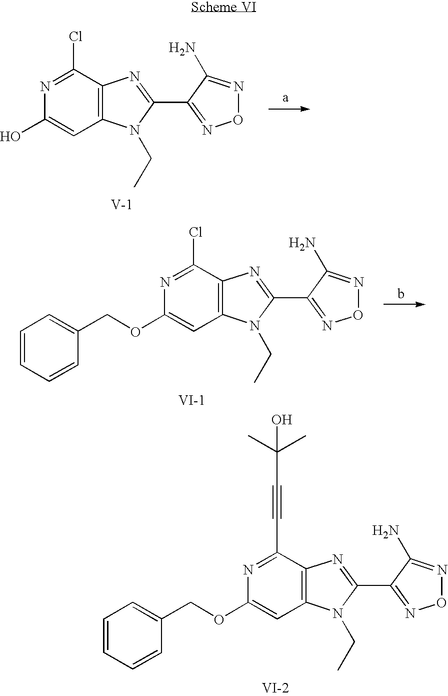

Reagents: (a) Benzyl bromide, Ag₂CO₃, THF, 60° C.; (b) 2-hydroxy-2-methyl-3-butyne, PdCl₂(PPh₃)₂, CuI, Et₃N, DMF 100° C.

Benzyl ether VI-1 results from selective 0 vs. N alkylation by heating pyridone V-1 with an appropriate alkyl halide in an ethereal solvent like THF using Ag₂CO₃ as base (VI-1) (Tieckelmann, H. Chem. Heterocycl. Compd. 1974, 14, 3, 597.). Subsequent Sonogashira coupling with an appropriate alkyne afforded analog VI-2.

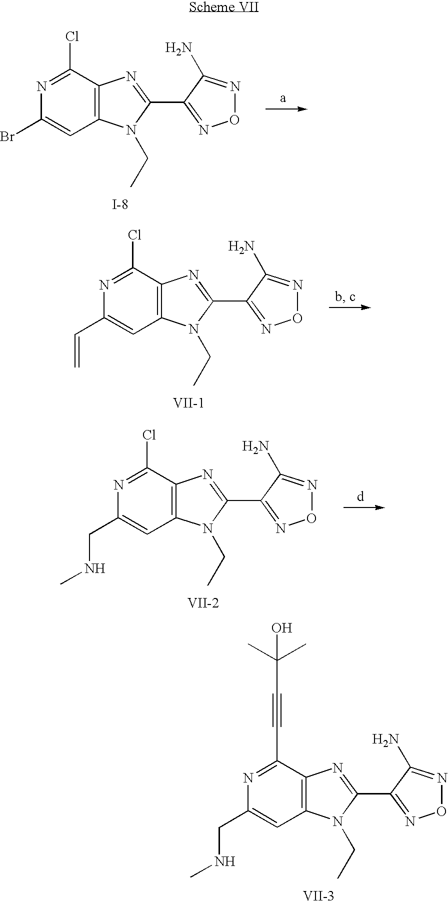

Reagents: (a) trivinyl boronate, K₂CO₃, Pd(PPh₃)₃, DME-H₂O, 70° C.; (b) O₃, DCM, −50° C.; (c) MeNH₂ (2M in THF), Na₂SO₄ then NaBH₄; (d) 2-hydroxy-2-methyl-3-butyne, PdCl₂(PPh₃)₂, CuI, Et₃N, DMF 100° C.

Selective Suzuki coupling of bromopyridine (I-8) with an appropriate vinylborane provides vinylpyridines like VII-1. Ozonolysis followed by reductive amination with an appropriate primary or secondary amine affords analogs related to VII-2. Standard Sonogashira coupling with an appropriate alkyne, copper and palladium source supplies final analogs like VII-3.

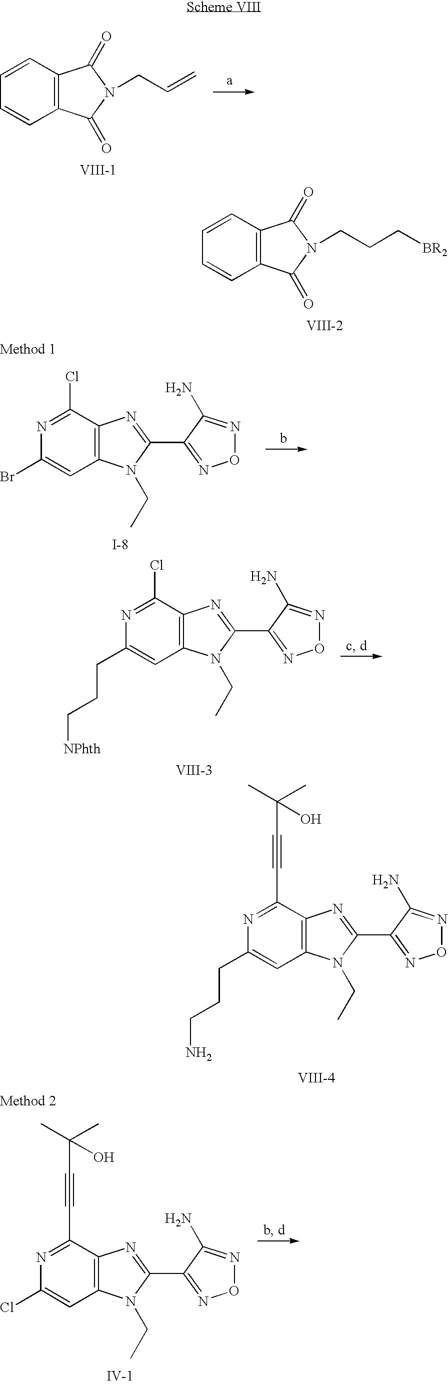

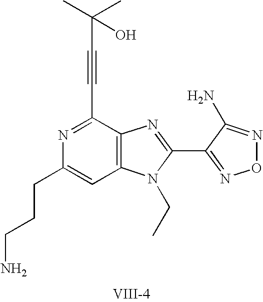

Reagents: (a) 9-BBN, toluene, 75° C.; (b) VIII-2, pre-heated solution of Pd(OAc)₂, DPPF, DMF, 75° C. 30 min., K₂CO₃, 75° C.; (c) 2-hydroxy-2-methyl-3-butyne, PdCl₂(PPh₃)₂, CuI, Et₃N, DMF 100° C.; (d) MeNH2 (40 wt % in H2O), MeOH, 25° C.

Alkylamine analogs like VIII-4 can be obtained using two independent procedures. Alkyl boranes like VIII-2 are prepared by treatment of an appropriate protected amino olefin like allyl phthalimide (VIII-1) with 9-BBN. This intermediate, used in situ, is treated with an appropriate palladium source and ligand, an appropriate base like K₂CO₃ and an appropriate bromopyridine like I-8. Subsequent Sonogashira coupling with an appropriate alkyne, copper and palladium source is followed by deprotection of the amine with an appropriate amine source like methylamine.

Alternatively, chloropyridine IV-1 can undergo the aforementioned Suzuki-Miyaura cross coupling reaction (Suzuki, A. Cross-coupling reaction of Organoboron Compounds with Organic Halides.) with an appropriate olefin like VIII-1. Subsequent amine deprotection occurs through use of an appropriate amine source like methylamine providing alkylamine analogs like VIII-4.

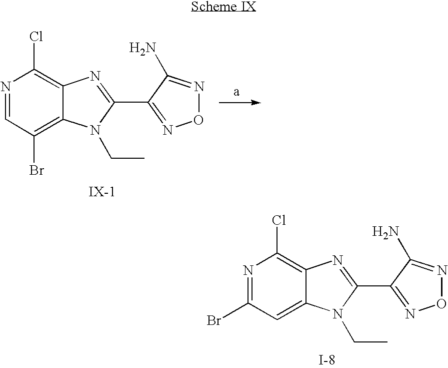

### Reagents: (a) LDA, THF, −40° C.

Alternatively, intermediate I-8 can be prepared using a halogen-dance reaction (Duan, Zhang Heterocycles 2005, 65(8), 2005-2012). Thus, a suitable halogen containing precursor like IX-1 (prepared according to WO2005011700 A1) is dissolved in a polar solvent like tetrahydrofuran and treated with a strong base like lithium diisopropyl amine to give I-8.

By the term “co-administering” and derivatives thereof as used herein is meant either simultaneous administration or any manner of separate sequential administration of an AKT inhibiting compound, as described herein, and a further active ingredient or ingredients, known to be useful in the treatment of cancer, including chemotherapy and radiation treatment, or to be useful in the treatment of arthritis. The term further active ingredient or ingredients, as used herein, includes any compound or therapeutic agent known to or that demonstrates advantageous properties when administered to a patient in need of treatment for cancer or arthritis. Preferably, if the administration is not simultaneous, the compounds are administered in a close time proximity to each other. Furthermore, it does not matter if the compounds are administered in the same dosage form, e.g. one compound may be administered topically and another compound may be administered orally.

Typically, any anti-neoplastic agent that has activity versus a susceptible tumor being treated may be co-administered in the treatment of cancer in the present invention. Examples of such agents can be found in Cancer Principles and Practice f Oncology by V. T. Devita and S. Hellman (editors), 6^{th} edition (Feb. 15, 2001), Lippincott Williams & Wilkins Publishers. A person of ordinary skill in the art would be able to discern which combinations of agents would be useful based on the particular characteristics of the drugs and the cancer involved. Typical anti-neoplastic agents useful in the present invention include, but are not limited to, anti-microtubule agents such as diterpenoids and vinca alkaloids; platinum coordination complexes; alkylating agents such as nitrogen mustards, oxazaphosphorines, alkylsulfonates, nitrosoureas, and triazenes; antibiotic agents such as anthracyclins, actinomycins and bleomycins; topoisomerase II inhibitors such as epipodophyllotoxins; antimetabolites such as purine and pyrimidine analogues and anti-folate compounds; topoisomerase I inhibitors such as camptothecins; hormones and hormonal analogues; signal transduction pathway inhibitors; non-receptor tyrosine kinase angiogenesis inhibitors; immunotherapeutic agents; proapoptotic agents; and cell cycle signaling inhibitors.

Examples of a further active ingredient or ingredients for use in combination or co-administered with the presently invented AKT inhibiting compounds are chemotherapeutic agents.

Anti-microtubule or anti-mitotic agents are phase specific agents active against the microtubules of tumor cells during M or the mitosis phase of the cell cycle. Examples of anti-microtubule agents include, but are not limited to, diterpenoids and vinca alkaloids.

Diterpenoids, which are derived from natural sources, are phase specific anti-cancer agents that operate at the G₂/M phases of the cell cycle. It is believed that the diterpenoids stabilize the β-tubulin subunit of the microtubules, by binding with this protein. Disassembly of the protein appears then to be inhibited with mitosis being arrested and cell death following. Examples of diterpenoids include, but are not limited to, paclitaxel and its analog docetaxel.

Paclitaxel, 5β,20-epoxy-1,2α,4,7β,10β,13α-hexa-hydroxytax-11-en-9-one 4,10-diacetate 2-benzoate 13-ester with (2R,3S)—N-benzoyl-3-phenylisoserine; is a natural diterpene product isolated from the Pacific yew tree Taxus brevifolia and is commercially available as an injectable solution TAXOL®. It is a member of the taxane family of terpenes. It was first isolated in 1971 by Wani et al. J. Am. Chem, Soc., 93:2325.1971), who characterized its structure by chemical and X-ray crystallographic methods. One mechanism for its activity relates to paclitaxel's capacity to bind tubulin, thereby inhibiting cancer cell growth. Schiff et al., Proc. Natl, Acad, Sci. USA, 77:1561-1565 (1980); Schiff et al., Nature, 277:665-667 (1979); Kumar, J. Biol, Chem, 256: 10435-10441 (1981). For a review of synthesis and anticancer activity of some paclitaxel derivatives see: D. G. I. Kingston et al., Studies in Organic Chemistry vol. 26, entitled “New trends in Natural Products Chemistry 1986”, Aftaur-Rahman, P. W. Le Quesne, Eds. (Elsevier, Amsterdam, 1986) pp 219-235.

Paclitaxel has been approved for clinical use in the treatment of refractory ovarian cancer in the United States (Markman et al., Yale Journal of Biology and Medicine, 64:583, 1991; McGuire et al., Ann. Intern, Med., 111:273, 1989) and for the treatment of breast cancer (Holmes et al., J. Nat. Cancer Inst., 83:1797, 1991.) It is a potential candidate for treatment of neoplasms in the skin (Einzig et. al., Proc. Am. Soc. Clin. Oncol., 20:46) and head and neck carcinomas (Forastire et. al., Sem. Oncol., 20:56, 1990). The compound also shows potential for the treatment of polycystic kidney disease (Woo et. al., Nature, 368:750.1994), lung cancer and malaria. Treatment of patients with paclitaxel results in bone marrow suppression (multiple cell lineages, Ignoff, R. J. et. al, Cancer Chemotherapy Pocket Guide, 1998) related to the duration of dosing above a threshold concentration (50 nM) (Kearns, C. M. et. al., Seminars in Oncology, 3(6) p. 16-23, 1995).

Docetaxel, (2R,3S)—N-carboxy-3-phenylisoserine, N-tert-butyl ester, 13-ester with 5β-20-epoxy-1,2α,4,7β,10β,13α-hexahydroxytax-11-en-9-one 4-acetate 2-benzoate, trihydrate; is commercially available as an injectable solution as TAXOTERE®. Docetaxel is indicated for the treatment of breast cancer. Docetaxel is a semisynthetic derivative of paclitaxel q.v., prepared using a natural precursor, 10-deacetyl-baccatin III, extracted from the needle of the European Yew tree. The dose limiting toxicity of docetaxel is neutropenia.

Vinca alkaloids are phase specific anti-neoplastic agents derived from the periwinkle plant. Vinca alkaloids act at the M phase (mitosis) of the cell cycle by binding specifically to tubulin. Consequently, the bound tubulin molecule is unable to polymerize into microtubules. Mitosis is believed to be arrested in metaphase with cell death following. Examples of vinca alkaloids include, but are not limited to, vinblastine, vincristine, and vinorelbine.

Vinblastine, vincaleukoblastine sulfate, is commercially available as VELBAN® as an injectable solution. Although, it has possible indication as a second line therapy of various solid tumors, it is primarily indicated in the treatment of testicular cancer and various lymphomas including Hodgkin's Disease; and lymphocytic and histiocytic lymphomas. Myelosuppression is the dose limiting side effect of vinblastine.

Vincristine, vincaleukoblastine, 22-oxo-, sulfate, is commercially available as ONCOVIN® as an injectable solution. Vincristine is indicated for the treatment of acute leukemias and has also found use in treatment regimens for Hodgkin's and non-Hodgkin's malignant lymphomas. Alopecia and neurologic effects are the most common side effect of vincristine and to a lesser extent myelosupression and gastrointestinal mucositis effects occur.

Vinorelbine, 3′,4′-didehydro-4′-deoxy-C′-norvincaleukoblastine [R—(R*,R*)-2,3-dihydroxybutanedioate(1:2)(salt)], commercially available as an injectable solution of vinorelbine tartrate (NAVELBINE®), is a semisynthetic vinca alkaloid. Vinorelbine is indicated as a single agent or in combination with other chemotherapeutic agents, such as cisplatin, in the treatment of various solid tumors, particularly non-small cell lung, advanced breast, and hormone refractory prostate cancers. Myelosuppression is the most common dose limiting side effect of vinorelbine.

Platinum coordination complexes are non-phase specific anti-cancer agents, which are interactive with DNA. The platinum complexes enter tumor cells, undergo, aquation and form intra- and interstrand crosslinks with DNA causing adverse biological effects to the tumor. Examples of platinum coordination complexes include, but are not limited to, cisplatin and carboplatin.

Cisplatin, cis-diamminedichloroplatinum, is commercially available as PLATINOL® as an injectable solution. Cisplatin is primarily indicated in the treatment of metastatic testicular and ovarian cancer and advanced bladder cancer. The primary dose limiting side effects of cisplatin are nephrotoxicity, which may be controlled by hydration and diuresis, and ototoxicity.

Carboplatin, platinum, diammine [1,1-cyclobutane-dicarboxylate(2-)-O,O′], is commercially available as PARAPLATIN® as an injectable solution. Carboplatin is primarily indicated in the first and second line treatment of advanced ovarian carcinoma. Bone marrow suppression is the dose limiting toxicity of carboplatin.

Alkylating agents are non-phase anti-cancer specific agents and strong electrophiles. Typically, alkylating agents form covalent linkages, by alkylation, to DNA through nucleophilic moieties of the DNA molecule such as phosphate, amino, sulfhydryl, hydroxyl, carboxyl, and imidazole groups. Such alkylation disrupts nucleic acid function leading to cell death. Examples of alkylating agents include, but are not limited to, nitrogen mustards such as cyclophosphamide, melphalan, and chlorambucil; alkyl sulfonates such as busulfan; nitrosoureas such as carmustine; and triazenes such as dacarbazine.

Cyclophosphamide, 2-[bis(2-chloroethyl)amino]tetrahydro-2H-1,3,2-oxazaphosphorine 2-oxide monohydrate, is commercially available as an injectable solution or tablets as CYTOXAN®. Cyclophosphamide is indicated as a single agent or in combination with other chemotherapeutic agents, in the treatment of malignant lymphomas, multiple myeloma, and leukemias. Alopecia, nausea, vomiting and leukopenia are the most common dose limiting side effects of cyclophosphamide.

Melphalan, 4-[bis(2-chloroethyl)amino]-L-phenylalanine, is commercially available as an injectable solution or tablets as ALKERAN®. Melphalan is indicated for the palliative treatment of multiple myeloma and non-resectable epithelial carcinoma of the ovary. Bone marrow suppression is the most common dose limiting side effect of melphalan.

Chlorambucil, 4-[bis(2-chloroethyl)amino]benzenebutanoic acid, is commercially available as LEUKERAN® tablets. Chlorambucil is indicated for the palliative treatment of chronic lymphatic leukemia, and malignant lymphomas such as lymphosarcoma, giant follicular lymphoma, and Hodgkin's disease. Bone marrow suppression is the most common dose limiting side effect of chlorambucil.

Busulfan, 1,4-butanediol dimethanesulfonate, is commercially available as MYLERAN® TABLETS. Busulfan is indicated for the palliative treatment of chronic myelogenous leukemia. Bone marrow suppression is the most common dose limiting side effects of busulfan.

Carmustine, 1,3-[bis(2-chloroethyl)-1-nitrosourea, is commercially available as single vials of lyophilized material as BiCNU®. Carmustine is indicated for the palliative treatment as a single agent or in combination with other agents for brain tumors, multiple myeloma, Hodgkin's disease, and non-Hodgkin's lymphomas. Delayed myelosuppression is the most common dose limiting side effects of carmustine.

Dacarbazine, 5-(3,3-dimethyl-1-triazeno)-imidazole-4-carboxamide, is commercially available as single vials of material as DTIC-Dome®. Dacarbazine is indicated for the treatment of metastatic malignant melanoma and in combination with other agents for the second line treatment of Hodgkin's Disease. Nausea, vomiting, and anorexia are the most common dose limiting side effects of dacarbazine.

Antibiotic anti-neoplastics are non-phase specific agents, which bind or intercalate with DNA. Typically, such action results in stable DNA complexes or strand breakage, which disrupts ordinary function of the nucleic acids leading to cell death. Examples of antibiotic anti-neoplastic agents include, but are not limited to, actinomycins such as dactinomycin, anthrocyclins such as daunorubicin and doxorubicin; and bleomycins.

Dactinomycin, also know as Actinomycin D, is commercially available in injectable form as COSMEGEN®. Dactinomycin is indicated for the treatment of Wilm's tumor and rhabdomyosarcoma. Nausea, vomiting, and anorexia are the most common dose limiting side effects of dactinomycin.

Daunorubicin, (8S-cis-)-8-acetyl-10-[(3-amino-2,3,6-trideoxy-α-L-lyxo-hexopyranosyl)oxy]-7,8,9,10-tetrahydro-6,8,11-trihydroxy-1-methoxy-5,12 naphthacenedione hydrochloride, is commercially available as a liposomal injectable form as DAUNOXOME® or as an injectable as CERUBIDINE®. Daunorubicin is indicated for remission induction in the treatment of acute nonlymphocytic leukemia and advanced HIV associated Kaposi's sarcoma. Myelosuppression is the most common dose limiting side effect of daunorubicin.

Doxorubicin, (8S,10S)-10-[(3-amino-2,3,6-trideoxy-α-L-lyxo-hexopyranosyl)oxy]-8-glycoloyl, 7,8,9,10-tetrahydro-6,8,11-trihydroxy-1-methoxy-5,12 naphthacenedione hydrochloride, is commercially available as an injectable form as RUBEX® or ADRIAMYCIN RDF®. Doxorubicin is primarily indicated for the treatment of acute lymphoblastic leukemia and acute myeloblastic leukemia, but is also a useful component in the treatment of some solid tumors and lymphomas. Myelosuppression is the most common dose limiting side effect of doxorubicin.

Bleomycin, a mixture of cytotoxic glycopeptide antibiotics isolated from a strain of Streptomyces verticillus, is commercially available as BLENOXANE®. Bleomycin is indicated as a palliative treatment, as a single agent or in combination with other agents, of squamous cell carcinoma, lymphomas, and testicular carcinomas. Pulmonary and cutaneous toxicities are the most common dose limiting side effects of bleomycin.

Topoisomerase II inhibitors include, but are not limited to, epipodophyllotoxins.

Epipodophyllotoxins are phase specific anti-neoplastic agents derived from the mandrake plant. Epipodophyllotoxins typically affect cells in the S and G₂ phases of the cell cycle by forming a ternary complex with topoisomerase II and DNA causing DNA strand breaks. The strand breaks accumulate and cell death follows. Examples of epipodophyllotoxins include, but are not limited to, etoposide and teniposide.

Etoposide, 4′-demethyl-epipodophyllotoxin 9[4,6-0-(R)-ethylidene-β-D-glucopyranoside], is commercially available as an injectable solution or capsules as VePESID® and is commonly known as VP-16. Etoposide is indicated as a single agent or in combination with other chemotherapy agents in the treatment of testicular and non-small cell lung cancers. Myelosuppression is the most common side effect of etoposide. The incidence of leucopenia tends to be more severe than thrombocytopenia.

Teniposide, 4′-demethyl-epipodophyllotoxin 9[4,6-0-(R)-thenylidene-β-D-glucopyranoside], is commercially available as an injectable solution as VUMON® and is commonly known as VM-26. Teniposide is indicated as a single agent or in combination with other chemotherapy agents in the treatment of acute leukemia in children. Myelosuppression is the most common dose limiting side effect of teniposide. Teniposide can induce both leucopenia and thrombocytopenia.

Antimetabolite neoplastic agents are phase specific anti-neoplastic agents that act at S phase (DNA synthesis) of the cell cycle by inhibiting DNA synthesis or by inhibiting purine or pyrimidine, base synthesis and thereby limiting DNA synthesis. Consequently, S phase does not proceed and cell death follows. Examples of antimetabolite anti-neoplastic agents include, but are not limited to, fluorouracil, methotrexate, cytarabine, mercaptopurine, thioguanine, and gemcitabine.

5-fluorouracil, 5-fluoro-2,4-(1H,3H) pyrimidinedione, is commercially available as fluorouracil. Administration of 5-fluorouracil leads to inhibition of thymidylate synthesis and is also incorporated into both RNA and DNA. The result typically is cell death. 5-fluorouracil is indicated as a single agent or in combination with other chemotherapy agents in the treatment of carcinomas of the breast, colon, rectum, stomach and pancreas. Myelosuppression and mucositis are dose limiting side effects of 5-fluorouracil. Other fluoropyrimidine analogs include 5-fluoro deoxyuridine (floxuridine) and 5-fluorodeoxyuridine monophosphate.

Cytarabine, 4-amino-1-β-D-arabinofuranosyl-2(1H)-pyrimidinone, is commercially available as CYTOSAR-U® and is commonly known as Ara-C. It is believed that cytarabine exhibits cell phase specificity at S-phase by inhibiting DNA chain elongation by terminal incorporation of cytarabine into the growing DNA chain. Cytarabine is indicated as a single agent or in combination with other chemotherapy agents in the treatment of acute leukemia. Other cytidine analogs include 5-azacytidine and 2′,2′-difluorodeoxycytidine (gemcitabine). Cytarabine induces leucopenia, thrombocytopenia, and mucositis.

Mercaptopurine, 1,7-dihydro-6H-purine-6-thione monohydrate, is commercially available as PURINETHOL®. Mercaptopurine exhibits cell phase specificity at S-phase by inhibiting DNA synthesis by an as of yet unspecified mechanism. Mercaptopurine is indicated as a single agent or in combination with other chemotherapy agents in the treatment of acute leukemia. Myelosuppression and gastrointestinal mucositis are expected side effects of mercaptopurine at high doses. A useful mercaptopurine analog is azathioprine.

Thioguanine, 2-amino-1,7-dihydro-6H-purine-6-thione, is commercially available as TABLOID®. Thioguanine exhibits cell phase specificity at S-phase by inhibiting DNA synthesis by an as of yet unspecified mechanism. Thioguanine is indicated as a single agent or in combination with other chemotherapy agents in the treatment of acute leukemia. Myelosuppression, including leucopenia, thrombocytopenia, and anemia, is the most common dose limiting side effect of thioguanine administration. However, gastrointestinal side effects occur and can be dose limiting. Other purine analogs include pentostatin, erythrohydroxynonyladenine, fludarabine phosphate, and cladribine.

Gemcitabine, 2′-deoxy-2′,2′-difluorocytidine monohydrochloride (β-isomer), is commercially available as GEMZAR®. Gemcitabine exhibits cell phase specificity at S-phase and by blocking progression of cells through the G1/S boundary. Gemcitabine is indicated in combination with cisplatin in the treatment of locally advanced non-small cell lung cancer and alone in the treatment of locally advanced pancreatic cancer. Myelosuppression, including leucopenia, thrombocytopenia, and anemia, is the most common dose limiting side effect of gemcitabine administration.

Methotrexate, N-[4-[[(2,4-diamino-6-pteridinyl)methyl]methylamino]benzoyl]-L-glutamic acid, is commercially available as methotrexate sodium. Methotrexate exhibits cell phase effects specifically at S-phase by inhibiting DNA synthesis, repair and/or replication through the inhibition of dyhydrofolic acid reductase which is required for synthesis of purine nucleotides and thymidylate. Methotrexate is indicated as a single agent or in combination with other chemotherapy agents in the treatment of choriocarcinoma, meningeal leukemia, non-Hodgkin's lymphoma, and carcinomas of the breast, head, neck, ovary and bladder. Myelosuppression (leucopenia, thrombocytopenia, and anemia) and mucositis are expected side effect of methotrexate administration.

Camptothecins, including, camptothecin and camptothecin derivatives are available or under development as Topoisomerase I inhibitors. Camptothecins cytotoxic activity is believed to be related to its Topoisomerase I inhibitory activity. Examples of camptothecins include, but are not limited to irinotecan, topotecan, and the various optical forms of 7-(4-methylpiperazino-methylene)-10,11-ethylenedioxy-20-camptothecin described below.

Irinotecan HCl, (4S)-4,11-diethyl-4-hydroxy-9-[(4-piperidinopiperidino) carbonyloxy]-1H-pyrano[3′,4′,6,7]indolizino[1,2-b]quinoline-3,14(4H,12H)-dione hydrochloride, is commercially available as the injectable solution CAMPTOSAR®.

Irinotecan is a derivative of camptothecin which binds, along with its active metabolite SN-38, to the topoisomerase I-DNA complex. It is believed that cytotoxicity occurs as a result of irreparable double strand breaks caused by interaction of the topoisomerase I: DNA: irintecan or SN-38 ternary complex with replication enzymes. Irinotecan is indicated for treatment of metastatic cancer of the colon or rectum. The dose limiting side effects of irinotecan HCl are myelosuppression, including neutropenia, and GI effects, including diarrhea.

Topotecan HCl, (S)-10-[(dimethylamino)methyl]-4-ethyl-4,9-dihydroxy-1H-pyrano[3′,4′,6,7]indolizino[1,2-b]quinoline-3,14-(4H,12H)-dione monohydrochloride, is commercially available as the injectable solution HYCAMTIN®. Topotecan is a derivative of camptothecin which binds to the topoisomerase I-DNA complex and prevents religation of singles strand breaks caused by Topoisomerase I in response to torsional strain of the DNA molecule. Topotecan is indicated for second line treatment of metastatic carcinoma of the ovary and small cell lung cancer. The dose limiting side effect of topotecan HCl is myelosuppression, primarily neutropenia.

Also of interest, is the camptothecin derivative of formula A following, currently under development, including the racemic mixture (R,S) form as well as the R and S enantiomers:

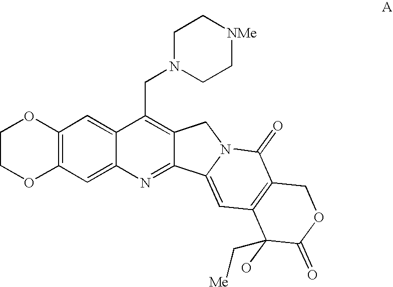

known by the chemical name “7-(4-methylpiperazino-methylene)-10,11-ethylenedioxy-20(R,S)-camptothecin (racemic mixture) or “7-(4-methylpiperazino-methylene)-10,11-ethylenedioxy-20(R)-camptothecin (R enantiomer) or “7-(4-methylpiperazino-methylene)-10,11-ethylenedioxy-20(S)-camptothecin (S enantiomer). Such compound as well as related compounds are described, including methods of making, in U.S. Pat. Nos. 6,063,923; 5,342,947; 5,559,235; 5,491,237 and pending U.S. patent application Ser. No. 08/977,217 filed Nov. 24, 1997.

Hormones and hormonal analogues are useful compounds for treating cancers in which there is a relationship between the hormone(s) and growth and/or lack of growth of the cancer. Examples of hormones and hormonal analogues useful in cancer treatment include, but are not limited to, adrenocorticosteroids such as prednisone and prednisolone which are useful in the treatment of malignant lymphoma and acute leukemia in children; aminoglutethimide and other aromatase inhibitors such as anastrozole, letrazole, vorazole, and exemestane useful in the treatment of adrenocortical carcinoma and hormone dependent breast carcinoma containing estrogen receptors; progestrins such as megestrol acetate useful in the treatment of hormone dependent breast cancer and endometrial carcinoma; estrogens, androgens, and anti-androgens such as flutamide, nilutamide, bicalutamide, cyproterone acetate and 5α-reductases such as finasteride and dutasteride, useful in the treatment of prostatic carcinoma and benign prostatic hypertrophy; anti-estrogens such as tamoxifen, toremifene, raloxifene, droloxifene, iodoxyfene, as well as selective estrogen receptor modulators (SERMS) such those described in U.S. Pat. Nos. 5,681,835, 5,877,219, and 6,207,716, useful in the treatment of hormone dependent breast carcinoma and other susceptible cancers; and gonadotropin-releasing hormone (GnRH) and analogues thereof which stimulate the release of leutinizing hormone (LH) and/or follicle stimulating hormone (FSH) for the treatment prostatic carcinoma, for instance, LHRH agonists and antagagonists such as goserelin acetate and luprolide.

Signal transduction pathway inhibitors are those inhibitors, which block or inhibit a chemical process which evokes an intracellular change. As used herein this change is cell proliferation or differentiation. Signal tranduction inhibitors useful in the present invention include inhibitors of receptor tyrosine kinases, non-receptor tyrosine kinases, SH2/SH3 domain blockers, serine/threonine kinases, phosphotidyl inositol-3 kinases, myo-inositol signaling, and Ras oncogenes.

Several protein tyrosine kinases catalyse the phosphorylation of specific tyrosyl residues in various proteins involved in the regulation of cell growth. Such protein tyrosine kinases can be broadly classified as receptor or non-receptor kinases.

Receptor tyrosine kinases are transmembrane proteins having an extracellular ligand binding domain, a transmembrane domain, and a tyrosine kinase domain. Receptor tyrosine kinases are involved in the regulation of cell growth and are generally termed growth factor receptors. Inappropriate or uncontrolled activation of many of these kinases, i.e. aberrant kinase growth factor receptor activity, for example by over-expression or mutation, has been shown to result in uncontrolled cell growth. Accordingly, the aberrant activity of such kinases has been linked to malignant tissue growth. Consequently, inhibitors of such kinases could provide cancer treatment methods. Growth factor receptors include, for example, epidermal growth factor receptor (EGFr), platelet derived growth factor receptor (PDGFr), erbB2, erbB4, vascular endothelial growth factor receptor (VEGFr), tyrosine kinase with immunoglobulin-like and epidermal growth factor homology domains (TIE-2), insulin growth factor-I (IGFI) receptor, macrophage colony stimulating factor (cfms), BTK, ckit, cmet, fibroblast growth factor (FGF) receptors, Trk receptors (TrkA, TrkB, and TrkC), ephrin (eph) receptors, and the RET protooncogene. Several inhibitors of growth receptors are under development and include ligand antagonists, antibodies, tyrosine kinase inhibitors and anti-sense oligonucleotides. Growth factor receptors and agents that inhibit growth factor receptor function are described, for instance, in Kath, John C., Exp. Opin. Ther. Patents (2000) 10(6):803-818; Shawver et al DDT Vol 2, No. 2 Feb. 1997; and Lofts, F. J. et al, “Growth factor receptors as targets”, New Molecular Targets for Cancer Chemotherapy, ed. Workman, Paul and Kerr, David, CRC press 1994, London.

Tyrosine kinases, which are not growth factor receptor kinases are termed non-receptor tyrosine kinases. Non-receptor tyrosine kinases useful in the present invention, which are targets or potential targets of anti-cancer drugs, include cSrc, Lck, Fyn, Yes, Jak, cAbl, FAK (Focal adhesion kinase), Brutons tyrosine kinase, and Bcr-Abl. Such non-receptor kinases and agents which inhibit non-receptor tyrosine kinase function are described in Sinh, S, and Corey, S. J., (1999) Journal of Hematotherapy and Stem Cell Research 8 (5): 465-80; and Bolen, J. B., Brugge, J. S., (1997) Annual review of Immunology. 15: 371-404.

SH2/SH3 domain blockers are agents that disrupt SH2 or SH3 domain binding in a variety of enzymes or adaptor proteins including, PI3-K p85 subunit, Src family kinases, adaptor molecules (Shc, Crk, Nck, Grb2) and Ras-GAP. SH2/SH3 domains as targets for anti-cancer drugs are discussed in Smithgall, T. E. (1995), Journal of Pharmacological and Toxicological Methods. 34(3) 125-32.

Inhibitors of Serine/Threonine Kinases including MAP kinase cascade blockers which include blockers of Raf kinases (rafk), Mitogen or Extracellular Regulated Kinase (MEKs), and Extracellular Regulated Kinases (ERKs); and Protein kinase C family member blockers including blockers of PKCs (alpha, beta, gamma, epsilon, mu, lambda, iota, zeta). IkB kinase family (IKKa, IKKb), PKB family kinases, akt kinase family members, and TGF beta receptor kinases. Such Serine/Threonine kinases and inhibitors thereof are described in Yamamoto, T., Taya, S., Kaibuchi, K., (1999), Journal of Biochemistry. 126 (5) 799-803; Brodt, P, Samani, A., and Navab, R. (2000), Biochemical Pharmacology, 60.1101-1107; Massague, J., Weis-Garcia, F. (1996) Cancer Surveys. 27:41-64; Philip, P. A., and Harris, A. L (1995), Cancer Treatment and Research. 78: 3-27, Lackey, K. et al Bioorganic and Medicinal Chemistry Letters, (10), 2000, 223-226; U.S. Pat. No. 6,268,391; and Martinez-lacaci, L., et al, Int. J. Cancer (2000), 88(1), 44-52.

Inhibitors of Phosphotidyl inositol-3 Kinase family members including blockers of PI3-kinase, ATM, DNA-PK, and Ku are also useful in the present invention. Such kinases are discussed in, Abraham, R. T. (1996), Current Opinion in Immunology. 8 (3) 412-8; Canman, C. E., Lim, D. S. (1998), Oncogene 17 (25) 3301-3308; Jackson, S. P. (1997), International Journal of Biochemistry and Cell Biology. 29 (7):935-8; and Zhong, H. et al, Cancer res, (2000) 60(6), 1541-1545.

Also useful in the present invention are Myo-inositol signaling inhibitors such as phospholipase C blockers and Myoinositol analogues. Such signal inhibitors are described in Powis, G., and Kozikowski A., (1994) New Molecular Targets for Cancer Chemotherapy ed., Paul Workman and David Kerr, CRC press 1994, London.

Another group of signal transduction pathway inhibitors are inhibitors of Ras Oncogene. Such inhibitors include inhibitors of farnesyltransferase, geranyl-geranyl transferase, and CAAX proteases as well as anti-sense oligonucleotides, ribozymes and immunotherapy. Such inhibitors have been shown to block ras activation in cells containing wild type mutant ras, thereby acting as antiproliferation agents. Ras oncogene inhibition is discussed in Scharovsky, O. G., Rozados, V. R., Gervasoni, S. I. Matar, P. (2000), Journal of Biomedical Science. 7(4) 292-8; Ashby, M. N. (1998), Current Opinion in Lipidology. 9 (2) 99-102; and BioChim. Biophys. Acta, (19899) 1423(3):19-30.

As mentioned above, antibody antagonists to receptor kinase ligand binding may also serve as signal transduction inhibitors. This group of signal transduction pathway inhibitors includes the use of humanized antibodies to the extracellular ligand binding domain of receptor tyrosine kinases. For example Imclone C225 EGFR specific antibody (see Green, M. C. et al, Monoclonal Antibody Therapy for Solid Tumors, Cancer Treat. Rev., (2000), 26(4), 269-286); Herceptin ® erbB2 antibody (see Tyrosine Kinase Signalling in Breast cancer:erbB Family Receptor Tyrosine Kniases, Breast cancer Res., 2000, 2(3), 176-183); and 2CB VEGFR2 specific antibody (see Brekken, R. A. et al, Selective Inhibition of VEGFR2 Activity by a monoclonal Anti-VEGF antibody blocks tumor growth in mice, Cancer Res. (2000) 60, 5117-5124).

Non-receptor kinase angiogenesis inhibitors may also find use in the present invention. Inhibitors of angiogenesis related VEGFR and TIE2 are discussed above in regard to signal transduction inhibitors (both receptors are receptor tyrosine kinases). Angiogenesis in general is linked to erbB2/EGFR signaling since inhibitors of erbB2 and EGFR have been shown to inhibit angiogenesis, primarily VEGF expression. Thus, the combination of an erbB2/EGFR inhibitor with an inhibitor of angiogenesis makes sense. Accordingly, non-receptor tyrosine kinase inhibitors may be used in combination with the EGFR/erbB2 inhibitors of the present invention. For example, anti-VEGF antibodies, which do not recognize VEGFR (the receptor tyrosine kinase), but bind to the ligand; small molecule inhibitors of integrin (alpha, beta₃) that will inhibit angiogenesis; endostatin and angiostatin (non-RTK) may also prove useful in combination with the disclosed erb family inhibitors. (See Bruns C J et al (2000), Cancer Res., 60: 2926-2935; Schreiber A B, Winkler M E, and Derynck R. (1986), Science, 232: 1250-1253; Yen L et al. (2000), Oncogene 19: 3460-3469).

Agents used in immunotherapeutic regimens may also be useful in combination with the compounds of formula (I). There are a number of immunologic strategies to generate an immune response against erbB2 or EGFR. These strategies are generally in the realm of tumor vaccinations. The efficacy of immunologic approaches may be greatly enhanced through combined inhibition of erbB2/EGFR signaling pathways using a small molecule inhibitor. Discussion of the immunologic/tumor vaccine approach against erbB2/EGFR are found in Reilly R T et al. (2000), Cancer Res. 60: 3569-3576; and Chen Y, Hu D, Eling D J, Robbins J, and Kipps T J. (1998), Cancer Res. 58: 1965-1971.

Agents used in proapoptotic regimens (e.g., bcl-2 antisense oligonucleotides) may also be used in the combination of the present invention. Members of the Bcl-2 family of proteins block apoptosis. Upregulation of bcl-2 has therefore been linked to chemoresistance. Studies have shown that the epidermal growth factor (EGF) stimulates anti-apoptotic members of the bcl-2 family (i.e., mcl-1). Therefore, strategies designed to downregulate the expression of bcl-2 in tumors have demonstrated clinical benefit and are now in Phase II/III trials, namely Genta's G3139 bcl-2 antisense oligonucleotide. Such proapoptotic strategies using the antisense oligonucleotide strategy for bcl-2 are discussed in Water J S et al. (2000), J. Clin. Oncol. 18: 1812-1823; and Kitada S et al. (1994), Antisense Res. Dev. 4: 71-79.

Cell cycle signalling inhibitors inhibit molecules involved in the control of the cell cycle. A family of protein kinases called cyclin dependent kinases (CDKs) and their interaction with a family of proteins termed cyclins controls progression through the eukaryotic cell cycle. The coordinate activation and inactivation of different cyclin/CDK complexes is necessary for normal progression through the cell cycle. Several inhibitors of cell cycle signalling are under development. For instance, examples of cyclin dependent kinases, including CDK2, CDK4, and CDK6 and inhibitors for the same are described in, for instance, Rosania et al, Exp. Opin. Ther. Patents (2000) 10(2):215-230.

In one embodiment, the cancer treatment method of the claimed invention includes the co-administration a compound of formula I and/or a pharmaceutically acceptable salt, hydrate, solvate or pro-drug thereof and at least one anti-neoplastic agent, such as one selected from the group consisting of anti-microtubule agents, platinum coordination complexes, alkylating agents, antibiotic agents, topoisomerase If inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, and cell cycle signaling inhibitors.

Because the pharmaceutically active compounds of the present invention are active as AKT inhibitors they exhibit therapeutic utility in treating cancer and arthritis.

Suitably, the present invention relates to a method for treating or lessening the severity of a cancer selected from brain (gliomas), glioblastomas, Bannayan-Zonana syndrome, Cowden disease, Lhermifte-Duclos disease, breast, colon, head and neck, kidney, lung, liver, melanoma, ovarian, pancreatic, prostate, sarcoma and thyroid.

Suitably, the present invention relates to a method for treating or lessening the severity of a cancer selected from ovarian, breast, pancreatic and prostate.

### Isolation and Purification of His-Tagged AKT1 (aa 136-480)

Insect cells expressing His-tagged AKT1 (aa 136-480) are lysed in 25 mM HEPES, 100 mM NaCl, 20 mM imidazole; pH 7.5 using a polytron (5 mLs lysis buffer/g cells). Cell debris are removed by centrifuging at 28,000×g for 30 minutes. The supernatant is filtered through a 4.5-micron filter then loaded onto a nickel-chelating column pre-equilibrated with lysis buffer. The column is washed with 5 column volumes (CV) of lysis buffer then with 5 CV of 20% buffer B, where buffer B is 25 mM HEPES, 100 mM NaCl, 300 mM imidazole; pH 7.5. His-tagged AKT1 (aa 136-480) is eluted with a 20-100% linear gradient of buffer B over 10 CV. His-tagged AKT1 (136-480) eluting fractions are pooled and diluted 3-fold with buffer C, where buffer C is 25 mM HEPES, pH 7.5. The sample is then chromatographed over a Q-Sepharose HP column pre-equilibrated with buffer C. The column is washed with 5 CV of buffer C then step eluted with 5 CV 10% D, 5 CV 20% D, 5 CV 30% D, 5 CV 50% D and 5 CV of 100% D; where buffer D is 25 mM HEPES, 1000 mM NaCl; pH 7.5. His-tagged AKT1 (aa 136-480) containing fractions are pooled and concentrated in a 10-kDa molecular weight cutoff concentrator. His-tagged AKT1 (aa 136-480) is chromatographed over a Superdex 75 gel filtration column pre-equilibrated with 25 mM HEPES, 200 mM NaCl, 1 mM DTT; pH 7.5. His-tagged AKT1 (aa 136-480) fractions are examined using SDS-PAGE and mass spec. The protein is pooled, concentrated and frozen at −80 C.

His-tagged AKT2 (aa 138-481) and His-tagged AKT3 (aa 135-479) are isolated and purified in a similar fashion.

### Cloning of Full-Length Human (FL) AKT1:

Full-length human AKT1 gene was amplified by PCR from a plasmid containing myristylated-AKT1-ER (gift from Robert T. Abraham, Duke University under MTA, described in Klippel et al. in Molecular and Cellular Biology 1998 Volume 18 p. 5699) using the 5′ primer: SEQ.ID NO: 1, 5′ TATATAGGATCCATGAGCGACGTGGC 3′ and the 3′ primer: SEQ.ID NO: 2, AAATTTCTCGAGTCAGGCCGTGCTGCTGG 3′. The 5′ primer included a BamHI site and the 3′primer included an XhoI site for cloning purposes. The resultant PCR product was subcloned in pcDNA3 as a BamHI/XhoI fragment. A mutation in the sequence (IGC) coding for a Cysteine²⁵ was converted to the wild-type AKT1 sequence (CGC) coding for an Arginine²⁵ by site-directed mutagenesis using the QuikChange® Site Directed Mutagenesis Kit (Stratagene). The AKT1 mutagenic primer: SEQ.ID NO: 3, 5′ ACCTGGCGGCCACGCTACTTCCTCC and selection primer: SEQ.ID NO: 4, 5′ CTCGAGCATGCAACTAGAGGGCC (designed to destroy an XbaI site in the multiple cloning site of pcDNA3) were used according to manufacturer's suggestions. For expression/purification purposes, AKT1 was isolated as a BamHI/XhoI fragment and cloned into the BamHI/XhoI sites of pFastbacHTb (Invitrogen).

### Expression of FL Human AKT1:

Expression was done using the BAC-to-BAC Baculovirus Expression System from Invitrogen (catalog #10359-016). Briefly 1) the cDNA was transferred from the FastBac vector into bacmid DNA, 2) the bacmid DNA was isolated and used to transfect Sf9 insect cells, 3) the virus was produced in Sf9 cells, 4) T. ni cells were infected with this virus and sen't for purification.

### Purification of FL Human AKT1:

For the purification of full-length AKT1, 130 g sf9 cells (batch # 41646WO2) were resuspended in lysis buffer (buffer A, 1 L, pH 7.5) containing 25 mM HEPES, 100 mM NaCl, and 20 mM imidazole. The cell lysis was carried out by Avestin (2 passes at 15K-20K psi). Cell debris was removed by centrifuging at 16K rpm for 1 hour and the supernatant was batch bound to 10 ml Nickel Sepharose HP beads at 4 C for over night. The beads were then transferred to column and the bound material was eluted with buffer B (25 mM HEPES, 100 mM NaCl, 300 mM imidazole, pH 7.5). AKT eluting fractions were pooled and diluted 3 fold using buffer C (25 mM HEPES, 5 mM DTT; pH 7.5). The sample was filtered and chromatographed over a 10 mL Q-HP column pre-equilibrated with buffer C at 2 mL/min.

The Q-HP column was washed with 3 column volume (CV) of buffer C, then step eluted with 5 CV 10% D, 5 CV 20% D, 5 CV 30% D, 5 CV 50% D and 5 CV of 100% D; where buffer D is 25 mM HEPES, 1000 mM NaCl, 5 mM DTT; pH 7.5. 5 mL fractions collected. AKT containing fractions were pooled and concentrated to 5 ml. The protein was next loaded to a 120 ml Superdex 75 sizing column that was pre-equilibrated with 25 mM HEPES, 200 mM NaCl, 5 mM DTT; pH 7.5.2.5 mL fractions were collected.

AKT 1 eluting fractions were pooled, aliquoted (1 ml) and stored at −80 C. Mass spec and SDS-PAGE analysis were used to confirm purity and identity of the purified full-length AKT1.

Full length AKT2 and full length AKT3 were cloned, expressed and purified in a similar fashion.

### AKT Enzyme Assay

Compounds of the present invention are tested for AKT 1, 2, and 3 protein serine kinase inhibitory activity in substrate phosphorylation assays. This assay examines the ability of small molecule organic compounds to inhibit the serine phosphorylation of a peptide substrate. The substrate phosphorylation assays use the catalytic domains of AKT 1, 2, or 3. AKT 1, 2 and 3 are also commercially available from Upstate USA, Inc. The method measures the ability of the isolated enzyme to catalyze the transfer of the gamma-phosphate from ATP onto the serine residue of a biotinylated synthetic peptide SEQ. ID NO: 5 (Biotin-ahx-ARKRERAYSFGHHA-amide). Substrate phosphorylation is detected by the following procedure:

Assays are performed in 384 well U-bottom white plates. 10 nM activated AKT enzyme is incubated for 40 minutes at room temperature in an assay volume of 20 ul containing 50 mM MOPS, pH 7.5, 20 mM MgCl₂, 4 uM ATP, 8 uM peptide, 0.04 uCi [g-³³P] ATP/well, 1 mM CHAPS, 2 mM DTT, and 1 ul of test compound in 100% DMSO. The reaction is stopped by the addition of 50 ul SPA bead mix (Dulbecco's PBS without Mg²⁺ and Ca²⁺, 0.1% Triton X-100, 5 mM EDTA, 50 uM ATP, 2.5 mg/ml Streptavidin-coated SPA beads.) The plate is sealed, the beads are allowed to settle overnight, and then the plate is counted in a Packard Topcount Microplate Scintillation Counter (Packard Instrument Co., Meriden, Conn.).

The data for dose responses are plotted as % Control calculated with the data reduction formula 100*(U1−C2)/(C1−C2) versus concentration of compound where U is the unknown value, C1 is the average control value obtained for DMSO, and C2 is the average control value obtained for 0.1M EDTA. Data are fitted to the curve described by: y=((Vmax*x)/(K+x)) where Vmax is the upper asymptote and K is the IC₅₀.

Compounds of the invention are tested for activity against AKT1, AKT2, and AKT3 in the above assay.

The compounds of Examples 43, 77, 86, 92, 93, 94, 106, 108, 117, 123, 124, 132, 143, 151 and 152 were tested in the above AKT enzyme assay and each exhibited an IC₅₀ value less than or equal to 0.5 uM against AKT1, AKT2 and AKT3.

The pharmaceutically active compounds within the scope of this invention are useful as AKT inhibitors in mammals, particularly humans, in need thereof.

The present invention therefore provides a method of treating cancer, arthritis and other conditions requiring AKT inhibition, which comprises administering an effective compound of Formula (I) or a pharmaceutically acceptable salt, hydrate, solvate or pro-drug thereof. The compounds of Formula (I) also provide for a method of treating the above indicated disease states because of their demonstrated ability to act as Akt inhibitors. The drug may be administered to a patient in need thereof by any conventional route of administration, including, but not limited to, intravenous, intramuscular, oral, subcutaneous, intradermal, and parenteral.

The pharmaceutically active compounds of the present invention are incorporated into convenient dosage forms such as capsules, tablets, or injectable preparations. Solid or liquid pharmaceutical carriers are employed. Solid carriers include, starch, lactose, calcium sulfate dihydrate, terra alba, sucrose, talc, gelatin, agar, pectin, acacia, magnesium stearate, and stearic acid. Liquid carriers include syrup, peanut oil, olive oil, saline, and water. Similarly, the carrier or diluent may include any prolonged release material, such as glyceryl monostearate or glyceryl distearate, alone or with a wax. The amount of solid carrier varies widely but, suitably, will be from about 25 mg to about 1 g per dosage unit. When a liquid carrier is used, the preparation will be in the form of a syrup, elixir, emulsion, soft gelatin capsule, sterile injectable liquid such as an ampoule, or an aqueous or nonaqueous liquid suspension.

The pharmaceutical preparations are made following conventional techniques of a pharmaceutical chemist involving mixing, granulating, and compressing, when necessary, for tablet forms, or mixing, filling and dissolving the ingredients, as appropriate, to give the desired oral or parenteral products.

Doses of the presently invented pharmaceutically active compounds in a pharmaceutical dosage unit as described above will be an efficacious, nontoxic quantity preferably selected from the range of 0.001-100 mg/kg of active compound, preferably 0.001-50 mg/kg. When treating a human patient in need of an Akt inhibitor, the selected dose is administered preferably from I-6 times daily, orally or parenterally. Preferred forms of parenteral administration include topically, rectally, transdermally, by injection and continuously by infusion. Oral dosage units for human administration preferably contain from 0.05 to 3500 mg of active compound. Oral administration, which uses lower dosages is preferred. Parenteral administration, at high dosages, however, also can be used when safe and convenient for the patient.

Optimal dosages to be administered may be readily determined by those skilled in the art, and will vary with the particular Akt inhibitor in use, the strength of the preparation, the mode of administration, and the advancement of the disease condition. Additional factors depending on the particular patient being treated will result in a need to adjust dosages, including patient age, weight, diet, and time of administration.

The method of this invention of inducing Akt inhibitory activity in mammals, including humans, comprises administering to a subject in need of such activity an effective Akt inhibiting amount of a pharmaceutically active compound of the present invention.

The invention also provides for the use of a compound of Formula (I) in the manufacture of a medicament for use as an Akt inhibitor.

The invention also provides for the use of a compound of Formula (I) in the manufacture of a medicament for use in therapy.

The invention also provides for the use of a compound of Formula (I) in the manufacture of a medicament for use in treating cancer.

The invention also provides for the use of a compound of Formula (I) in the manufacture of a medicament for use in treating arthritis.

The invention also provides for a pharmaceutical composition for use as an Akt inhibitor which comprises a compound of Formula (I) and a pharmaceutically acceptable carrier.

The invention also provides for a pharmaceutical composition for use in the treatment of cancer which comprises a compound of Formula (I) and a pharmaceutically acceptable carrier.

The invention also provides for a pharmaceutical composition for use in treating arthritis which comprises a compound of Formula (I) and a pharmaceutically acceptable carrier.

No unacceptable toxicological effects are expected when compounds of the invention are administered in accordance with the present invention.

In addition, the pharmaceutically active compounds of the present invention can be co-administered with further active ingredients, such as other compounds known to treat cancer or arthritis, or compounds known to have utility when used in combination with an Akt inhibitor.

Without further elaboration, it is believed that one skilled in the art can, using the preceding description, utilize the present invention to its fullest extent. The following Examples are, therefore, to be construed as merely illustrative and not a limitation of the scope of the present invention in any way.

### EXPERIMENTAL DETAILS

The compounds of Examples 1 to 183 are readily made according to Schemes I to IX or by analogous methods.

### Preparation 1

### Preparation of 4-(6-bromo-4-chloro-1-ethyl-1H-imidazo[4,5-c]pyridin-2-yl)-1,2,5-oxadiazol-3-amine

### a) 4-(methyloxy)-5-nitro-2-pyridinol

Into a 1 L flask containing 250 mL of dry THF cooled to −78° C. was condensed NH₃ (100-150 mL). Solid t-BuOK (200 mmole, 22.5 g) was added to the THF and was completely dissolved after 10 min of vigorous stirring. In a separate 500 mL flask, 100 mL of dry THF containing 4-methoxy-3-nitropyridine (12.3 g, 80 mmole) was cooled to 0° C. To this solution was added t-BuOOH (88 mmole, 16 mL). The t-BuOOH/THF solution was then added to the −78° C. ammonia solution over 20 min via dropping funnel. The reaction solution was allowed to warm to −40° C. and then stirred at this temperature for 1 h. The reaction was quenched with saturated NH₄Cl solution (20 mL) and the cooling bath removed. The reaction solution was allowed to stir overnight at RT. The precipitate was filtered and dried under vacuum to give (9.5 g, 70%) of product as a tan solid: LC/MS: m/z 171 [M+H]⁺, single component.

### b) 2,4-dibromo-5-nitropyridine

To a solution of 2-hydroxy-4-methoxy-5-nitropyridine (10 g, 59 mmole) in acetonitrile (60 mL) was added POBr₃ (33.7 g, 117.6 mmole). The reaction mixture was heated to 90° C. for 4 h. The mixture was allowed to cool to RT and then poured onto saturated K₂CO₃/ice water. The product was extracted with EtOAc (2×250 mL), washed with brine and dried over Na₂SO₄. Concentration of the EtOAc solution under vacuum provided a light brown solid (10.9 g, 66%) which was used without further purification: LC/MS: m/z 283 [M+H]⁺, single component.

### c) 2-bromo-N-ethyl-5-nitro-4-pyridinamine

A solution of ethylamine (21 mL, 43 mmol, 2.0 M in MeOH, Aldrich) was added to a solution of 2-bromo-4-bromo-5-nitropyridine (10.9 g, 38.8 mmol) and Et₃N (43 mmole, 6 mL) in THF (100 mL) at 5° C. The addition was mildly exothermic. The resulting yellow solution was stirred at 5° C. for 1 h. TLC analysis indicated that the starting material was consumed (20% EtOAc/hexane, silica gel). The solvent was removed in vacuo and the residue was partitioned between EtOAc (500 mL) and H₂O (50 mL). The organic layer was washed with H₂O (50 mL), brine (50 mL) and dried over Na₂SO₄. The solvent was evaporated to give 9.6 g of the desired material as an orange/yellow oil that solidified under vacuum. This material was used without further purification: LC/MS: m/z 247 [M+H]⁺, single component.

### d) 6-bromo-2-chloro-N⁴-ethyl-3,4-pyridinediamine

A solution of 2-bromo-N-ethyl-5-nitro-4-pyridinamine (9.6 g, 39 mmol) in conc HCl (100 mL) was heated to 90° C. SnCl₂-2H₂O (44 g, 195 mmol, Aldrich) was added portionwise. The resulting mixture was stirred at 90° C. for 45 min. and allowed to cool to RT. The acidic solution was cooled in an ice bath and made basic (pH˜10) with cautious addition of 50% aqueous NaOH. The use of efficient stirring is required. The suspension was extracted with CH₂Cl₂ (3×200 mL) and the combined organic extracts were dried over Na₂SO₄. The solvent was evaporated to give (5.9 g, 60%) of the desired material as a low melting brown solid. This was used without further purification: LC/MS: m/z 251.4 [M+H]⁺, single component.

### e) N-[6-bromo-2-chloro-4-(ethylamino)-3-pyridinyl]-2-cyanoacetamide

To a solution of 6-bromo-2-chloro-N⁴-ethyl-3,4-pyridinediamine (5.8 g, 23.2 mmol) and cyanoacetic acid (4.93 g, 58 mmol) in DMF (150 mL) was added EDC (11.1 g 58 mmol) and N-methylmorpholine (11.5 mL, 104 mmol). After stirring at RT for 16 h, the solvent was removed under reduced pressure. The resulting residue was partitioned between EtOAc (500 mL) and H₂O (50 mL). The organic layer was washed with 5% NaHCO₃ (50 mL), H₂O (50 mL), brine (50 mL) and dried over Na₂SO₄. The solvent was evaporated to give (7.4 g, quant) of the desired material as a beige solid. LC/MS: m/z 317.0 [M+H]⁺, single component

### f) (2E)-(6-bromo-4-chloro-1-ethyl-1H-imidazo[4,5-c]pyridin-2-yl)(hydroxyimino)ethanenitrile

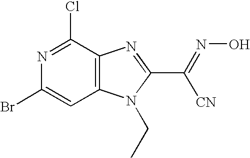

A mixture of N-[6-bromo-2-chloro-4-(ethylamino)-3-pyridinyl]-2-cyanoacetamide (7.3 g, 23.0 mmol) and glacial acetic acid (85 mL) was stirred at 100° C. After 24 h, LC/MS analysis indicated that a single major component consistent with (6-bromo-4-chloro-1-ethyl-1H-imidazo[4,5-c]pyridin-2-yl)acetonitrile was present (m/z 299.0 [M+H]⁺). The reaction mixture was allowed to cool to RT and NaNO₂ (3.65 g, 52.9 mmol) was added in one portion. A vigorous evolution of NO₂ was noted. After 16 h at RT, the resulting suspension was collected by filtration. The solid was dried to a constant weight under vacuum (50° C. @ 1 mbar for 3-4 h) to give 6.4 g of the desired material. This material was used without further purification.

LC/MS: m/z 328.2 [M+H]⁺

### g) 4-(6-bromo-4-chloro-1-ethyl-1H-imidazo[4,5-c]pyridin-2-yl)-1,2,5-oxadiazol-3-amine

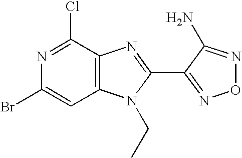

A mixture of (2E)-(6-bromo-4-chloro-1-ethyl-1H-imidazo[4,5-c]pyridin-2-yl)(hydroxyimino)ethanenitrile (3.8 g, 11.6 mmol), 50% hydroxylamine (17.5 mmol, 0.53 mL), Et₃N (9 mL) and dioxane (75 mL) was heated to 100° C. in sealed flask for 16 h. The reaction mixture was filtered while warm (˜50° C.) and the filtrate concentrated in vacuo. The residue was triturated with 95/5 DCM/MeOH (100 mL) and the solid product (2.0 g, 51%) filtered off as a yellow solid: LC/MS: m/z 343.0 [M+H]⁺

### Preparation 2

### Preparation of 4-(4,6-dichloro-1-ethyl-1H-imidazo[4,5-c]pyridin-2-yl)-1,2,5-oxadiazol-3-amine

### a) 4-(ethyloxy)-5-nitro-2-pyridinol

Into a 1 L flask containing 250 mL of dry THF cooled to −78° C. was condensed NH₃ (100-150 mL). Neat t-BuOK (182 mmol, 20.5 g) was added to the THF which completely dissolved after 10 min of vigorous stirring. In a separate 500 mL flask, 100 mL of dry THF containing 4-(ethyloxy)-3-nitropyridine (12.3 g, 72.9 mmol) was cooled to 0° C. To this solution was added t-BuOOH (80.2 mmole, 14 mL). The t-BuOOH/THF solution was then added to the −78° C. ammonia solution over 20 min via dropping funnel. The reaction solution was allowed to warm to −40° C. and then stirred at this temperature for 1 h. The reaction was quenched with saturated NH₄Cl solution (20 mL) and the cooling bath removed. The reaction solution was allowed to stir overnight at RT. The precipitate was filtered and dried under vacuum using a toluene azeotrope: LCMS: m/z 185 [M+H]⁺.

### b) 2,4-dichloro-5-nitropyridine

A solution of 4-(ethyloxy)-5-nitro-2-pyridinol (8.7 g, 47 mmol) in POCl₃ (79 mL) was heated in a sealed tube at 100° C. for 12 h. The excess POCl₃ was removed in vacuo and the residue neutralized by dropwise addition of a saturated solution of NaHCO₃. The resultant aqueous phase was extracted several times with EtOAc and the combined organic fractions were dried over Na₂SO₄ and concentrated affording a yellow solid (6 g, 66%) which was used directly without further purification: LC/MS: m/z 194 [M+H]⁺.

### c) 2-chloro-N-ethyl-5-nitro-4-pyridinamine

A solution of ethylamine (19 mL, 33.6 mmol, 1.7 M in MeOH) was added dropwise to a solution of 2,4-dichloro-5-nitropyridine (5.4 g, 27.9 mmol) and Et₃N (5.1 mL, 36.4 mmol) in THF (22 mL) at 0° C. After 2 h, the solution was partitioned between H₂O-EtOAc. The aqueous phase was extracted several times with EtOAc and the combined organic fractions were dried over Na2SO4 and concentrated affording a yellow solid (5.4 g, 96%) which was used without further purification: LC/MS: m/z 202 [M+H]⁺.

### d) 2,6-dichloro-N⁴-ethyl-3,4-pyridinediamine

A solution of 2-chloro-N-ethyl-5-nitro-4-pyridinamine (5.4 g, 26.9 mmol) in conc HCl (77 mL) was heated to 90° C. SnCl₂ (30 g, 132 mmol, Aldrich) was added portionwise. The resulting mixture was stirred at 90° C. for 45 min. and allowed to cool to RT. The acidic solution was cooled in an ice bath and made alkaline (pH˜10) with addition of 6N NaOH. The suspension was extracted several times with EtOAc and the combined organic extracts were dried over Na₂SO₄ and concentrated yielding the title compound (4.8 g, 87%) as a brown solid which was used without further purification: LC/MS: m/z 207 [M+H]⁺.

### e) 2-cyano-N-[2,6-dichloro-4-(ethylamino)-3-pyridinyl]acetamide

To a solution of 2,6-dichloro-N⁴-ethyl-3,4-pyridinediamine (4.3 g, 20.9 mmol) and cyanoacetic acid (4.4 g, 52.2 mmol) in DMF (200 mL) at 25° C. were added EDC (10 g, 52.2 mmol) and N-methylmorpholine (10 mL, 93.9 mmol). After 12 h, the solvent was removed in vacuo and the residue partitioned between H₂O-EtOAc. The aqueous phase was extracted several times with EtOAc and the combined organic fractions were dried over Na₂SO₄ and concentrated affording the title compound (5.7 g, quant.) as a yellow solid which was used without further purification: LC/MS: m/z 274 [M+H]⁺.

### f) (2E)-(4,6-dichloro-1-ethyl-1H-imidazo[4,5-c]pyridin-2-yl)(hydroxyimino)ethanenitrile

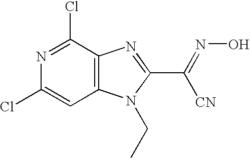

A mixture of 2-cyano-N-[2,6-dichloro-4-(ethylamino)-3-pyridinyl]acetamide (5.7 g, 20.9 mmol) and glacial acetic acid (77 mL) was stirred at 100° C. After 24 h, the reaction mixture was allowed to cool to RT and NaNO₂ (3.3 g, 48 mmol) was added in one portion, whereupon a vigorous evolution of NO₂ was noted. After 16 h at RT, the resulting suspension was collected by filtration. The solid was dried to a constant weight under vacuum (50° C. @ 1 mbar for 3-4 h) affording the title compound (5 g, 84%) as a yellow solid which was used without further purification: LC/MS: m/z 285 [M+H]⁺

### g) 4-(4,6-dichloro-1-ethyl-1H-imidazo[4,5-c]pyridin-2-yl)-1,2,5-oxadiazol-3-amine

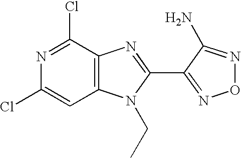

A mixture of (2E)-(4,6-dichloro-1-ethyl-1H-imidazo[4,5-c]pyridin-2-yl)(hydroxyimino)ethanenitrile (5.3 g, 18.7 mmol), hydroxylamine hydrochloride (1.95 g, 27.9 mmol), Et₃N (13 mL, 93.3) and dioxane (120 mL) was heated to 100° C. in sealed flask for 12 h. The reaction mixture was filtered and the filtrate concentrated. The residue was triturated with 3% MeOH in DCM affording the title compound (3.1 g, 55%) as a yellow solid: LC/MS: m/z 299 [M+H]⁺, ¹H NMR (CD₃)₂SO, 400 MHz) δ 8.23 (s, 1H), 6.94 (bs, 2H), 4.68 (q, J=7.13 Hz, 2H), 1.39 (t, J=7.10 Hz, 3H).

### Example 1

### Preparation of 44′-[2-(4-amino-1,2,5-oxadiazol-3-yl)-1-ethyl-1H-imidazo[4,5-c]pyridine-4,6-diyl]bis(2-methyl-3-butyn-2-ol)

To a solution of 4-(6-bromo-4-chloro-1-ethyl-1H-imidazo[4,5-c]pyridin-2-yl)-1,2,5-oxadiazol-3-amine (0.25 g, 0.73 mmole) in DMF (2 mL) was added CuI (7 mg, 0.04 mmole), 2-hydroxy-2-methyl-3-butyne (70 mg, 0.88 mmole), triethylamine (0.20 mL, 1.46 mmole) and dichlorobistriphenylphosphine palladium (II) (51 mg, 0.07 mmole). The reaction was heated to 80° C. in a sealed tube for 4 h. The reaction solution was concentrated under vacuum and purified on silica gel (hexanes/EtOAc, 1:1) to give the title compound (200 mg, 68%) as a yellow solid: LC-MS (ES) m/z=395 (M+H)⁺. ¹H NMR (CD₃OD, 400 MHz) δ 8.49 (s, 1H), 4.91 (q, J=6.3 Hz, 2H), 1.7 (s, 6H), 1.67 (s, 6H), 1.55 (t, J=6.3 Hz, 3H).

The purified compound was converted into its corresponding HCl salt by dissolving the free base material in MeOH, adding 4M HCl in dioxane, and concentrating under vacuum.

### Example 2

### Preparation of 4-[2-(4-amino-1,2,5-oxadiazol-3-yl)-6-(3-aminophenyl)-1-ethyl-1H-imidazo[4,5-c]pyridin-4-yl]-2-methyl-3-butyn-2-ol

### a) 4-[6-(3-aminophenyl)-4-chloro-1-ethyl-1H-imidazo[4,5-c]pyridin-2-yl]-1,2,5-oxadiazol-3-amine

A mixture of dioxane (5 mL) and 2M K₂CO₃ (0.90 mL) was deoxygenated by purging with nitrogen. To this solution was added 4-(6-bromo-4-chloro-1-ethyl-1H-imidazo[4,5-c]pyridin-2-yl)-1,2,5-oxadiazol-3-amine (200 mg, 0.58 mmol), 3-aminophenylboronic acid (110 mg, 0.70 mmol), and tetrakis(triphenylphosphine)palladium (33 mg, 0.03 mmol) and the mixture was heated to 70° C. for 20 h under an atmosphere of N₂. After cooling to RT, the reaction was concentrated in vacuo. Flash chromatography (silica gel, MeOH/CH₂Cl₂ gradient) gave the title compound (175 mg, 85%). LCMS (ES) m/z 356 (M+H)⁺.

### b) 4-[2-(4-amino-1,2,5-oxadiazol-3-yl)-6-(3-aminophenyl)-1-ethyl-1H-imidazo[4,5-c]pyridin-4-yl]-2-methyl-3-butyn-2-ol

To a solution of 4-[6-(3-aminophenyl)-4-chloro-1-ethyl-1H-imidazo[4,5-c]pyridin-2-yl]-1,2,5-oxadiazol-3-amine (0.17 g, 0.50 mmole) in DMF (2 mL) was added CuI (5 mg, 0.02 mmole), 2-hydroxy-2-methyl-3-butyne (0.11 g, 1.25 mmole), triethylamine (0.14 mL, 1.0 mmole) and dichlorobistriphenylphosphine palladium (II) (35 mg, 0.05 mmole). The reaction was heated to 80° C. in a sealed tube for 2 h. The reaction solution was concentrated under vacuum and purified on silica gel (hexanes/EtOAc, 1:1) to give the title compound (0.15 mg, 75%) as a brown solid: LC-MS (ES) m/z=404 (M+H)⁺. ¹H NMR (d6-DMSO, 400 MHz) δ 8.57 (s, 1H), 8.28 (m, 1H), 7.65 (t, J=7.3 Hz, 1H), 7.49 (d, J=7.2 Hz, 1H), 4.30 (q, J=7.0 Hz, 2H), 1.61 (s, 6H), 1.45 (t, J=7.0 Hz, 3H).

The purified compound was converted into its corresponding HCl salt by dissolving the free base material in MeOH, adding 4M HCl in dioxane, and concentrating under vacuum.

### Example 3

### Preparation of 4-[2-(4-amino-1,2,5-oxadiazol-3-yl)-6-(2-aminophenyl)-1-ethyl-1H-imidazo[4,5-c]pyridin-4-yl]-2-methyl-3-butyn-2-ol

The title compound (80 mg, 58%) was prepared as a yellow solid according to the preparation of Example 2, except substituting 2-aminophenylboronic acid (110 mg, 0.70 mmol) for 3-aminophenylboronic acid to afford: LCMS (ES) m/e 356 (M+H)⁺; ¹H NMR (CD₃OD, 400 MHz) δ 7.82 (s, 1H), 7.50 (d, J=7.6 Hz, 1H), 7.16 (t, J=7.3 Hz, 1H), 6.85 (d, J=8.0 Hz, 1H), 6.79 (t, J=7.3 Hz, 2H), 4.78 (q, J=7.0 Hz, 2H), 1.68 (s, 6H), 1.48 (t, J=7.0 Hz, 3H).

### Example 4

### Preparation of 4-[6-[3-(aminomethyl)phenyl]-2-(4-amino-1,2,5-oxadiazol-3-yl)-1-ethyl-1H-imidazo[4,5-c]pyridin-4-yl]-2-methyl-3-butyn-2-ol

The title compound was prepared as a tan solid according to the preparation of Example 2, except substituting 3-aminomethylphenylboronic acid (66 mg, 0.35 mmol) for 3-aminophenylboronic acid to afford: LCMS (ES) m/e 418 (M+H)⁺; ¹H NMR (d6-DMSO, 400 MHz) δ 8.57 (s, 1H), 8.45 (m, 1H), 8.22 (m, 1H), 7.59 (m, 1H), 4.79 (q, J=7.0 Hz, 2H), 4.13 (m, 2H), 1.61 (s, 6H), 1.45 (t, J=7.0 Hz, 3H).

### Example 5

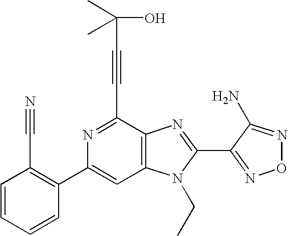

### Preparation of 2-[2-(4-amino-1,2,5-oxadiazol-3-yl)-1-ethyl-4-(3-hydroxy-3-methyl-1-butyn-1-yl)-1H-imidazo[4,5-c]pyridin-6-yl]benzonitrile

The title compound (112 mg, 46%) was prepared as a brown solid according to Example 2, except substituting (2-cyanophenyl)boronic acid (131 mg, 0.892 mmol) for 3-aminophenylboronic acid: LCMS (ES) m/e 414 (M+H)⁺; ¹H NMR (CD₃OD, 400 MHz) δ 7.81 (s, 1H), 7.52-7.65 (m, 4H), 4.78 (q, J=7.2 Hz, 2H), 1.50 (t, J=7.2 Hz, 3H).

### Example 6

### Preparation of 4-{2-(4-amino-1,2,5-oxadiazol-3-yl)-1-ethyl-6-[2-(hydroxymethyl)phenyl]-1H-imidazo[4,5-c]pyridin-4-yl}-2-methyl-3-butyn-2-ol

The title compound (390 mg, 72%) was prepared as a brown solid according to Example 2, except substituting 2-(bromomethyl)phenylboronic acid (340 mg, 1.56 mmol) for 3-aminophenylboronic acid: LCMS (ES) m/e 419 (M+H)⁺; LCMS (ES) m/e 356 (M+H)⁺; ¹H NMR (CD₃OD, 400 MHz) δ 8.01 (s, 1H), 7.66 (m, 2H), 7.60 (m, 1H), 7.49 (m, 1H), 4.87 (q, J=7.0 Hz, 2H), 1.70 (s, 6H), 1.53 (t, J=7.0 Hz, 3H).

### Example 7

### Preparation of N-{4-[2-(4-amino-1,2,5-oxadiazol-3-yl)-1-ethyl-4-(3-hydroxy-3-methyl-1-butyn-1-yl)-1H-imidazo[4,5-c]pyridin-6-yl]phenyl}acetamide

The title compound (14.7 mg, 30%) was prepared as an off-white solid according to Example 2, except substituting N-[4-(4,4,5,5-tetramethyl-1,3,2-dioxaborolan-2-yl)phenyl]acetamide (129 mg, 0.64 mmol) for 3-aminophenylboronic acid: LCMS (ES) m/z 446 (M+H)⁺. ¹H NMR (CD₃OD, 400 MHz) δ 8.17 (s, 1H), 8.02 (d, J=8.8 Hz, 2H), 7.75 (d, J=8.7 Hz, 2H), 4.91-4.98 (m, 2H), 2.19 (s, 3H), 1.72 (bs, 6H), 1.54 (t, 3H).

### Example 8

### Preparation of 4-[2-(4-amino-1,2,5-oxadiazol-3-yl)-1-ethyl-6-(1H-indol-5-yl)-1H-imidazo[4,5-c]pyridin-4-yl]-2-methyl-3-butyn-2-ol

The title compound (15.5 mg, 35%) was prepared as a brown solid according to Example 2, except substituting 5-(4,4,5,5-tetramethyl-1,3,2-dioxaborolan-2-yl)-1H-indole (103 mg, 0.64 mmol) for 3-aminophenylboronic acid and adopting Gilson reverse column (0-80% MeOH/H₂O with 1% TFA) for purification: LCMS (ES) m/z 428 (M+H)⁺. ¹H NMR (CD₃OD, 400 MHz) δ 8.34 (s, 1H), 8.20 (s, 1H), 7.72 (d, J=8.0 Hz, 1H), 7.62 (d, J=8.5 Hz, 1H), 7.40-7.42 (m, 1H), 6.64-6.66 (m, 1H), 4.90-4.94 (m, 2H), 1.74 (bs, 6H), 1.58 (t, 3H).

### Example 9

### Preparation of N-{3-[2-(4-amino-1,2,5-oxadiazol-3-yl)-1-ethyl-4-(3-hydroxy-3-methyl-1-butyn-1-yl)-1H-imidazo[4,5-c]pyridin-6-yl]phenyl}acetamide

The title compound (58.3 mg, 30%) was prepared as a white solid according to Example 2, except substituting N-[3-(4,4,5,5-tetramethyl-1,3,2-dioxaborolan-2-yl)phenyl]acetamide (93.1 mg, 0.52 mmol) for 3-aminophenylboronic acid: LCMS (ES) m/z 446 (M+H)⁺. ¹H NMR (CD₃OD, 400 MHz) δ 8.26 (s, 1H), 8.10 (s, 1H), 7.77-7.82 (m, 1H), 7.64-7.66 (m, 1H), 7.44-7.48 (m, 1H), 4.90-4.94 (m, 2H), 2.20 (s, 3H), 1.71 (bs, 6H), 1.53 (t, 3H).

### Example 10

### Preparation of 4-[6-[3-(aminomethyl)phenyl]-1-ethyl-4-(1H-pyrrol-3-yl)-1H-imidazo[4,5-c]pyridin-2-yl]-1,2,5-oxadiazol-3-amine

### a) 4-{6-[3-(aminomethyl)phenyl]-4-chloro-1-ethyl-1H-imidazo[4,5-c]pyridin-2-yl}-1,2,5-oxadiazol-3-amine

A solution of 4-(6-bromo-4-chloro-1-ethyl-1H-imidazo[4,5-c]pyridin-2-yl)-1,2,5-oxadiazol-3-amine (255 mg, 0.742 mmol), [3-(aminomethyl)phenyl]boronic acid hydrochloride (170 mg, 0.907 mmol), K₂CO₃ (328 mg, 2.37 mmol) and tetrakis(triphenylphosphine)palladium (76 mg, 66 μmol) in dioxane/H₂O (12 mL, 5:1) was deoxygenated by purging with nitrogen then heated to 70° C. over 12 h. This solution was then concentrated and purified via column chromatography (5% MeOH in DCM (0.5% NH₄OH)) yielding the title compound (114 mg, 42%) as a yellow solid: LCMS (ES) m/z 370 (M+H)⁺.

### b) 4-(6-[3-(aminomethyl)phenyl]-1-ethyl-4-{1-[tris(1-methylethyl)silyl]-1H-pyrrol-3-yl}-1H-imidazo[4,5-c]pyridin-2-yl)-1,2,5-oxadiazol-3-amine

A solution of 4-{6-[3-(aminomethyl)phenyl]-4-chloro-1-ethyl-1H-imidazo[4,5-c]pyridin-2-yl}-1,2,5-oxadiazol-3-amine (114 mg, 0.308 mmol), {1-[tris(1-methylethyl)silyl]-1H-pyrrol-3-yl}boronic acid (100 mg, 0.374 mmol), K₂CO₃ (128 mg, 0.926 mmol) and tetrakis(triphenylphosphine)palladium (27 mg, 23.4 μmol) in dioxane/H₂O (12 mL, 5:1) was deoxygenated by purging with nitrogen then heated to 70° C. over 12 h. This solution was then concentrated and purified via column chromatography (5% MeOH in DCM (0.5% NH₄OH)) yielding the title compound (115 mg, 67%) as a yellow solid: LCMS (ES) m/z 557 (M+H)⁺.

### c) 4-[6-[3-(aminomethyl)phenyl]-1-ethyl-4-(1H-pyrrol-3-yl)-1H-imidazo[4,5-c]pyridin-2-yl]-1,2,5-oxadiazol-3-amine

To a solution of 4-(6-[3-(aminomethyl)phenyl]-1-ethyl-4-{1-[tris(1-methylethyl)silyl]-1H-pyrrol-3-yl}-1H-imidazo[4,5-c]pyridin-2-yl)-1,2,5-oxadiazol-3-amine (115 mg, 0.207 mmol) in THF (20 mL) at 25° C. was added TBAF (0.5 mL) dropwise. After 2 h, the solution was concentrated and purified via column chromatography (silica, 10% MeOH—CHCl₃ (0.5% NH₄OH) yielding the title compound (75 mg, 91%) as a yellow solid: LCMS (ES) m/z 401 (M+H)⁺. ¹H NMR (CD₃)₂SO), 400 MHz) δ 11.29 (s, 1H), 8.29 (s, 1H), 8.19 (d, J=7.7 Hz, 1H), 8.15 (s, 1H), 7.99 (s, 1H), 7.46 (t, J=7.5 Hz, 1H), 7.40 (d, J=7.6 Hz, 1H), 7.15 (d, J=2.5 Hz, 1H), 7.02 (bs, 2H), 6.95 (d, J=3.1 Hz, 1H), 4.79 (q, J=7.6 Hz, 2H), 3.87 (s, 2H), 1.46 (t, J=7.5 Hz, 3H).

### Example 11

### Preparation of N¹-{3-[2-(4-amino-1,2,5-oxadiazol-3-yl)-1-ethyl-4-(3-hydroxy-3-methyl-1-butyn-1-yl)-1H-imidazo[4,5-c]pyridin-6-yl]phenyl}glycinamide

### a) 4-[2-(4-amino-1,2,5-oxadiazol-3-yl)-6-chloro-1-ethyl-1H-imidazo[4,5-c]pyridin-4-yl]-2-methyl-3-butyn-2-ol

To a solution of 4-(4,6-dichloro-1-ethyl-1H-imidazo[4,5-c]pyridin-2-yl)-1,2,5-oxadiazol-3-amine (1 g, 3.34 mmol) in DMF/Et₃N (33 mL, 2:1) was added CuI (64 mg, 0.334 mmol), 2-hydroxy-2-methyl-3-butyne (392 μL, 4.01 mmol) and dichlorobistriphenylphosphine palladium (II) (235 mg, 0.334 mmol). The reaction was heated to 70° C. in a sealed tube over 12 h. The solution was concentrated under vacuum and purified on silica gel (2% MeOH in DCM) affording the title compound (850 mg, 73%) as a brown solid: LC-MS (ES) m/z=347 (M+H)⁺.

### b) 4-[2-(4-amino-1,2,5-oxadiazol-3-yl)-6-(3-aminophenyl)-1-ethyl-1H-imidazo[4,5-c]pyridin-4-yl]-2-methyl-3-butyn-2-ol

A solution of 4-[2-(4-amino-1,2,5-oxadiazol-3-yl)-6-chloro-1-ethyl-1H-imidazo[4,5-c]pyridin-4-yl]-2-methyl-3-butyn-2-ol (100 mg, 0.289 mmol), 3-aminophenylboronic acid (54 mg, 0.347 mmol), K₂CO₃ (159 mg, 1.16 mmol) and tetrakis(triphenylphosphine)palladium (17 mg, 14.5 mmol) in dioxane/H₂O (2.8 mL, 5:1) was deoxygenated by purging with nitrogen then heated to 70° C. over 12 h. This solution was then concentrated and purified via column chromatography (2% MeOH in DCM) yielding the title compound (72 mg, 62%) as a yellow solid which was identical in all respects to that prepared in Example 2: LCMS (ES) m/z 404 (M+H)⁺.

### c) 1,1-dimethylethyl[2-({3-[2-(4-amino-1,2,5-oxadiazol-3-yl)-1-ethyl-4-(3-hydroxy-3-methyl-1-butyn-1-yl)-1H-imidazo[4,5-c]pyridin-6-yl]phenyl}amino)-2-oxoethyl]carbamate

A solution of 4-[2-(4-amino-1,2,5-oxadiazol-3-yl)-6-(3-aminophenyl)-1-ethyl-1H-imidazo[4,5-c]pyridin-4-yl]-2-methyl-3-butyn-2-ol (93 mg, 0.231 mmol), N-{[(1,1-dimethylethyl)oxy]carbonyl}glycine (49 mg, 0.277 mmol), EDC (53 mg, 0.277 mmol) and 4-methylmorpholine (51 μL, 0.462 mmol) in DMF (1 mL) were stirred at 25° C. over 12 h. The solution was concentrated and the residue purified via column chromatography (1.5-3% MeOH in DCM (1% NH4OH)) yielding the title compound (93 mg, 72%) as a yellow oil: LCMS (ES) m/z 561 (M+H)⁺.

### d) N¹-{3-[2-(4-amino-1,2,5-oxadiazol-3-yl)-1-ethyl-4-(3-hydroxy-3-methyl-1-butyn-1-yl)-1H-imidazo[4,5-c]pyridin-6-yl]phenyl}glycinamide

A solution of 1,1-dimethylethyl[2-({3-[2-(4-amino-1,2,5-oxadiazol-3-yl)-1-ethyl-4-(3-hydroxy-3-methyl-1-butyn-1-yl)-1H-imidazo[4,5-c]pyridin-6-yl]phenyl}amino)-2-oxoethyl]carbamate (93 mg, 0.166 mmol) in DCE-TFA (2 mL, 4:1) was stirred at ambient temperature over 30 min. The resulting solution was concentrated using a toluene azeotrope affording the title compound as the di-TFA salt (98 mg, 81%) as a beige solid: LCMS (ES) m/z 461 (M+H)⁺. ¹H NMR ((CD₃)₂SO, 400 MHz) δ10.63 (s, 1H), 8.46 (s, 1H), 8.36 (s, 1H), 8.15 (bs, 2H), 7.96 (d, J=7.98 Hz, 1H), 7.83 (d, J=9.43, 1H), 7.54 (dd, J=7.96, 7.96, 1H), 7.09 (bs, 2H), 4.80 (q, J=7.05, 2H), 3.83 (bs, 2H), 2.51 (s, 6H), 1.44 (t, J=7.05, 3H).

### Example 12

### Preparation of 4-[2-(4-amino-1,2,5-oxadiazol-3-yl)-6-(4-aminophenyl)-1-ethyl-1H-imidazo[4,5-c]pyridin-4-yl]-2-methyl-3-butyn-2-ol

The title compound (44 mg, 23%) was prepared as a brown solid according to Example 11, except substituting (4-Boc-aminophenyl)boronic acid (74 mg, 0.312 mmol) for 3-aminophenylboronic acid: LCMS (ES) m/z 404 (M+H)⁺. ¹H NMR ((CD₃)₂SO, 400 MHz) δ 8.32 (s, 1H), 8.07 (d, J=8.60 Hz, 2H), 7.08 (bs, 2H), 6.96 (d, J=8.36 Hz, 2H), 4.77 (q, J=7.12 Hz, 2H), 1.55 (s, 6H), 1.42 (t, J=7.01 Hz, 3H).

### Example 13

### Preparation of 3-[2-(4-amino-1,2,5-oxadiazol-3-yl)-1-ethyl-4-(3-hydroxy-3-methyl-1-butyn-1-yl)-1H-imidazo[4,5-c]pyridin-6-yl]phenol

The title compound (230 mg, 44%) was prepared as a yellow solid according to Example 11, except substituting 3-(4,4,5,5-tetramethyl-1,3,2-dioxaborolan-2-yl)phenol (343 mg, 1.56 mmol) for 3-aminophenylboronic acid: LCMS (ES) m/z 405 (M+H)⁺; ¹H NMR ((CD₃)₂SO, 400 MHz) δ 9.58 (s, 1H), 8.41 (s, 1H), 7.72 (s, 1H), 7.64 (d, J=7.31 Hz, 1H), 7.31 (dd, J=7.25, 7.25 Hz, 1H), 7.08 (bs, 2H), 6.87 (d, J=7.45 Hz, 1H), 4.12 (q, J=7.12 Hz, 2H), 1.58 (s, 6H), 1.43 (t, 7.02 Hz, 3H).

### Example 14

### Preparation of 4-(2-(4-amino-1,2,5-oxadiazol-3-yl)-6-{3-[(3-aminopropyl)oxy]phenyl}-1-ethyl-1H-imidazo[4,5-c]pyridin-4-yl)-2-methyl-3-butyn-2-ol

### a) 3-({[(1,1-dimethylethyl)oxy]carbonyl}amino)propyl 4-methylbenzenesulfonate

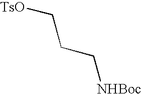

To a solution of hydroxypropyl Boc-carbamate (976 μL, 5.7 mmol) in DCM (57 mL) and triethyl amine (954 μL, 6.85 mmol) at 25° C. were added tosyl chloride (1.3 g, 6.85 mmol) and DMAP (70 mg, 0.571 mmol). After 12 h, the solution was concentrated and purified via companion (dry load, 1-50% ethyl acetate:hexane) affording the title compound (1.4 g, 75%) as a white solid: LCMS (ES) m/z 330 (M+H)⁺.

### b) 1,1-dimethylethyl[3-({3-[2-(4-amino-1,2,5-oxadiazol-3-yl)-1-ethyl-4-(3-hydroxy-3-methyl-1-butyn-1-yl)-1H-imidazo[4,5-c]pyridin-6-yl]phenyl}oxy)propyl]carbamate

A solution of 3-[2-(4-amino-1,2,5-oxadiazol-3-yl)-1-ethyl-4-(3-hydroxy-3-methyl-1-butyn-1-yl)-1H-imidazo[4,5-c]pyridin-6-yl]phenol (90 mg, 0.223 mmol), 3-({[(1,1-dimethylethyl)oxy]carbonyl}amino)propyl 4-methylbenzenesulfonate (88 mg, 0.267 mmol) and cesium carbonate (109 mg, 0.334 mmol) in DMF (1 mL) were heated to 75° C. in a sealed tube over 2 h. The resulting solution was concentrated and the residue purified via column chromatography (silica, 1-2% MeOH in DCM) affording the title compound (51 mg, 41%) as a yellow foam: LCMS (ES) m/z 562 (M+H)⁺.

### c) 4-(2-(4-amino-1,2,5-oxadiazol-3-yl)-6-{3-[(3-aminopropyl)oxy]phenyl}-1-ethyl-1H-imidazo[4,5-c]pyridin-4-yl)-2-methyl-3-butyn-2-ol (di-TFA salt, GSK834047)

A solution of 1,1-dimethylethyl[3-({3-[2-(4-amino-1,2,5-oxadiazol-3-yl)-1-ethyl-4-(3-hydroxy-3-methyl-1-butyn-1-yl)-1H-imidazo[4,5-c]pyridin-6-yl]phenyl}oxy)propyl]carbamate (51 mg, 90.9 mmol) in DCM (1 mL) and TFA (200 μL) was stirred at ambient temperature. After 30 min, the solution was concentrated using a toluene azeotrope affording the title compound (51 mg, 81%) as a tan solid: LCMS (ES) m/z 462 (M+H)⁺; ¹H NMR ((CD₃)₂SO, 400 MHz) δ 8.48 (s, 1H), 7.83 (d, J=8.20 Hz, 1H), 7.79 (s, 1H), 7.78 (bs, 2H), 7.46 (dd, J=7.99, 7.99 Hz, 1H), 7.08 (bs, 2H), 7.04 (d, J=6.03 Hz, 1H), 4.80 (q, J=7.13 Hz, 2H), 4.19 (t, J=6.0 Hz, 2H), 3.03 (m, 2H), 2.07 (t, 7.24 Hz, 2H), 1.59 (s, 6H), 1.44 (t, J=7.03 Hz, 3H).

### Example 15

### Preparation of 4-[6-{3-[(2-aminoethyl)oxy]phenyl}-2-(4-amino-1,2,5-oxadiazol-3-yl)-1-ethyl-1H-imidazo[4,5-c]pyridin-4-yl]-2-methyl-3-butyn-2-ol

The title compound (30 mg, 87%) was prepared as a brown solid according to Example 14, except substituting 2-({[(1,1-dimethylethyl)oxy]carbonyl}amino)ethyl 4-methylbenzenesulfonate (80 mg, 0.252 mmol) for 3-({[(1,1-dimethylethyl)oxy]carbonyl}amino)propyl 4-methylbenzenesulfonate: LCMS (ES) m/z 448 (M+H)⁺; ¹H NMR ((CD₃)₂SO, 400 MHz) δ 8.49 (s, 1H), 8.02 (bs, 2H), 7.87 (s, 1H), 7.86 (d, J=7.42, 1H), 7.49 (dd, J=8.26, 8.26 Hz, 1H), 7.10 (d, J=7.23 Hz, 1H), 7.09 (bs, 2H), 4.80 (q, J=7.11 Hz, 2H), 4.28 (t, J=5.12 Hz, 2H), 3.29 (t, J=4.81 Hz, 2H), 1.59 (s, 6H), 1.44 (t, J=7.06 Hz, 3H).

### Example 16

### Preparation of 2-[2-(4-amino-1,2,5-oxadiazol-3-yl)-1-ethyl-4-(3-hydroxy-3-methyl-1-butyn-1-yl)-1H-imidazo[4,5-c]pyridin-6-yl]phenol

The title compound (47 mg, 9%) was prepared as an orange solid according to Example 11, except substituting 2-(4,4,5,5-tetramethyl-1,3,2-dioxaborolan-2-yl)phenol (343 mg, 1.56 mmol) for 3-aminophenylboronic acid: LCMS (ES) m/z 405 (M+H)⁺; ¹H NMR ((CD₃)₂SO, 400 MHz) δ 8.70 (s, 1H), 8.25 (d, J=7.42 Hz, 1H), 7.36 (dd, J=7.41, 7.34 Hz, 1H), 7.06 (bs, 2H), 7.01 (d, J=7.40 Hz, 1H), 6.85 (dd, J=7.20, 7.40 Hz, 1H), 4.83 (q, J=7.21 Hz, 2H), 1.59 (s, 6H), 1.45 (t, J=7.0 Hz, 3H).

### Example 17

### Preparation of 4-[6-[(4-aminobutyl)oxy]-2-(4-amino-1,2,5-oxadiazol-3-yl)-1-ethyl-1H-imidazo[4,5-c]pyridin-4-yl]-2-methyl-3-butyn-2-ol

### a) 1,1-dimethylethyl (4-hydroxybutyl)carbamate

To a solution of 4-amino-1-butanol (2.0 g, 22.5 mmole) in THF at RT was added Boc anhydride (4.90 g, 22.5 mmole). After 3 h, the reaction solution was concentrated under vacuum and the residue purified on silica gel (hexanes/EtOAc, 1:1) to give the title compound (quant.) as a white solid: LCMS (ES) m/z=190 (M+H)⁺

### b) 2-(4-amino-1,2,5-oxadiazol-3-yl)-4-chloro-1-ethyl-1,5-dihydro-6H-imidazo[4,5-c]pyridin-6-one

To a solution of 4-(6-bromo-4-chloro-1-ethyl-1H-imidazo[4,5-c]pyridin-2-yl)-1,2,5-oxadiazol-3-amine (1.05 g, 3.06 mmol) in THF (130 mL) at −100° C. under an atmosphere of nitrogen was added trimethyl borate (1.14 mL, 10.1 mmol). After 5 minutes with stirring, n-Butyl lithium (3.9 mL, 9.8 mmol, 2.5 M in hexanes) was added dropwise over 4 minutes. After an additional 15 min at −100° C. the cooling bath was removed and the mixture was allowed to warm to RT. After 2 h, a solution of 30% aqueous hydrogen peroxide (6.3 mL) in 2M NaOH (2.1 mL) was added. After an additional 1 h, the reaction solution was partitioned between EtOAc and H₂O. The aqueous layer was extracted with additional EtOAc and the combined organic extracts were washed with brine and dried over Na₂SO₄. The solvent was removed in vacuo and the residue was triturated with 3% MeOH/CH₂Cl₂ to give the desired material as a pale yellow solid (0.75 g). LC-MS (ES) m/z 281.0 [M+H]⁺.

### c) 1,1-dimethylethyl (4-{[2-(4-amino-1,2,5-oxadiazol-3-yl)-4-chloro-1-ethyl-1H-imidazo[4,5-c]pyridin-6-yl]oxy}butyl)carbamate

To a solution of 2-(4-amino-1,2,5-oxadiazol-3-yl)-4-chloro-1-ethyl-1,5-dihydro-6H-imidazo[4,5-c]pyridin-6-one (0.17 g, 0.60 mmole), 1,1-dimethylethyl (4-hydroxybutyl)carbamate (0.14 g, 0.75 mmole) and PPh₃ (0.23 g, 0.9 mmole) in THF (20 mL) at 0° C. was added DEAD (0.14 mL, 0.9 mmole) dropwise. After 1 h, MeOH (1 mL) was added and stirring continued for 30 min. The reaction solution was concentrated under vacuum and purified on silica gel (hexanes/EtOAc, 2:1) to give the title compound (0.17 g, 63%) as a colorless oil. LC-MS (ES) m/z 452 [M+H]⁺.

### d) 1,1-dimethylethyl (4-{[2-(4-amino-1,2,5-oxadiazol-3-yl)-1-ethyl-4-(3-hydroxy-3-methyl-1-butyn-1-yl)-1-H-imidazo[4,5-c]pyridin-6-yl]oxy}butyl)carbamate

To a solution of 1,1-dimethylethyl (4-{[2-(4-amino-1,2,5-oxadiazol-3-yl)-4-chloro-1-ethyl-1H-imidazo[4,5-c]pyridin-6-yl]oxy}butyl)carbamate (0.17 g, 0.38 mmole) in DMF (2 mL) was added CuI (4 mg, 0.02 mmole), 2-hydroxy-2-methyl-3-butyne (0.08 g, 0.95 mmole), triethylamine (0.11 mL, 0.76 mmole) and dichlorobistriphenylphosphine palladium (II) (30 mg, 0.04 mmole). The reaction was heated to 80° C. in a sealed tube for 2 h. The reaction solution was concentrated under vacuum and purified on silica gel (hexanes/EtOAc, 1:1) to give the title compound (110 mg, 58%) as a tan foam: LC-MS (ES) m/z=500 (M+H)⁺.

### e) 4-[6-[(4-aminobutyl)oxy]-2-(4-amino-1,2,5-oxadiazol-3-yl)-1-ethyl-1H-imidazo[4,5-c]pyridin-4-yl]-2-methyl-3-butyn-2-ol

To a solution of 1,1-dimethylethyl (4-{[2-(4-amino-1,2,5-oxadiazol-3-yl)-1-ethyl-4-(3-hydroxy-3-methyl-1-butyn-1-yl)-1H-imidazo[4,5-c]pyridin-6-yl]oxy}butyl)carbamate (0.10 g, 0.20 mmole) in methanol (15 mL) was added 4 M HCl (4 mL). After 2 h at RT, the reaction solution was concentrated under vacuum to give the title compound (71 mg, 89%) as a tan solid: LC-MS (ES) m/z=400 (M+H)⁺. ¹H NMR (d6-DMSO, 400 MHz) δ 8.10 (br s, 2H), 7.21 (s, 1H), 4.62 (q, J=7.0 Hz, 2H), 4.31 (t, J=5.9 Hz, 2H), 2.85 (m, 2H), 1.80 (m, 4H), 1.55 (s, 6H), 1.44 (t, J=7.0 Hz, 3H).

### Example 18

### Preparation of 4-[2-(4-amino-1,2,5-oxadiazol-3-yl)-6-[(3-aminopropyl)oxy]-1-ethyl-1H-imidazo[4,5-c]pyridin-4-yl]-2-methyl-3-butyn-2-ol

The title compound was prepared as a tan solid according to the preparation of Example 17, except substituting 1,1-dimethylethyl (4-hydroxypropyl)carbamate (170 mg, 0.98 mmol) for 1,1-dimethylethyl (4-hydroxybutyl)carbamate: LCMS (ES) m/e 386 (M+H)⁺; ¹H NMR (d6-DMSO, 400 MHz) δ 8.12 (br s, 2H), 7.25 (s, 1H), 4.62 (q, J=7.0 Hz, 2H), 4.39 (t, J=5.9 Hz, 2H), 2.98 (m, 2H), 2.17 (m, 2H), 1.53 (s, 6H), 1.38 (t, J=7.0 Hz, 3H).

### Example 19

### Preparation of 4-{2-(4-amino-1,2,5-oxadiazol-3-yl)-6-[(5-aminopentyl)oxy]-1-ethyl-1H-imidazo[4,5-c]pyridin-4-yl}-2-methyl-3-butyn-2-ol

The title compound was prepared as a tan solid according to the preparation of Example 17, except substituting 1,1-dimethylethyl (4-hydroxypentyl)carbamate (130 mg, 0.64 mmol) for 1,1-dimethylethyl (4-hydroxybutyl)carbamate: LCMS (ES) m/e 414 (M+H)⁺; ¹H NMR (d6-DMSO, 400 MHz) δ 8.10 (br s, 2H), 7.22 (s, 1H), 4.62 (q, J=7.0 Hz, 2H), 4.38 (t, J=5.9 Hz, 2H), 2.76 (m, 2H), 2.65 (m, 2H), 1.55 (s, 6H), 1.50 (m, 4H), 1.36 (t, J=7.0 Hz, 3H).

### Example 20

### Preparation of 4-(2-(4-amino-1,2,5-oxadiazol-3-yl)-1-ethyl-6-{[2-(4-piperidinyl)ethyl]oxy}-1H-imidazo[4,5-c]pyridin-4-yl)-2-methyl-3-butyn-2-ol

The title compound was prepared as a tan solid according to the preparation of Example 17, except substituting 1,1-dimethylethyl 4-(2-hydroxyethyl)-1-piperidinecarboxylate (0.31 g, 1.38 mmol) for 1,1-dimethylethyl (2-hydroxyethyl)carbamate (193 mg, 1.2 mmol) for 1,1-dimethylethyl (4-hydroxybutyl)carbamate and: LCMS (ES) m/e 440 (M+H)⁺; ¹H NMR (d6-DMSO, 400 MHz) δ 8.78 (br s, 2H), 7.21 (s, 1H), 4.62 (q, J=7.0 Hz, 2H), 4.33 (t, J=5.9 Hz, 2H), 3.71 (m, 2H), 2.85 (m, 2H), 1.90 (m, 2H), 1.76 (m, 3H), 1.55 (s, 6H), 1.48 (m, 2H), 1.36 (t, J=7.0 Hz, 3H).

### Example 21

### Preparation of 4-(2-(4-amino-1,2,5-oxadiazol-3-yl)-1-ethyl-6-{[2-(3-pyrrolidinyl)ethyl]oxy}-1H-imidazo[4,5-c]pyridin-4-yl)-2-methyl-3-butyn-2-ol

The title compound was prepared as a tan solid according to the preparation of Example 17, except substituting 1,1-dimethylethyl 3-(2-hydroxyethyl)-1-pyrrolidinecarboxylate [J. Med. Chem. 1997, 40, 3497-3500] (0.33 g, 1.56 mmol) for 1,1-dimethylethyl (4-hydroxybutyl)carbamate: LCMS (ES) m/e 426 (M+H)⁺; ¹H NMR (d6-DMSO, 400 MHz) δ 9.35 (br s, 2H), 7.24 (s, 1H), 4.62 (q, J=7.0 Hz, 2H), 4.32 (m, 2H), 3.36 (m, 1H), 3.24 (m, 1H), 3.09 (m, 1H), 2.80 (m, 1H), 2.39 (m, 1H), 2.14 (m, 1H), 1.85 m (2H), 1.60 (m, 1H), 1.55 (s, 6H), 1.35 (t, J=7.0 Hz, 3H).

### Example 22

### Preparation of 4-[2-(4-amino-1,2,5-oxadiazol-3-yl)-1-ethyl-6-(methyloxy)-1H-imidazo[4,5-c]pyridin-4-yl]-2-methyl-3-butyn-2-ol

The title compound was prepared as a tan solid according to the preparation of Example 17, except substituting methanol (0.14 mL, 3.5 mmol) for 1,1-dimethylethyl (4-hydroxybutyl)carbamate: LCMS (ES) m/e 343 (M+H)⁺; ¹H NMR (CD₃OD, 400 MHz) δ 6.87 (s, 1H), 4.62 (q, J=7.0 Hz, 2H), 3.95 (s, 3H), 1.69 (s, 6H), 1.43 (t, J=7.0 Hz, 3H).

### Example 23

### Preparation of 4-[2-(4-amino-1,2,5-oxadiazol-3-yl)-1-ethyl-6-[(2-morpholinylmethyl)oxy]-1H-imidazo[4,5-c]pyridin-4-yl]-2-methyl-3-butyn-2-ol

The title compound was prepared as a tan solid according to the preparation of Example 17, except substituting 1,1-dimethylethyl 2-(hydroxymethyl)-4-morpholinecarboxylate [J. Med. Chem. 1994, 37, 2791-2796] (0.21 g, 0.95 mmol) for 1,1-dimethylethyl (4-hydroxybutyl)carbamate: LCMS (ES) m/e 428 (M+H)⁺; ¹H NMR (d6-DMSO, 400 MHz) δ 7.30 (s, 1H), 7.05 (br s, 2H), 4.62 (q, J=7.0 Hz, 2H), 4.39 (m, 2H), 4.16 (m, 2H), 4.01 (m, 1H), 3.82 (m, 1H), 3.36 (m, 1H), 3.24 (m, 1H), 2.99 (m, 1H), 1.55 (s, 6H), 1.38 (t, J=7.0 Hz, 3H).

### Example 24

### Preparation of 4-[2-(4-amino-1,2,5-oxadiazol-3-yl)-1-ethyl-6-(3-pyrrolidinyloxy)-1H-imidazo[4,5-c]pyridin-4-yl]-2-methyl-3-butyn-2-ol

The title compound was prepared as a tan solid according to the preparation of Example 17, except substituting 1,1-dimethylethyl 3-hydroxy-1-pyrrolidinecarboxylate (0.21 g, 1.11 mmol) for 1,1-dimethylethyl (4-hydroxybutyl)carbamate: LCMS (ES) m/e 398 (M+H)⁺; ¹H NMR (d6-DMSO, 400 MHz) δ 9.61 (br s, 2H), 7.22 (s, 1H), 7.05 (br s, 2H), 5.62 (m, 1H), 4.62 (q, J=7.0 Hz, 2H), 3.55 (m, 2H), 3.43 (m, 2H), 2.29 (m, 1H), 2.19 (m, 1H), 1.55 (s, 6H), 1.36 (t, J=7.0 Hz, 3H).

### Example 25

### Preparation of 4-[2-(4-amino-1,2,5-oxadiazol-3-yl)-1-ethyl-6-[(3S)-3-pyrrolidinyloxy]-1H-imidazo[4,5-c]pyridin-4-yl]-2-methyl-3-butyn-2-ol

The title compound was prepared as a tan solid according to the preparation of Example 17, except substituting 1,1-dimethylethyl (3S)-3-hydroxy-1-pyrrolidinecarboxylate (0.25 g, 1.33 mmol) for 1,1-dimethylethyl (4-hydroxybutyl)carbamate: LCMS (ES) m/e 398 (M+H)⁺; LCMS (ES) m/e 398 (M+H)⁺; ¹H NMR (d6-DMSO, 400 MHz) δ 9.61 (br s, 2H), 7.22 (s, 1H), 7.05 (br s, 2H), 5.62 (m, 1H), 4.62 (q, J=7.0 Hz, 2H), 3.55 (m, 2H), 3.43 (m, 2H), 2.29 (m, 1H), 2.19 (m, 1H), 1.55 (s, 6H), 1.36 (t, J=7.0 Hz, 3H).

### Example 26

### Preparation of 4-{2-(4-amino-1,2,5-oxadiazol-3-yl)-1-ethyl-6-[(3R)-3-pyrrolidinyloxy]-1H-imidazo[4,5-c]pyridin-4-yl}-2-methyl-3-butyn-2-ol

The title compound was prepared as a tan solid according to the preparation of Example 17, except substituting 1,1-dimethylethyl (3R)-3-hydroxy-1-pyrrolidinecarboxylate (0.25 g, 1.33 mmol) for 1,1-dimethylethyl (4-hydroxybutyl)carbamate: LCMS (ES) m/e 398 (M+H)⁺; ¹H NMR (d6-DMSO, 400 MHz) δ 9.61 (br s, 2H), 7.22 (s, 1H), 7.05 (br s, 2H), 5.62 (m, 1H), 4.62 (q, J=7.0 Hz, 2H), 3.55 (m, 2H), 3.43 (m, 2H), 2.29 (m, 1H), 2.19 (m, 1H), 1.55 (s, 6H), 1.36 (t, J=7.0 Hz, 3H).

### Example 27

### Preparation of 4-(2-(4-amino-1,2,5-oxadiazol-3-yl)-6-{[(2R)-2-amino-3-(3-thienyl)propyl]oxy}-1-ethyl-1H-imidazo[4,5-c]pyridin-4-yl)-2-methyl-3-butyn-2-ol

The title compound was prepared as a tan solid according the Example 17, except substituting 1,1-dimethylethyl[(1R)-2-hydroxy-1-(3-thienylmethyl)ethyl]carbamate (450 mg, 1.75 mmole) for 1,1-dimethylethyl (4-hydroxybutyl)carbamate: LCMS (ES) m/e 468 (M+H)⁺; ¹H NMR (d6-dmso, 400 MHz) δ 7.57 (m, 1H), 7.45 (m, 1H), 7.22 (s, 1H), 7.13, (m, 1H), 4.65 (m, 2H), 4.43 (m, 1H), 4.27 (m, 1H), 3.88 (m, 1H), 3.09 (m, 2H), 1.57 (s, 6H), 1.39 (t, J=7.0 Hz, 3H).

### Example 28

### Preparation of 4-[6-{[(2S)-2-amino-3-(1H-indol-3-yl)propyl]oxy}-2-(4-amino-1,2,5-oxadiazol-3-yl)-1-ethyl-1H-imidazo[4,5-c]pyridin-4-yl]-2-methyl-3-butyn-2-ol

The title compound was prepared as a tan solid according to the preparation of Example 17, except substituting 1,1-dimethylethyl[(1S)-2-hydroxy-1-(1H-indol-3-ylmethyl)ethyl]carbamate (0.80 g, 1.07 mmol) for 1,1-dimethylethyl (4-hydroxybutyl)carbamate: LCMS (ES) m/e 501 (M+H)⁺; ¹H NMR (d6-DMSO, 400 MHz) δ 11.09 (br s, 1H), 8.41 (br s, 2H), 7.64 (d, J=8.0 Hz, 1H), 7.39 (d, J=8.0 Hz, 1H), 7.25 (s, 1H), 7.11 (t, J=7.2 Hz, 1H), 7.05 (t, J=7.2 Hz, 1H), 4.62 (q, J=7.0 Hz, 2H), 4.38 (m, 2H), 3.81 (m, 1H), 3.19 (m, 2H), 1.55 (s, 6H), 1.39 (t, J=7.0 Hz, 3H).

### Example 29

### Preparation of 4-[6-{[(1R,2S)-2-aminocyclopentyl]oxy}-2-(4-amino-1,2,5-oxadiazol-3-yl)-1-ethyl-1H-imidazo[4,5-c]pyridin-4-yl]-2-methyl-3-butyn-2-ol

The title compound was prepared as a tan solid according to the preparation of Example 17, except substituting 1,1-dimethylethyl[(1S,2R)-2-hydroxycyclopentyl]carbamate (0.38 g, 1.87 mmol) for 1,1-dimethylethyl (4-hydroxybutyl)carbamate: LCMS (ES) m/e 412 (M+H)⁺; ¹H NMR (d6-DMSO, 400 MHz) δ 8.29 (br s, 2H), 7.22 (s, 1H), 7.06 (br s, 2H), 5.44 (m, 1H), 4.62 (q, J=7.0 Hz, 2H), 3.70 (m, 1H), 2.12 (m, 2H), 1.86 (m, 2H), 1.67 (m, 2H), 1.55 (s, 6H), 1.36 (t, J=7.0 Hz, 3H).

### Example 30

### Preparation of 4-(2-(4-amino-1,2,5-oxadiazol-3-yl)-1-ethyl-6-{[2-(methylamino)ethyl]oxy}-1H-imidazo[4,5-c]pyridin-4-yl)-2-methyl-3-butyn-2-ol

The title compound was prepared as a tan solid according to the preparation of Example 17, except substituting 1,1-dimethylethyl (2-hydroxyethyl)methylcarbamate (0.28 g, 1.61 mmol) for 1,1-dimethylethyl (4-hydroxybutyl)carbamate: LCMS (ES) m/e 386 (M+H)⁺; ¹H NMR (d6-DMSO, 400 MHz) δ 9.14 (br s, 2H), 7.22 (s, 1H), 7.04 (br s, 2H), 5.44 (m, 1H), 4.62 (q, J=7.0 Hz, 2H), 4.59 (t, J=5.1 Hz, 1H), 3.45 (m, 2H), 2.64 (m, 3H), 1.55 (s, 6H), 1.36 (t, J=7.0 Hz, 3H).

### Example 31

### Preparation of 4-[6-{[(1S,2R)-2-aminocyclopentyl]oxy}-2-(4-amino-1,2,5-oxadiazol-3-yl)-1-ethyl-1H-imidazo[4,5-c]pyridin-4-yl]-2-methyl-3-butyn-2-ol

The title compound was prepared as a tan solid according to the preparation of Example 17, except substituting 1,1-dimethylethyl[(1S,2R)-2-hydroxycyclopentyl]carbamate (0.38 g, 1.87 mmol) for 1,1-dimethylethyl (4-hydroxybutyl)carbamate: LCMS (ES) m/e 412 (M+H)⁺; ¹H NMR (d6-DMSO, 400 MHz) δ 8.29 (br s, 2H), 7.22 (s, 1H), 7.06 (br s, 2H), 5.44 (m, 1H), 4.62 (q, J=7.0 Hz, 2H), 3.70 (m, 1H), 2.12 (m, 2H), 1.86 (m, 2H), 1.67 (m, 2H), 1.55 (s, 6H), 1.36 (t, J=7.0 Hz, 3H).

### Example 32

### Preparation of 4-{2-(4-amino-1,2,5-oxadiazol-3-yl)-1-ethyl-6-[(phenylmethyl)oxy]-1H-imidazo[4,5-c]pyridin-4-yl}-2-methyl-3-butyn-2-ol

### a) 4-{4-chloro-1-ethyl-6-[(phenylmethyl)oxy]-1H-imidazo[4,5-c]pyridin-2-yl}-1,2,5-oxadiazol-3-amine

A solution of 2-(4-amino-1,2,5-oxadiazol-3-yl)-4-chloro-1-ethyl-1,5-dihydro-6H-imidazo[4,5-c]pyridin-6-one (100 mg, 0.179 mmol, [prepared in Example 1]) benzyl bromide (25 μL, 0.214 mmol) and silver carbonate (59 mg, 0.214 mmol) in THF (1.8 mL) was refluxed in a sealed tube. After 12 h, the solution was concentrated and the residue purified via column chromatography (silica, 0.5% MeOH in DCM) affording the title compound (50 mg, 38%) as a white powder: LCMS (ES) m/e 371 (M+H)⁺.

### b) 4-{2-(4-amino-1,2,5-oxadiazol-3-yl)-1-ethyl-6-[(phenylmethyl)oxy]-1H-imidazo[4,5-c]pyridin-4-yl}-2-methyl-3-butyn-2-ol (GSK847101)

A solution of 4-{4-chloro-1-ethyl-6-[(phenylmethyl)oxy]-1H-imidazo[4,5-c]pyridin-2-yl}-1,2,5-oxadiazol-3-amine (50 mg, 0.120 mmol), 2-methyl-3-butyne-2-ol (14 μL, 0.145 mmol), CuI (2 mg, 12 μmol) and Pd(PPh₃)₂Cl₂ (4 mg, 6 μmol) in DMF/Et₃N (2.2 mL, 2:1) was heated to 70° C. in a sealed tube. After 2 h, the solution was concentrated and the residue purified via column chromatography (silica, 0.5% MeOH in DCM) yielding the title compound (25 mg, 44%) as a white solid: LCMS (ES) m/e 419 (M+H)⁺; ¹H NMR (CD₃OD, 400 MHz) δ 7.51 (d, J=8.6 Hz, 2H), 7.32-7.40 (m, 3H), 7.05 (s, 1H), 5.42 (bs, 2H), 4.68 (q, J=7.2 Hz, 2H), 1.68 (s, 6H), 1.45 (t, J=7.2 Hz, 3H).

### Example 33

### Preparation of 4-{2-(4-amino-1,2,5-oxadiazol-3-yl)-1-ethyl-6-[(4-piperidinylmethyl)oxy]-1H-imidazo[4,5-c]pyridine-4-yl}-2-methyl-3-butyn-2-ol

The title compound was prepared as a tan solid according to Example 17, except substituting 4-piperidinylmethanol (2 g, 17.4 mmol) for 1,1-dimethylethyl (4-hydroxybutyl)carbamate: LCMS (ES) m/e 426 (M+H)⁺; ¹H NMR ((CD₃)₂SO, 400 MHz) δ 8.80 (bs, 1H), 8.52 (bs, 1H), 7.22 (s, 1H), 7.13 (bs, 2H), 4.52-4.63 (m, 2H), 4.16-4.26 (m, 2H), 3.22-3.36 (m, 2H), 2.87-2.99 (m, 2H), 2.05-2.10 (m, 1H), 1.89-1.99 (m, 2H), 1.54 (s, 6H), 1.48-1.50 (m, 2H), 1.27-1.35 (m, 3H).

### Example 34

### Preparation of 4-(2-(4-amino-1,2,5-oxadiazol-3-yl)-1-ethyl-6-{[2-(3-piperidinyl)ethyl]oxy}-1H-imidazo[4,5-c]pyridin-4-yl)-2-methyl-3-butyn-2-ol

The title compound was prepared as a yellow solid according to Example 17, except substituting 2-(3-piperidinyl)ethanol (1 g, 7.74 mmol) for 1,1-dimethylethyl (4-hydroxybutyl)carbamate: LCMS (ES) m/e 440 (M+H)⁺; ¹H NMR ((CD₃)₂SO, 400 MHz) δ 9.15 (bs, 1H), 8.73 (bs, 1H), 7.23 (s, 1H), 7.15 (bs, 2H), 4.52-4.65 (m, 2H), 4.32-4.41 (m, 2H), 3.15-3.31 (m, 2H), 2.70-2.81 (m, 1H), 2.54-2.59 (m, 1H), 1.81-1.99 (m, 2H), 1.63-1.80 (m, 4H), 1.55 (s, 6H), 1.34-1.41 (m, 3H), 1.13-1.40 (m, 1H).

### Example 35

### Preparation of 4-[2-(4-amino-1,2,5-oxadiazol-3-yl)-1-ethyl-6-[(3-piperidinylmethyl)oxy]-1H-imidazo[4,5-c]pyridin-4-yl]-2-methyl-3-butyn-2-ol

The title compound was prepared as a tan solid according the Example 17, except substituting 3-piperidinylmethanol (2 g, 17.4 mmol) for 1,1-dimethylethyl (4-hydroxybutyl)carbamate: LCMS (ES) m/e 426 (M+H)⁺; ¹H NMR ((CD₃)₂SO, 400 MHz) δ 9.21 (bs, 1H), 9.15 (bs, 1H), 7.26 (s, 1H), 7.12 (bs, 2H), 4.52-4.63 (m, 2H), 4.13-4.27 (m, 2H), 3.17-3.45 (m, 2H), 2.72-2.83 (m, 2H), 2.21-2.39 (m, 1H), 1.73-1.84 (m, 2H), 1.61-1.75 (m, 1H), 1.54 (s, 6H), 1.33-1.40 (m, 4H).

### Example 36

### Preparation of 4-[6-{[(2R)-2-amino-3-(1H-indol-3-yl)propyl]oxy}-2-(4-amino-1,2,5-oxadiazol-3-yl)-1-ethyl-1H-imidazo[4,5-c]pyridin-4-yl]-2-methyl-3-butyn-2-ol

The title compound was prepared as a tan solid according to the preparation of Example 17, except substituting 1,1-dimethylethyl[(1R)-2-hydroxy-1-(1H-indol-3-ylmethyl)ethyl]carbamate (0.80 g, 1.07 mmol) for 1,1-dimethylethyl (4-hydroxybutyl)carbamate: LCMS (ES) m/e 501 (M+H)⁺; ¹H NMR (d6-DMSO, 400 MHz) δ 11.09 (br s, 1H), 8.41 (br s, 2H), 7.64 (d, J=8.0 Hz, 1H), 7.39 (d, J=8.0 Hz, 1H), 7.25 (s, 1H), 7.11 (t, J=7.2 Hz, 1H), 7.05 (t, J=7.2 Hz, 1H), 4.62 (q, J=7.0 Hz, 2H), 4.38 (m, 2H), 3.81 (m, 1H), 3.19 (m, 2H), 1.55 (s, 6H), 1.39 (t, J=7.0 Hz, 3H).

### Example 37

### Preparation of 4-(2-(4-amino-1,2,5-oxadiazol-3-yl)-1-ethyl-6-{[(2R)-2-pyrrolidinylmethyl]oxy}-1H-imidazo[4,5-c]pyridin-4-yl)-2-methyl-3-butyn-2-ol

The title compound was prepared as a tan solid according the Example 17, except substituting 1,1-dimethylethyl (2R)-2-(hydroxymethyl)-1-pyrrolidinecarboxylate (187 mg, 0.926 mmol) for 1,1-dimethylethyl (4-hydroxybutyl)carbamate: LCMS (ES) m/e 412 (M+H)⁺; ¹H NMR ((CD₃)₂SO, 400 MHz) δ 9.72 (bs, 1H), 9.01 (bs, 1H), 7.29 (s, 1H), 7.00 (bs, 2H), 4.66 (q, J=7.3 Hz, 2H), 4.52-4.58 (m, 1H), 4.48-4.50 (m, 1H), 3.98-4.01 (m, 1H), 3.17-3.25 (m, 2H), 2.12-2.17 (m, 1H), 1.89-2.03 (m, 2H), 1.72-1.79 (m, 1H), 1.55 (s, 6H), 1.09 (t, J=7.2 Hz, 3H).

### Example 38

### Preparation of 4-(2-(4-amino-1,2,5-oxadiazol-3-yl)-1-ethyl-6-{[(2S)-2-pyrrolidinylmethyl]oxy}-1H-imidazo[4,5-c]pyridin-4-yl)-2-methyl-3-butyn-2-ol

The title compound was prepared as a yellow solid according the Example 17, except substituting 1,1-dimethylethyl (2S)-2-(hydroxymethyl)-1-pyrrolidinecarboxylate (211 mg, 1.05 mmol) for 1,1-dimethylethyl (4-hydroxybutyl)carbamate: LCMS (ES) m/e 412 (M+H)⁺; ¹H NMR (CD₃OD, 400 MHz) δ 7.06 (s, 1H), 4.7-4.74 (m, 2H), 4.43-4.48 (m, 1H), 4.29-4.42 (m, 1H), 3.61-3.68 (m, 1H0, 3.07-3.11 (m, 1H), 3.01-3.06 (m, 1H), 2.05-2.12 (m, 1H), 1.89-1.99 (m, 2H), 1.68-1.73 (m, 1H), 1.65 (s, 6H), 1.45-1.52 (t, 3H).

### Example 39

### Preparation of 4-[2-(4-amino-1,2,5-oxadiazol-3-yl)-1-ethyl-6-[(1H-indol-3-ylmethyl)oxy]-1H-imidazo[4,5-c]pyridin-4-yl]-2-methyl-3-butyn-2-ol

The title compound was prepared as a yellow solid according the Example 17, except substituting 1,1-dimethylethyl 3-(2-hydroxyethyl)-1H-indole-1-carboxylate (214 mg, 0.81 mmol) for 1,1-dimethylethyl (4-hydroxybutyl)carbamate: LCMS (ES) m/e 472 (M+H)⁺; ¹H NMR (CD₃OD, 400 MHz) δ 7.69-7.67 (m, 1H), 7.35-7.33 (m, 1H), 7.18 (s, 1H), 7.03-7.09 (m, 3H), 4.63-4.69 (m, 4H), 3.40-3.43 (m, 2H), 1.68 (s, 6H), 1.41-1.44 (t, 3H).

### Example 40

### Preparation of 4-[6-[(4-amino-2-methylbutyl)oxy]-2-(4-amino-1,2,5-oxadiazol-3-yl)-1-ethyl-1H-imidazo[4,5-c]pyridin-4-yl]-2-methyl-3-butyn-2-ol

The title compound was prepared as a yellow solid according the Example 17, except substituting 1,1-dimethylethyl (4-hydroxy-3-methylbutyl)carbamate (434 mg, 2.14 mmol) for 1,1-dimethylethyl (4-hydroxybutyl)carbamate: LCMS (ES) m/e 414 (M+H)⁺; ¹H NMR (CD₃OD, 400 MHz) δ 7.03 (s, 1H), 7.67-7.43 (m, 2H), 4.20-4.25 (m, 2H), 2.87-2.91 (m, 2H), 2.11-2.15 (m, 1H), 1.81-1.84 (m, 1H), 1.68 (s, 6H), 1.56-1.63 (m, 1H), 1.48 (t, 3H), 1.12-1.14 (m, 3H).

### Example 41

### Preparation of 4-(2-(4-amino-1,2,5-oxadiazol-3-yl)-6-{[(2S)-2-amino-2-phenylethyl]oxy}-1-ethyl-1H-imidazo[4,5-c]pyridin-4-yl)-2-methyl-3-butyn-2-ol

The title compound was prepared as a yellow solid according the Example 17, except substituting 1,1-dimethylethyl[(1R)-2-hydroxy-1-phenylethyl]carbamate (283 mg, 1.2 mmol) for 1,1-dimethylethyl (4-hydroxybutyl)carbamate: LCMS (ES) m/e 448 (M+H)⁺; ¹H NMR (CD₃OD, 400 MHz) δ 7.51-7.54 (m, 2H), 7.47-7.49 (m, 2H), 7.3-7.33 (m, 1H), 7.05 (s, 1H), 7.65-4.71 (m, 2H), 4.52-4.54 (1H), 4.38-4.5 (m, 2H), 1.68 (s, 6H), 1.47-1.50 (t, 3H).

### Example 42

### Preparation of 4-(2-(4-amino-1,2,5-oxadiazol-3-yl)-6-{[(2R)-2-amino-2-phenylethyl]oxy}-1-ethyl-1H-imidazo[4,5-c]pyridin-4-yl)-2-methyl-3-butyn-2-ol

The title compound was prepared as a brown solid according the Example 17, except substituting [(1R)-2-hydroxy-1-phenylethyl]carbamate (283 mg, 1.2 mmol) for 1,1-dimethylethyl (4-hydroxybutyl)carbamate: LCMS (ES) m/e 448 (M+H)⁺; ¹H NMR (CD₃OD, 400 MHz) δ 7.51-7.54 (m 2H), 7.47-7.49 (m, 2H), 7.3-7.33 (m, 1H), 7.05 (s, 1H), 7.65-4.71 (m, 2H), 4.52-4.54 (1H), 4.38-4.5 (m, 2H), 1.68 (s, 6H), 1.47-1.50 (t, 3H).

### Example 43

### Preparation of 4-(2-(4-amino-1,2,5-oxadiazol-3-yl)-6-{[(2R)-2-amino-3-phenylpropyl]oxy}-1-ethyl-1H-imidazo[4,5-c]pyridin-4-yl)-2-methyl-3-butyn-2-ol

The title compound was prepared as a brown solid according the Example 17, except substituting 1,1-dimethylethyl[(1R)-2-hydroxy-1-(phenylmethyl)ethyl]carbamate (374 mg, 1.49 mmol) for 1,1-dimethylethyl (4-hydroxybutyl)carbamate: LCMS (ES) m/e 462 (M+H)⁺; ¹H NMR (CD₃OD, 400 MHz) δ 7.73-7.74 (m, 4H), 7.28-7.30 (m, 1H), 7.05 (s, 1H), 4.69-4.71 (m, 2H), 4.3-4.35 (m, 1H), 4.17-4.22 (m, 1H), 3.43-3.51 (m, 1H), 2.98-3.02 (M, 1H), 2.79-2.84 (m, 1H), 1.68 (s, 6H), 1.45 (t, 3H).

### Example 44

### Preparation of 4-(2-(4-amino-1,2,5-oxadiazol-3-yl)-6-{[(2S)-2-amino-3-phenylpropyl]oxy}-1-ethyl-1H-imidazo[4,5-c]pyridin-4-yl)-2-methyl-3-butyn-2-ol

The title compound was prepared as a yellow solid according the Example 17, except substituting 1,1-dimethylethyl[(1S)-2-hydroxy-1-(phenylmethyl)ethyl]carbamate (374 mg, 1.49 mmol) for 1,1-dimethylethyl (4-hydroxybutyl)carbamate: LCMS (ES) m/e 462 (M+H)⁺; ¹H NMR (CD₃OD, 400 MHz) δ 7.73-7.74 (m, 4H), 7.28-7.30 (m, 1H), 7.05 (s, 1H), 4.69-4.71 (M, 2H), 4.3-4.35 (M, 1H), 4.17-4.22 (M, 1H), 3.43-3.51 (m, 1H), 2.98-3.02 (M, 1H), 2.79-2.84 (m, 1H), 1.68 (s, 6H), 1.45 (t, 3H).

### Example 45

### Preparation of 4-[6-{[(2S)-2-amino-3-methylbutyl]oxy}-2-(4-amino-1,2,5-oxadiazol-3-yl)-1-ethyl-1H-imidazo[4,5-c]pyridin-4-yl]-2-methyl-3-butyn-2-ol

The title compound was prepared as a white solid according the Example 17, except substituting 1,1-dimethylethyl[(1S)-1-(hydroxymethyl)-2-methylpropyl]carbamate (152 mg, 0.75 mmol) for 1,1-dimethylethyl (4-hydroxybutyl)carbamate: LCMS (ES) m/e 414 (M+H)⁺; ¹H NMR (CD₃OD, 400 MHz) δ 7.06 (s, 1H), 4.68-4.72 m, 2H), 4.41-4.47 (m, 1H), 4.18-4.4.22 (m, 1H), 2.95-3.01 (m, 1H), 1.85-1.91 (m, 1H), 1.67 (s, 6H), 1.48 (t, 3H), 1.08 (d, J=6.8 Hz, 6H).

### Example 46

### Preparation of 4-[6-[(2-aminoethyl)oxy]-2-(4-amino-1,2,5-oxadiazol-3-yl)-1-ethyl-1H-imidazo[4,5-c]pyridin-4-yl]-3-butyn-2-ol

The title compound was prepared as a light brown solid according the Example 17, except substituting 1,1-dimethylethyl (2-hydroxyethyl)carbamate (193 mg, 1.2 mmol) for 1,1-dimethylethyl (4-hydroxybutyl)carbamate and 3-butyn-2-ol (29.4 mg, 0.42 mmol) for 2-methyl-3-butyn-2-ol: LCMS (ES) m/e 358 (M+H)⁺; ¹H NMR (CD₃OD, 400 MHz) δ 7.07 (s, 1H), 4.81-4.84 (m, 1H), 4.68-4.74 (m, 2H), 4.37-4.43 (m, 1H), 3.06-3.11 (m, 2H), 1.60 (d, J=8 Hz, 3H), 1.48 (t, 3H).

### Example 47

### Preparation of 3-[6-[(2-aminoethyl)oxy]-2-(4-amino-1,2,5-oxadiazol-3-yl)-1-ethyl-1H-imidazo[4,5-c]pyridin-4-yl]-2-propyn-1-ol

The title compound was prepared as a brown solid according the Example 17, except substituting 1,1-dimethylethyl (2-hydroxyethyl)carbamate (193 mg, 1.2 mmol) for 1,1-dimethylethyl (4-hydroxybutyl)carbamate and 2-propyn-1-ol (24 mg, 0.42 mmol) for 2-methyl-3-butyn-2-ol: LCMS (ES) m/e 344 (M+H)⁺; ¹H NMR (CD₃OD, 400 MHz) δ 7.08 (s, 1H), 4.74-4.77 (m, 2H), 4.61 (s, 2H), 4.42 (t, 2H), 3.1 (t, 2H), 1.52 (t, 3H).

### Example 48

### Preparation of 4-(2-(4-amino-1,2,5-oxadiazol-3-yl)-6-{[(2R)-2-amino-3-phenylpropyl]oxy}-1-ethyl-1H-imidazo[4,5-c]pyridin-4-yl)-2-methyl-3-butyn-2-ol

The title compound was prepared as a brown solid according the Example 17, except substituting 1,1-dimethylethyl 2-(hydroxymethyl)-2,3-dihydro-1H-indole-1-carboxylate (299 mg, 1.2 mmol) for 1,1-dimethylethyl (4-hydroxybutyl)carbamate: LCMS (ES) m/e 460 (M+H)⁺; ¹H NMR (CD₃OD, 400 MHz) δ 7.09-7.11 (m, 1H), 7.03 (s, 1H), 6.96-7.02 (m, 1H), 6.64-6.7 (m, 2H), 4.65-4.7 (m, 2H), 4.39-4.42 (m, 1H), 4.3-4.32 (m, 1H), 4.22-4.26 (m, 1H), 3.21-3.29 (m, 1H), 2.89-2.96 (m, 1H), 1.69 (s, 6H), 1.45 (t, 3H).

### Example 49

### Preparation of 4-(2-(4-amino-1,2,5-oxadiazol-3-yl)-6-{[(2S)-2-azetidinylmethyl]oxy}-1-ethyl-1H-imidazo[4,5-c]pyridin-4-yl)-2-methyl-3-butyn-2-ol

The title compound was prepared as a brown solid according the Example 17, except substituting 1,1-dimethylethyl (2S)-2-(hydroxymethyl)-1-azetidinecarboxylate (0.50 g, 2.67 mmol) for 1,1-dimethylethyl (4-hydroxybutyl)carbamate: LCMS (ES) m/e 398 (M+H)⁺; ¹H NMR (dmso-d6, 400 MHz) δ 7.32 (s, 1H), 7.03 (s, 2H), 5.78 (m, 1H), 4.55 (m, 2H), 3.95 (m, 2H), 3.42 (m, 2H), 2.51 (m, 2H), 1.55 (s, 6H), 1.36 (t, J=7.0 Hz, 3H).

### Example 50

### Preparation of 4-[6-{[(1R,2S)-2-aminocyclohexyl]oxy}-2-(4-amino-1,2,5-oxadiazol-3-yl)-1-ethyl-1H-imidazo[4,5-c]pyridin-4-yl]-2-methyl-3-butyn-2-ol

The title compound was prepared as a bisque solid according the Example 17, except substituting 1,1-dimethylethyl[(1S,2R)-2-hydroxycyclohexyl]carbamate (268 mg, 1.25 mmol) for 1,1-dimethylethyl (4-hydroxybutyl)carbamate: LCMS (ES) m/e 419 (M+H)⁺; ¹H NMR (CD₃OD, 400 MHz) δ 7.09 (s, 1H), 5.23-5.34 (m, 1H), 4.70 (q, J=7.2 Hz, 2H), 2.99-3.07 (m, 1H), 2.15-2.19 (m, 1H), 1.71-1.81 (m, 3H), 1.79 (s, 6H), 1.55-1.65 (m, 1H), 1.45-1.49 (m, 6H).

### Example 51

### Preparation of 4-[6-{[(1S,2R)-2-aminocyclohexyl]oxy}-2-(4-amino-1,2,5-oxadiazol-3-yl)-1-ethyl-1H-imidazo[4,5-c]pyridin-4-yl]-2-methyl-3-butyn-2-ol

The title compound was prepared as a yellow solid according the Example 17, except substituting 1,1-dimethylethyl[(1R,2S)-2-hydroxycyclohexyl]carbamate (222 mg, 1.03 mmol) for 1,1-dimethylethyl (4-hydroxybutyl)carbamate: LCMS (ES) m/e 419 (M+H)⁺; ¹H NMR (CD₃OD, 400 MHz) δ 7.09 (s, 1H), 5.23-5.34 (m, 1H), 4.70 (q, J=7.2 Hz, 2H), 2.99-3.07 (m, 1H), 2.15-2.19 (m, 1H), 1.71-1.81 (m, 3H), 1.79 (s, 6H), 1.55-1.65 (m, 1H), 1.45-1.49 (m, 6H).

### Example 52

### Preparation of (racemic)4-[6-{[(1S,2S)-2-aminocyclohexyl]oxy}-2-(4-amino-1,2,5-oxadiazol-3-yl)-1-ethyl-1H-imidazo[4,5-c]pyridin-4-yl]-2-methyl-3-butyn-2-ol

The title compound was prepared as a yellow solid according the Example 17, except substituting (rac)1,1-dimethylethyl[(1S,2S)-2-hydroxycyclohexyl]carbamate (313 mg, 1.5 mmol) for 1,1-dimethylethyl (4-hydroxybutyl)carbamate: LCMS (ES) m/e 426 (M+H)⁺; ¹H NMR (CD₃OD, 400 MHz) δ 7.05 (s, 1H), 4.71-4.71 (m, 1H), 4.67-4.72 (m, 2H), 2.99-3.05 (m, 1H), 2.31-2.37 (m, 1H), 2.00-2.11 (m, 1H), 1.75-1.82 (m, 2H), 1.67 (s, 6H), 1.48-1.57 (m, 3H), 1.36-1.48 (m, 4H).

### Example 53

### Preparation of 4-(2-(4-amino-1,2,5-oxadiazol-3-yl)-1-ethyl-6-{[2-(2-morpholinyl)ethyl]oxy}-1H-imidazo[4,5-c]pyridin-4-yl)-2-methyl-3-butyn-2-ol

The title compound was prepared as a beige solid according the Example 17, except substituting 1,1-dimethylethyl 2-(2-hydroxyethyl)-4-morpholinecarboxylate (335 mg, 1.45 mmol) [prepared according to Kato, S.; et al. J. Med. Chem. 1990, 33, 5, 1406.] for 1,1-dimethylethyl (4-hydroxybutyl)carbamate: LCMS (ES) m/e 442 (M+H)⁺; ¹H NMR ((CD₃)₂SO, 400 MHz) δ 9.51-9.62 (m, 2H), 7.24 (s, 1H), 5.61-5.82 (m, 2H), 4.55-4.63 (m, 2H), 4.35-4.42 (m, 2H), 3.89-3.99 (m, 1H), 3.61-3.70 (m, 2H), 3.42-3.51 (m, 1H), 3.22-3.26 (m, 1H), 2.97-2.99 (m, 1H), 2.73-2.80 (m, 1H), 1.82-2.01 (m, 2H), 1.55 (s, 6H), 1.36-1.47 (m, 3H).

### Example 54

### Preparation of 4-[2-(4-amino-1,2,5-oxadiazol-3-yl)-1-ethyl-6-({3-[(2S)-2-pyrrolidinyl]propyl}oxy)-1H-imidazo[4,5-c]pyridin-4-yl]-2-methyl-3-butyn-2-ol

### a) 1,1-dimethylethyl (2S)-2-[(1E)-3-(ethyloxy)-3-oxo-1-propen-1-yl]-1-pyrrolidinecarboxylate

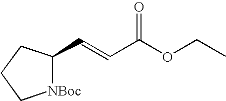

To a solution of NaH (782 mg, 19.6 mmol) in THF (15 mL) at 25° C. was added dropwise triethylphosphono acetate (3.6 mL, 18.1 mmol). After 0.5 h, the solution was cooled to 0° C. and Boc-L-prolinal (3 g, 15.1 mmol) in THF (60 mL) was added dropwise. After an additional 2 h at 0° C., the solution was concentrated and the residue partitioned between DCM-H₂O. The aqueous phase was back-extracted several times with DCM and the combined organic fractions were dried over Na₂SO₄ and concentrated. The resulting yellow oil (4 g, quant.) was used directly without further purification: LCMS (ES) m/e 270 (M+H)⁺.

### b) 1,1-dimethylethyl (2S)-2-(3-hydroxypropyl)-1-pyrrolidinecarboxylate

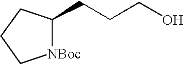

A solution of 1,1-dimethylethyl (2S)-2-[(1E)-3-(ethyloxy)-3-oxo-1-propen-1-yl]-1-pyrrolidinecarboxylate (4 g, 14.9 mmol) and Pd(OH)₂ (1.2 g, 30 wt. %) in MeOH (74 mL) underwent hydrogenolysis at 60 psi using a parr shaker. After 2 h, the solution was filtered through Celite® and concentrated affording the ester that was used directly without further purification: LCMS (ES) m/e 272 (M+H)⁺.

To the above ethyl ester in THF (32 mL) at 0° C. was added dropwise a 1M LAH-THF solution (32 ml, 32 mmol). After 2 h, the solution was quenched with a saturated solution of sodium potassium tartrate and extracted with DCM. The combined organic fractions were dried over Na₂SO₄, concentrated and purified via column chromatography (silica, 2% MeOH in DCM) yielding the title compound (1.6 g, 47%-2 steps) as a clear oil: LCMS (ES) m/e 174 (M+H)⁺.

### c) 4-[2-(4-amino-1,2,5-oxadiazol-3-yl)-1-ethyl-6-({3-[(2S)-2-pyrrolidinyl]propyl}oxy)-1H-imidazo[4,5-c]pyridin-4-yl]-2-methyl-3-butyn-2-ol

The title compound was prepared as a tan solid according the Example 17, except substituting 1,1-dimethylethyl (2S)-2-(3-hydroxypropyl)-1-pyrrolidinecarboxylate (349 mg, 1.53 mmol) 1,1-dimethylethyl (4-hydroxybutyl)carbamate: LCMS (ES) m/e 440 (M+H)⁺; ¹H NMR ((CD₃)₂SO, 400 MHz) δ 9.42 (s, 1H), 8.74 (s, 1H), 7.24 (s, 1H), 7.00 (bs, 2H), 4.53-4.61 (m, 2H), 4.36-4.39 (m, 2H), 3.49-3.52 (m, 1H), 3.21-3.33 (m, 2H), 2.15-2.19 (m, 1H), 1.72-1.99 (m, 5H), 1.52 (s, 6H), 1.36-1.39 (m, 2H), 1.31-1.36 (m, 3H).

### Example 55

### Preparation of 2-(4-amino-1,2,5-oxadiazol-3-yl)-1-ethyl-4-(3-hydroxy-3-methyl-1-butyn-1-yl)-1,5-dihydro-6H-imidazo[4,5-c]pyridin-6-one

### a) 4-[2-(4-amino-1,2,5-oxadiazol-3-yl)-1-ethyl-6-({[4-(methyloxy)phenyl]methyl}oxy)-1H-imidazo[4,5-c]pyridin-4-yl]-2-methyl-3-butyn-2-ol

The title compound was prepared as a yellow solid according the Example 17, except substituting [4-(methyloxy)phenyl]methanol (131 mg, 0.946 mmol) for 1,1-dimethylethyl (4-hydroxybutyl)carbamate: LCMS (ES) m/e 449 (M+H)⁺.

### b) 2-(4-amino-1,2,5-oxadiazol-3-yl)-1-ethyl-4-(3-hydroxy-3-methyl-1-butyn-1-yl)-1,5-dihydro-6H-imidazo[4,5-c]pyridin-6-one

TFA (1 mL) was added dropwise to a solution of 4-[2-(4-amino-1,2,5-oxadiazol-3-yl)-1-ethyl-6-({[4-(methyloxy)phenyl]methyl}oxy)-1H-imidazo[4,5-c]pyridin-4-yl]-2-methyl-3-butyn-2-ol (100 mg, 0.223 mmol) in DCM (1 mL) at 25° C. After 1 h, the solution was concentrated and the residue triturated with ether affording the title compound (32 mg, 26%) as a yellow solid: LCMS (ES) m/e 329 (M+H)⁺; ¹H NMR ((CD₃)₂SO, 400 MHz) δ 7.00 (s, 2H), 6.89 (s, 1H), 5.70 (bs, 1H), 4.52-2.61 (m, 2H), 1.53 (s, 6H), 1.32-1.39 (m, 3H).

### Example 56

### Preparation of 4-(2-(4-amino-1,2,5-oxadiazol-3-yl)-6-{[(2S)-2-aminopropyl]oxy}-1-ethyl-1H-imidazo[4,5-c]pyridin-4-yl)-2-methyl-3-butyn-2-ol

### a) (2S)-2-({[(1,1-dimethylethyl)oxy]carbonyl}amino)propyl 4-methylbenzenesulfonate

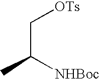

A solution of 1,1-dimethylethyl[(1S)-2-hydroxy-1-methylethyl]carbamate (500 mg, 2.85 mmol), p-toluenesulfonyl chloride (653 mg, 3.42 mmol), triethylamine (517 μL, 3.71 mmol) and dimethylamino pyridine (35 mg, 0.285 mmol) in DCM (14 mL) stirred at 25° C. for 5 h. The mixture was then concentrated and dry loaded onto silica gel (10-30% Ethyl acetate in hexanes) yielding the title compound (640 mg, 68%) as a white solid: LCMS (ES) m/e 330 (M+H)⁺;

### b) 1,1-dimethylethyl((1S)-2-{[2-(4-amino-1,2,5-oxadiazol-3-yl)-4-chloro-1-ethyl-1H-imidazo[4,5-c]pyridin-6-yl]oxy}-1-methyl ethyl)carbamate

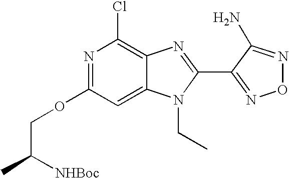

A solution of 2-(4-amino-1,2,5-oxadiazol-3-yl)-4-chloro-1-ethyl-1,5-dihydro-6H-imidazo[4,5-c]pyridin-6-one (290 mg, 1.04 mmol), (2S)-2-({[(1,1-dimethylethyl)oxy]carbonyl}amino)propyl 4-methylbenzenesulfonate (375 mg, 1.14 mmol) and cesium carbonate (506 mg, 1.55 mmol) in DMF (6.5 mL) were heated in a sealed tube at 70° C. for 12 h. The solution was concentrated and the residue purified via column chromatography (silica, 1% MeOH in DCM) affording the title compound (91 mg, 40% (based on 50% purity of the pyridine)) as an orange oil: LCMS (ES) m/e 438 (M+H)⁺;

### c) 1,1-dimethylethyl((1S)-2-{[2-(4-amino-1,2,5-oxadiazol-3-yl)-1-ethyl-4-(3-hydroxy-3-methyl-1-butyn-1-yl)-1H-imidazo[4,5-c]pyridin-6-yl]oxy}-1-methylethyl)carbamate

A solution of 1,1-dimethylethyl((1S)-2-{[2-(4-amino-1,2,5-oxadiazol-3-yl)-4-chloro-1-ethyl-1H-imidazo[4,5-c]pyridin-6-yl]oxy}-1-methylethyl)carbamate (91 mg, 0.208 mmol), 2-methyl-3-butyne-2-ol (65 μL, 0.666 mmol), CuI (4 mg, 20.8 μmol) and Pd(PPh₃)₂Cl₂ (15 mg, 20.8 μmol) in DMF/Et₃N (2.1 mL, 2:1) was heated to 70° C. in a sealed tube. After 12 h, the solution was concentrated and the residue purified via column chromatography (silica, 1% MeOH in DCM (1% NH₄OH)) yielding the title compound (64 mg, 63%) as an orange oil: LCMS (ES) m/e 419 (M+H)⁺.

### d) 4-(2-(4-amino-1,2,5-oxadiazol-3-yl)-6-{[(2S)-2-aminopropyl]oxy}-1-ethyl-1H-imidazo[4,5-c]pyridin-4-yl)-2-methyl-3-butyn-2-ol

A 4M HCl-dioxane solution (660 μL, 2.64 mmol) was added dropwise to 1,1-dimethylethyl((1S)-2-{[2-(4-amino-1,2,5-oxadiazol-3-yl)-1-ethyl-4-(3-hydroxy-3-methyl-1-butyn-1-yl)-1H-imidazo[4,5-c]pyridin-6-yl]oxy}-1-methylethyl)carbamate (64 mg, 0.132 mmol) in MeOH (2 mL) at 25° C. After 3 h, the solution was concentrated and the residue dissolved in a solution of DCM-MeOH—NH₄OH (90:10:1) and run through a plug of silica affording the title compound (26 mg, 51%) as a beige solid: LCMS (ES) m/e 386 (M+H)⁺; ¹H NMR (CD₃OD, 400 MHz) δ 7.07 (s, 1H), 4.70 (q, J=7.3 Hz, 2H), 4.21-4.36 (m, 1H), 4.05-4.17 (m, 1H), 3.31-3.42 (m, 1H), 1.67 (s, 6H), 1.50 (t, J=7.2 Hz, 3H), 1.19 (d, J=7.2 Hz, 3H).

### Example 57

### Preparation of 4-[6-{[(2R)-2-amino-3-methylbutyl]oxy}-2-(4-amino-1,2,5-oxadiazol-3-yl)-1-ethyl-1H-imidazo[4,5-c]pyridin-4-yl]-2-methyl-3-butyn-2-ol

The title compound was prepared as a yellow solid according the Example 56, except substituting 1,1-dimethylethyl[(1R)-1-(hydroxymethyl)-2-methylpropyl]carbamate (244 mg, 1.2 mmol) for 1,1-dimethylethyl[(1S)-2-hydroxy-1-methylethyl]carbamate: LCMS (ES) m/e 414 (M+H)⁺; ¹H NMR (CD₃OD, 400 MHz) δ 7.09 (s, 1H), 4.65-4.77 (m, 3H), 4.47-4.49 (m, 1H), 4.25-4.27 (m, 1H), 3.02-3.07 (m, 1H), 1.96-1.99 (m, 1H), 1.71 (s, 6H), 1.5 (t, 3H), 1.06 (d, J=6.84 Hz, 6H).

### Example 58

### Preparation of 4-[6-{[(2R)-2-amino-4-methylpentyl]oxy}-2-(4-amino-1,2,5-oxadiazol-3-yl)-1-ethyl-1H-imidazo[4,5-c]pyridin-4-yl]-2-methyl-3-butyn-2-ol

The title compound was prepared as a brown solid according the Example 56, except substituting 1,1-dimethylethyl[(1R)-1-(hydroxymethyl)-3-methylbutyl]carbamate (260 mg, 1.2 mmol) for 1,1-dimethylethyl[(1S)-2-hydroxy-1-methylethyl]carbamate: LCMS (ES) m/e 428 (M+H)⁺; ¹H NMR (CD₃OD, 400 MHz) δ 6.96 (s, 1H), 4.61-4.67 (m, 2H), 4.31-4.35 (m, 1H), 4.02-4.06 (m, 1H), 3.25-3.31 (m, 1H), 1.83-1.9 (m, 1H), 1.68 (s, 6H), 1.40-1.46 (m, 5H), 0.98-1.04 (m, 6H).

### Example 59

### Preparation of 4-[6-{[(2S)-2-amino-4-methylpentyl]oxy}-2-(4-amino-1,2,5-oxadiazol-3-yl)-1-ethyl-1H-imidazo[4,5-c]pyridin-4-yl]-2-methyl-3-butyn-2-ol

The title compound was prepared as a brown solid according the Example 56, except substituting 1,1-dimethylethyl[(1S)-1-(hydroxymethyl)-3-methylbutyl]carbamate (260 mg, 1.2 mmol) for 1,1-dimethylethyl[(1S)-2-hydroxy-1-methylethyl]carbamate: LCMS (ES) m/e 428 (M+H)⁺; ¹H NMR (CD₃OD, 400 MHz) δ 6.96 (s, 1H), 4.61-4.67 (m, 2H), 4.31-4.35 (m, 1H), 4.02-4.06 (m, 1H), 3.25-3.31 (m, 1H), 1.83-1.9 (m, 1H), 1.68 (s, 6H), 1.40-1.46 (m, 5H), 0.98-1.04 (m, 6H).

### Example 60

### Preparation of 4-(2-(4-amino-1,2,5-oxadiazol-3-yl)-6-{[(2R)-2-aminopropyl]oxy}-1-ethyl-1H-imidazo[4,5-c]pyridin-4-yl)-2-methyl-3-butyn-2-ol

The title compound was prepared as a yellow solid according the Example 56, except substituting 1,1-dimethylethyl[(1R)-2-hydroxy-1-methylethyl]carbamate (210 mg, 1.2 mmol) for 11,1-dimethylethyl[(1S)-2-hydroxy-1-methylethyl]carbamate: LCMS (ES) m/e 386 (M+H)⁺; ¹H NMR (CD₃OD, 400 MHz) δ 7.04 (s, 1H), 4.69-4.72 (m, 2H), 4.29-4.31 (m, 1H), 4.10-4.12 (m, 1H), 3.31-3.33 (m, 1H), 1.68 (s, 6H), 1.48 (t, 3H), 1.23 (d, J=6.56 Hz, 3H).

### Example 61

### Preparation of 4-[6-{[(2S)-2-amino-3-cyclohexylpropyl]oxy}-2-(4-amino-1,2,5-oxadiazol-3-yl)-1-ethyl-1H-imidazo[4,5-c]pyridin-4-yl]-2-methyl-3-butyn-2-ol

The title compound was prepared as an off-white solid according the Example 56, except substituting 1,1-dimethylethyl[(1R)-2-cyclohexyl-1-(hydroxymethyl)ethyl]carbamate (308 mg, 1.2 mmol) for 1,1-dimethylethyl[(1S)-2-hydroxy-1-methylethyl]carbamate: LCMS (ES) m/e 520 (M+H)⁺; ¹H NMR (CD₃OD, 400 MHz) δ 7.12 (s, 1H), 4.64-4.77 (m, 3H), 4.37-4.44 (m, 1H), 3.74-3.82 (m, 1H), 1.88-1.96 (m, 1H), 1.69-1.87 (m, 5H), 1.68 (s, 6H), 1.53-1.65 (m, 2H) 1.46-1.52 (m, 3H), 1.23-1.40 (m, 3H), 0.97-1.11 (m, 2H).

### Example 62

### Preparation of 4-(2-(4-amino-1,2,5-oxadiazol-3-yl)-6-{[(2S)-2-amino-4-phenylbutyl]oxy}-1-ethyl-1H-imidazo[4,5-c]pyridin-4-yl)-2-methyl-3-butyn-2-ol

### a) 1,1-dimethylethyl[(1S)-1-(hydroxymethyl)-3-phenylpropyl]carbamate

TMSCl (5.7 mL, 44.6 mmol) was added dropwise to a solution of LiBH₄ (486 mg, 22.3 mmol) in THF (22 mL) at 25° C. After 10 min., solid (2S)-2-amino-4-phenylbutanoic acid (2 g, 11.2 mmol) was added portion-wise and complete reduction of the acid was observed after an additional 1 h at 25° C. MeOH was added to quench the excess reagent and the solvent was removed in vacuo. The residue was made alkaline with 1N NaOH and extracted several times with DCM. The combined organic fractions were dried over Na₂SO₄, concentrated and used directly in the following protection.

The crude residue in THF (22 mL) at 25° C. was added Boc₂O dropwise. After 30 min. the solution was concentrated and the residue purified via column chromatography (silica, 2% MeOH in DCM) yielding the title compound (1 g, 34%) as a white solid: LCMS (ES) m/e 266 (M+H)⁺.

### b) 4-(2-(4-amino-1,2,5-oxadiazol-3-yl)-6-{[(2S)-2-amino-4-phenylbutyl]oxy}-1-ethyl-1H-imidazo[4,5-c]pyridin-4-yl)-2-methyl-3-butyn-2-ol

The title compound was prepared as a tan solid according the Example 17, except substituting 1,1-dimethylethyl[(1S)-1-(hydroxymethyl)-3-phenylpropyl]carbamate (284 mg, 1.1 mmol) for 1,1-dimethylethyl (4-hydroxybutyl)carbamate: LCMS (ES) m/e 476 (M+H)⁺; ¹H NMR (CD₃OD, 400 MHz) δ 7.26-7.36 (m, 4H), 7.13-7.20 (m 1H), 7.08 (s, 1H), 4.71 (q, J=7.3 Hz, 2H), 4.41-4.48 (m, 1H), 4.19-4.28 (m, 1H), 3.22-3.31 (m, 1H), 2.71-2.89 (m, 2H), 1.81-2.01 (m, 1H), 1.76-1.83 (m, 1H), 1.67 (s, 6H), 1.42-1.49 (t, J=7.2 Hz, 3H).

### Example 63

### 4-[6-[(2-aminoethyl)oxy]-1-ethyl-4-(3-furanyl)-1H-imidazo[4,5-c]pyridin-2-yl]-1,2,5-oxadiazol-3-amine

### a) 1,1-dimethylethyl (2-{[2-(4-amino-1,2,5-oxadiazol-3-yl)-4-chloro-1-ethyl-1H-imidazo[4,5-c]pyridin-6-yl]oxy}ethyl)carbamate

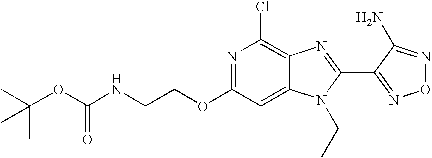

To a solution of 2-(4-amino-1,2,5-oxadiazol-3-yl)-4-chloro-1-ethyl-1,5-dihydro-6H-imidazo[4,5-c]pyridin-6-one (1.37 g, 4.88 mmole), 1,1-dimethylethyl (2-hydroxyethyl)carbamate (1.04 g, 6.46 mmole) and Polystyrene bound PPh₃ (3.43 g, 7.37 mmole) in THF (80 mL) at 5 C was added DEAD (1.4 mL, 7.1 mmole) dropwise. After 1 h, MeOH (1 mL) was added and stirring continued for 30 min. The reaction solution was filtered, concentrated under vacuum and purified on silica gel (hexanes/EtOAc, 2:1) to give the title compound (1.7 g, 35%) as a colorless oil. LC-MS (ES) m/z 424 [M+H]⁺.

### b) 1,1-dimethylethyl (2-{[2-(4-amino-1,2,5-oxadiazol-3-yl)-1-ethyl-4-(3-furanyl)-1H-imidazo[4,5-c]pyridin-6-yl]oxy}ethyl)carbamate

A solution of 1,1-dimethylethyl (2-{[2-(4-amino-1,2,5-oxadiazol-3-yl)-4-chloro-1-ethyl-1H-imidazo[4,5-c]pyridin-6-yl]oxy}ethyl)carbamate (136 mg, 0.321 mmol), 3-furanylboronic acid (47.5 mg, 0.425 mmol), K₂CO₃ (146 mg, 1.056 mmol) and tetrakis(triphenylphosphine)palladium (38 mg, 32.9 μmol) in dioxane/H₂O (10 mL, 5:1) was deoxygenated by purging with nitrogen then heated to 80° C. over 12 h. This solution was then concentrated and purified via column chromatography (5% MeOH in DCM (0.5% NH₄OH)) yielding the title compound (35 mg, 24%) as a yellow solid: LCMS (ES) m/z 456 (M+H)⁺.

### b) 4-[6-[(2-aminoethyl)oxy]-1-ethyl-4-(3-furanyl)-1H-imidazo[4,5-c]pyridin-2-yl]-1,2,5-oxadiazol-3-amine

To a solution of 1,1-dimethylethyl (2-{[2-(4-amino-1,2,5-oxadiazol-3-yl)-1-ethyl-4-(3-furanyl)-1H-imidazo[4,5-c]pyridin-6-yl]oxy}ethyl)carbamate (64 mg, 0.14 mmole) in methanol (15 mL) was added TFA (2 mL). After 2 h at RT, the reaction solution was concentrated under vacuum to give the title compound (13 mg, 89%) as a yellow solid: LCMS (ES) m/z 356 (M+H)⁺. ¹H NMR (d3-MeOH, 400 MHz) δ 8.53 (s, 1H), 7.66 (s, 1H), 7.27 (s, 1H), 6.87 (s, 1H), 4.70-4.59 (m, 4H), 3.58-3.42 (m, 2H), 1.54-1.40 (m, 3H).

### Example 64

### Preparation of 4-[6-{[(2S)-2-amino-3-(1H-imidazol-4-yl)propyl]oxy}-2-(4-amino-1,2,5-oxadiazol-3-yl)-1-ethyl-1H-imidazo[4,5-c]pyridin-4-yl]-2-methyl-3-butyn-2-ol

The title compound was prepared as a tan solid according to the preparation of Example 17, except substituting 1,1-dimethylethyl 4-((2S)-2-({[(1,1-dimethylethyl)oxy]carbonyl}amino)-3-{[(4-methylphenyl)sulfonyl]oxy}propyl)-1H-imidazole-1-carboxylate (0.30 g, 0.92 mmole) for 1,1-dimethylethyl (4-hydroxybutyl)carbamate: LCMS (ES) m/e 452 (M+H)⁺; ¹H NMR (d6-MeOH, 400 MHz) δ 7.67 (s, 1H), 7.05 (s, 1H), 6.98 (s, 1H), 4.59 (m, 2H), 4.47-4.16 (m, 2H), 3.59 (m, 1H), 3.02-2.78 (m, 2H), 1.69 (s, 6H), 1.46 (m, 3H).

### Example 65

### Preparation of (5S)-5-({[2-(4-amino-1,2,5-oxadiazol-3-yl)-1-ethyl-4-(3-hydroxy-3-methyl-1-butyn-1-yl)-1H-imidazo[4,5-c]pyridin-6-yl]oxy}methyl)-2-pyrrolidinone

The title compound was prepared as a brown solid according the Example 17, except substituting (5S)-5-(hydroxymethyl)-2-pyrrolidinone (182 mg, 1.58 mmole) for 1,1-dimethylethyl (4-hydroxybutyl)carbamate: LCMS (ES) m/e 426 (M+H)⁺; ¹H NMR (CD₃OD, 400 MHz) δ 7.63 (s, 1H), 4.82 (m, 2H), 4.62-4.41 (m, 2H), 4.24 (m, 1H), 2.65-2.08 (m, 4H), 1.71 (s, 6H), 1.52 (m, 3H).

### Example 66

### Preparation of (5R)-5-({[2-(4-amino-1,2,5-oxadiazol-3-yl)-1-ethyl-4-(3-hydroxy-3-methyl-1-butyn-1-yl)-1H-imidazo[4,5-c]pyridin-6-yl]oxy}methyl)-2-pyrrolidinone

The title compound was prepared as a tan solid according the Example 17, except substituting (5R)-5-(hydroxymethyl)-2-pyrrolidinone (174 mg, 1.51 mmole) for 1,1-dimethylethyl (4-hydroxybutyl)carbamate: LCMS (ES) m/e 426 (M+H)⁺; ¹H NMR (CD₃OD, 400 MHz) δ 7.06 (s, 1H), 4.71 (m, 2H), 4.52-4.21 (m, 2H), 4.14 (m, 1H), 2.61-2.01 (m, 4H), 1.69 (s, 6H), 1.48 (m, 3H).

### Example 67

### Preparation of 4-(2-(4-amino-1,2,5-oxadiazol-3-yl)-1-ethyl-6-{[2-(1-pyrrolidinyl)ethyl]oxy}-1H-imidazo[4,5-c]pyridin-4-yl)-2-methyl-3-butyn-2-ol

The title compound was prepared as a tan solid according the Example 17, except substituting 2-(1-pyrrolidinyl)ethanol (226 mg, 1.97 mmole) for 1,1-dimethylethyl (4-hydroxybutyl)carbamate: LCMS (ES) m/e 426 (M+H)⁺; ¹H NMR (CD₃OD, 400 MHz) δ 7.20 (s, 1H), 4.74 (m, 4H), 3.88 (m, 2H), 3.74 (m, 2H), 3.29 (m, 2H), 2.32-2.04 (m, 4H), 1.68 (s, 6H), 1.49 (m, 3H).

### Example 68

### Preparation of 4-(2-(4-amino-1,2,5-oxadiazol-3-yl)-1-ethyl-6-{[2-({[4-(methyloxy)phenyl]methyl}amino)ethyl]oxy}-1H-imidazo[4,5-c]pyridin-4-yl)-2-methyl-3-butyn-2-ol

### a) 2-aminoethanol

HCl in dioxane (12 mL, 4 M in dioxane) was added to a solution of 1,1-dimethylethyl (2-hydroxyethyl)carbamate (2.2 g, 13.7 mmole) in THF (20 mL) and the mixture was stirred overnight. The solvents were removed under vacuum and the resulting solid was used in the next step without further purification.

### b) 2-({[4-(methyloxy)phenyl]methyl}amino)ethanol

A solution of 2-aminoethanol (421 mg, 4.32 mmole) and 4-(methyloxy)benzaldehyde (593 mg, 4.36 mmole) were stirred overnight in DCM/EtOH (15 mL, 10:2) with Na₂SO₄ (3.0 g, 21 mmole). To this mixture was added NaB(OAc)₃H (1.37 g, 6.46 mmole) and the reaction stirred for 3 hours. The mixture was concentrated and purified via column chromatography (silica, 0-20% MeOH in DCM) yielding the title compound (433 mg, 55%) as a yellow oil: LCMS (ES) m/e 182 (M+H)⁺.

### c) 1,1-dimethylethyl (2-hydroxyethyl){[4-(methyloxy)phenyl]methyl}carbamate

To a solution of 2-({[4-(methyloxy)phenyl]methyl}amino)ethanol (433 mg, 2.39 mmole) in THF at RT was added 1M Boc anhydride in THF (2.6 mL, 2.6 mmole). After 3 h, the reaction solution was concentrated under vacuum and the residue purified on silica gel (hexanes/EtOAc, 1:1) to give the title compound (235 mg, 35%) as a waxy yellow solid: LCMS (ES) m/z=282 (M+H)⁺.

### b) 4-(2-(4-amino-1,2,5-oxadiazol-3-yl)-1-ethyl-6-{[2-({[4-(methyloxy)phenyl]methyl}amino)ethyl]oxy}-1H-imidazo[4,5-c]pyridin-4-yl)-2-methyl-3-butyn-2-ol (GSK949686)

The title compound was prepared as a tan solid according the Example 17, except substituting 1,1-dimethylethyl (2-hydroxyethyl){[4-(methyloxy)phenyl]methyl}carbamate (235 mg, 0.835 mmole) for 1,1-dimethylethyl (4-hydroxybutyl)carbamate: LCMS (ES) m/e 492 (M+H)⁺; ¹H NMR (CD₃OD, 400 MHz) δ 7.49 (d, J=8.6 Hz, 2H), 7.26 (s, 1H), 7.01 (d, J=8.7 Hz, 2H), 4.74 (m, 4H), 4.33 (s, 2H), 3.82 (s, 3H), 3.80-3.53 (m, 4H), 1.68 (s, 6H), 1.49 (m, 3H).

### Example 69

### Preparation of 4-(2-(4-amino-1,2,5-oxadiazol-3-yl)-1-ethyl-6-{[2-({[4-(trifluoromethyl)phenyl]methyl}amino)ethyl]oxy}-1H-imidazo[4,5-c]pyridin-4-yl)-2-methyl-3-butyn-2-ol

The title compound was prepared as a tan solid according the Example 68, except substituting 1,1-dimethylethyl (2-hydroxyethyl){[4-(trifluoromethyl)phenyl]methyl}carbamate (157 mg, 492 mmole) for 1,1-dimethylethyl (2-hydroxyethyl){[4-(methyloxy)phenyl]methyl}carbamate: LCMS (ES) m/e 530 (M+H)⁺; ¹H NMR (CD₃OD, 400 MHz) δ 7.94-7.74 (m, 4H), 7.15 (d, J=3.7 Hz, 2H), 4.73 (m, 4H), 4.50 (m, 2H), 3.62 (m, 2H), 1.64 (s, 6H), 1.49 (m, 3H).

### Example 70

### Preparation of 4-[2-(4-amino-1,2,5-oxadiazol-3-yl)-1-ethyl-6-(4-piperidinyloxy)-1H-imidazo[4,5-c]pyridin-4-yl]-2-methyl-3-butyn-2-ol

The title compound was prepared as a tan solid according the Example 17, except substituting 1,1-dimethylethyl 4-hydroxy-1-piperidinecarboxylate (286 mg, 1.42 mmole) for 1,1-dimethylethyl (4-hydroxybutyl)carbamate: LCMS (ES) m/e 412 (M+H)⁺; ¹H NMR (CD₃OD, 400 MHz) δ 7.42 (s, 1H), 5.41 (m, 1H), 4.77 (s, 2H), 3.50 (m, 2H), 3.41-3.29 (m, 2H), 2.41-2.11 (m, 4H), 1.69 (s, 6H), 1.49 (m, 3H).

### Example 71

### Preparation of 4-(2-(4-amino-1,2,5-oxadiazol-3-yl)-1-ethyl-6-{[2-(4-morpholinyl)ethyl]oxy}-1H-imidazo[4,5-c]pyridin-4-yl)-2-methyl-3-butyn-2-ol

The title compound was prepared as a green solid according the Example 17, except substituting 2-(4-morpholinyl)ethanol (325 mg, 2.48 mmole) for 1,1-dimethylethyl (4-hydroxybutyl)carbamate: LCMS (ES) m/e 442 (M+H)⁺; ¹H NMR (CD₃OD, 400 MHz) δ 7.00 (s, 1H), 4.69 (m, 2H), 4.52 (m, 2H), 3.76 (m, 4H), 2.88 (m, 2H), 2.67 (m, 4H), 1.67 (s, 6H), 1.47 (m, 3H).

### Example 72

### Preparation of 4-(2-(4-amino-1,2,5-oxadiazol-3-yl)-1-ethyl-6-{[2-(phenylamino)ethyl]oxy}-1H-imidazo[4,5-c]pyridin-4-yl)-2-methyl-3-butyn-2-ol

The title compound was prepared as a green solid according the Example 17, except substituting 1,1-dimethylethyl (2-hydroxyethyl)carbamate (304 mg, 2.21 mmole) for 1,1-dimethylethyl (4-hydroxybutyl)carbamate: LCMS (ES) m/e 448 (M+H)⁺; ¹H NMR (CD Cl₃, 400 MHz) δ 7.38 (m, 2H), 7.23 (m, 2H), 7.16 (m, 1H), 6.79 (s, 1H), 4.69 (m, 2H), 4.61 (m, 2H), 3.71 (m, 2H), 1.68 (s, 6H), 1.46 (m, 3H).

### Example 73

### Preparation of 4-[6-{[(2S)-2-amino-3-(1-methyl-1H-indol-3-yl)propyl]oxy}-2-(4-amino-1,2,5-oxadiazol-3-yl)-1-ethyl-1H-imidazo[4,5-c]pyridin-4-yl]-2-methyl-3-butyn-2-ol

The title compound was prepared as a tan solid according the Example 17, except substituting 1,1-dimethylethyl {(1S)-2-hydroxy-1-[(1-methyl-1H-indol-2-yl)methyl]ethyl}carbamate (614 mg, 2.02 mmole) for 1,1-dimethylethyl (4-hydroxybutyl)carbamate: LCMS (ES) m/e 515 (M+H)⁺; ¹H NMR (CD₃OD, 400 MHz) δ 7.65 (d, J=7.9 Hz, 1H), 7.38 (d, J=8.2 Hz, 1H), 7.30-7.17 (m, 2H), 7.08 (m, 1H), 4.79-4.46 (m, 4H), 4.02 (m, 1H), 3.82 (s, 3H), 3.42-3.22 (m, 3H), 1.69 (s, 6H), 1.49 (m, 3H).

### Example 74

### Preparation of 4-[2-(4-amino-1,2,5-oxadiazol-3-yl)-6-({(2S)-2-amino-3-[(phenylmethyl)thio]propyl}oxy)-1-ethyl-1H-imidazo[4,5-c]pyridin-4-yl]-2-methyl-3-butyn-2-ol

The title compound was prepared as a orange solid according the Example 17, except substituting 1,1-dimethylethyl((1S)-2-hydroxy-1-{[(phenylmethyl)thio]methyl}ethyl)carbamate (637 mg, 2.14 mmole) for 1,1-dimethylethyl (4-hydroxybutyl)carbamate: LCMS (ES) m/e 508 (M+H)⁺; ¹H NMR (CDCl₃, 400 MHz) δ 7.39-7.21 (m, 5H), 6.71 (s, 1H), 4.63 (m, 2H), 4.38 (m, 2H), 3.79 (s, 2H), 3.38 (m, 1H), 2.66 (m, 2H), 1.72 (s, 6H), 1.49 (m, 3H).

### Example 75

### Preparation of 4-(2-(4-amino-1,2,5-oxadiazol-3-yl)-6-{[(2R)-2-amino-3-(3-pyridinyl)propyl]oxy}-1-ethyl-1H-imidazo[4,5-c]pyridin-4-yl)-2-methyl-3-butyn-2-ol

The title compound was prepared as a brown solid according the Example 62, except substituting 3-(3-pyridinyl)-D-alanine (1 g, 6.02 mmol) for (2S)-2-amino-4-phenylbutanoic acid: LCMS (ES) m/e 463 (M+H)⁺; ¹H NMR (CD₃OD, 400 MHz) δ 8.51 (s, 1H), 8.43 (d, J=4.9 Hz, 1H), 7.82 (d, J=5.0 Hz, 1H), 7.46 (dd, J=4.8, 5.1 Hz, 1H), 7.05 (s, 1H), 4.65-4.71 (m, 2H), 4.29-4.39 (m, 1H), 4.17-4.22 (m, 1H), 3.45-3.52 (m, 1H), 3.00-3.17 (m, 1H), 2.88-2.91 (m, 1H), 1.67 (s, 6H), 1.46 (dd, J=7.0, 7.1 Hz, 3H).

### Example 76

### Preparation of 4-(2-(4-amino-1,2,5-oxadiazol-3-yl)-6-{[(2R)-2-amino-4-phenylbutyl]oxy}-1-ethyl-1H-imidazo[4,5-e]pyridin-4-yl)-2-methyl-3-butyn-2-ol

The title compound was prepared as a yellow solid according the Example 62, except substituting (2R)-2-amino-4-phenylbutanoic acid (2 g, 11.2 mmol) for (2S)-2-amino-4-phenylbutanoic acid: LCMS (ES) m/e 476 (M+H)⁺; ¹H NMR (CD₃OD, 400 MHz) δ 7.26-7.36 (m, 4H), 7.13-7.20 (m 1H), 7.08 (s, 1H), 4.71 (q, J=7.3 Hz, 2H), 4.41-4.48 (m, 1H), 4.19-4.28 (m, 1H), 3.22-3.31 (m, 1H), 2.71-2.89 (m, 2H), 1.81-2.01 (m, 1H), 1.76-1.83 (m, 1H), 1.67 (s, 6H), 1.42-1.49 (t, J=7.2 Hz, 3H).

### Example 77

### Preparation of 4-{2-(4-amino-1,2,5-oxadiazol-3-yl)-6-[(2-amino-1-phenylethyl)oxy]-1-ethyl-1H-imidazo[4,5-c]pyridin-4-yl}-2-methyl-3-butyn-2-ol

The title compound was prepared as an orange solid according to Example 17, except substituting 2-amino-1-phenylethanol (1 g, 7.29 mmol) for 4-amino-1-butanol: LCMS (ES) m/e 448 (M+H)⁺; ¹H NMR ((CD₃OD, 400 MHz) δ 7.27-7.50 (m, 5H), 7.03 (s, 1H), 5.49-5.52 (m, 1H), 4.63-4.66 (m, 2H), 3.14-3.17 (m, 1H), 3.07-3.12 (m, 1H), 1.65 (s, 6H), 1.38-1.42 (m, 3H).

### Example 78

### Preparation of 4-[6-(aminomethyl)-2-(4-amino-1,2,5-oxadiazol-3-yl)-1-ethyl-1H-imidazo[4,5-c]pyridin-4-yl]-2-methyl-3-butyn-2-ol

### a) 4-(4-chloro-6-ethenyl-1-ethyl-1H-imidazo[4,5-c]pyridin-2-yl)-1,2,5-oxadiazol-3-amine

A solution of 4-(6-bromo-4-chloro-1-ethyl-1H-imidazo[4,5-c]pyridin-2-yl)-1,2,5-oxadiazol-3-amine (1 g, 2.92 mmol), trivinylboronate (352 mg, 1.46 mmol), K₂CO₃(403 mg, 2.92 mmol) and Pd(PPh₃)₄ (168 mg, 0.146 mmol) in dioxane (24 mL) and H₂O (8 mL) were heated at 70° C. in a sealed tube. After 3 h, the solution was concentrated then tritrated using 3% MeOH in DCM affording the title compound (848 mg, quant.) as a yellow solid: LCMS (ES) m/e 292 (M+H)⁺.

### b) [2-(4-amino-1,2,5-oxadiazol-3-yl)-4-chloro-1-ethyl-1H-imidazo[4,5-c]pyridin-6-yl]methanol

O₃ was bubbled through a −78° C. solution of 4-(4-chloro-6-ethenyl-1-ethyl-1H-imidazo[4,5-c]pyridin-2-yl)-1,2,5-oxadiazol-3-amine (390 mg, 1.34 mmol) in DCM (20 mL). After 5 min the excess O₃ was removed by bubbling through a stream of N₂. MeOH (5 mL) was then added as a cosolvent followed by NaBH₄ (254 mg, 6.72 mmol) in one portion. The solution warmed to 0° C. and was partitioned between H₂O-DCM. The aqueous phase was back-extracted several times with DCM and the combined organic fractions were dried over Na₂SO₄ and concentrated affording the alcohol (161 mg) as a yellow solid which was used directly without further purification: LCMS (ES) m/e 295 (M+H)⁺.

### c) 2-{[2-(4-amino-1,2,5-oxadiazol-3-yl)-4-chloro-1-ethyl-1H-imidazo[4,5-c]pyridin-6-yl]methyl}-1H-isoindole-1,3(2H)-dione

diethyl azodicarboxylate (128 μL, 0.814 mmol) was added dropwise to a solution of [2-(4-amino-1,2,5-oxadiazol-3-yl)-4-chloro-1-ethyl-1H-imidazo[4,5-c]pyridin-6-yl]methanol (160 mg, 0.542 mmol), phthalimide (80 mg, 0.542 mmol) and triphenylphosphine (213 mg, 0.814 mmol) in THF (5 mL) at 25° C. After 1 h, the solution was partitioned between H₂O-DCM and the aqueous phase was back-extracted several times with DCM. The combined organic fractions were dried over Na₂SO₄, concentrated and purified via column chromatography (silica, 1% MeOH in DCM) yielding the title compound (170 mg, 74%) as a white solid: LCMS (ES) m/e 424 (M+H)⁺.

### d) 2-{[2-(4-amino-1,2,5-oxadiazol-3-yl)-1-ethyl-4-(3-hydroxy-3-methyl-1-butyn-1-yl)-1H-imidazo[4,5-c]pyridin-6-yl]methyl}-1H-isoindole-1,3(2H)-dione

A solution of 2-{[2-(4-amino-1,2,5-oxadiazol-3-yl)-4-chloro-1-ethyl-1H-imidazo[4,5-c]pyridin-6-yl]methyl}-1H-isoindole-1,3(2H)-dione (170 mg, 0.402 mmol), 2-methyl-3-butyne-2-ol (126 μL, 1.3 mmol), CuI (8 mg, 40 μmol) and Pd(PPh₃)Cl₂ (28 mg, 40 μmol) in DMF-Et₃N (2:1, 2 mL) was stirred at 70° C. in a sealed tube. After 3 h, the solution was concentrated and purified via column chromatography (silica, 1-2% MeOH in DCM) yielding the title compound (93 mg, 49%) as an orange solid: LCMS (ES) m/e 472 (M+H)⁺.

### e) 4-[6-(aminomethyl)-2-(4-amino-1,2,5-oxadiazol-3-yl)-1-ethyl-1H-imidazo[4,5-c]pyridin-4-yl]-2-methyl-3-butyn-2-ol

Methylamine (40 wt % in H₂O, 10 mL, 3.94 mmol) was added dropwise to a solution of 2-{[2-(4-amino-1,2,5-oxadiazol-3-yl)-1-ethyl-4-(3-hydroxy-3-methyl-1-butyn-1-yl)-1H-imidazo[4,5-c]pyridin-6-yl]methyl}-1H-isoindole-1,3(2H)-dione (93 mg, 0.197 mmol) in MeOH (2 mL) at 25° C. After 30 min, the solution was concentrated using a toluene azeotrope and purified on silica (5% MeOH in DCM (1% NH₄OH)) affording the title compound (47 mg, 70%) as a yellow solid: LCMS (ES) m/e 342 (M+H)⁺; ¹H NMR (CD₃OD, 400 MHz) δ 7.76 (s, 1H), 4.79 (q, J=7.2 Hz, 2H), 4.01-4.12 (m, 2H), 1.69 (s, 6H), 1.51 (t, J=7.3 Hz, 3H).

### Example 79

### Preparation of 4-[2-(4-amino-1,2,5-oxadiazol-3-yl)-1-ethyl-6-[(methylamino)methyl]-1H-imidazo[4,5-c]pyridin-4-yl]-2-methyl-3-butyn-2-ol

### a) 4-{4-chloro-1-ethyl-6-[(methylamino)methyl]-1H-imidazo[4,5-c]pyridin-2-yl}-1,2,5-oxadiazol-3-amine

O₃ was passed through a −78° C. solution of 4-(4-chloro-6-ethenyl-1-ethyl-1H-imidazo[4,5-c]pyridin-2-yl)-1,2,5-oxadiazol-3-amine (240 mg, 0.828 mmol)[prepared in Example 79] in DCM (12 mL). After 5 min the excess O₃ was removed by bubbling through a stream of N₂. Dimethyl sulfide (85 μL, 1.16 mmol) was added and the solution warmed to 25° C. over 1 h. The DCM was removed in vacuo and the residue was dissolved in THF (8 mL) and cooled to 0° C. Methylamine (2M in THF, 200 μL, 0.911 mmol) was added followed by Na₂SO₄ (117 mg, 1.66 mmol) and the solution stirred at 25° C. for 12 h. After cooling to 0° C., MeOH (2 mL) was added as cosolvent followed by NaBH₄ (19 mg, 0.502 mmol) in one portion. After 2 h, the solution was partitioned between H₂O-DCM. The aqueous phase was back-extracted several times with DCM and the combined organic fractions were dried over Na₂SO₄, concentrated and purified via column chromatography (silica, 2% MeOH in DCM (1% NH₄OH)) yielding the title compound (23 mg, 18%-2 steps) as a white powder: LCMS (ES) m/e 308 (M+H)⁺.

### b) 4-{2-(4-amino-1,2,5-oxadiazol-3-yl)-1-ethyl-6-[(methylamino)methyl]-1H-imidazo[4,5-c]pyridin-4-yl}-2-methyl-3-butyn-2-ol

A solution of 4-{4-chloro-1-ethyl-6-[(methylamino)methyl]-1H-imidazo[4,5-c]pyridin-2-yl}-1,2,5-oxadiazol-3-amine (23 mg, 75 μmol), 2-methyl-3-butyne-2-ol (23 μL, 0.239 mmol), CuI (1 mg, 7.5 μmol) and Pd(PPh₃)Cl₂ (5 mg, 7.5 μmol) in DMF-Et₃N (2:1, 1.5 mL) was stirred at 70° C. in a sealed tube. After 3 h, the solution was concentrated and purified via column chromatography (silica, 2-5% MeOH in DCM) yielding the title compound (14 mg, 52%) as a brown solid: LCMS (ES) m/e 356 (M+H)⁺, ¹H NMR (CD₃OD, 400 MHz) δ 7.76 (s, 1H), 4.79 (q, J=7.2 Hz, 2H), 4.00 (bs, 2H), 2.49 (s, 3H), 1.68 (s, 6H), 1.51 (t, J=7.1 Hz, 3H).

### Example 80

### Preparation of 4-(2-(4-amino-1,2,5-oxadiazol-3-yl)-1-ethyl-6-{[(phenylmethyl)amino]methyl}-1H-imidazo[4,5-c]pyridin-4-yl)-2-methyl-3-butyn-2-ol

The title compound was prepared as an orange solid according to Example 79, except substituting benzylamine (56 μL, 0.50 mmol) for methylamine: LCMS (ES) m/e 356 (M+H)⁺, ¹H NMR (CD₃OD, 400 MHz) δ 7.72 (s, 1H), 7.26-7.37 (m, 5H), 4.75 (q, J=7.4 Hz, 2H), 4.00 (bs, 2H), 3.84 (bs, 2H), 1.68 (s, 6H), 1.47 (t, J=7.1 Hz, 3H).

### Example 81

### Preparation of 4-[2-(4-amino-1,2,5-oxadiazol-3-yl)-6-(3-aminopropyl)-1-ethyl-1H-imidazo[4,5-c]pyridin-4-yl]-2-methyl-3-butyn-2-ol

### a) 2-{3-[2-(4-amino-1,2,5-oxadiazol-3-yl)-1-ethyl-4-(3-hydroxy-3-methyl-1-butyn-1-yl)-1H-imidazo[4,5-c]pyridin-6-yl]propyl}-1H-isoindole-1,3(2H)-dione

### Method 1

A solution of 2-(2-propen-1-yl)-1H-isoindole-1,3(2H)-dione (65 mg, 0.347 mmol) and 9-BBN dimmer (106 mg, 0.434 mmol) were heated at 75° C. for 30 min where TLC indicated disappearance of starting phthalimide. Potassium carbonate (80 mg, 0.578 mmol) and 4-[2-(4-amino-1,2,5-oxadiazol-3-yl)-6-chloro-1-ethyl-1H-imidazo[4,5-c]pyridin-4-yl]-2-methyl-3-butyn-2-ol [prepared in Preparation 1] (100 mg, 0.289 mmol) were then added in one portion followed by a pre-heated (75° C. for 30 min) solution of Pd(OAc)₂ (6 mg, 28.9 μmol), DPPF (25 mg, 43.4 μmol) in DMF (1 mL). The resulting solution stirred for 5 h and was then partitioned between H₂O-DCM. The aqueous phase was back-extracted several times with DCM and the combined organic fractions were dried over Na₂SO₄, concentrated and columned (silica, 1% MeOH in DCM (1% NH₄OH)) affording the title compound (25 mg, 17%) as an orange oil: LCMS (ES) m/e 500 (M+H)⁺.

### Method 2

### i) 2-{3-[2-(4-amino-1,2,5-oxadiazol-3-yl)-4-chloro-1-ethyl-1H-imidazo[4,5-c]pyridin-6-yl]propyl}-1H-isoindole-1,3(2H)-dione

The title compound was prepared as a yellow foam according to Method 1, except substituting 4-(6-bromo-4-chloro-1-ethyl-1H-imidazo[4,5-c]pyridin-2-yl)-1,2,5-oxadiazol-3-amine (300 mg, 0.875 mmol) for 4-[2-(4-amino-1,2,5-oxadiazol-3-yl)-6-chloro-1-ethyl-1H-imidazo[4,5-c]pyridin-4-yl]-2-methyl-3-butyn-2-ol: LCMS (ES) m/e 452 (M+H)⁺.

### ii) 2-{3-[2-(4-amino-1,2,5-oxadiazol-3-yl)-1-ethyl-4-(3-hydroxy-3-methyl-1-butyn-1-yl)-1H-imidazo[4,5-c]pyridin-6-yl]propyl}-1H-isoindole-1,3(2H)-dione

A solution of 2-{3-[2-(4-amino-1,2,5-oxadiazol-3-yl)-4-chloro-1-ethyl-1H-imidazo[4,5-c]pyridin-6-yl]propyl}-1H-isoindole-1,3(2H)-dione (95 mg, 0.211 mmol), 2-methyl-3-butyne-2-ol (66 μL, 0.674 mmol), CuI (4 mg, 21.1 μmol) and Pd(PPh₃)Cl₂ (15 mg, 21.1 μmol) in DMF-Et₃N (2:1, 2.2 mL) was stirred at 70° C. in a sealed tube. After 3 h, the solution was concentrated and purified via column chromatography (silica, 1% MeOH in DCM (1% NH₄OH) yielding the title compound (62 mg, 59%) as an orange oil: LCMS (ES) m/e 500 (M+H)⁺.

### b) 4-[2-(4-amino-1,2,5-oxadiazol-3-yl)-6-(3-aminopropyl)-1-ethyl-1H-imidazo[4,5-c]pyridin-4-yl]-2-methyl-3-butyn-2-ol

Methylamine (40 wt % in H₂O, 8.7 mL, 3.48 mmol) was added dropwise to a solution of 2-{3-[2-(4-amino-1,2,5-oxadiazol-3-yl)-1-ethyl-4-(3-hydroxy-3-methyl-1-butyn-1-yl)-1H-imidazo[4,5-c]pyridin-6-yl]propyl}-1H-isoindole-1,3(2H)-dione (87 mg, 0.174 mmol) in MeOH (1.7 mL) at 25° C. After 30 min, the solution was concentrated using a toluene azeotrope then purified via column chromatography (silica, 90:10:1, DCM:MeOH:NH₄OH) affording the title compound (38 mg, 59%) as a yellow powder: LCMS (ES) m/e 370 (M+H)⁺; ¹H NMR (CD₃OD, 400 MHz) δ 7.63 (s, 1H), 4.76 (q, J=7.2 Hz, 2H), 2.95-2.99 (m, 2H), 2.76-2.80 (m, 2H), 1.93-2.12 (m, 2H), 1.69 (s, 6H), 1.49 (t, J=7.3 Hz, 3H).

### Example 82

### Preparation of 4-[6-(2-aminoethyl)-2-(4-amino-1,2,5-oxadiazol-3-yl)-1-ethyl-1H-imidazo[4,5-c]pyridin-4-yl]-2-methyl-3-butyn-2-ol

The title compound was prepared as a yellow solid according to Example 81 (Method 1), except substituting 2-ethenyl-1H-isoindole-1,3(2H)-dione (240 mg, 1.39 mmol) for 2-(2-propen-1-yl)-1H-isoindole-1,3(2H)-dione: LCMS (ES) m/e 356 (M+H)⁺; ¹H NMR (CD₃OD, 400 MHz) δ 7.64 (s, 1H), 4.78 (q, J=7.1 Hz, 2H), 3.10 (bs, 2H), 1.51 (t, J=7.1 Hz, 3H).

### Example 83

### Preparation of 4-[2-(4-amino-1,2,5-oxadiazol-3-yl)-1-ethyl-6-(4-morpholinylmethyl)-1H-imidazo[4,5-c]pyridin-4-yl]-2-methyl-3-butyn-2-ol

The title compound was prepared as an orange solid according to Example 79, except substituting morpholine (0.13 g, 1.5 mmol) for methylamine: LCMS (ES) m/e 412 (M+H)⁺, ¹H NMR (d6-dmso, 400 MHz) δ 7.72 (s, 1H), 7.05 (s, 1H), 4.61 (q, J=7.4 Hz, 2H), 3.87 (bs, 2H), 3.31 (bs, 2H), 1.58 (s, 6H), 1.47 (t, J=7.1 Hz, 3H).

### Example 84

### Preparation of 4-(2-(4-amino-1,2,5-oxadiazol-3-yl)-1-ethyl-6-{[methyl(phenylmethyl)amino]methyl}-1H-imidazo[4,5-c]pyridin-4-yl)-2-methyl-3-butyn-2-ol

The title compound was prepared as an orange solid according to Example 79, except substituting N-methylbenzylamine (0.31 g, 2.6 mmol) for methylamine: LCMS (ES) m/e 446 (M+H)⁺, ¹H NMR (CD₃OD, 400 MHz) δ 8.15 (s, 1H), 7.65 (s, 2H), 7.49 (m, 2H), 7.02 (s, 2H), 4.73 (q, J=7.4 Hz, 2H), 4.52 (brs, 2H), 4.41 (bs, 2H), 2.71 (s, 3H), 1.60 (s, 6H), 1.47 (t, J=7.1 Hz, 3H).

### Example 85

### Preparation of 4-(2-(4-amino-1,2,5-oxadiazol-3-yl)-1-ethyl-6-{[methyl(2-phenylethyl)amino]methyl}-1H-imidazo[4,5-c]pyridin-4-yl)-2-methyl-3-butyn-2-ol

The title compound was prepared as an orange solid according to Example 79, except substituting N-methylphenethylamine (0.35 g, 2.57 mmol) for methylamine: LCMS (ES) m/e 460 (M+H)⁺, ¹H NMR (d6-DMSO, 400 MHz) δ 8.28 (s, 1H), 7.50-7.65 (m, 2H), 7.35-7.45 (m, 4H), 7.02 (s, 2H), 4.71 (q, J=7.4 Hz, 2H), 3.80 (br s, 4H), 3.45 (br s, 2H), 2.84 (s, 3H), 1.60 (s, 6H), 1.47 (t, J=7.1 Hz, 3H).

### Example 86

### Preparation of 4-(2-(4-amino-1,2,5-oxadiazol-3-yl)-6-{[(1R)-2-amino-1-phenylethyl]oxy}-1-ethyl-1H-imidazo[4,5-c]pyridin-4-yl)-2-methyl-3-butyn-2-ol

The title compound was prepared as a tan solid according to the preparation of Example 17, except substituting 1,1-dimethylethyl[(2R)-2-hydroxy-2-phenylethyl]carbamate (320 mg, 1.34 mmol) for 1,1-dimethylethyl (4-hydroxybutyl)carbamate: LCMS (ES) m/e 448 (M+H)⁺; ¹H NMR (d6-DMSO, 400 MHz) δ 8.45 (br s, 2H), 7.51 (m, 2H), 7.42 (m, 2H), 7.37 (m, 2H), 7.00 (brs, 2H), 6.49 (m, 1H), 4.62 (q, J=7.0 Hz, 2H), 3.67 (m, 2H), 1.53 (s, 6H), 1.38 (t, J=7.0 Hz, 3H). [α]_{D}=−30.0° (CH₃OH, C=1.0, 20° C.)

### Example 87

### Preparation of 4-(2-(4-amino-1,2,5-oxadiazol-3-yl)-1-ethyl-6-{[2-(methylamino)-1-phenylethyl]oxy}-1H-imidazo[4,5-c]pyridin-4-yl)-2-methyl-3-butyn-2-ol

The title compound was prepared as a tan solid according to the preparation of Example 17, except substituting 1,1-dimethylethyl (2-hydroxy-2-phenylethyl)methylcarbamate (270 mg, 1.07 mmol) for 1,1-dimethylethyl (4-hydroxybutyl)carbamate: LCMS (ES) m/e 462 (M+H)⁺; ¹H NMR (d6-DMSO, 400 MHz) δ 8.45 (br s, 2H), 7.51 (m, 2H), 7.42 (m, 2H), 7.37 (m, 2H), 7.00 (br s, 2H), 6.49 (m, 1H), 4.62 (q, J=7.0 Hz, 2H), 3.67 (m, 2H), 2.60 (s, 3H), 1.53 (s, 6H), 1.38 (t, J=7.0 Hz, 3H).

### Example 88

### Preparation of 4-[6-[(2-amino-1-cyclohexylethyl)oxy]-2-(4-amino-1,2,5-oxadiazol-3-yl)-1-ethyl-1H-imidazo[4,5-c]pyridin-4-yl]-2-methyl-3-butyn-2-ol

### a) cyclohexyl(hydroxy)acetonitrile

To a suspension of potassium cyanide (6.82 g, 105 mmole) in anhydrous diethyl ether (100 mL) at 5° C. was added dropwise a solution of cyclohexyl carboxaldehyde (5.0 g, 44.6 mmole) in conc. acetic acid (6 mL). The suspension was allowed to warm to RT overnight with vigourous stirring. The potassium acetate was filtered from the reaction and the mother liquor concentrated at RT. The residue was placed under high vacuum at RT for 3 h and used directly in the proceeding step.

### b) 1,1-dimethylethyl (2-cyclohexyl-2-hydroxyethyl)carbamate

Crude cyclohexyl(hydroxy)acetonitrile (3.5 g, 25.3 mmole) was dissolved in dry THF (100 mL) and the solution cooled to 0° C. LiAlH₄ (30 mL, 1M in THF) was added and the reaction solution was allowed to warm to RT overnight. The reaction was quenched with an aqueous basic work-up: (1.3 mL H₂O; 1 mL 6N NaOH; 4.6 mL H₂O). The aluminum salts were filtered and washed with diethyl ether. The filtrate was concentrated under vacuum and dried under high vacuum at RT.

The crude amino alcohol (3.3 g, 23.7 mmole), from above, was dissolved in THF (50 mL) and Boc anhydride (5.17 g, 23.7 mmole) was added. The reaction solution was allowed to stir at RT for 4 h and was then concentrated under vacuum. Purification on silica (hexanes/EtOAc, 4/1) provided the title compound as a white solid: LCMS (ES) m/z=243 (M+H)⁺

### c) 4-[6-[(2-amino-1-cyclohexylethyl)oxy]-2-(4-amino-1,2,5-oxadiazol-3-yl)-1-ethyl-1H-imidazo[4,5-c]pyridin-4-yl]-2-methyl-3-butyn-2-ol

The title compound was prepared as a tan solid according to the preparation of Example 17, except substituting 1,1-dimethylethyl (2-cyclohexyl-2-hydroxyethyl)carbamate (310 mg, 1.28 mmol) for 1,1-dimethylethyl (4-hydroxybutyl)carbamate: LCMS (ES) m/e 453 (M+H)⁺; ¹H NMR (d6-DMSO, 400 MHz) δ 8.21 (br s, 2H), 7.30 (s, 1H), 7.02 (br s, 2H), 5.26 (m, 1H), 4.65 (q, J=7.0 Hz, 2H), 3.12 (m, 2H), 1.75 (m, 6H), 1.53 (s, 6H), 1.38 (t, J=7.0 Hz, 3H), 1.16 (m, 4H).

### Example 89

### Preparation of 4-(2-(4-amino-1,2,5-oxadiazol-3-yl)-6-{[2-amino-1-(tetrahydro-2H-pyran-4-yl)ethyl]oxy}-1-ethyl-1H-imidazo[4,5-c]pyridin-4-yl)-2-methyl-3-butyn-2-ol

The title compound was prepared as a tan solid according to the preparation of Example 17, except substituting 1,1-dimethylethyl[2-hydroxy-2-(tetrahydro-2H-pyran-4-yl)ethyl]carbamate (310 mg, 1.28 mmol) [prepared according to the procedure of Example 88] for 1,1-dimethylethyl (4-hydroxybutyl)carbamate: LCMS (ES) m/e 456 (M+H)⁺; ¹H NMR (d6-DMSO, 400 MHz) δ 8.37 (br s, 2H), 7.31 (m, 1H), 7.00 (br s, 2H), 5.38 (m, 1H), 4.62 (q, J=7.0 Hz, 2H), 3.88 (m, 2H), 3.70 (m, 2H), 3.49 (m, 2H), 3.27 (m, 2H), 3.12 (m, 2H), 2.05 (m, 1H), 1.53 (s, 6H), 1.38 (t, J=7.0 Hz, 3H).

### Example 90

### Preparation of 4-(2-(4-amino-1,2,5-oxadiazol-3-yl)-6-{[2-amino-1-(3-Pyridinyl)ethyl]oxy}-1-ethyl-1H-imidazo[4,5-c]pyridin-4-yl)-2-methyl-3-butyn-2-ol

The title compound was prepared as a tan solid according to the preparation of Example 17, except substituting 1,1-dimethylethyl[2-hydroxy-2-(3-pyridinyl)ethyl]carbamate (300 mg, 1.28 mmol) [prepared according to the procedure of Example 88] for 1,1-dimethylethyl (4-hydroxybutyl)carbamate: LCMS (ES) m/e 449 (M+H)⁺; ¹H NMR (d6-DMSO, 400 MHz) δ 8.65 (m, 2H), 8.45 (br s, 2H), 8.08 (br s, 1H), 7.49 (s, 1H), 7.00 (br s, 2H), 6.56 (m, 1H), 4.62 (q, J=7.0 Hz, 2H), 3.70 (m, 1H), 3.54 (m, 1H), 1.53 (s, 6H), 1.38 (t, J=7.0 Hz, 3H).

### Example 91

### Preparation of 4-[6-[(2-amino-1-cyclopropylethyl)oxy]-2-(4-amino-1,2,5-oxadiazol-3-yl)-1-ethyl-1H-imidazo[4,5-c]pyridin-4-yl]-2-methyl-3-butyn-2-ol

The title compound was prepared as a tan solid according to the preparation of Example 17, except substituting 1,1-dimethylethyl (2-cyclopropyl-2-hydroxyethyl)carbamate (260 mg, 1.29 mmol) [prepared according to the procedure of Example 88] for 1,1-dimethylethyl (4-hydroxybutyl)carbamate: LCMS (ES) m/e 412 (M+H)⁺; ¹H NMR (d6-DMSO, 400 MHz) δ 8.15 (br s, 2H), 7.25 (s, 1H), 7.03 (br s, 2H), 4.91 (m, 1H), 4.62 (q, J=7.0 Hz, 2H), 3.55 (m, 2H), 1.53 (s, 6H), 1.38 (t, J=7.0 Hz, 3H), 1.15 (m, 1H), 0.56 (m, 4H).

### Example 92

### Preparation of 4-[6-{[2-amino-1-(1,3-benzodioxol-4-yl)ethyl]oxy}-2-(4-amino-1,2,5-oxadiazol-3-yl)-1-ethyl-1H-imidazo[4,5-c]pyridin-4-yl]-2-methyl-3-butyn-2-ol

The title compound was prepared as a tan solid according to the preparation of Example 17, except substituting 1,1-dimethylethyl[2-(1,3-benzodioxol-4-yl)-2-hydroxyethyl]carbamate-(360 mg, 1.28 mmol) [prepared according to the procedure of Example 88] for 1,1-dimethylethyl (4-hydroxybutyl)carbamate: LCMS (ES) m/e 492 (M+H)⁺; ¹H NMR (d6-DMSO, 400 MHz) δ 8.39 (br s, 2H), 7.31 (s, 1H), 7.04 (m, 1H), 6.95 (m, 1H), 6.89 (m, 1H), 6.45 (m, 1H), 6.14 (d, J=16.1 Hz, 2H), 4.62 (q, J=7.0 Hz, 2H), 3.47 (m, 2H), 1.53 (s, 6H), 1.38 (t, J=7.0 Hz, 3H).

### Example 93

### Preparation of 4-[6-{[2-amino-1-(1,3-benzodioxol-4-yl)ethyl]oxy}-2-(4-amino-1,2,5-oxadiazol-3-yl)-1-ethyl-1H-imidazo[4,5-c]pyridin-4-yl]-2-methyl-3-butyn-2-ol

The title compound was prepared as a tan solid according to the preparation of Example 17, except substituting 1,1-dimethylethyl[2-(1,3-benzodioxol-4-yl)-2-hydroxyethyl]carbamate (E1 enantiomer) (360 mg, 1.28 mmol) [prepared according to the procedure of Example 88] for 1,1-dimethylethyl (4-hydroxybutyl)carbamate: LCMS (ES) m/e 492 (M+H)⁺; ¹H NMR (d6-DMSO, 400 MHz) δ 8.39 (br s, 2H), 7.31 (s, 1H), 7.04 (m, 1H), 6.95 (m, 1H), 6.89 (m, 1H), 6.45 (m, 1H), 6.14 (d, J=16.1 Hz, 2H), 4.62 (q, J=7.0 Hz, 2H), 3.47 (m, 2H), 1.53 (s, 6H), 1.38 (t, J=7.0 Hz, 3H).

### Example 94

### Preparation of 4-[6-{[2-amino-1-(1,3-benzodioxol-4-yl)ethyl]oxy}-2-(4-amino-1,2,5-oxadiazol-3-yl)-1-ethyl-1H-imidazo[4,5-c]pyridin-4-yl]-2-methyl-3-butyn-2-ol

The title compound was prepared as a tan solid according to the preparation of Example 17, except substituting 1,1-dimethylethyl[2-(1,3-benzodioxol-4-yl)-2-hydroxyethyl]carbamate (E2 enantiomer) (360 mg, 1.28 mmol) [prepared according to the procedure of Example 88] for 1,1-dimethylethyl (4-hydroxybutyl)carbamate: LCMS (ES) m/e 492 (M+H)⁺; ¹H NMR (d6-DMSO, 400 MHz) δ 8.39 (br s, 2H), 7.31 (s, 1H), 7.04 (m, 1H), 6.95 (m, 1H), 6.89 (m, 1H), 6.45 (m, 1H), 6.14 (d, J=16.1 Hz, 2H), 4.62 (q, J=7.0 Hz, 2H), 3.47 (m, 2H), 1.53 (s, 6H), 1.38 (t, J=7.0 Hz, 3H).

### Example 95

### Preparation of 4-(2-(4-amino-1,2,5-oxadiazol-3-yl)-6-{[2-(dimethylamino)-1-phenylethyl]oxy}-1-ethyl-1H-imidazo[4,5-c]pyridin-4-yl)-2-methyl-3-butyn-2-ol

### a) 2-(dimethylamino)-1-phenylethanol

To a MeOH (10 mL) solution of styrene oxide (3.0 g, 25 mmole) in a sealed tube was added dimethyl amine (37.5 mmole, 2M in MeOH). The reaction contents were heated to 60° C. for 12 h, cooled to RT and concentrate under vacuum. The residue was purified on silica (CHCl₃/MeOH/NH₄OH, 90/9/1) to give a light yellow oil; LCMS (ES) m/e 166 (M+H)⁺

### b) 4-(2-(4-amino-1,2,5-oxadiazol-3-yl)-6-{[2-(dimethylamino)-1-phenylethyl]oxy}-1-ethyl-1H-imidazo[4,5-c]pyridin-4-yl)-2-methyl-3-butyn-2-ol

The title compound was prepared as a tan solid according to the preparation of Example 17, except substituting 2-(dimethylamino)-1-phenylethanol (210 mg, 1.28 mmol) for 1,1-dimethylethyl (4-hydroxybutyl)carbamate: LCMS (ES) m/e 476 (M+H)⁺; ¹H NMR (d6-DMSO, 400 MHz) δ 7.51 (m, 2H), 7.42 (m, 4H), 7.37 (m, 1H), 7.00 (br s, 2H), 6.59 (m, 1H), 4.65 (q, J=7.0 Hz, 2H), 3.76 (m, 1H), 3.65 (m, 1H), 2.97 (s, 3H), 2.92 (s, 3H), 1.53 (s, 6H), 1.38 (t, J=7.0 Hz, 3H).

### Example 96

### Preparation of 4-[6-{[(cis)-1-amino-2,3-dihydro-1H-inden-2-yl]oxy}-2-(4-amino-1,2,5-oxadiazol-3-yl)-1-ethyl-1H-imidazo[4,5-c]pyridin-4-yl]-2-methyl-3-butyn-2-ol

The title compound was prepared as a tan solid according to the preparation of Example 17, except substituting 1,1-dimethylethyl[(trans)-2-hydroxy-2,3-dihydro-1H-inden-1-yl]carbamate (320 mg, 1.28 mmol) for 1,1-dimethylethyl (4-hydroxybutyl)carbamate: LCMS (ES) m/e 460 (M+H)⁺; ¹H NMR (d6-DMSO, 400 MHz) δ 8.76 (br s, 2H), 7.71 (m, 2H), 7.40 (m, 2H), 7.21 (m, 1H), 7.00 (br s, 2H), 6.49 (m, 1H), 5.01 (m, 1H), 4.62 (q, J=7.0 Hz, 2H), 3.45 (m, 1H), 3.21 (m, 1H), 1.53 (s, 6H), 1.38 (t, J=7.0 Hz, 3H).

### Example 97

### Preparation of 4-(2-(4-amino-1,2,5-oxadiazol-3-yl)-1-ethyl-6-{[(S)-(2R)-2-morpholinyl(phenyl)methyl]oxy}-1H-imidazo[4,5-c]pyridin-4-yl)-2-methyl-3-butyn-2-ol a) (R)-(2R)-2-morpholinyl(phenyl)methanol

To a solution of 1,1-dimethylethyl (2R)-2-formyl-4-morpholinecarboxylate (1.1 g, 5.1 mmole) in THF (50 mL) at −78° C. was added phenylmagnesium bromide (25 mmole, 1M in THF). The reaction was stirred at −78° C. for 2 h and then allowed to warm to 0° C. and stir for 1 more hour. The reaction was quenched with H₂O (10 mL) and extracted with DCM. The organics were dried over Na₂SO₄ and concentrated under vacuum. The residue was purified on silica (hexanes/EtOAc, 4:1) to give a light yellow solid. 1H NMR and LC-MS indicate >15:1 dr; LCMS (ES) m/e 294 (M+H)⁺

### b) 4-(2-(4-amino-1,2,5-oxadiazol-3-yl)-1-ethyl-6-{[(2R)-2-morpholinyl(phenyl)methyl]oxy}-1H-imidazo[4,5-c]pyridin-4-yl)-2-methyl-3-butyn-2-ol

The title compound was prepared as a tan solid according to the preparation of Example 17, except substituting (S)-(2R)-2-morpholinyl(phenyl)methanol (210 mg, 1.28 mmol) for 1,1-dimethylethyl (4-hydroxybutyl)carbamate: LCMS (ES) m/e 504 (M+H)⁺; ¹H NMR (CD₃OD, 400 MHz) δ 7.59 (m, 2H), 7.46 (m, 4H), 7.47 (m, 1H), 6.34 (m, 1H), 4.72 (q, J=7.0 Hz, 2H), 4.33 (m, 1H), 4.14 (m, 1H), 3.90 (m, 1H), 3.54 (m, 1H), 3.34 (m, 3H), 1.68 (s, 6H), 1.43 (t, J=7.0 Hz, 3H).

### Example 98

### Preparation of 4-(2-(4-amino-1,2,5-oxadiazol-3-yl)-1-ethyl-6-{[(R)-(2S)-2-morpholinyl(phenyl)methyl]oxy}-1H-imidazo[4,5-c]pyridin-4-yl)-2-methyl-3-butyn-2-ol

### a) (S)-(2S)-2-morpholinyl(phenyl)methanol

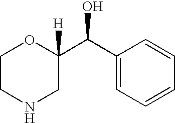

To a solution of 1,1-dimethylethyl (2S)-2-formyl-4-morpholinecarboxylate (1.1 g, 5.1 mmole) in THF (50 mL) at −78° C. was added phenylmagnesium bromide (25 mmole, 1M in THF). The reaction was stirred at −78° C. for 2 h and then allowed to warm to 0° C. and stir for 1 more hour. The reaction was quenched with H₂O (10 mL) and extracted with DCM. The organics were dried over Na₂SO₄ and concentrated under vacuum. The residue was purified on silica (hexanes/EtOAc, 4:1) to give a light yellow solid. 1H NMR and LC-MS indicate >15:1 dr; LCMS (ES) m/e 294 (M+H)⁺

### b) 4-(2-(4-amino-1,2,5-oxadiazol-3-yl)-1-ethyl-6-{[(2S)-2-morpholinyl(phenyl)methyl]oxy}-1H-imidazo[4,5-c]pyridin-4-yl)-2-methyl-3-butyn-2-ol

The title compound was prepared as a tan solid according to the preparation of Example 17, except substituting (R)-(2S)-2-morpholinyl(phenyl)methanol (210 mg, 1.28 mmol) for 1,1-dimethylethyl (4-hydroxybutyl)carbamate: LCMS (ES) m/e 504 (M+H)⁺; ¹H NMR (D6-DMSO, 400 MHz) δ 7.59 (m, 2H), 7.49 (m, 4H), 7.37 (m, 1H), 6.30 (m, 1H), 4.62 (q, J=7.0 Hz, 2H), 4.21 (m, 1H), 4.02 (m, 1H), 3.90 (m, 1H), 3.65 (m, 2H), 3.31 (m, 1H), 3.19 (m, 1H), 2.99 (m, 1H), 1.53 (s, 6H), 1.40 (t, J=7.0 Hz, 3H).

### Example 99

### Preparation of 4-[2-(4-amino-1,2,5-oxadiazol-3-yl)-1-ethyl-6-(1-pyrrolidinylmethyl)-1H-imidazo[4,5-c]pyridin-4-yl]-2-methyl-3-butyn-2-ol

The title compound was prepared as an orange solid according to Example 79, except substituting pyrrolidine (74 μL, 0.89 mmol) for methylamine: LCMS (ES) m/e 396 (M+H)⁺, ¹H NMR (CD₃OD, 400 MHz) δ 7.78 (s, 1H), 4.78 (quart., J=7.3 Hz, 2H), 3.93 (s, 2H), 2.68 (bs, 4H), 1.88 (bs, 4H), 1.68 (s, 6H), 1.48 (t, J=7.1 Hz, 3H)

### Example 100

### Preparation of 4-[2-(4-amino-1,2,5-oxadiazol-3-yl)-6-[(dimethylamino)methyl]-1-ethyl-1H-imidazo[4,5-c]pyridin-4-yl]-2-methyl-3-butyn-2-ol

The title compound was prepared as an orange solid according to Example 79, except substituting dimethyl amine (260 μL, 0.515 mmol) for methylamine: LCMS (ES) m/e 370 (M+H)⁺, ¹H NMR (CD₃OD, 400 MHz) δ 7.79 (s, 1H), 4.80 (quart., J=7.4 Hz, 2H), 3.77 (s, 2H), 2.36 (s, 6H), 1.68 (s, 6H), 1.47 (t, J=7.1 Hz, 3H)

### Example 101

### Preparation of 4-[2-(4-amino-1,2,5-oxadiazol-3-yl)-1-ethyl-6-(1-piperidinylmethyl)-1H-imidazo[4,5-c]pyridin-4-yl]-2-methyl-3-butyn-2-ol

The title compound was prepared as an orange solid according to Example 79, except substituting piperidine (59 μL, 0.690 mmol) for methylamine: LCMS (ES) m/e 410 (M+H)⁺, ¹H NMR (CD₃OD, 400 MHz) δ 7.81 (s, 1H), 4.79 (quart., J=7.2 Hz, 2H), 3.78 (s, 2H), 2.57 (bs, 4H), 1.68 (s, 6H), 1.63-1.68 (m, 4H), 1.49-1.53 (m, 3H), 1.46-1.49 (m, 2H)

### Example 102

### Preparation of N-{[2-(4-amino-1,2,5-oxadiazol-3-yl)-1-ethyl-4-(3-hydroxy-3-methyl-1-butyn-1-yl)-1H-imidazo[4,5-c]pyridin-6-yl]methyl}-N-methylacetamide

A solution of 4-{2-(4-amino-1,2,5-oxadiazol-3-yl)-1-ethyl-6-[(methylamino)methyl]-1H-imidazo[4,5-c]pyridin-4-yl}-2-methyl-3-butyn-2-ol [prepared in Example 79] (28 mg, 78.8 μmol), AcOH (5 μL, 94.6 μmol), NMM (17 μL, 0.158 mmol) and EDCl (18 mg, 95 μmol) in DMF (2 mL) was stirred at 25° C. over 12 h. The resulting solution was concentrated and purified via column chromatography (silica, 2% MeOH in DCM (1% NH₄OH)) yielding the title compound (22 mg, 70%) as a white powder: LCMS (ES) m/e 398 (M+H)⁺, ¹H NMR (CD₃OD, 400 MHz) δ 7.61 (s, 1H), 4.72-4.81 (m, 4H), 3.01/3.18 (s, 3H, rotameric methyl), 2.27 (s, 3H), 1.68 (s, 6H), 1.48-1.53 (m, 3H)

### Example 103

### Preparation of 4-(2-(4-amino-1,2,5-oxadiazol-3-yl)-6-{[(2R)-2-amino-3-(4-Pyridinyl)propyl]oxy}-1-ethyl-1H-imidazo[4,5-c]pyridin-4-yl)-2-methyl-3-butyn-2-ol

The title compound was prepared as a yellow solid according to Example 62, except substituting 1,1-dimethylethyl[(1R)-2-hydroxy-1-(4-pyridinylmethyl)ethyl]carbamate (1 g, 6.02 mmol) for 1,1-dimethylethyl[(1S)-1-(hydroxymethyl)-3-phenylpropyl]carbamate: LCMS (ES) m/e 463 (M+H)⁺, ¹H NMR (CD₃OD, 400 MHz) δ 8.48 (d, J=7.1 Hz, 2H), 7.41 (d, J=7.2 Hz, 2H), 7.06 (s, 1H), 4.70 (quart., J=7.2 Hz, 2H), 4.31-4.34 (m, 1H), 4.19-4.23 (m, 1H), 3.52-3.57 (m, 1H), 3.00-3.09 (m, 1H), 2.89-2.95 (m, 1H), 1.67 (s, 6H), 1.48 (t, J=7.3 Hz, 3H)

### Example 104

### Preparation of 4-(2-(4-amino-1,2,5-oxadiazol-3-yl)-6-{[(1S)-2-amino-1-phenylethyl]oxy}-1-ethyl-1H-imidazo[4,5-c]pyridin-4-yl)-2-methyl-3-butyn-2-ol

The title compound was prepared as a yellow solid according to Example 17, except substituting (1R)-2-amino-1-phenylethanol (5.7 g, 42 mmol) for 4-amino-1-butanol: LCMS (ES) m/e 448 (M+H)⁺, ¹H NMR (d-DMSO, 400 MHz) δ 8.22 (bs, 2H), 7.49 (d, J=7.2 Hz, 2H), 7.40 (t, J=6.9 Hz, 2H), 7.33-7.35 (m, 1H), 7.32 (s, 1H), 6.99 (bs, 1H), 6.42-6.48 (m, 1H), 4.63 (quart., J=7.1 Hz, 2H), 3.35-3.37 (m, 2H), 1.53 (s, 6H), 1.36 (t, J=7.1 Hz, 3H)

### Example 105

### Preparation of 4-[6-[(2-amino-1-methylethyl)oxy]-2-(4-amino-1,2,5-oxadiazol-3-yl)-1-ethyl-1H-imidazo[4,5-c]pyridin-4-yl]-2-methyl-3-butyn-2-ol

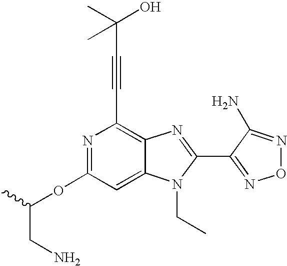

The title compound was prepared as a yellow solid according to Example 17, except substituting 1-amino-2-propanol (1 g, 13.3 mmol) for 4-amino-1-butanol: LCMS (ES) m/e 386 (M+H)⁺, ¹H NMR (CD₃OD, 400 MHz) δ 7.03 (s, 1H), 5.23-5.25 (m, 1H), 4.71 (quart., J=6.9 Hz, 2H), 2.95-2.97 (m, 2H), 1.67 (s, 6H), 1.47 (t, J=7.3 Hz, 3H), 1.37 (d, J=6.2 Hz, 3H)

### Example 106

### Preparation of 4-(2-(4-amino-1,2,5-oxadiazol-3-yl)-6-{[2-amino-1-(phenylmethyl)ethyl]oxy}-1-ethyl-1H-imidazo[4,5-c]pyridin-4-yl)-2-methyl-3-butyn-2-ol

The title compound was prepared as a yellow solid according to Example 17, except substituting 1-amino-3-phenyl-2-propanol [prepared according to Gensler, W. J; Dheer, S. K. J. Org. Chem. 1981, 46, 20, 4051.](2 g, 14.9 mmol) for 4-amino-1-butanol: LCMS (ES) m/e 462 (M+H)⁺, ¹H NMR (CD₃OD, 400 MHz) δ 7.36 (d, J=7.1 Hz, 2H), 7.28 (t, J=7.9 Hz, 2H), 7.18 (t, J=7.8 Hz, 1H), 6.96 (s, 1H), 5.39-5.42 (m, 1H), 4.67 (quart., J=7.4 Hz, 2H), 3.15-3.21 (m, 1H), 2.91-3.09 (m, 3H), 1.69 (s, 6H), 1.46 (t, J=7.6 Hz, 3H)

### Example 107

### Preparation of 4-{2-(4-amino-1,2,5-oxadiazol-3-yl)-6-[(3-amino-1-phenylpropyl)oxy]-1-ethyl-1H-imidazo[4,5-c]pyridin-4-yl}-2-methyl-3-butyn-2-ol

### (a) 1,1-dimethylethyl (3-hydroxy-3-phenylpropyl)carbamate

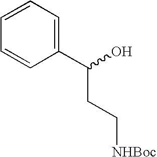

i) Benzoylacetonitrile (2 g, 13.8 mmol) in THF (35 mL) was added dropwise via addition funnel to a 0° C. solution of LAH (1.6 g, 41.3 mmol) in THF (35 mL). The resulting solution warmed to 25° C. and then was heated to 60° C. for an additional 2 h. After cooling to 0° C., a saturated solution of sodium potassium tartrate was added dropwise and the solution was extracted several times with DCM. The combined organic fractions were dried (Na₂SO₄), concentrated and purified via column chromatography (silica, 5-8% MeOH in DCM (1% NH₄OH)) affording the amino alcohol (1.4 g, 67%).

ii) The amino alcohol was re-dissolved in THF (50 mL) and Boc₂O (2.4 g, 11.1 mmol) was added in one portion. After 30 min., the solution was concentrated and the residue purified through a silica plug (0.5-1% MeOH in DCM (1% NH₄OH)) affording the title compound (1.6 g, 69%) as a pale white solid:

### (b) 4-{2-(4-amino-1,2,5-oxadiazol-3-yl)-6-[(3-amino-1-phenylpropyl)oxy]-1-ethyl-1H-imidazo[4,5-c]pyridin-4-yl}-2-methyl-3-butyn-2-ol

The title compound was prepared as a tan solid according to Example 17, except substituting 1,1-dimethylethyl (3-hydroxy-3-phenylpropyl)carbamate (289 mg, 1.2 mmol) for 1-dimethylethyl (4-hydroxybutyl)carbamate: LCMS (ES) m/e 462 (M+H)⁺, ¹H NMR (CD₃OD, 400 MHz) δ 7.50 (d, J=7.2 Hz, 2H), 7.34 (t, J=5.1 Hz, 2H), 7.25 (d, J=7.4 Hz, 1H), 6.93 (s, 1H), 6.12-6.17 (m, 1H), 4.57-4.61 (m, 2H), 2.69-2.72 (m, 2H), 2.22-2.26 (m, 1H), 2.05-2.11 (m, 1H), 1.66 (s, 6H), 1.37 (t, J=7.1 Hz, 3H).

### Example 108

### Preparation of 4-(2-(4-amino-1,2,5-oxadiazol-3-yl)-6-{[(1S)-3-amino-1-phenylpropyl]oxy}-1-ethyl-1H-imidazo[4,5-c]pyridin-4-yl)-2-methyl-3-butyn-2-ol

The title compound was prepared as an orange solid according to Example 107, except substituting 1,1-dimethylethyl[(3R)-3-hydroxy-3-phenylpropyl]carbamate [E1 from chiral HPLC, stereochemistry established using VCD](218 mg, 0.866 mmol) for the racemic alcohol: LCMS (ES) m/e 462 (M+H)⁺, ¹H NMR (CD₃OD, 400 MHz) δ 7.50 (d, J=7.2 Hz, 2H), 7.34 (t, J=5.1 Hz, 2H), 7.25 (d, J=7.4 Hz, 1H), 6.93 (s, 1H), 6.12-6.17 (m, 1H), 4.57-4.61 (m, 2H), 2.69-2.72 (m, 2H), 2.22-2.26 (m, 1H), 2.05-2.11 (m, 1H), 1.66 (s, 6H), 1.37 (t, J=7.1 Hz, 3H)

### Example 109

### Preparation of 4-(2-(4-amino-1,2,5-oxadiazol-3-yl)-6-{[(1R)-3-amino-1-phenylpropyl]oxy}-1-ethyl-1H-imidazo[4,5-c]pyridin-4-yl)-2-methyl-3-butyn-2-ol

The title compound was prepared as an orange solid according to Example 107, except substituting 1,1-dimethylethyl[(3S)-3-hydroxy-3-phenylpropyl]carbamate [E2 from chiral HPLC, stereochemistry established using VCD](252 mg, 1.01 mmol) for the racemic alcohol: LCMS (ES) m/e 462 (M+H)⁺, ¹H NMR (CD₃OD, 400 MHz) δ 7.50 (d, J=7.2 Hz, 2H), 7.34 (t, J=5.1 Hz, 2H), 7.25 (d, J=7.4 Hz, 1H), 6.93 (s, 1H), 6.12-6.17 (m, 1H), 4.57-4.61 (m, 2H), 2.69-2.72 (m, 2H), 2.22-2.26 (m, 1H), 2.05-2.11 (m, 1H), 1.66 (s, 6H), 1.37 (t, J=7.1 Hz, 3H)

### Example 110

### Preparation of 4-[6-[(3-amino-1-cyclohexylpropyl)oxy]-2-(4-amino-1,2,5-oxadiazol-3-yl)-1-ethyl-1H-imidazo[4,5-c]pyridin-4-yl]-2-methyl-3-butyn-2-ol

### (a) Preparation of 1,1-dimethylethyl (3-cyclohexyl-3-hydroxypropyl)carbamate

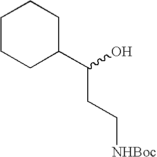 [0618] i) NaH (3.1 g, 77.4 mmol) in THF (70 mL) was heated to 75° C. and stirred for 15 min. before adding dropwise a solution of methyl cyclohexane carboxylate (10 g, 70.3 mmol and MeCN (4.8 mL, 91.4 mmol) in THF (70 mL). The resulting slurry was stirred for an additional 4 h and was then partitioned between EtOAc/1N HCl. The organic fraction was dried (Na₂SO₄), concentrated and used directly.
ii) The crude cyanoketone in THF (100 mL) was added dropwise to a solution of LAH (6.2 g, 0.16 mol) in THF (100 mL) at 0° C. After warming to 25° C. over 12 h. the solution was quenched through sequential addition of H₂O (7 mL), 6N NaOH (5.5 mL) and H₂O (26 mL). The solid precipitate was filtered off and the filtrate was concentrated and used directly.
iii) To the crude amino alcohol in THF (100 mL) was added Boc₂O (12 g, 55.6 mmol) in one portion. After 30 min., the solution was concentrated and the residue purified by column chromatography (silica, 1% MeOH in DCM (1% NH₄OH)) yielding the title compound (1.8 g) as a yellow oil

### (b) 4-[6-[(3-amino-1-cyclohexylpropyl)oxy]-2-(4-amino-1,2,5-oxadiazol-3-yl)-1-ethyl-1H-imidazo[4,5-c]pyridin-4-yl]-2-methyl-3-butyn-2-ol

The title compound was prepared as a tan solid according to Example 17, except substituting 1,1-dimethylethyl (3-cyclohexyl-3-hydroxypropyl)carbamate (334 mg, 1.3 mmol) for 1-dimethylethyl (4-hydroxybutyl)carbamate: LCMS (ES) m/e 468 (M+H)⁺, ¹H NMR (CD₃OD, 400 MHz) δ 7.06 (s, 1H), 5.12-5.17 (m, 1H), 4.69 (quart., J=7.1 Hz, 2H), 2.89-3.01 (m, 2H), 1.82-2.05 (m, 3H), 1.73-1.82 (m, 3H), 1.62-1.69 (m, 1H), 1.68 (s, 6H), 1.47 (t, J=7.2 Hz, 3H), 1.12-1.42 (m, 6H)

### Example 111

### Preparation of 4-(2-(4-amino-1,2,5-oxadiazol-3-yl)-6-{[2-amino-1-(4-Pyridinyl)ethyl]oxy}-1-ethyl-1H-imidazo[4,5-c]pyridin-4-yl)-2-methyl-3-butyn-2-ol

### (a) Preparation of 1,1-dimethylethyl[2-hydroxy-2-(4-pyridinyl)ethyl]carbamate

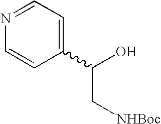

i) Potassium t-butoxide (3 g, 24.3 mmol) was added to a solution of trimethylsulfonium iodide (5.1 g, 25.2 mmol) in DMSO (17 mL) at 25° C. After 30 min. the solution clarified and 4-pyridine carboxaldehyde was added in one portion. After an additional 2 h, ice H₂O was added and the solution was extracted several times with Et₂O. The combined ethereal fractions were dried (Na₂SO₄), concentrated and used directly

ii) The crude epoxide was dissolved in 7N NH₃/MeOH (20 mL) and stirred at room temperature for 5 d. in a sealed tube. The resulting solution was concentrated and purified via column chromatography (silica, 5% MeOH in DCM (1% NH₄OH)) yielding the title compound (300 mg) as an orange oil.

iii) To a solution of the amino alcohol in MeOH (10 mL) was added Boc₂O (568 mg, 2.6 mmol). After 30 min. the solution was concentrated and purified via column chromatography (silica, 3% MeOH in DCM (1% NH₄OH)) yielding the title compound (250 mg) as an orange oil.

### (b) Preparation of 4-(2-(4-amino-1,2,5-oxadiazol-3-yl)-6-{[2-amino-1-(4-pyridinyl)ethyl]oxy}-1-ethyl-1H-imidazo[4,5-c]pyridin-4-yl)-2-methyl-3-butyn-2-ol

The title compound was prepared as a tan solid according to Example 17, except substituting 1,1-dimethylethyl[2-hydroxy-2-(4-pyridinyl)ethyl]carbamate (250 mg, 1.05 mmol) for 1-dimethylethyl (4-hydroxybutyl)carbamate: LCMS (ES) m/e 449 (M+H)⁺, ¹H NMR (CD₃OD, 400 MHz) δ 8.52 (d, J=7.2 Hz, 2H), 7.54 (d, J=7.0 Hz, 2H), 7.17 (s, 1H), 6.14-6.16 (m, 1H), 4.69 (quart., J=7.3 Hz, 2H), 3.13-3.15 (m, 2H), 1.63 (s, 6H), 1.46 (t, J=7.1 Hz, 3H).

### Example 112

### Preparation of 4-(2-(4-amino-1,2,5-oxadiazol-3-yl)-6-{[2-amino-1-(2-Pyridinyl)ethyl]oxy}-1-ethyl-1H-imidazo[4,5-c]pyridin-4-yl)-2-methyl-3-butyn-2-ol

The title compound was prepared as a tan solid according to Example 111, except substituting 2-pyridine carboxaldehyde (5 g, 46.7 mmol) for 4-pyridine carboxaldehyde: LCMS (ES) m/e 449 (M+H)⁺, ¹H NMR (CD₃OD, 400 MHz) δ 8.55 (d, J=7.2 Hz, 1H), 7.81 (d, J=6.9 Hz, 1H), 7.56 (d, J=7.9 Hz, 1H), 7.31-7.35 (m, 1H), 7.12 (s, 1H), 6.11-6.17 (m, 1H), 4.66 (quart., J=7.1 Hz, 2H), 3.20-3.22 (m, 2H), 1.62 (s, 6H), 1.44 (t, J=7.3 Hz, 3H)

### Example 113

### Preparation of 4-[6-{[1-(aminomethyl)-3-phenylpropyl]oxy}-2-(4-amino-1,2,5-oxadiazol-3-yl)-1-ethyl-1H-imidazo[4,5-c]pyridin-4-yl]-2-methyl-3-butyn-2-ol

### (a) Preparation of 1,1-dimethylethyl (2-hydroxy-4-phenylbutyl)carbamate

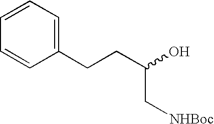 [0629] i) mCPBA (4 g, 22.7 mmol) was added in one portion to a solution of 4-phenyl-1-butene (2 g, 15.1 mmol) in DCM (76 mL) at 0° C. After warming to 25° C. over 12 h, the solution was partitioned between a saturated aqueous solution of NaHCO₃ and DCM. The combined organic fractions were dried (Na₂SO₄), concentrated and purified through a silica plug (10% EtOAc-hexanes) affording the epoxide (2 g, 90%) as a clear oil.
ii) The epoxide was dissolved in 7N NH₃/MeOH (30 mL) and stirred at 70° C. for 2 h. The resulting solution was concentrated and used directly
iii) To the crude amino alcohol in MeOH (68 mL) was added Boc2O (3.5 g, 16.2 mmol) in one portion. After 30 min., the solution was concentrated and the residue purified by column chromatography (silica, 1% MeOH in DCM (1% NH4OH)) yielding the title compound (2 g) as a clear oil.

### (b) Preparation of 4-[6-{[1-(aminomethyl)-3-phenylpropyl]oxy}-2-(4-amino-1,2,5-oxadiazol-3-yl)-1-ethyl-1H-imidazo[4,5-c]pyridin-4-yl]-2-methyl-3-butyn-2-ol

The title compound was prepared as a tan solid according to Example 17, except substituting 1,1-dimethylethyl (2-hydroxy-4-phenylbutyl)carbamate (284 mg, 1.07 mmol) for 1-dimethylethyl (4-hydroxybutyl)carbamate: LCMS (ES) m/e 476 (M+H)⁺, ¹H NMR (CD₃OD, 400 MHz) δ 7.15-7.25 (m, 5H), 7.00 (s, 1H), 5.19-5.26 (m, 1H), 4.65-4.69 (m, 2H), 2.98-3.01 (m, 2H), 2.77-2.81 (m, 2H), 2.01-2.19 (m, 2H), 1.63 (s, 6H), 1.47 (t, J=7.2 Hz, 3H).

### Example 114

### Preparation of 4-[2-(4-amino-1,2,5-oxadiazol-3-yl)-6-[(4-amino-1-phenylbutyl)oxy]-1-ethyl-1H-imidazo[4,5-c]pyridin-4-yl]-2-methyl-3-butyn-2-ol

The title compound was prepared as a tan solid according to Example 107, except substituting 4-oxo-4-phenylbutanenitrile [prepared according to Marshall, D. R. J. Chem. Soc., Perkin Trans. 21977, 1898.](3 g, 18.9 mmol) for benzoylacetonitrile: LCMS (ES) m/e 476 (M+H)⁺, ¹H NMR (CD₃OD, 400 MHz) δ 7.48 (d, J=7.2 Hz, 2H), 7.34 (t, J=7.2 Hz, 2H), 7.25 (t, J=7.3 Hz, 1H), 6.97 (s, 1H), 6.01-6.11 (m, 1H), 4.61-4.66 (m, 2H), 2.77-2.81 (m, 2H), 2.15-2.19 (m, 1H), 1.97-2.05 (m, 1H), 1.65-1.72 (m, 2H), 1.66 (s, 6H), 1.40 (t, J=7.2 Hz, 3H).

### Example 115

### Preparation of Rac-4-(2-(4-amino-1,2,5-oxadiazol-3-yl)-6-{[(1R,2S)-1-amino-1,2,3,4-tetrahydro-2-naphthalenyl]oxy}-1-ethyl-1H-imidazo[4,5-c]pyridin-4-yl)-2-methyl-3-butyn-2-ol

### (a) Preparation of Rac-1,1-dimethylethyl[(1R,2R)-2-hydroxy-1,2,3,4-tetrahydro-1-naphthalenyl]carbamate

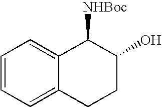 [0635] i) mCPBA (3.2 mg, 18.4 mmol) was added directly to an ice cold solution of 1,2-dihydronaphthalene (2 mg, 15.3 mmol) in NaHCO₃ sat./DCM (1:1, 150 mL). After warming to ambient temperature over 12 h, the solution was partitioned between sat. NaHSO₃/DCM. The organic phase was separated and the aqueous phase was back extracted several times with DCM. The combined organic fractions were dried (Na₂SO₄) and concentrated affording Rac-1a,2,3,7b-tetrahydronaphtho[1,2-b]oxirene which was used without further purification.
ii) Rac-1a,2,3,7b-tetrahydronaphtho[1,2-b]oxirene was dissolved in NH₄OH (20 mL) and heated 70° C. in a sealed tube. After 12 h, the solution was concentrated, redissolved in MeOH (50 mL) and treated with Boc₂O (4 g, 18.4 mmol) in one portion. After 30 min, the solution was concentrated and purified by column chromatography (silica, 1% MeOH in DCM (1% NH₄OH)) yielding the title compound (950 mg, 23%-3 steps) as a yellow solid.

### (b) Preparation of Rac-4-(2-(4-amino-1,2,5-oxadiazol-3-yl)-6-{[(1R,2S)-1-amino-1,2,3,4-tetrahydro-2-naphthalenyl]oxy}-1-ethyl-1H-imidazo[4,5-c]pyridin-4-yl)-2-methyl-3-butyn-2-ol

The title compound was prepared as a tan solid according to Example 17, except substituting 1,1-dimethylethyl[(1R,2R)-2-hydroxy-1,2,3,4-tetrahydro-1-naphthalenyl]carbamate (213 mg, 0.810 mmol) for 1,1-dimethylethyl (4-hydroxybutyl)carbamate: LCMS (ES) m/e 474 (M+H)⁺, ¹H NMR (CD₃OD, 400 MHz) δ 7.44-7.47 (m, 1H), 7.21-7.23 (m, 2H), 7.16-7.18 (m, 1H), 7.08 (s, 1H), 5.41-5.52 (m, 1H), 4.68 (quart., J=7.1 Hz, 2H), 4.30-4.32 (m, 1H), 2.87-3.02 (m, 2H), 2.31-2.43 (m, 1H), 2.07-2.13 (m, 1H), 1.66 (s, 6H), 1.46 (t, J=7.2 Hz, 3H).

### Example 116

### Preparation of 4-[2-(4-amino-1,2,5-oxadiazol-3-yl)-1-ethyl-6-({(R)-phenyl[(2S)-2-pyrrolidinyl]methyl}oxy)-1H-imidazo[4,5-c]pyridin-4-yl]-2-methyl-3-butyn-2-ol

### (a) Preparation of 1,1-dimethylethyl {(2S)-2-[(S)-hydroxy(phenyl)methyl]-1-pyrrolidinyl}acetate

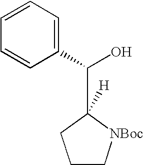

Phenylmagnesium bromide (30 mL, 30 mmol) was added dropwise to a −78° C. solution of 1,1-dimethylethyl[(2S)-2-formyl-1-pyrrolidinyl]acetate-(1.2 g, 6.02 mmol) in THF (20 mL). After 2 h, the solution warmed to 0° C. and was quenched with H₂O. The phases were separated and the aqueous phase was back extracted several times with DCM. The combined organic phases were dried (Na₂SO₄), concentrated and purified via column chromatography (silica, 5% EtOAc in hexane affording the title compound as a single enantiomer (the stereochemistry of which was assigned based on literature precedent, Reed, P. E.; Katzenellenbogen J. Org. Chem. 1991, 56, 2624 and confirmed through VCD).

### (b) Preparation of 4-[2-(4-amino-1,2,5-oxadiazol-3-yl)-1-ethyl-6-({(R)-phenyl[(2S)-2-pyrrolidinyl]methyl}oxy)-1H-imidazo[4,5-c]pyridin-4-yl]-2-methyl-3-butyn-2-ol

The title compound was prepared as a tan solid according to Example 17, except substituting 1,1-dimethylethyl {(2S)-2-[(S)-hydroxy(phenyl)methyl]-1-pyrrolidinyl}acetate (319 g, 1.2 mmol) for 1,1-dimethylethyl (4-hydroxybutyl)carbamate: LCMS (ES) m/e 488 (M+H)⁺, ¹H NMR (CD₃OD, 400 MHz) δ 7.52 (d, J=7.2 Hz, 2H), 7.36 (t, J=7.2 Hz, 2H), 7.28 (t, J=7.4 Hz, 1H), 7.01 (s, 1H), 6.15 (d, J=5.4 Hz, 1H), 4.58-4.64 (m, 2H), 3.61-3.68 (m, 1H), 3.05-3.16 (m, 1H), 2.89-2.92 (m, 1H), 1.75-2.00 (m, 4H), 1.67 (s, 6H), 1.39 (t, J=7.2 Hz, 3H)

### Example 117

### Preparation of 4-[2-(4-amino-1,2,5-oxadiazol-3-yl)-1-ethyl-6-([(S)-phenyl[(2R)-2-pyrrolidinyl]methyl]oxy)-1H-imidazo[4,5-c]pyridin-4-yl]-2-methyl-3-butyn-2-ol

The title compound was prepared as a tan solid according to Example 17, except substituting 1,1-dimethylethyl {(2R)-2-[(R)-hydroxy(phenyl)methyl]-1-pyrrolidinyl}acetate (360 mg, 1.3 mmol) [The absolute stereochemistry of which was confirmed by VCD] for 1,1-dimethylethyl {(2S)-2-[(S)-hydroxy(phenyl)methyl]-1-pyrrolidinyl}acetate: LCMS (ES) m/e 488 (M+H)⁺, ¹H NMR (CD₃OD, 400 MHz) δ 7.52 (d, J=7.2 Hz, 2H), 7.36 (t, J=7.2 Hz, 2H), 7.28 (t, J=7.4 Hz, 1H), 7.01 (s, 1H), 6.15 (d, J=5.4 Hz, 1H), 4.58-4.64 (m, 2H), 3.61-3.68 (m, 1H), 3.05-3.16 (m, 1H), 2.89-2.92 (m, 1H), 1.75-2.00 (m, 4H), 1.67 (s, 6H), 1.39 (t, J=7.2 Hz, 3H).

### Example 118

### Preparation of 4-(2-(4-amino-1,2,5-oxadiazol-3-yl)-6-{[2-amino-1-(4-piperidinyl)ethyl]oxy}-1-ethyl-1H-imidazo[4,5-c]pyridin-4-yl)-2-methyl-3-butyn-2-ol

### (a) Preparation of 1,1-dimethylethyl 4-[2-({[(1,1-dimethylethyl)oxy]carbonyl}amino)-1-hydroxyethyl]-1-piperidinecarboxylate

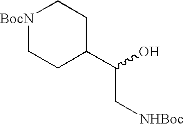 [0643] i) To oxalyl chloride (2.7 mL, 30.2 mmol) in DCM (120 mL) at −78° C. was added DMSO (4.3 mL, 60.38 mmol) dropwise. After 1 h, 1,1-dimethylethyl 4-(hydroxymethyl)-1-piperidinecarboxylate (5 g, 23.2 mmol) was added in one portion. After an additional 1 h, Et₃N (19 mL, 0.14 mol) was added dropwise and the solution warmed to ambient temperature of 12 h, was partitioned between H₂O/DCM and the phases were separated. The aqueous phase was back extracted several times with DCM and the combined organic fractions were dried (Na₂SO₄), concentrated and purified by column chromatography (silica, 20% EtOAc in hexanes) yielding 1,1-dimethylethyl 4-formyl-1-piperidinecarboxylate (2.9 g) as a yellow oil.
ii) To a solution of 1,1-dimethylethyl 4-formyl-1-piperidinecarboxylate (1 g, 4.6 mmol) in THF (66 mL) at ambient temperature was added diethyl aluminiumcyanide, 2M in toluene (9 mL, 9.2 mmol). After 2 h, the solution was partitioned between H₂O/DCM and the phases were separated. The aqueous phase was back extracted several times with DCM and the combined organic fractions were dried (Na₂SO₄) and concentrated yielding 1,1-dimethylethyl 4-[cyano(hydroxy)methyl]-1-piperidinecarboxylate (1.1 g) as a yellow oil which was used directly.
iii) A solution of 1,1-dimethylethyl 4-[cyano(hydroxy)methyl]-1-piperidinecarboxylate (1.1 g, 4.6 mmol) in THF (20 mL) was added dropwise to a 0° C. solution of LAH (523 mg, 13.8 mmol) in THF (20 mL). After 2 h warming to ambient temperature, the solution was quenched sequentially by dropwise addition of H₂O (0.626 mL), 6N NaOH (0.475 mL) and H₂O (2.3 mL). After 2 h, the precipitate was filtered and the pad was washed with bCM. The filtrate was concentrated affording 1,1-dimethylethyl 4-(2-amino-1-hydroxyethyl)-1-piperidinecarboxylate which was used directly.
iv) A solution 1,1-dimethylethyl 4-(2-amino-1-hydroxyethyl)-1-piperidinecarboxylate (425 mg, 1.73 mmol) dissolved in THF (10 mL) was treated with Boc₂O (454 mg, 2.08 mmol). After 30 min, the solution was concentrated and purified by column chromatography (silica, 1% MeOH in DCM (1% NH₄OH) yielding 1,1-dimethylethyl 4-[2-({[(1,1-dimethylethyl)oxy]carbonyl}amino)-1-hydroxyethyl]-1-piperidinecarboxylate (305 mg).

### (b) Preparation of 4-(2-(4-amino-1,2,5-oxadiazol-3-yl)-6-{[2-amino-1-(4-piperidinyl)ethyl]oxy}-1-ethyl-1H-imidazo[4,5-c]pyridin-4-yl)-2-methyl-3-butyn-2-ol

The title compound was prepared as a tan solid according to Example 17, except substituting 1,1-dimethylethyl 4-[2-({[(1,1-dimethylethyl)oxy]carbonyl}amino)-1-hydroxyethyl]-1-piperidinecarboxylate (305 mg, 1.3 mmol) for, 1-dimethylethyl (4-hydroxybutyl)carbamate: LCMS (ES) m/e 455 (M+H)⁺, ¹H NMR (CD₃OD, 400 MHz) δ 7.04 (s, 1H), 5.12-5.17 (m, 1H), 4.69 (quart., J=7.4 Hz, 2H), 3.09-3.15 (m, 2H), 2.87-3.05 (m, 2H), 2.52-2.61 (m, 2H), 1.92-2.01 (m, 1H), 1.82-1.89 (m, 1H), 1.70-1.75 (m, 1H), 1.67 (s, 6H), 1.46 (t, J=7.2 Hz, 3H), 1.36-1.48 (m, 2H).

### Example 119

### Preparation of 4-[6-{[2-amino-1-(1-methyl-4-piperidinyl)ethyl]oxy}-2-(4-amino-1,2,5-oxadiazol-3-yl)-1-ethyl-1H-imidazo[4,5-c]pyridin-4-yl]-2-methyl-3-butyn-2-ol

The title compound was prepared as a pale yellow solid according to Example 118, except substituting 2-amino-1-(1-methyl-4-piperidinyl)ethanol [prepared by heating the solution in section a-iii to reflux] (315 mg, 1.35 mmol) for 1,1-dimethylethyl 4-(2-amino-1-hydroxyethyl)-1-piperidinecarboxylate: LCMS (ES) m/e 469 (M+H)⁺, ¹H NMR (CD₃OD, 400 MHz) δ 7.07 (s, 1H), 5.15-5.20 (m, 1H), 4.69 (quart, J=7.1 Hz, 2H), 2.85-3.17 (m, 4H), 2.28 (s, 3H), 1.99-2.12 (m, 2H), 1.71-1.82 (m, 3H), 1.67 (s, 6H), 1.42-1.51 (m, 2H), 1.48 (t, J=7.2 Hz, 3H)

### Example 120

### Preparation of 4-(2-(4-amino-1,2,5-oxadiazol-3-yl)-6-{[3-amino-1-(4-piperidinyl)propyl]oxy}-1-ethyl-1H-imidazo[4,5-c]pyridin-4-yl)-2-methyl-3-butyn-2-ol

### (a) Preparation of 1,1-dimethylethyl 4-[3-({[(1,1-dimethylethyl)oxy]carbonyl}amino)-1-hydroxypropyl]-1-piperidinecarboxylate

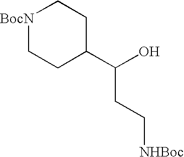 [0650] i) To a solution of LDA prepared by adding nBuLi 2.5 M in hexanes (2.5 mL, 6.2 mmol) to diisopropylamine (0.093 mL, 6.6 mmol) in THF (6 mL) at −78° C. After 30 min, MeCN (0.325 mL, 6.2 mmol) was added dropwise. After an additional 30 min., 1,1-dimethylethyl 4-formyl-1-piperidinecarboxylate [prepared in Example 118 (900 mg, 4.1 mmol) in THF (7 mL) was added and the solution stirred for 2 h, was quenched with H₂O and washed several times with DCM. The combined organic fractions were dried (Na₂SO₄), concentrated and purified by column chromatography (silica, 30% EtOAc in hexanes) yielding 1,1-dimethylethyl 4-(2-cyano-1-hydroxyethyl)-1-piperidinecarboxylate (700 mg, 66%) as a clear oil.
ii) A solution of 1,1-dimethylethyl 4-(2-cyano-1-hydroxyethyl)-1-piperidinecarboxylate (700 mg, 2.76 mmol) in THF (14 mL) was added dropwise to a 0° C. solution of LAH (314 mg, 8.3 mmol) in THF (14 mL). After 1 h at this temperature, the solution was quenched by sequential addition of H₂O (0.376 mL), 6N NaOH (0.285 mL) and H₂O (1.4 mL). After 2 h, the precipitate was filtered and the pad was washed with DCM. The filtrate was concentrated affording 1,1-dimethylethyl 4-(3-amino-1-hydroxypropyl)-1-piperidinecarboxylate which was used directly.
iii) A solution 1,1-dimethylethyl 4-(3-amino-1-hydroxypropyl)-1-piperidinecarboxylate dissolved in THF (14 mL) was treated with Boc₂O (721 mg, 3.31 mmol). After 30 min, the solution was concentrated and purified by column chromatography (silica, 3% MeOH in DCM (1% NH₄OH)) yielding 1,1-dimethylethyl 4-[3-({[(1,1-dimethylethyl)oxy]carbonyl}amino)-1-hydroxypropyl]-1-piperidinecarboxylate (338 mg, 34%-2 steps) as a white foam.

### (b) Preparation of 4-(2-(4-amino-1,2,5-oxadiazol-3-yl)-6-{[3-amino-1-(4-piperidinyl)propyl]oxy}-1-ethyl-1H-imidazo[4,5-c]pyridin-4-yl)-2-methyl-3-butyn-2-ol

The title compound was prepared as a yellow solid according to Example 17, except substituting 4-[3-({[(1,1-dimethylethyl)oxy]carbonyl}amino)-1-hydroxypropyl]-1-piperidinecarboxylate (338 mg, 0.944 mmol) for 1-dimethylethyl (4-hydroxybutyl)carbamate: LCMS (ES) m/e 469 (M+H)⁺, ¹H NMR (CD₃OD, 400 MHz) δ 7.10 (s, 1H), 5.39-5.42 (m, 1H), 4.71 (quart., J=7.1 Hz, 2H), 3.41-3.48 (m, 2H), 2.89-3.13 (m, 4H), 2.01-2.16 (m, 4H), 1.98-2.00 (m, 1H), 1.68 (s, 6H), 1.51-1.62 (m, 2H), 1.48 (t, J=7.3 Hz, 3H)

### Example 121

### Preparation of 4-[6-{[3-amino-1-(1-methyl-4-piperidinyl)propyl]oxy}-2-(4-amino-1,2,5-oxadiazol-3-yl)-1-ethyl-1H-imidazo[4,5-c]pyridin-4-yl]-2-methyl-3-butyn-2-ol

The title compound was prepared as an off-white solid according to Example 120, except substituting 3-amino-1-(1-methyl-4-piperidinyl)-1-propanol (214 mg, 0.919 mmol) [prepared by heating the solution in section a-ii to reflux then working up in the described manner] for 1,1-dimethylethyl 4-(3-amino-1-hydroxypropyl)-1-piperidinecarboxylate: LCMS (ES) m/e 483 (M+H)⁺, ¹H NMR (CD₃OD, 400 MHz) δ 7.03 (s, 1H), 5.25-5.29 (m, 1H), 4.69 (quart., J=7.1 Hz, 2H), 2.79-2.98 (m, 4H), 2.41-2.48 (m, 1H), 2.28 (s, 3H), 2.20-2.28 (m, 1H), 1.89-2.01 (m, 4H), 1.62-1.65 (m, 1H), 1.67 (s, 6H), 1.45-11.52 (m, 2H), 1.47 (t, J=7.2 Hz, 3H)

### Example 122

### Preparation of 4-[6-{[3-amino-1-(1-methyl-4-piperidinyl)propyl]oxy}-2-(4-amino-1,2,5-oxadiazol-3-yl)-1-ethyl-1H-imidazo[4,5-c]pyridin-4-yl]-2-methyl-3-butyn-2-ol

### (a) Preparation of [2-(4-amino-1,2,5-oxadiazol-3-yl)-4-chloro-1-ethyl-1H-imidazo[4,5-c]pyridin-6-yl](phenyl)methanol

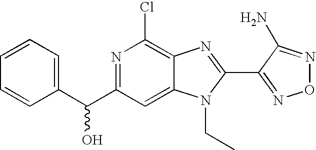

To a solution of 4-(6-bromo-4-chloro-1-ethyl-1H-imidazo[4,5-c]pyridin-2-yl)-1,2,5-oxadiazol-3-amine (300 mg, 0.875 mmol) in THF (43 mL) at −105° C. was added nBuLi, 2.5M in hexanes (1.4 mL, 3.5 mmol). After 5 min, benzaldehyde (354 μL, 3.5 mmol) was added dropwise and the solution stirred an additional 1 h, was quenched with H₂O and washed several times with DCM. The combined organic fractions were dried (Na₂SO₄), concentrated and purified by column chromatography (silica, 0.5% MeOH in DCM) yielding the title compound (100 mg, 30%) as a white foam.

### (b) Preparation of 2-(2-{[[2-(4-amino-1,2,5-oxadiazol-3-yl)-4-chloro-1-ethyl-1H-imidazo[4,5-c]pyridin-6-yl](phenyl)methyl]oxy}ethyl)-1H-isoindole-1,3(2H)-dione

A solution of [2-(4-amino-1,2,5-oxadiazol-3-yl)-4-chloro-1-ethyl-1H-imidazo[4,5-c]pyridin-6-yl](phenyl)methanol (256 mg, 0.692 mmol), hydroxyethyl phthalimide (265 mg, 1.38 mmol) and TsOH (9 mg, 48.4 μmol) in toluene (7 mL) was heated to 85° C. using a Dean Stark trap for azeotropic removal of H₂O. After 6 h, ice chunks were added and the solution was washed several times with DCM. The combined organic fractions were dried (Na₂SO₄), concentrated and purified by column chromatography (silica, 0.5% MeOH in DCM) yielding the title compound (100 mg).

### (c) Preparation of 2-(2-{[[2-(4-amino-1,2,5-oxadiazol-3-yl)-1-ethyl-4-(3-hydroxy-3-methyl-1-butyn-1-yl)-1H-imidazo[4,5-c]pyridin-6-yl](phenyl)methyl]oxy}ethyl)-1H-isoindole-1,3(2H)-dione

To a solution of 2-(2-{[[2-(4-amino-1,2,5-oxadiazol-3-yl)-4-chloro-1-ethyl-1H-imidazo[4,5-c]pyridin-6-yl](phenyl)methyl]oxy}ethyl)-1H-isoindole-1,3(2H)-dione (100 mg, 0.184 mmole) in DMF/Et₃N (2:1, 3 mL) was added CuI (7 mg, 36.8 μmol), 2-hydroxy-2-methyl-3-butyne (58 μL, 0.589 mmol) and dichlorobistriphenylphosphine palladium (II) (26 mg, 36.8 μmol). The reaction was heated to 80° C. in a sealed tube for 2 h and was concentrated under vacuum and purified on silica gel (2% MeOH in DCM (1% NH₄OH)) to give the title compound 116 mg) as an orange/brown oil.

### (d) Preparation of 4-[6-{[3-amino-1-(1-methyl-4-piperidinyl)propyl]oxy}-2-(4-amino-1,2,5-oxadiazol-3-yl)-1-ethyl-1H-imidazo[4,5-c]pyridin-4-yl]-2-methyl-3-butyn-2-ol

To a solution of 2-(2-{[[2-(4-amino-1,2,5-oxadiazol-3-yl)-1-ethyl-4-(3-hydroxy-3-methyl-1-butyn-1-yl)-1H-imidazo[4,5-c]pyridin-6-yl](phenyl)methyl]oxy}ethyl)-1H-isoindole-1,3(2H)-dione (116 mg, 0.197 mmol) in MeOH (2 mL) was added MeNH₂—H₂O (40 wt %, 9.8 mL, 3.95 mmol). After 30 min, the solution was concentrated and the residue purified via column chromatography (silica, 2% MeOH in DCM (1% NH4OH)) yielding the title compound as a yellow solid: LCMS (ES) m/e 462 (M+H)⁺, ¹H NMR (CD₃OD, 400 MHz) δ 7.95 (s, 1H), 7.93-7.95 (m, 2H), 7.49-7.51 (m, 2H), 7.26-7.28 (m, 1H), 5.65 (s, 1H), 4.78-4.81 (m, 2H), 3.61-3.63 (m, 2H), 2.92-2.95 (m, 2H), 1.67 (s, 6H), 1.46-1.50 (m, 3H)

### Example 123

### Preparation of 4-[6-[(2-amino-1-cyclohexylethyl)oxy]-2-(4-amino-1,2,5-oxadiazol-3-yl)-1-ethyl-1H-imidazo[4,5-c]pyridin-4-yl]-2-methyl-3-butyn-2-ol

### a) 1-cyclohexyl-2-({[(1,1-dimethylethyl)oxy]carbonyl}amino)ethyl benzoate

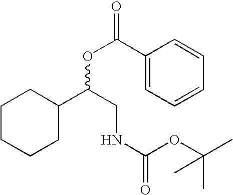

To a solution of 1,1-dimethylethyl (2-cyclohexyl-2-hydroxyethyl)carbamate (4.3 g, 17.7 mmoles) [from Example 88] in DCM (100 mL) at RT was added benzoyl chloride (4.1 mL, 35.4 mmoles), pyridine (5.7 mL, 70.8 mmoles) and DMAP (0.21 g, 1.77 mmoles). After 4 hours, the reaction solution was concentrated and the residue purified on silica (hexanes:EtOAc, 4:1) to give the title compound (5.7 g) as a white solid. LCMS (ES) m/e 348 (M+H)⁺.

The racemic material was resolved by chiral HPLC to give the corresponding pure enantiomers designated as E1 and E2.

### b) 1,1-dimethylethyl (2-cyclohexyl-2-hydroxyethyl)carbamate

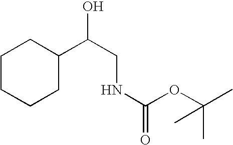

To a solution of 1-cyclohexyl-2-({[(1,1-dimethylethyl)oxy]carbonyl}amino)ethyl benzoate [E1 enantiomer] in MeOH (80 mL) and THF (20 mL) at RT was added 1N NaOH (33 mL). After 24 h, the reaction solution was concentrated under vacuum and extracted with DCM. The organics were dried over Na₂SO₄, concentrated and used without further purification. LCMS (ES) m/e 244 (M+H)⁺.

### c) 4-[6-[(2-amino-1-cyclohexylethyl)oxy]-2-(4-amino-1,2,5-oxadiazol-3-yl)-1-ethyl-1H-imidazo[4,5-c]pyridin-4-yl]-2-methyl-3-butyn-2-ol

The title compound was prepared as a tan solid according to the preparation of Example 88, except substituting 1,1-dimethylethyl (2-cyclohexyl-2-hydroxyethyl)carbamate [E1 enantiomer] (310 mg, 1.28 mmol) for 1,1-dimethylethyl (4-hydroxybutyl)carbamate: LCMS (ES) m/e 453 (M+H)⁺; ¹H NMR (d6-DMSO, 400 MHz) δ 8.21 (br s, 2H), 7.30 (s, 1H), 7.02 (br s, 2H), 5.26 (m, 1H), 4.65 (q, J=7.0 Hz, 2H), 3.1-2 (m, 2H), 1.75 (m, 6H), 1.53 (s, 6H), 1.38 (t, J==7.0 Hz, 3H), 1.16 (m, 4H).

### Example 124

### Preparation of 4-[6-[(2-amino-1-cyclohexylethyl)oxy]-2-(4-amino-1,2,5-oxadiazol-3-yl)-1-ethyl-1H-imidazo[4,5-c]pyridin-4-yl]-2-methyl-3-butyn-2-ol

The title compound was prepared as a tan solid according to the preparation of Example 88, except substituting 1,1-dimethylethyl (2-cyclohexyl-2-hydroxyethyl)carbamate [E2 enantiomer] (310 mg, 1.28 mmol) for 1,1-dimethylethyl (4-hydroxybutyl)carbamate: LCMS (ES) m/e 453 (M+H)⁺; ¹H NMR (d6-DMSO, 400 MHz) δ 8.21 (br s, 2H), 7.30 (s, 1H), 7.02 (br s, 2H), 5.26 (m, 1H), 4.65 (q, J=7.0 Hz, 2H), 3.12 (m, 2H), 1.75 (m, 6H), 1.53 (s, 6H), 1.38 (t, J=7.0 Hz, 3H), 1.16 (m, 4H).

### Example 125

### Preparation of 1-[6-[(2-aminoethyl)oxy]-2-(4-amino-1,2,5-oxadiazol-3-yl)-1-ethyl-1H-imidazo[4,5-c]pyridin-4-yl]-3-methyl-1-pentyn-3-ol

The title compound was prepared as a pale yellow solid according to Example 17, except substituting 1,1-dimethylethyl (2-hydroxyethyl)carbamate (1 mL, 6.5 mmol) for 1,1-dimethylethyl (4-hydroxybutyl)carbamate and 3-methyl-1-pentyn-3-ol (0.190 mL, 1.68 mmol) for 2-methyl-3-butyn-2-ol: LCMS (ES) m/e 386 (M+H)⁺; ¹H NMR (400 MHz, DMSO-d₆) δ ppm 1.11 (t, J=7.45 Hz, 3H) 1.36 (t, J=7.07 Hz, 3H) 1.50 (s, 3H) 1.58 (s, 2H) 1.67-1.79 (m, 2H) 2.90 (t, J=5.94 Hz, 2H) 4.24 (t, J=5.94 Hz, 2H) 4.62 (q, J=6.99 Hz, 2H) 5.63 (s, 1H) 7.03 (s, 2H) 7.21 (s, 1H).

### Example 126

### Preparation of 4-(2-(4-amino-1,2,5-oxadiazol-3-yl)-6-{[(1R,2S)-2-amino-1-phenylpropyl]oxy}-1-ethyl-1H-imidazo[4,5-c]pyridin-4-yl)-2-methyl-3-butyn-2-ol

The title compound was prepared as a tan solid according to Example 17, except substituting 1,1-dimethylethyl[(1S,2R)-2-hydroxy-1-methyl-2-phenylethyl]carbamate (822 mg, 3.27 mmol) for 1,1-dimethylethyl (4-hydroxybutyl)carbamate: LCMS (ES) m/e 462 (M+H)⁺; ¹H NMR (400 MHz, CDCl₃) δ ppm 1.10 (d, J=6.57 Hz, 3H) 1.39 (t, J=7.20 Hz, 3H) 1.69-1.72 (m, 6H) 2.27 (s, 2H) 3.40-3.48 (m, 1H) 4.53 (dt, J=12.69, 7.17 Hz, 2H) 5.79 (d, J=7.07 Hz, 1H) 5.84-5.92 (m, 2H) 6.67 (s, 1H) 7.26-7.29 (m, 1H) 7.34 (t, J=7.33 Hz, 2H) 7.46 (d, J=7.07 Hz, 2H).

### Example 127

### Preparation of 4-(2-(4-amino-1,2,5-oxadiazol-3-yl)-1-ethyl-6-{[3-(methylamino)-1-phenylpropyl]oxy}-1H-imidazo[4,5-c]pyridin-4-yl)-2-methyl-3-butyn-2-ol

The title compound was prepared as a brown solid according to Example 17, except substituting 1,1-dimethylethyl (3-hydroxy-3-phenylpropyl)methylcarbamate (362 mg, 1.36 mmol) for 1,1-dimethylethyl (4-hydroxybutyl)carbamate: LCMS (ES) m/e 476 (M+H)⁺; ¹H NMR (400 MHz, CDCl₃) δ ppm 1.43 (t, J=7.20 Hz, 3H) 1.68-1.74 (m, 6H) 2.13-2.24 (m, 2H) 2.24-2.35 (m, 2H) 2.48 (s, 3H) 2.71-2.79 (m, 2H) 4.58 (dt, J=12.38, 7.20 Hz, 2H) 5.87 (s, 2H) 6.23 (dd, J=8.21, 5.18 Hz, 1H) 6.71 (s, 1H) 7.25-7.30 (m, 1H) 7.35 (t, J=7.45 Hz, 2H) 7.49 (d, J=7.33 Hz, 2H).

### Example 128

### Preparation of 4-(2-(4-amino-1,2,5-oxadiazol-3-yl)-6-{[3-(dimethylamino)-1-phenylpropyl]oxy}-1-ethyl-1H-imidazo[4,5-c]pyridin-4-yl)-2-methyl-3-butyn-2-ol

### a) 3-(dimethylamino)-1-phenyl-1-propanone

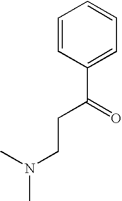

Combine 1-phenylethanone (4.46 g, 37.12 mmol), paraformaldehyde (3.08 g 103 mmol), and N-methylmethanamine (3.01 g, 36.9 mmol) in ethanol (100 mL) and stir, add several drops HCl_{(conc.)} (0.5 mL) and reflux 3 hours. Cool to room temperature and stir overnight. Remove solvent and chromatograph with ethyl acetate/hexane to obtain 3-(dimethylamino)-1-phenyl-1-propanone (3.8 g, 21.4 mmol). LCMS (ES) m/e 178 (M+H)⁺.

### b) 3-(dimethylamino)-1-phenyl-1-propanol

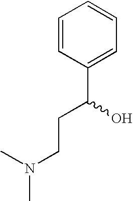

A solution of 3-(dimethylamino)-1-phenyl-1-propanone (3.8 g, 21.4 mmol) in ethanol (20 mL) is added dropwise to an ethanol (100 ML) mixture of NaBH₄ (1.1 g, 28.3 mmol) at 0° C. The mixture is allowed to warm to room temperature and stirred overnight. The mixture is quenched with 2.5 N HCl and made basic with 6N NaOH. The volume is reduced and partitioned between CHCl₃ and H₂O. LCMS (ES) m/e 180 (M+H)⁺.

### c) Preparation of 4-(2-(4-amino-1,2,5-oxadiazol-3-yl)-6-{[3-(dimethylamino)-1-phenylpropyl]oxy}-1-ethyl-1H-imidazo[4,5-c]pyridin-4-yl)-2-methyl-3-butyn-2-ol

The title compound was prepared as a brown solid according to Example 17, except substituting 3-(dimethylamino)-1-phenyl-1-propanol (207 mg, 1.15 mmol) for 1,1-dimethylethyl (4-hydroxybutyl)carbamate: LCMS (ES) m/e 490 (M+H)⁺; ¹H NMR (400 MHz, CDCl₃) δ ppm 1.37-1.49 (m, 3H) 1.67-1.78 (m, 6H) 2.04-2.16 (m, 1H) 2.26-2.43 (m, 9H) 4.55 (dq, J=14.78, 7.37 Hz, 2H) 5.90 (s, 2H) 6.16 (t, J=6.44 Hz, 1H) 6.66 (s, 1H) 7.25-7.31 (m, 1H) 7.31-7.39 (m, 2H) 7.47-7.54 (m, 2H).

### Example 129

### Preparation of 4-[6-{[3-amino-1-(4-chlorophenyl)propyl]oxy}-2-(4-amino-1,2,5-oxadiazol-3-yl)-1-ethyl-1H-imidazo[4,5-c]pyridin-4-yl]-2-methyl-3-butyn-2-ol

### a) 1,1-dimethylethyl[3-(4-chlorophenyl)-3-hydroxypropyl]carbamate

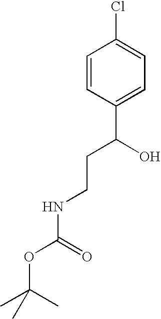

The title compound was prepared as an orange oil according to Example 107, except substituting 3-(4-chlorophenyl)-3-oxopropanenitrile (2.5 g, 13.9 mmole) for benzoylacetonitrile: LCMS (ES) m/e 286 (M+H)⁺;

### b) Preparation of 4-[6-{[3-amino-1-(4-chlorophenyl)propyl]oxy}-2-(4-amino-1,2,5-oxadiazol-3-yl)-1-ethyl-1H-imidazo[4,5-c]pyridin-4-yl]-2-methyl-3-butyn-2-ol

The title compound was prepared as a yellow solid according to Example 17, except substituting 1,1-dimethylethyl[3-(4-chlorophenyl)-3-hydroxypropyl]carbamate (481 mg, 1.68 mmol) for 1,1-dimethylethyl (4-hydroxybutyl)carbamate: LCMS (ES) m/e 496 (M+H)⁺; ¹H NMR (CDCl₃, 400 MHz) δ ppm 1.41 (t, J=7.20 Hz, 3H) 1.69 (s, 6H) 2.00 (qd, J=7.12, 4.93 Hz, 1H) 2.15-2.25 (m, 1H) 2.86 (t, J=6.44 Hz, 2H) 4.53 (ddd, J=8.97, 7.20, 7.07 Hz, 2H) 5.96 (s, 2H) 6.27 (dd, J=8.72, 4.67 Hz, 1H) 6.67 (s, 1H) 7.25-7.30 (m, 2H) 7.35-7.41 (m, 2H).

### Example 130

### Preparation of 4-[6-{[3-amino-1-(3-chlorophenyl)propyl]oxy}-2-(4-amino-1,2,5-oxadiazol-3-yl)-1-ethyl-1H-imidazo[4,5-c]pyridin-4-yl]-2-methyl-3-butyn-2-ol

### a) 1,1-dimethylethyl[3-(3-chlorophenyl)-3-hydroxypropyl]carbamate

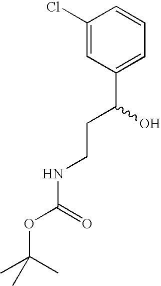

The title compound was prepared as an orange oil according to Example 107, except substituting 3-(3-chlorophenyl)-3-oxopropanenitrile (3.3 g, 18.4 mmol) for Benzoylacetonitrile: LCMS (ES) m/e 286 (M+H)⁺.

### b) Preparation of 4-[6-{[3-amino-1-(3-chlorophenyl)propyl]oxy}-2-(4-amino-1,2,5-oxadiazol-3-yl)-1-ethyl-1H-imidazo[4,5-c]pyridin-4-yl]-2-methyl-3-butyn-2-ol

The title compound was prepared as a yellow solid according to Example 17, except substituting 1,1-dimethylethyl[3-(3-chlorophenyl)-3-hydroxypropyl]carbamate (480 mg, 1.68 mmol) for 1,1-dimethylethyl (4-hydroxybutyl)carbamate: LCMS (ES) m/e 496 (M+H)⁺; ¹H NMR (CDCl₃, 400 MHz) δ ppm 1.42 (t, J=7.20 Hz, 3H) 1.70 (s, 6H) 2.03 (ddd, J=14.34, 7.39, 5.05 Hz, 1H) 2.16-2.25 (m, 1H) 2.83 (s, 2H) 2.86-2.90 (m, 2H) 4.51-4.59 (m, J=7.20, 7.20, 7.07, 2.02 Hz, 2H) 5.94 (s, 2H) 6.28 (dd, J=8.72, 4.67 Hz, 1H) 6.70 (s, 1H) 7.20-7.27 (m, 2H) 7.31-7.35 (m, 1H) 7.48 (s, 1H).

### Example 131

### Preparation of 4-[6-{[3-amino-1-(2-chlorophenyl)propyl]oxy}-2-(4-amino-1,2,5-oxadiazol-3-yl)-1-ethyl-1H-imidazo[4,5-c]pyridin-4-yl]-2-methyl-3-butyn-2-ol

### a) 1,1-dimethylethyl[3-(2-chlorophenyl)-3-hydroxypropyl]carbamate

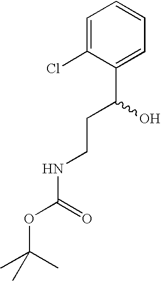

The title compound was prepared as an orange oil according the Example 107, except substituting 3-(2-chlorophenyl)-3-oxopropanenitrile (3.0 g, 16.9 mmol) for Benzoylacetonitrile: LCMS (ES) m/e 286 (M+H)⁺.

### b) Preparation of 4-[6-{[3-amino-1-(2-chlorophenyl)propyl]oxy}-2-(4-amino-1,2,5-oxadiazol-3-yl)-1-ethyl-1H-imidazo[4,5-c]pyridin-4-yl]-2-methyl-3-butyn-2-ol

The title compound was prepared as a yellow solid according to Example 17, except substituting 1,1-dimethylethyl[3-(2-chlorophenyl)-3-hydroxypropyl]carbamate (383 mg, 1.34 mmol) for 1,1-dimethylethyl (4-hydroxybutyl)carbamate: LCMS (ES) m/e 496 (M+H)⁺; ¹H NMR (CDCl₃, 400 MHz) δ ppm 1.43 (t, J=7.20 Hz, 3H) 1.65-1.73 (m, 6H) 2.11 (ddd, J=14.27, 7.20, 4.29 Hz, 1H) 2.17-2.21 (m, 1H) 2.58 (s, 2H) 2.98 (d, J=5.81 Hz, 2H) 4.59 (q, J=7.41 Hz, 2H) 5.89 (s, 2H) 6.50 (dd, J=8.84, 4.04 Hz, 1H) 6.71 (s, 1H) 7.17-7.27 (m, 2H) 7.36 (d, J=7.83 Hz, 1H) 7.59 (dd, J=7.71, 1.89 Hz, 1H).

### Example 132

### Preparation of 4-[6-({2-amino-1-[3-(methyloxy)phenyl]ethyl}oxy)-2-(4-amino-1,2,5-oxadiazol-3-yl)-1-ethyl-1H-imidazo[4,5-c]pyridin-4-yl]-2-methyl-3-butyn-2-ol

### a) 1,1-dimethylethyl {2-hydroxy-2-[3-(methyloxy)phenyl]ethyl}carbamate

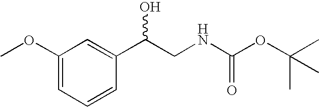

The title compound was prepared as an oil according the Example 88, except substituting 3-(methyloxy)benzaldehyde (8.53 g, 52.3 mmol) for cyclohexyl carboxaldehyde: LCMS (ES) m/e 282 (M+H)⁺.

### b) Preparation of 4-[6-({2-amino-1-[3-(methyloxy)phenyl]ethyl}oxy)-2-(4-amino-1,2,5-oxadiazol-3-yl)-1-ethyl-1H-imidazo[4,5-c]pyridin-4-yl]-2-methyl-3-butyn-2-ol

The title compound was prepared as a yellow solid according to Example 17, except substituting 1,1-dimethylethyl {2-hydroxy-2-[3-(methyloxy)phenyl]ethyl}carbamate (319 mg, 1.19 mmol) for 1,1-dimethylethyl (4-hydroxybutyl)carbamate: LCMS (ES) m/e 478 (M+H)⁺; ¹H NMR (CDCl₃, 400 MHz) δ ppm 1.41 (t, J=7.20 Hz, 3H) 1.70 (s, 6H) 2.21 (br.s, 3H) 3.24 (d, J=7.07 Hz, 2H) 3.80 (s, 3H) 4.55 (qd, J=7.20, 2.15 Hz, 2H) 5.90 (s, 2H) 6.05-6.12 (m, 1H) 6.71 (s, 1H) 6.76-6.83 (m, 1H) 7.01-7.07 (m, 2H) 7.23-7.30 (m, 1H).

### Example 133

### Preparation of 4-[6-{[(1S,2S)-2-amino-3-(methyloxy)-1-phenylpropyl]oxy}-2-(4-amino-1,2,5-oxadiazol-3-yl)-1-ethyl-1H-imidazo[4,5-c]pyridin-4-yl]-2-methyl-3-butyn-2-ol

The title compound was prepared as a yellow solid according to Example 17, except substituting 1 4-[6-{[(1S,2S)-2-amino-3-(methyloxy)-1-phenylpropyl]oxy}-2-(4-amino-1,2,5-oxadiazol-3-yl)-1-ethyl-1H-imidazo[4,5-c]pyridin-4-yl]-2-methyl-3-butyn-2-ol (794 mg, 2.82 mmol) for 1,1-dimethylethyl (4-hydroxybutyl)carbamate: LCMS (ES) m/e 492 (M+H)⁺; ¹H NMR (400 MHz, DMSO-d₆) δ ppm 1.38 (t, J=7.07 Hz, 3H) 1.51 (s, 6H) 3.26 (s, 3-H) 3.47 (dd, J=10.48, 3.41 Hz, 1H) 3.70 (dd, J=10.48, 8.46 Hz, 1H) 3.92 (s, 1H) 4.65 (q, J=6.99 Hz, 2H) 5.69 (s, 1H) 6.51 (d, J=4.04 Hz, 1H) 6.99 (s, 2H) 7.34 (t, J=7.20 Hz, 1H), 7.34-7.47 (m, 6H) 8.25-8.33 (m, 2H).

### Example 134

### Preparation of 4-[6-({3-amino-1-[2-fluoro-3-(methyloxy)phenyl]propyl}oxy)-2-(4-amino-1,2,5-oxadiazol-3-yl)-1-ethyl-1H-imidazo[4,5-c]pyridin-4-yl]-2-methyl-3-butyn-2-ol

### a) 1,1-dimethylethyl {3-[2-fluoro-3-(methyloxy)phenyl]-3-hydroxypropyl}carbamate

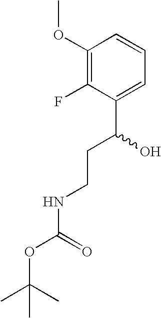

The title compound was prepared as an oil according to Example 120, except substituting 2-fluoro-3-(methyloxy)benzaldehyde (5.14 g, 33.3 mmol) for 1,1-dimethylethyl 4-formyl-1-piperidinecarboxylate: LCMS (ES) m/e 300 (M+H)⁺.

### b) Preparation of 4-[6-({3-amino-1-[2-fluoro-3-(methyloxy)phenyl]propyl}oxy)-2-(4-amino-1,2,5-oxadiazol-3-yl)-1-ethyl-1H-imidazo[4,5-c]pyridin-4-yl]-2-methyl-3-butyn-2-ol

The title compound was prepared as a yellow solid according to Example 17, except substituting 1,1-dimethylethyl {3-[2-fluoro-3-(methyloxy)phenyl]-3-hydroxypropyl}carbamate (553 mg, 1.85 mmol) for 1,1-dimethylethyl (4-hydroxybutyl)carbamate: LCMS (ES) m/e 510 (M+H)⁺; ¹H NMR (CDCl₃, 400 MHz) δ ppm 1.44 (t, J=7.20 Hz, 3H) 1.71 (s, 6H) 2.05-2.32 (m, 5H) 2.91 (t, J=6.44 Hz, 2H) 3.90 (s, 3H) 4.55-4.65 (m, 2H) 5.88 (s, 2H) 6.52 (dd, J=8.34, 4.80 Hz, 1H) 6.73 (s, 1H) 6.88 (td, J=8.08, 1.52 Hz, 1H) 7.01-7.12 (m, 2H).

### Example 135

### Preparation of 4-[6-({3-amino-1-[3-(methyloxy)phenyl]propyl}oxy)-2-(4-amino-1,2,5-oxadiazol-3-yl)-1-ethyl-1H-imidazo[4,5-c]pyridin-4-yl]-2-methyl-3-butyn-2-ol

### a) 1,1-dimethylethyl {3-hydroxy-3-[3-(methyloxy)phenyl]propyl}carbamate

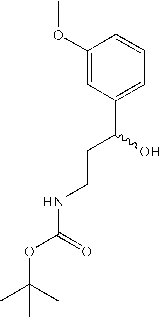

The title compound was prepared as an oil according to Example 120, except substituting 3-(methyloxy)benzaldehyde (6.11 g, 44.9 mmol) for cyclohexyl carboxaldehyde: LCMS (ES) m/e 282 (M+H)⁺.

### b) Preparation of 4-[6-({3-amino-1-[3-(methyloxy)phenyl]propyl}oxy)-2-(4-amino-1,2,5-oxadiazol-3-yl)-1-ethyl-1H-imidazo[4,5-c]pyridin-4-yl]-2-methyl-3-butyn-2-ol

The title compound was prepared as a yellow solid according to Example 17, except substituting 1,1-dimethylethyl {3-hydroxy-3-[3-(methyloxy)phenyl]propyl}carbamate (441 mg, 1.57 mmol) for 1,1-dimethylethyl (4-hydroxybutyl)carbamate: LCMS (ES) m/e 492 (M+H)⁺; ¹H NMR (400 MHz, MeOD) δ ppm 1.41 (t, J=7.20 Hz, 3H) 1.66 (s, 6H) 2.18-2.29 (m, 1H) 2.31-2.41 (m, 1-H) 3.00-3.11 (m, 2H) 3.80 (s, 3H) 4.60-4.70 (m, 2H) 6.14 (dd, J=8.59, 4.55 Hz, 1H) 6.85 (dd, J=8.21, 2.40 Hz, 1H) 7.03 (s, 1H) 7.05-7.09 (m, 2H) 7.28 (t, J=8.08 Hz, 1H).

### Example 136

### Preparation of 4-[6-({2-amino-1-[2-fluoro-3-(methyloxy)phenyl]ethyl}oxy)-2-(4-amino-1,2,5-oxadiazol-3-yl)-1-ethyl-1H-imidazo[4,5-c]pyridin-4-yl]-2-methyl-3-butyn-2-ol

### a) 1,1-dimethylethyl {2-[2-fluoro-3-(methyloxy)phenyl]-2-hydroxyethyl}carbamate

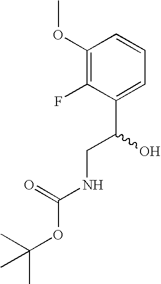

The title compound was prepared as an oil according to the Example 88, except substituting 2-fluoro-3-(methyloxy)benzaldehyde (5.18 g, 33.6 mmol) for cyclohexyl carboxaldehyde: LCMS (ES) m/e 286 (2M+H)⁺.

### b) Preparation of 4-[6-({2-amino-1-[2-fluoro-3-(methyloxy)phenyl]ethyl}oxy)-2-(4-amino-1,2,5-oxadiazol-3-yl)-1-ethyl-1H-imidazo[4,5-c]pyridin-4-yl]-2-methyl-3-butyn-2-ol

The title compound was prepared as a yellow solid according to Example 17, except substituting 1,1-dimethylethyl {2-[2-fluoro-3-(methyloxy)phenyl]-2-hydroxyethyl}carbamate (538 mg, 1.89 mmol) for 1,1-dimethylethyl (4-hydroxybutyl)carbamate: LCMS (ES) m/e 496 (M+H)⁺; ¹H NMR (MeOH, 400 MHz) δ ppm 1.43 (t, J=7.07 Hz, 3H) 1.59-1.67 (m, 6H) 3.20 (d, J=10.11 Hz, 2H) 3.88 (s, 3H) 4.59-4.70 (m, 2H) 6.39 (dd, J=7.45, 3.92 Hz, 1H) 7.00-7.10 (m, 4H).

### Example 137

### Preparation of 4-[6-{[3-amino-1-(2-fluorophenyl)propyl]oxy}-2-(4-amino-1,2,5-oxadiazol-3-yl)-1-ethyl-1H-imidazo[4,5-c]pyridin-4-yl]-2-methyl-3-butyn-2-ol

### a) 1,1-dimethylethyl[3-(2-fluorophenyl)-3-hydroxypropyl]carbamate

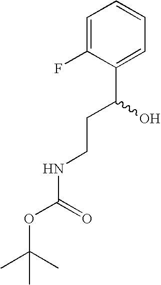

The title compound was prepared as an oil according to the Example 120, except substituting 2-fluorobenzaldehyde (7.18 g, 57.9 mmol) for cyclohexyl carboxaldehyde: LCMS (ES) m/e 270 (M+H)⁺.

### b) Preparation of 4-[6-{[3-amino-1-(2-fluorophenyl)propyl]oxy}-2-(4-amino-1,2,5-oxadiazol-3-yl)-1-ethyl-1H-imidazo[4,5-c]pyridin-4-yl]-2-methyl-3-butyn-2-ol

The title compound was prepared as a tan solid according to Example 17, except substituting 1,1-dimethylethyl[3-(2-fluorophenyl)-3-hydroxypropyl]carbamate (504 mg, 1.87 mmol) for 1,1-dimethylethyl (4-hydroxybutyl)carbamate: LCMS (ES) m/e 480 (M+H)⁺; ¹H NMR (CDCl₃, 400 MHz) δ ppm 1.43 (t, J=7.20 Hz, 3H) 1.65-1.75 (m, 6H) 2.39 (ddd, J=19.58, 9.73, 4.80 Hz, 2H) 3.09-3.20 (m, 1H) 3.21-3.30 (m, 1H) 4.59 (q, J=7.07 Hz, 2H) 5.89 (s, 2H) 5.98 (s, 3H) 6.41 (dd, J=9.47, 3.92 Hz, 1H) 6.77 (s, 1H) 7.02-7.11 (m, 1H) 7.17 (t, J=7.58 Hz, 1H) 7.25-7.33 (m, 1H) 7.52-7.61 (m, 1H).

### Example 138

### Preparation of 4-[6-{[(2R)-2-amino-3-(4-fluorophenyl)propyl]oxy}-2-(4-amino-1,2,5-oxadiazol-3-yl)-1-ethyl-1H-imidazo[4,5-c]pyridin-4-yl]-2-methyl-3-butyn-2-ol

The title compound was prepared as a brown solid according to Example 62, except substituting 4-fluoro-D-phenylalanine (500 mg, 2.73 mmol) for (2S)-2-amino-4-phenylbutanoic acid: LCMS (ES) m/e 480 (M+H)⁺; ¹H NMR (MeOD, 400 MHz) δ ppm 1.46 (t, J=7.20 Hz, 3H) 1.67 (S, 6H), 2.79-2.84 (m, 1H), 2.95-3.01 (m, 1H), 3.43-3.49 (m, 1H), 4.15-4.21 (m, 1H), 4.6-4.66 (m, 1H), 4.69 (q, J=7.07 Hz, 2H), 7.03-7.1 (m, 3H), 7.28-7.32 (m, 2H).

### Example 139

### Preparation of 4-[6-{[(2R)-2-amino-3-(2-fluorophenyl)propyl]oxy}-2-(4-amino-1,2,5-oxadiazol-3-yl)-1-ethyl-1H-imidazo[4,5-c]pyridin-4-yl]-2-methyl-3-butyn-2-ol

The title compound was prepared as a brown solid according to Example 62, except substituting 2-fluoro-D-phenylalanine (500 mg, 2.73 mmol) for (2S)-2-amino-4-phenylbutanoic acid: LCMS (ES) m/e 480 (M+H)⁺; ¹H NMR (MeOD, 400 MHz) δ ppm 1.47 (t, J=7.20 Hz, 3H) 1.67 (S, 6H), 2.9-2.96 (m, 1H), 3.01-3.07 (m, 1H), 3.52-3.61 (m, 1H), 4.2-4.25 (m, 1H), 4.33-4.38 (m, 1H), 4.7 (q, J=7.07 Hz, 2H), 7.05 (s, 1H), 7.09-7.17 (m, 2H), 7.28-7.37 (m, 2H).

### Example 140

### Preparation of 4-[2-(4-amino-1,2,5-oxadiazol-3-yl)-6-({(2R)-2-amino-3-[2-(trifluoromethyl)phenyl]propyl}oxy)-1-ethyl-1H-imidazo[4,5-c]pyridin-4-yl]-2-methyl-3-butyn-2-ol

The title compound was prepared as a yellow solid according to Example 62, except substituting 4-trifluoromethyl-D-phenylalanine (600 mg, 2.55 mmol) for (2S)-2-amino-4-phenylbutanoic acid: LCMS (ES) m/e 529 (M+H)⁺; 1H NMR (MeOD, 400 MHz) δ ppm 1.44 (t, J=7.20 Hz, 3H) 1.67 (S, 6H), 2.95-3.02 (m, 1H), 3.17-3.23 (m, 1H), 3.5-3.58 (m, 1H), 4.2-4.25 (m, 1H), 4.32-4.38 (m, 1H), 4.66 (q, J=7.07 Hz, 2H), 6.96 (s, 1H), 7.49-7.53 (m, 1H), 7.57-7.61 (m, 2H), 7.69-7.73 (m, 1H).

### Example 141

### Preparation of 4-[6-{[(2R)-2-amino-3-(4-chlorophenyl)propyl]oxy}-2-(4-amino-1,2,5-oxadiazol-3-yl)-1-ethyl-1H-imidazo[4,5-c]pyridin-4-yl]-2-methyl-3-butyn-2-ol

The title compound was prepared as a yellow solid according to Example 62, except substituting methyl 4-chloro-D-phenylalaninate (600 mg, 3.0 mmol) for (2S)-2-amino-4-phenylbutanoic acid: LCMS (ES) m/e 496 (M+H)⁺; ¹H NMR (MeOD, 400 MHz) δ ppm 1.45 (t, J=7.20 Hz, 3H) 1.67 (S, 6H), 2.76-2.85 (m, 1H), 2.90-3.0 (m, 1H), 3.4-3.48 (m, 1H), 4.1-4.2 (m, 1H), 4.24-4.33 (m, 1H), 4.66 (q, J=7.07 Hz, 2H), 6.96 (s, 1H), 7.23-7.36 (m, 4H).

### Example 142

### Preparation of 4-[6-{[(2R)-2-amino-3-(3-chlorophenyl)propyl]oxy}-2-(4-amino-1,2,5-oxadiazol-3-yl)-1-ethyl-1H-imidazo[4,5-c]pyridin-4-yl]-2-methyl-3-butyn-2-ol

The title compound was prepared as a yellow solid according to Example 62, except substituting methyl 3-chloro-D-phenylalaninate (600 mg, 3.0 mmol) for (2S)-2-amino-4-phenylbutanoic acid: LCMS (ES) m/e 496 (M+H)⁺; ¹H NMR (MeOD, 400 MHz) δ ppm 1.46 (t, J=7.20 Hz, 3H) 1.69 (S, 6H), 2.77-2.86 (m, 1H), 2.93-3.03 (m, 1H), 3.44-3.51 (m, 1H), 4.15-4.22 (m, 1H), 4.28-4.34 (m, 1H), 4.66 (q, J=7.07 Hz, 2H), 7.01 (s, 1H), 7.22-7.27 (m, 2H), 7.29-7.35 (m, 2H).

### Example 143

### Preparation of 4-[6-{[(2R)-2-amino-3-(2-chlorophenyl)propyl]oxy}-2-(4-amino-1,2,5-oxadiazol-3-yl)-1-ethyl-1H-imidazo[4,5-c]pyridin-4-yl]-2-methyl-3-butyn-2-ol

The title compound was prepared as a yellow solid according to Example 62, except substituting methyl 2-chloro-D-phenylalaninate (600 mg, 3.0 mmol) for (2S)-2-amino-4-phenylbutanoic acid: LCMS (ES) m/e 496 (M+H)⁺; ¹H NMR (MeOD, 400 MHz) δ ppm 1.42 (t, J=7.20 Hz, 3H) 1.63 (S, 6H), 3.26-3.28 (m, 2H), 3.97-4.06 (m, 1H), 4.27-4.36 (m, 1H), 4.48-4.54 (m, 1H), 4.64 (q, J=7.07 Hz, 2H), 7.04 (s, 1H), 7.24-7.31 (m, 2H), 7.36-7.47 (m, 2H).

### Example 144

### Preparation of 4-[2-(4-amino-1,2,5-oxadiazol-3-yl)-6-({(2R)-2-amino-3-[3-(trifluoromethyl)phenyl]propyl}oxy)-1-ethyl-1H-imidazo[4,5-c]pyridin-4-yl]-2-methyl-3-butyn-2-ol

The title compound was prepared as a yellow solid according to Example 62, except substituting 3-trifluoromethyl-D-phenylalaninate (500 mg, 2.13 mmol) for (2S)-2-amino-4-phenylbutanoic acid: LCMS (ES) m/e 530 (M+H)⁺; ¹H NMR (MeOD, 400 MHz) δ ppm 1.49 (t, J=7.20 Hz, 3H) 1.66 (S, 6H), 3.22-3.31 (m, 2H), 3.97-4.06 (m, 1H), 4.37-4.45 (m, 1H), 4.56-4.62 (m, 1H), 4.72 (q, J=7.07 Hz, 2H), 7.14 (s, 1H), 7.59-7.71 (m, 4H).

### Example 145

### Preparation of 4-[6-{[(2R)-2-amino-3-(3-fluorophenyl)propyl]oxy}-2-(4-amino-1,2,5-oxadiazol-3-yl)-1-ethyl-1H-imidazo[4,5-c]pyridin-4-yl]-2-methyl-3-butyn-2-ol

The title compound was prepared as a yellow solid according to Example 62, except substituting 3-trifluoromethyl-D-phenylalaninate (500 mg, 2.73 mmol) for (2S)-2-amino-4-phenylbutanoic acid: LCMS (ES) m/e 480 (M+H)⁺; ¹H NMR (MeOD, 400 MHz) δ ppm 1.49 (t, J=7.20 Hz, 3H) 1.67 (S, 6H), 3.12-3.22 (m, 2H), 3.93-4.01 (m, 1H), 4.36-4.44 (m, 1H), 4.55-4.62 (m, 1H), 4.73 (q, J=7.07 Hz, 2H), 7.05-7.15 (m, 3H), 7.16-7.22 (m, 1H), 7.37-7.46 (m, 1H).

### Example 146

### Preparation of 4-[2-(4-amino-1,2,5-oxadiazol-3-yl)-6-({(2R)-2-amino-3-[4-(trifluoromethyl)phenyl]propyl}oxy)-1-ethyl-1H-imidazo[4,5-c]pyridin-4-yl]-2-methyl-3-butyn-2-ol

The title compound was prepared as a yellow solid according to Example 62, except substituting 4-trifluoromethyl-D-phenylalaninate (600 mg, 2.55 mmol) for (2S)-2-amino-4-phenylbutanoic acid: LCMS (ES) m/e 530 (M+H)⁺; ¹H NMR (MeOD, 400 MHz) δ ppm 1.49 (t, J=7.20 Hz, 3H) 1.67 (S, 6H), 3.22-3.29 (m, 2H), 3.99-4.07 (m, 1H), 4.38-4.46 (m, 1H), 4.56-4.63 (m, 1H), 4.73 (q, J=7.07 Hz, 2H), 7.14 (s, 1H), 7.54-7.61 (m, 2H), 7.69-7.75 (m, 2H).

### Example 147

### Preparation of 4-[6-{[(2R)-2-amino-3-(1-benzothien-2-yl)propyl]oxy}-2-(4-amino-1,2,5-oxadiazol-3-yl)-1-ethyl-1H-imidazo[4,5-c]pyridin-4-yl]-2-methyl-3-butyn-2-ol

The title compound was prepared as a brown solid according to Example 62, except substituting 3-(1-benzothien-3-yl)-D-alanine (553 mg, 2.5 mmol) for (2S)-2-amino-4-phenylbutanoic acid: LCMS (ES) m/e 518 (M+H)⁺; ¹H NMR (MeOD, 400 MHz) δ ppm 1.49 (t, J=7.20 Hz, 3H) 1.69 (S, 6H), 3.37-3.43 (m, 1H), 3.43-3.54 (m, 1H), 4.07-4.20 (m, 1H), 4.41-4.54 (m, 1H), 4.61-4.80 (m, 3H), 7.12 (s, 1H), 7.36-7.5 (m, 2H), 7.61 (s, 1H), 7.87-7.98 (m, 2H).

### Example 148

### Preparation of 4-[6-{[(2R)-2-amino-3-cyclohexylpropyl]oxy}-2-(4-amino-1,2,5-oxadiazol-3-yl)-1-ethyl-1H-imidazo[4,5-c]pyridin-4-yl]-2-methyl-3-butyn-2-ol

The title compound was prepared as a brown solid according to Example 62, except substituting 3-cyclohexyl-N-{[(1,1-dimethylethyl)oxy]carbonyl}-D-alanine (879 mg, 3 mmol) for (2S)-2-amino-4-phenylbutanoic acid: LCMS (ES) m/e 468 (M+H)⁺; ¹H NMR (MeOD, 400 MHz) δ ppm 0.98-1.12 (m, 2H), 1.23-1.94 (m, 20H), 3.73-3.83 (m, 1H), 4.36-4.46 (m, 1H), 4.64-4.77 (m, 3H), 7.11 (s, 1H).

### Example 149

### Preparation of 4-{2-(4-amino-1,2,5-oxadiazol-3-yl)-6-[(3-amino-3-phenylpropyl)oxy]-1-ethyl-1H-imidazo[4,5-c]pyridin-4-yl}-2-methyl-3-butyn-2-ol

The title compound was prepared as a yellow solid according to Example 62, except substituting 3-amino-3-phenylpropanoic acid (496 mg, 3 mmol) for (2S)-2-amino-4-phenylbutanoic acid: LCMS (ES) m/e 462 (M+H)⁺; ¹H NMR (MeOD, 400 MHz) δ ppm 1.47 (t, J=7.20 Hz, 3H) 1.69 (S, 6H), 2.41-2.53 (m, 1H), 2.58-2.68 (m, 1H), 4.17-4.27 (m, 1H), 4.43-4.52 (m, 1H), 4.60-4.67 (m, 1H), 4.72 (q, J=7.07 Hz, 2H), 7.07 (s, 1H), 7.46-7.55 (m, 5H).

### Example 150

### Preparation of 4-{2-(4-amino-1,2,5-oxadiazol-3-yl)-6-[(3-amino-2-phenylpropyl)oxy]-1-ethyl-1H-imidazo[4,5-c]pyridin-4-yl}-2-methyl-3-butyn-2-ol

The title compound was prepared as a yellow solid according to Example 107, except substituting ethyl cyano(phenyl)acetate (756 mg, 4 mmol) for benzoylacetonitrile: LCMS (ES) m/e 462 (M+H)⁺; ¹H NMR (MeOD, 400 MHz) δ ppm 1.50 (t, J=7.20 Hz, 3H) 1.68 (S, 6H), 3.42-3.53 (m, 1H), 3.61-3.71 (m, 2H), 4.65-4.74 (m, 2H), 4.74-4.82 (m, 2H), 7.42-7.55 (m, 6H).

### Example 151

### Preparation of 4-[6-{[2-amino-1-(3-chlorophenyl)ethyl]oxy}-2-(4-amino-1,2,5-oxadiazol-3-yl)-1-ethyl-1H-imidazo[4,5-c]pyridin-4-yl]-2-methyl-3-butyn-2-ol

The title compound was prepared as a yellow solid according to Example 88, except substituting 3-chlorobenzaldehyde (2.8 g, 20 mmol) for cyclohexyl carboxaldehyde: LCMS (ES) m/e 482 (M+H)⁺; ¹H NMR (DMSO, 400 MHz) δ ppm 1.37 (t, J=7.20 Hz, 3H) 1.52 (S, 6H), 3.27-3.31 (m, 2H), 4.65 (q, J=7.07 Hz, 2H), 6.35-6.41 (m, 1H), 7.01 (s, 2H), 7.35 (s, 1H), 7.40-7.50 (m, 3H), 7.58 (s, 1H), 7.67-7.75 (m, 1H), 8.23 (s, 2H).

### Example 152

### Preparation of 4-[6-{[2-amino-1-(2-chlorophenyl)ethyl]oxy}-2-(4-amino-1,2,5-oxadiazol-3-yl)-1-ethyl-1H-imidazo[4,5-c]pyridin-4-yl]-2-methyl-3-butyn-2-ol

The title compound was prepared as a yellow solid according to Example 88, except substituting 2-chlorobenzaldehyde (2.8 g, 20 mmol) for cyclohexyl carboxaldehyde: LCMS (ES) m/e 482 (M+H)⁺; ¹H NMR (MeOD, 400 MHz) δ ppm 1.46 (t, J=7.20 Hz, 3H) 1.64 (S, 6H), 3.46-3.50 (m, 2H), 4.73 (q, J=7.07 Hz, 2H), 6.77-6.8 (m, 1H), 7.22 (s, 1H), 7.35-7.36 (m, 2H), 7.50-7.52 (m, 1H), 7.63-7.65 (m, 1H).

### Example 153

### Preparation of 4-[6-{[2-amino-1-(4-chlorophenyl)ethyl]oxy}-2-(4-amino-1,2,5-oxadiazol-3-yl)-1-ethyl-1H-imidazo[4,5-c]pyridin-4-yl]-2-methyl-3-butyn-2-ol

The title compound was prepared as a brown solid according to Example 88, except substituting 4-chlorobenzaldehyde (2.8 g, 20 mmol) for cyclohexyl carboxaldehyde: LCMS (ES) m/e 482 (M+H)⁺; ¹H NMR (MeOD, 400 MHz) δ ppm 1.46 (t, J=7.20 Hz, 3H) 1.65 (S, 6H), 3.46-3.51 (m, 2H), 4.71 (q, J=7.07 Hz, 2H), 6.41-6.44 (m, 1H), 7.17 (s, 1H), 7.41-7.43 (m, 2H), 7.53-7.55 (m, 2H).

### Example 154

### Preparation of 4-[6-{[3-amino-1-(3-fluorophenyl)propyl]oxy}-2-(4-amino-1,2,5-oxadiazol-3-yl)-1-ethyl-1H-imidazo[4,5-c]pyridin-4-yl]-2-methyl-3-butyn-2-ol

The title compound was prepared as a yellow solid according to Example 107, except substituting 3-(3-fluororphenyl)-3-oxopropanenitrile (398 mg, 2.35 mmol) for benzoylacetonitrile: LCMS (ES) m/e 480 (M+H)⁺; ¹H NMR (MeOD, 400 MHz) δ ppm 1.46 (t, J=7.20 Hz, 3H) 1.68 (S, 6H), 2.35-2.38 (m, 1H), 2.48-2.53 (m, 1H), 3.22-3.27 (m, 2H), 4.73 (q, J=7.07 Hz, 2H), 6.27-6.3 (m, 1H), 7.08-7011 (m, 1H), 7.39-7.41 (m, 4H).

### Example 155

### Preparation of 4-[6-{[3-amino-1-(4-fluorophenyl)propyl]oxy}-2-(4-amino-1,2,5-oxadiazol-3-yl)-1-ethyl-1H-imidazo[4,5-c]pyridin-4-yl]-2-methyl-3-butyn-2-ol

The title compound was prepared as a yellow solid according to Example 107, except substituting 3-(4-fluororphenyl)-3-oxopropanenitrile (398 mg, 2.35 mmol) for benzoylacetonitrile: LCMS (ES) m/e 480 (M+H)⁺; ¹H NMR (MeOD, 400 MHz) δ ppm 1.41-1.47 (m, 3H) 1.68 (S, 6H), 2.23-2.31 (m, 1H), 2.4-2.47 (m, 1H), 3.12-3.2 (m, 2H), 4.6-4.67 (m, 2H), 6.17-6.2 (m, 1H), 7.06-7.12 (m, 3H), 7.5-7.54 (m, 2H).

### Example 156

### Preparation of 4-[6-{[2-amino-1-(2-fluorophenyl)ethyl]oxy}-2-(4-amino-1,2,5-oxadiazol-3-yl)-1-ethyl-1H-imidazo[4,5-c]pyridin-4-yl]-2-methyl-3-butyn-2-ol

The title compound was prepared as a white solid according to Example 88, except substituting 2-fluorobenzaldehyde (4.96 g, 40 mmol) for cyclohexyl carboxaldehyde: LCMS (ES) m/e 466 (M+H)⁺; ¹H NMR (MeOD, 400 MHz) δ ppm 1.47 (t, J=7.20 Hz, 3H) 1.65 (S, 6H), 3.46-3.60 (m, 2H), 4.7 (q, J=7.07 Hz, 2H), 6.66-6.72 (m, 1H), 7.16-7.27 (m, 3H), 7.37-7.47 (m, 1H), 7.54-7.63 (m, 1H).

### Example 157

### Preparation of 4-[6-{[2-amino-1-(3-fluorophenyl)ethyl]oxy}-2-(4-amino-1,2,5-oxadiazol-3-yl)-1-ethyl-1H-imidazo[4,5-c]pyridin-4-yl]-2-methyl-3-butyn-2-ol

The title compound was prepared as a yellow solid according to Example 88, except substituting 3-fluorobenzaldehyde (4.96 g, 40 mmol) for cyclohexyl carboxaldehyde: LCMS (ES) m/e 466 (M+H)⁺; ¹H NMR (MeOD, 400 MHz) δ ppm 1.47 (t, J=7.20 Hz, 3H) 1.66 (S, 6H), 3.5-3.52 (m, 2H), 4.72 (q, J=7.07 Hz, 2H), 6.43-6.46 (m, 1H), 7.08-7.12 (m, 1H), 7.22 (s, 1H), 7.41-7.47 (m, 3H).

### Example 158

### Preparation of 4-[6-{[2-amino-1-(4-fluorophenyl)ethyl]oxy}-2-(4-amino-1,2,5-oxadiazol-3-yl)-1-ethyl-1H-imidazo[4,5-c]pyridin-4-yl]-2-methyl-3-butyn-2-ol

The title compound was prepared as a yellow solid according to Example 88, except substituting 3-fluorobenzaldehyde (4.96 g, 40 mmol) for cyclohexyl carboxaldehyde: LCMS (ES) m/e 466 (M+H)⁺; ¹H NMR (MeOD, 400 MHz) δ ppm 1.47 (t, J=7.20 Hz, 3H) 1.66 (S, 6H), 3.48-3.51 (m, 2H), 4.72 (q, J=7.07 Hz, 2H), 6.44-6.47 (m, 1H), 7.11-7.13 (m, 2H), 7.22 (s, 1H), 7.41-7.45 (m, 2H).

### Example 159

### Preparation of 4-[6-({3-amino-1-[4-fluoro-3-(methyloxy)phenyl]propyl}oxy)-2-(4-amino-1,2,5-oxadiazol-3-yl)-1-ethyl-1H-imidazo[4,5-c]pyridin-4-yl]-2-methyl-3-butyn-2-ol

The title compound was prepared as a yellow solid according to Example 120, except substituting 4-fluoro-3-(methyloxy)benzaldehyde (2.5 g, 16.3 mmol) for 1,1-dimethylethyl 4-formyl-1-piperidinecarboxylate: LCMS (ES) m/e 510 (M+H)⁺; ¹H NMR (MeOD, 400 MHz) δ ppm 1.42 (t, J=7.20 Hz, 3H) 1.67 (S, 6H), 2.28-2.37 (m, 1H), 2.43-2.51 (m, 1H), 3.18-3.21 (m, 2H), 3.91 (s, 3H), 4.72 (q, J=7.07 Hz, 2H), 6.19-6.22 (m, 1H), 7.07-7.1 (m, 3H), 7.27-7.29 (M, 1H).

### Example 160

### Preparation of 4-[6-({2-amino-1-[4-fluoro-3-(methyloxy)phenyl]ethyl}oxy)-2-(4-amino-1,2,5-oxadiazol-3-yl)-1-ethyl-1H-imidazo[4,5-c]pyridin-4-yl]-2-methyl-3-butyn-2-ol

The title compound was prepared as a yellow solid according to Example 88, except substituting 4-fluoro-3-(methyloxy)benzaldehyde (2.5 g, 16.3 mmol) for cyclohexyl carboxaldehyde: LCMS (ES) m/e 496 (M+H)⁺; ¹H NMR (MeOD, 400 MHz) δ ppm 1.43 (t, J=7.20 Hz, 3H) 1.67 (S, 6H), 2.25-2.37 (m, 1H), 2.38-2.50 (m, 1H), 3.12 (s, 3H), 4.67 (q, J=7.07 Hz, 2H), 6.13-6.22 (m, 1H), 7.04-7.13 (m, 3H), 7.25-7.32 (m, 1H).

### Example 161

### Preparation of 4-[6-{[(2R)-2-Amino-3-(2-furanyl)propyl]oxy}-2-(4-amino-1,2,5-oxadiazol-3-yl)-1-ethyl-1H-imidazo[4,5-c]pyridin-4-yl]-2-methyl-3-butyn-2-ol

### a) 1,1-Dimethylethyl[(1R)-2-(2-furanyl)-1-(hydroxymethyl)ethyl]carbamate

Borane-tetrahydrofuran (1.0M in tetrahydrofuran, 37 mL) was added dropwise to a stirred solution of N-Boc-D-2-furylalanine (1.6 g, 6.3 mmol) in tetrahydrofuran (35 mL) at 0° C. The solution was kept 16 hours at −10° C., quenched with 9:1 methanol/acetic acid (17 mL) and solvents evaporated. The residue was partitioned between ethyl acetate (350 mL) and saturated NaHCO₃ (80 mL). The organic layer was washed with brine (80 mL), dried (Na₂SO₄) and evaporated to give the title compound (1.15 g, 77%). MS (ES+) m/z 242.3 (M+H)⁺.

### b) 1,1-Dimethylethyl[(1R)-2-{[2-(4-amino-1,2,5-oxadiazol-3-yl)-4-chloro-1-ethyl-1H-imidazo[4,5-c]pyridin-6-yl]oxy}-1-(2-furanylmethyl)ethyl]carbamate

Diisopropylazodicarboxylate (0.151 g, 0.75 mmol) was added to a stirred solution of the compound of Example 163(a) (0.167 g, 0.66 mmol), the compound of Example 17 (b) (0.150 g, 0.53 mmol) and triphenylphosphine (0.19 g, 0.75 mmol) in tetrahydrofuran (9.0 mL) at ambient temperature for 1 min. The solution was then stirred for 30 minutes at 0° C., quenched with methanol (3.0 mL) and evaporated to give the crude product, which was purified by flash chromatography (Silica Gel 60, 40:1 CH₂Cl₂:CH₃OH) to give the title compound (0.115 g, 80%). MS (ES+) m/z 504.3 (M+H)⁺.

### c) 1,1-Dimethylethyl[(1R)-2-{[2-(4-amino-1,2,5-oxadiazol-3-yl)-1-ethyl-4-(3-hydroxy-3-methyl-1-butyn-1-yl)-1H-imidazo[4,5-c]pyridin-6-yl]oxy}-1-(2-furanylmethyl)ethyl]carbamate

A suspension of the compound of Example 161(b) (0.115 g., 0.22 mmol), zinc dust (0.009 g, 0.12 mmol), sodium iodide (0.018 g., 0.12 mmol), DBU (0.143 g., 0.94 mmol) and triethylamine (0.144 g, 1.4 mmol) in DMSO (5.0 mL) was sonicated and purged with nitrogen. 2-methyl-3-butyn-2-ol (0.049 g, 0.58 mmol) was added, followed by tetrakis-(triphenylphosphine)palladium(0) (0.042 g, 0.036 mmol). The mixture was stirred at 80° C. in a sealed flask for 2 h, cooled, concentrated and the residue partitioned between ethyl acetate (200 mL and water (50 mL). The organic layer was washed with water (3×20 mL) and brine (25 mL), dried (Na₂SO₄) and evaporated to a yellow solid, which was purified by flash chromatography [Silica Gel 60; gradient: CH₂Cl₂:CH₃OH; 50:1 (300 mL) to 30:1 (250 mL) to 20:1)] to give the title compound (0.080 g, 56%). MS (ES+) m/z 552.4 (M+H)⁺.

### d) 4-[6-{[(2R)-2-Amino-3-(2-furanyl)propyl]oxy}-2-(4-amino-1,2,5-oxadiazol-3-yl)-1-ethyl-1H-imidazo[4,5-c]pyridin-4-yl]-2-methyl-3-butyn-2-ol

Trifluoroacetic acid (2.0 mL) was added to a solution of the compound of Example 161(c) (0.080 g, 0.14 mmol) in methylene chloride (5.0 mL). After one hour, the solution was concentrated and the residue purified by preparative HPLC (YMC Pack ODS-A, 75 mm×30 mm i.d., 20 mL/min, gradient, A: water-0.1% trifluoroacetic acid, B: acetonitrile-0.1% trifluoroacetic acid, 10-90% acetonitrile over 12 min, UV detection at 214 nm) to give the title compound as the di-TFA salt (0.038 g, 42%). MS (ES+) m/z 452.5 (M+H)⁺. ¹H NMR (400 MHz, DMSO-d₆) δ ppm 1.37 (t, J=7.07 Hz, 3H), 1.55 (s, 6H) 3.06-3.16 (m, 2H), 3.81-3.94 (m, 1H), 4.33 (dd, J=11.37, 6.06 Hz, 1H), 4.48 (dd, J=11.49, 3.66 Hz, 1H), 4.65 (q, J=7.07 Hz, 2H), 5.76 (s, 1H), 6.37 (d, J=2.78 Hz, 1H), 6.45 (dd, J=3.16, 1.89 Hz, 1H), 7.03 (s, 3H), 7.22 (s, 1H), 7.62-7.68 (m, 1H), 8.22 (s, 3H).

### Example 162

### Preparation of 3-(3-Amino-1-{[2-(4-amino-1,2,5-oxadiazol-3-yl)-1-ethyl-4-(3-hydroxy-3-methyl-1-butyn-1-yl)-1H-imidazo[4,5-c]pyridin-6-yl]oxy}propyl)phenol

### a) 1,1-Dimethylethyl[2-hydroxy-2-(3-hydroxyphenyl)ethyl]carbamate

Di-t-butyldicarbonate (1.74 g, 8.0 mmol) was added portionwise to a stirred solution of norphenylephrine hydrochloride (1.5 g, 8.0 mmol) in a mixture of 2N NaOH (9.0 mL) and t-butanol (6.0 mL) at ambient temperature. After 16 hours, the mixture was extracted with pentane (2×40 mL), the combined organic layers evaporated and the residue taken up in water (10 mL). The solution was acidified to pH 3 with saturated KHSO₄ and the suspension extracted with ethyl acetate (200 mL). The organic layer was dried (Na₂SO₄) and evaporated to give the title compound (1.1 g, 56%). MS (ES+) m/z 254.3 (M+H)⁺.

### b) 1,1-Dimethylethyl[2-hydroxy-2-(3-{[tris(1-methylethyl)silyl]oxy}phenyl)-ethyl]carbamate

Triisopropylsilyl chloride (0.64 g, 3.3 mmol) was added to a stirred solution of the compound of Example 162(a) and DBU (0.55 g., 3.6 mmol) in dimethylformamide (20 mL) at ambient temperature. After 16 hours, the solution was concentrated and the residue partitioned between ethyl acetate (250 mL) and water (25 mL). The organic layer was washed with water (2×25 mL) and brine (25 mL), dried (Na₂SO₄) and evaporated to give the title compound (1.2 g, 89%). MS (ES+) m/z 410.6 (M+H)⁺.

### c) 3-(3-Amino-1-{[2-(4-amino-1,2,5-oxadiazol-3-yl)-1-ethyl-4-(3-hydroxy-3-methyl-1-butyn-1-yl)-1H-imidazo[4,5-c]pyridin-6-yl]oxy}propyl)phenol

Following the procedure of Example 161, except substituting the compound of Example 162(b) for the compound of Example 161(a), the title compound was prepared as the di-TFA salt (0.03 g, 35%). MS (ES+) m/z 464.4 (M+H)⁺. ¹H NMR (400 MHz, DMSO-d₆) δ ppm 1.36 (t, J=7.20 Hz, 3H), 1.53 (s, 6H), 3.26-3.36 (m, 2H), 4.64 (q, J=7.20 Hz, 3H), 5.65-5.84 (m, 1H), 6.32 (t, J=6.44 Hz, 1H), 6.71 (dd, J=8.08, 1.52 Hz, 1H), 6.83-6.86 (m, 1H), 6.90 (d, J=7.58 Hz, 1H), 7.01 (s, 2H), 7.18 (t, J=7.83 Hz, 1H), 7.31 (s, 1H), 8.11 (s, 3H), 9.58 (s, 1H).

### Example 163

### Preparation of 4-((2R)-2-Amino-3-{[2-(4-amino-1,2,5-oxadiazol-3-yl)-1-ethyl-4-(3-hydroxy-3-methyl-1-butyn-1-yl)-1H-imidazo[4,5-c]pyridin-6-yl]oxy}propyl)phenol

### a) O-(1,1-Dimethylethyl)-N-{[(1,1-dimethylethyl)oxy]carbonyl}-D-tyrosine

Di-t-butyl dicarbonate (0.97 g., 4.46 mmol) was added portionwise to a stirred solution of O-t-butyl-D-tyrosine (1.06 g, 4.46 mmol) in a mixture of 1N NaOH (5.5 mL) and t-butanol (5.0 mL) at ambient temperature. After 16 hours, the solution was extracted with pentane (2×40 mL) and the aqueous layer acidified to pH 2 with saturated KHSO₄. The suspension was extracted with diethyl ether (2×50 mL) and the organic layers dried-(Na₂SO₄) and evaporated to give the title compound (1.45 g, 95%). MS (ES+) m/z 338.5 (M+H)⁺.

### b) 4-((2R)-2-Amino-3-{[2-(4-amino-1,2,5-oxadiazol-3-yl)-1-ethyl-4-(3-hydroxy-3-methyl-1-butyn-1-yl)-1H-imidazo[4,5-c]pyridin-6-yl]oxy}propyl)phenol

Following the procedure, of Example 161, except substituting the compound of Example 163(a) for N-Boc-D-2-furylalanine, the title compound was prepared as the di-TFA salt. MS (ES+) m/z 478.3 (M+H)⁺. ¹H NMR (400 MHz, DMSO-d₆) δ ppm 1.37 (t, J=7.07 Hz, 3H), 1.55 (s, 6H), 2.84-3.01 (m, 2H), 4.25 (dd, J=11.37, 5.81 Hz, 1H), 4.38 (dd, J=11.49, 3.16 Hz, 1H), 4.65 (q, J=6.91 Hz, 3H), 5.58-5.93 (m, 1H), 6.74 (d, J=8.34 Hz, 2H), 7.03 (s, 2H), 7.11 (d, J=8.59 Hz, 2H), 7.22 (s, 1H), 8.14 (s, 3H), 9.39 (s, 1H).

### Example 164

### Preparation of 4-((2S)-2-Amino-3-{[2-(4-amino-1,2,5-oxadiazol-3-yl)-1-ethyl-4-(3-hydroxy-3-methyl-1-butyn-1-yl)-1H-imidazo[4,5-c]pyridin-6-yl]oxy}propyl)phenol

Following the procedure of Example 161, except substituting N-Boc-O-t-butyl-L-tyrosine for N-Boc-D-2-furylalanine, the title compound was prepared as the di-TFA salt. MS (ES+) m/z 478.3 (M+H)⁺. ¹H NMR (400 MHz, DMSO-d₆) δ ppm 1.37 (t, J=7.07 Hz, 3H), 1.55 (s, 6H), 2.84-3.01 (m, 2H), 4.25 (dd, J=11.37, 5.81 Hz, 1H), 4.38 (dd, J=11.49, 3.16 Hz, 1H), 4.65 (q, J=6.91 Hz, 3H), 5.58-5.93 (m, 1H), 6.74 (d, J=8.34 Hz, 2H), 7.03 (s, 2H), 7.11 (d, J=8.59 Hz, 2H), 7.22 (s, 1H), 8.14 (s, 3H), 9.39 (s, 1H).

### Example 165

### Preparation of 4-(2-(4-Amino-1,2,5-oxadiazol-3-yl)-1-ethyl-6-{[(3R)-1,2,3,4-tetrahydro-3-isoquinolinylmethyl]-oxy}-1H-imidazo[4,5-c]pyridin-4-yl)-2-methyl-3-butyn-2-ol

Following the procedure of Example 161, except substituting N-Boc-D-tetrahydro-iso-quinoline-2-carboxylic acid for N-Boc-D-2-furylalanine, the title compound was prepared as the di-TFA salt. MS (ES+) m/z 474.6 (M+H)⁺. ¹H NMR (400 MHz, MeOD) δ ppm 1.49 (t, J=7.07 Hz, 3H), 1.68 (s, 6H), 3.25-3.30 (m, 3H), 4.05-4.15 (m, 1H), 4.53 (q, 2H), 4.65 (dd, J=12.00, 6.44 Hz, 1H), 4.73 (q, J=7.07 Hz, 2H), 4.83 (dd, J=12.13, 3.28 Hz, 1H), 7.12-7.19 (m, 1H), 7.26-7.40 (m, 4H).

### Example 166

### Preparation of 4-[6-({(2R)-2-Amino-3-[3-(methyloxy)phenyl}propyl]oxy)-2-(4-amino-1,2,5-oxadiazol-3-yl)-1-ethyl-1H-imidazo[4,5-c]pyridin-4-yl]-2-methyl-3-butyn-2-ol

### a) N-{[(1,1-dimethylethyl)oxy]carbonyl}-3-(methyloxy)-D-phenylalanine

Following the procedure of Example 162(a), except substituting 3-methoxy-D-phenylalanine for norphenylephrine hydrochloride, the title compound was prepared (1.3 g, 87%). MS (ES+) m/z 296.3 (M+H)⁺.

### b) 4-[6-({(2R)-2-Amino-3-[3-(methyloxy)phenyl]propyl}oxy)-2-(4-amino-1,2,5-oxadiazol-3-yl)-1-ethyl-1H-imidazo[4,5-c]pyridin-4-yl]-2-methyl-3-butyn-2-ol

Following the procedure of Example 161, except substituting the compound of Example 166(a) for N-Boc-D-2-furylalanine, the title compound was prepared as the di-TFA salt. MS (ES+) m/z 492.4 (M+H)⁺. ¹H NMR (400 MHz, DMSO-d₆) δ ppm 1.37 (t, J=7.07 Hz, 3H), 1.54 (s, 6H), 2.95-3.09 (m, 2H), 3.73 (s, 3H), 3.83-3.95 (m, 1H), 4.27 (dd, J=11.37, 5.81 Hz, 1H), 4.41 (dd, J=11.37, 3.28 Hz, 1H), 4.65 (q, J=7.07 Hz, 2H), 5.74 (s, 1H), 6.82-6.94 (m, 3H), 7.02 (s, 2H), 7.23 (s, 1H), 7.28 (t, J=7.96 Hz, 1H), 8.19 (s, 3H).

### Example 167

### Preparation of 3-(3-Amino-1-{[2-(4-amino-1,2,5-oxadiazol-3-yl)-1-ethyl-4-(3-hydroxy-3-methyl-1-butyn-1-yl)-1H-imidazo[4,5-c]pyridin-6-yl]oxy}propyl)phenol

### a) 3-[(Phenylmethyl)oxy]benzaldehyde

A mixture of 3-hydroxybenzaldehyde (3.0 g, 24.6 mmol), 4-methoxybenzyl chloride (3.9 g, 25.0 mmol), potassium carbonate (6.8 g, 49.2 mmol) and dimethylformamide (35 mL) was stirred for 16 hours at ambient temperature. The reaction mixture was diluted with water (700 mL) and the suspension extracted with ethyl acetate (2×200 mL). The combined organic layers were washed with water (3×25 mL) and brine (25 mL), dried (Na₂SO₄) and evaporated to a colorless solid, which was recrystallized from ethanol to give the title compound (4.2 g, 72%). ¹H NMR (400 MHz, CDCl₃) δ ppm 3.82-3.87 (s, 3H), 5.05-5.09 (m, 2H), 6.95 (d, J=8.59 Hz, 2H), 7.24-7.29 (m, 1H), 7.36-7.42 (d, J=8.59 Hz, 3H), 9.98-10.02 (m, 1H).

### b) 3-Hydroxy-3-{3-[(phenylmethyl)oxy]phenyl}propanenitrile

Acetonitrile (0.130 mL, 2.5 mmol) was added to a stirred solution of lithium diisopropylamide [2.0 M in heptane/THF/ethyl benzene (1.13 mL, 2.27 mmol) in 20 mL THF at −78° C. After 1 hour, a solution of the compound of Example 7(a) (0.50 g, 2.06 mmol) in THF (5 mL) was added dropwise and the mixture stirred 90 minutes at −78° C. Saturated ammonium chloride (5.0 mL) was added and the mixture warmed to ambient temperature. The resulting suspension was partitioned between diethyl ether (125 mL) and water (50 mL). The organic layer was dried (Na₂SO₄) and evaporated to give the crude product, which was purified by flash chromatography (Silica Gel 60; 3:2, hexanes/EtOAc) to give the title compound (0.39 g, 67%). ¹H NMR (400 MHz, CDCl₃) δ ppm 2.78 (d, J=5.56 Hz, 2H), 3.81-3.87 (s, 3H), 6.92-7.01 (m, 4H), 7.04-7.07 (m, 1H), 7.33 (t, J=7.83 Hz, 1H), 7.38 (d, J=8.84 Hz, 2H).

### c) 3-Amino-1-{3-[(phenylmethyl)oxy]phenyl}-1-propanol

Lithium aluminum hydride (1.0 M in THF, 4.14 mL) was added to a stirred solution of the compound of Example 7(b) (0.39 g, 1.38 mmol) in diethyl ether at 0° C. After 5 hours, the cold solution was added dropwise to 0.1 M NaOH (28 mL) at ambient temperature. Saturated potassium sodium tartrate (28 mL) was added followed by diethyl ether (25 mL). The mixture was stirred 30 minutes and the aqueous layer extracted with methylene chloride (3×30 mL). The combined organic layers were dried (Na₂SO₄) and evaporated to give the title compound (0.38 g., 98%). MS (ES+) m/z 288.2 (M+H)⁺.

### d) 3-(3-Amino-1-{[2-(4-amino-1,2,5-oxadiazol-3-yl)-1-ethyl-4-(3-hydroxy-3-methyl-1-butyn-1-yl)-1H-imidazo[4,5-c]pyridin-6-yl]oxy}propyl)phenol

Following the procedure of Example 163, except substituting the compound of Example 167(c) for N-Boc-D-2-furylalanine, the title compound was prepared as the di-TFA salt. MS (ES+) m/z 478.5 (M+H)⁺. ¹H NMR (400 MHz, DMSO-d₆) δ ppm 1.35 (t, J=7.20 Hz, 3H), 1.53 (s, 6H), 2.10-2.28 (m, 2H), 2.84-2.98 (m, 2H), 4.62 (q, J=7.24 Hz, 2H), 5.62-5.81 (m, 1H), 6.14 (dd, J=7.58, 5.05 Hz, 1H), 6.67 (dd, J=8.08, 3.03 Hz, 1H), 6.81-6.84 (m, 1H), 6.87 (d, J=7.83 Hz, 1H), 7.01 (s, 2H), 7.16 (t, J=7.96 Hz, 1H), 7.30 (s, 1H), 7.74 (s, 3H), 9.43-9.56 (m, 1H).

### Example 168

### Preparation of 4-[2-(4-amino-1,2,5-oxadiazol-3-yl)-1-ethyl-6-({2-[(3R)-1,2,3,4-tetrahydro-3-isoquinolinyl]ethyl}oxy)-1H-imidazo[4,5-c]pyridin-4-yl]-2-methyl-3-butyn-2-ol

### a) 1,1-Dimethylethyl (3S)-3-(2-hydroxyethyl)-3,4-dihydro-2(1H)-isoquinolinecarboxylate

Following the procedure of Example 161, except substituting N-Boc-D-tetrahydroisoquinoline-2-carboxylic acid for N-Boc-D-2-furylalanine, the title compound was prepared (0.93 g, 94%). ¹H NMR (400 MHz, CDCl₃) δ ppm 1.55 (s, 9H), 1.57-1.68 (m, 2H), 2.67 (dd, J=15.92, 2.02 Hz, 1H), 3.02 (s, 1H), 3.21 (dd, J=15.79, 5.68 Hz, 1H), 3.38-3.53 (m, 1H), 3.61 (dt, J=7.89, 3.92 Hz, 1H), 4.67-4.86 (m, 2H), 7.09-7.24 (m, 4H).

### b) 1,1-Dimethylethyl (3S)-3-(2-bromoethyl)-3,4-dihydro-2(1-isoquinolinecarboxylate

Triphenylphosphine (1.08 g, 4.12 mmol) was added portionwise to a stirred, cold (0° C.) solution of the compound of Example 168(a) (0.92 g, 3.3 mmol) and carbon tetrabromide (1.3 g., 3.96 mmol) in methylene chloride (40 mL). After the solution was evaporated and the residue purified by flash chromatography (Silica Gel 60, 40:1: methylene chloride:methanol) to give the title compound (0.75 g, 67%). ¹H NMR (400 MHz, CDCl₃) δ ppm 1.53 (s, 9H), 1.76-1.93 (m, 1H), 2.00-2.13 (m, 1H), 2.66 (d, J=16.17 Hz, 2H), 3.09-3.20 (m, J=16.04, 5.68 Hz, 2H), 4.14-4.31 (m, 1H), 4.71 (s, 1H), 4.88 (s, 1H), 7.10-7.25 (m, 4H).

### c) 1,1-Dimethylethyl (3R)-3-(2-{[2-(4-amino-1,2,5-oxadiazol-3-yl)-4-chloro-1-ethyl-1H-imidazo[4,5-c]pyridin-6-yl]oxy}ethyl)-3,4-dihydro-2(1H)-isoquinolinecarboxylate

A mixture of the compound of Example 168(b) (0.139 g, 0.41 mmol), the compound of Example 17(b) (0.115 g, 0.41 mmol), cesium carbonate (0.40 g, 1.23 mmol) and dimethylformamide (5.0 mL) was stirred at 35° C. for 16 hours. The suspension was concentrated and the residue partitioned between ethyl acetate (200 mL) and water (20 mL). The organic layer was washed with water (3×20 mL) and brine (20 mL), dried (Na₂SO₄) and evaporated to give the title compound (0.19 g, 85%). MS (ES+) m/z 540.4 (M+H)⁺.

### d) 4-[2-(4-Amino-1,2,5-oxadiazol-3-yl)-1-ethyl-6-({2-[(3R)-1,2,3,4-tetrahydro-3-isoquinolinyl]-ethyl}oxy)-1H-imidazo[4,5-c]pyridin-4-yl]-2-methyl-3-butyn-2-ol

Following the procedure of Example 161, except substituting the compound of Example 168(c) for the compound of 161(b), the title compound was prepared as the di-TFA salt. MS (ES+) m/z 488.6 (M+H)⁺. ¹H NMR (400 MHz, DMSO-d₆) δ ppm 1.36 (t, J=7.07 Hz, 3H) 1.56 (s, 6H) 2.05-2.17 (m, 1H) 2.26-2.39 (m, 1H) 2.98 (dd, J=17.31, 11.24 Hz, 1H) 3.25 (dd, J=17.18, 4.29 Hz, 1H) 3.67-3.81 (m, 1H) 4.34-4.46 (m, 2H) 4.49-4.59 (m, 2H) 4.63 (q, J=6.91 Hz, 2H) 5.75 (s, 1H) 7.03 (s, 2H) 7.21-7.35 (m, 5H) 9.05-9.29 (m, 2H).

### Example 169

### Preparation of 4-(2-(4-amino-1,2,5-oxadiazol-3-yl)-6-{[(2S)-2-amino-3-(3-pyridinyl)propyl]oxy}-1-ethyl-1H-imidazo[4,5-c]pyridin-4-yl)-2-methyl-3-butyn-2-ol

### a) 1,1-dimethylethyl[(1S)-2-{[2-(4-amino-1,2,5-oxadiazol-3-yl)-1-ethyl-4-(3-hydroxy-3-methyl-1-butyn-1-yl)-1H-imidazo[4,5-c]pyridin-6-yl]oxy}-1-(3-pyridinylmethyl)ethyl]carbamate

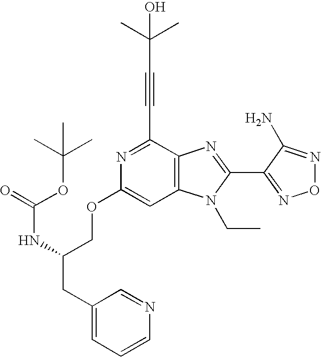

The title compound was prepared as a glassy yellow solid according to the procedures of Example 161, except substituting N-{[(1,1-dimethylethyl)oxy]carbonyl}-3-(3-pyridinyl)-L-alanine (1.0 g, 3.76 mmol) for N-Boc-D-2-furylalanine. LCMS (ES) m/z=563.3 (M+H)⁺.

### b) 4-(2-(4-amino-1,2,5-oxadiazol-3-yl)-6-{[(2S)-2-amino-3-(3-pyridinyl)propyl]oxy}-1-ethyl-1H-imidazo[4,5-c]pyridin-4-yl)-2-methyl-3-butyn-2-ol

To a solution of the compound of Example 169(a) (76 mg, 0.135 mmol) in MeOH (3 mL) at ambient temperature was added ethereal HCl (1.0 M solution, 2 mL), and the resultant mixture was stirred for 16 h. The precipitated title material was filtered off, washed with cold ether, and dried on high vacuum to provide a pale yellow solid (72 mg, 74% yield). LCMS (ES) m/z=463.4 (M+H)⁺; ¹H NMR [(CD₃)₂SO, 400 MHz] δ 9.01 (broad s, 1H), 8.87 (broad d, J=3.6 Hz, 1H), 8.68 (broad s, 3H), 8.63 (d, J=8.0 Hz, 1H), 8.08 (dd, J=7.6, 5.6 Hz, 1H), 7.22 (s, 1H), 7.03 (broad s, 2H), 4.65 (q, J=6.8, 2H), 4.53 (dd, 11.6, 4.4 Hz, 1H), 4.46 (dd, J=11.6, 5.2 Hz, 1H), 4.06-3.94 (m, 1H), 3.43-3.30 (m, 2H), 1.54 (s, 6H), 1.37 (t, 7.2 Hz, 3H).

### Example 170

### Preparation of 4-(2-(4-amino-1,2,5-oxadiazol-3-yl)-6-{[(2S)-2-amino-3-(4-Pyridinyl)propyl]oxy}-1-ethyl-1H-imidazo[4,5-c]pyridin-4-yl)-2-methyl-3-butyn-2-ol

The title compound was prepared as a pale tan solid according to the procedures of Example 161(a)-(c) and Example 169(b), except substituting N-{[(1,1-dimethylethyl)oxy]carbonyl}-3-(3-pyridinyl)-D-alanine (1.0 g, 3.76 mmol) for N-Boc-D-2-furylalanine: LCMS (ES) m/e 463.4 (M+H)⁺; ¹H NMR [(CD₃)₂SO, 400 MHz] δ 8.92 (broad d, J=4.8 Hz, 2H), 8.74 (broad s, 3H), 8.12 (broad d, J=6.0 Hz, 2H), 7.19 (s, 1H), 7.01 (broad s, 2H), 4.65 (q, J=6.8 Hz, 2H), 4.51 (dd, J=11.6, 4.0 Hz, 1H), 4.43 (dd, J=11.6, 6.0 Hz, 1H), 4.06-3.94 (m, 1H), 3.48 (dd, J=14.0, 6.8 Hz, 1H), 3.39 (dd, J=13.6, 7.2 Hz, 1H), 1.54 (s, 6H), 1.37 (t, J=6.8 Hz, 3H).

### Example 171

### Preparation of 4-[6-({(2R)-2-amino-3-[4-(methyloxy)phenyl]propyl}oxy)-2-(4-amino-1,2,5-oxadiazol-3-yl)-1-ethyl-1H-imidazo[4,5-c]pyridin-4-yl]-2-methyl-3-butyn-2-ol

The title compound was prepared as a pale tan solid according to the procedures of Example 161, except substituting N-{[(1,1-dimethylethyl)oxy]carbonyl}-O-methyl-D-tyrosine (1.5 g, 5.08 mmol) for N-Boc-D-2-furylalanine: LCMS (ES) m/e 492.6 (M+H)⁺; ¹H NMR [(CD₃)₂SO, 400 MHz] δ 8.18 (broad s, 3H), 7.24 (d, J=8.4 Hz, 2H), 7.22 (s, 1H), 7.02 (broad s, 2H), 6.92 (d, J=8.4 Hz, 2H), 5.75 (broad s, 1H), 4.65 (q, J=6.8 Hz, 2H), 4.39 (dd, J=10.8, 2.0 Hz, 1H), 4.24 (dd, J=11.2, 5.6 Hz, 1H), 3.87-3.74 (m, 1H), 3.73 (s, 3H), 3.04-2.90 (m, 2H), 1.54 (s, 6H), 1.36 (t, J=6.8 Hz, 3H).

### Example 172

### Preparation of 4-[6-{[(2S)-2-amino-3-(2-furanyl)propyl]oxy}-2-(4-amino-1,2,5-oxadiazol-3-yl)-1-ethyl-1H-imidazo[4,5-c]pyridin-4-yl]-2-methyl-3-butyn-2-ol

The title compound was prepared as a yellow solid according to the procedure of Example 162(a), except substituting 3-(2-furanyl)-L-alanine (1.0 g, 6.45 mmol) for norphenylephrine hydrochloride, and procedures of Example 161: LCMS (ES) m/e 452.4 (M+H)⁺; ¹H NMR [(CD3)₂SO, 400 MHz] δ 8.28 (broad s, 3H), 7.64 (s, 1H), 7.21 (s, 1H), 7.02 (broad s, 2H), 6.48-6.40 (m, 1H), 6.30 (broad d, J=2.8 Hz, 1H), 5.75 (broad s, 1H), 4.64 (q, J=6.8 Hz, 2H), 4.47 (dd, J=10.8, 3.2 Hz, 1H), 4.32 (dd, J=11.2, 6.0 Hz, 1H), 3.94-3.80 (m, 1H), 3.18-3.05 (m, 2H), 1.54 (s, 6H), 1.36 (t, J=7.2 Hz, 3H).

### Example 173

### Preparation of 4-[6-({(2R)-2-amino-3-[2-(methyloxy)phenyl]propyl}oxy)-2-(4-amino-1,2,5-oxadiazol-3-yl)-1-ethyl-1H-imidazo[4,5-c]pyridin-4-yl]-2-methyl-3-butyn-2-ol

The title compound was prepared as a pale tan solid according to the procedure of Example 162(a), except substituting 2-(methyloxy)-D-phenylalanine (1.0 g, 5.12 mmol) for norphenylephrine hydrochloride, and procedures of Example 161: LCMS (ES) m/e 492.6 (M+H)⁺; ¹H NMR [(CD₃)₂SO, 400 MHz] δ 8.20 (broad s, 3H), 7.28 (app. broad t, J=8.0 Hz, 1H), 7.23 (broad d, J=8.0 Hz, 1H), 7.19 (s, 1H), 7.06-6.98 (m, 3H), 6.92 (t, J=7.2 Hz, 1H), 4.64 (q, J=6.8 Hz, 2H), 4.37 (dd, J=11.2, 3.2 Hz, 1H), 4.23 (dd, J=11.6, 6.0 Hz, 1H), 3.87-3.74 (m, 1H), 3.78 (s, 3H), 3.08-2.96 (m, 2H), 1.54 (s, 6H), 1.36 (t, J=7.2 Hz, 3H).

### Example 174

### Preparation of 4-[6-[(3-amino-3-cyclohexylpropyl)oxy]-2-(4-amino-1,2,5-oxadiazol-3-yl)-1-ethyl-1H-imidazo[4,5-c]pyridin-4-yl]-2-methyl-3-butyn-2-ol

The title compound was prepared as a pale yellow solid according to the procedure of Example 168(c), except substituting 1,1-dimethylethyl (3-bromo-1-cyclohexylpropyl)carbamate (126 mg, 0.392 mol) for 1,1-Dimethylethyl (3S)-3-(2-bromoethyl)-3,4-dihydro-2(1H)-isoquinolinecarboxylate, and procedures of Example 161(c)-(d): LCMS (ES) m/e 468.5 (M+H)⁺; ¹H NMR [(CD₃)₂SO, 400 MHz] δ 7.83 (broad s, 3H), 7.23 (s, 1H), 7.02 (broad s, 2H), 5.74 (broad s, 1H), 4.63 (q, J=7.2 Hz, 2H), 4.46-4.36 (m, 2H), 3.24-3.14 (m, 1H), 2.16-2.04 (m, 1H), 2.00-1.87 (m, 1H), 1.87-1.50 (complex m, 6H), 1.54 (s, 6H), 1.35 (t, J=7.2 Hz, 3H), 1.30-1.00 (complex m, 5H).

### Example 175

### Preparation of 4-(2-(4-amino-1,2,5-oxadiazol-3-yl)-6-{[3-amino-3-(tetrahydro-2H-pyran-4-yl)propyl]oxy}-1-ethyl-1H-imidazo[4,5-c]pyridin-4-yl)-2-methyl-3-butyn-2-ol

The title compounds was prepared as pale yellow solid according to the procedure of Example 168(c), except substituting 1,1-dimethylethyl[3-bromo-1-(tetrahydro-2H-pyran-4-yl)propyl]carbamate [E-1 enantiomer] (126 mg, 0.391 mmol) for 1,1-dimethylethyl (3S)-3-(2-bromoethyl)-3,4-dihydro-2(1H)-isoquinolinecarboxylate, and procedures of Example 161(c)-(d): LCMS (ES) m/e 470.4 (M+H)⁺; ¹H NMR [(CD₃)₂SO, 400 MHz] δ 7.91 (broad s, 3H), 7.23 (s, 1H), 7.02 (broad s, 2H), 4.63 (q, J=6.8 Hz, 2H), 4.47-4.37 (m, 2H), 3.97-3.87 (m, 2H), 3.30 (app. dd, J=11.2, 11.2, 5.2 Hz, 2H), 3.25-3.15 (m, 1H), 2.19-2.06 (m, 1H), 2.02-1.84 (m, 2H), 1.61 (app. d, J=12.4, 2H)—, 1.54 (s, 6H), 1.45-1.39 (m, 1H), 1.39-1.30 (m, 4H).

### Example 176

### Preparation of 4-(2-(4-amino-1,2,5-oxadiazol-3-yl)-6-{[3-amino-3-(tetrahydro-2H-pyran-4-yl)propyl]oxy}-1-ethyl-1H-imidazo[4,5-c]pyridin-4-yl)-2-methyl-3-butyn-2-ol

The title compounds was prepared as pale yellow solid according to the procedure of Example 168(c), except substituting 1,1-dimethylethyl[3-bromo-1-(tetrahydro-2H-pyran-4-yl)propyl]carbamate [E-2 enantiomer] (126 mg, 0.391 mmol) for 1,1-dimethylethyl (3S)-3-(2-bromoethyl)-3,4-dihydro-2(1H)-isoquinolinecarboxylate, and procedures of Example 161(c)-(d): LCMS (ES) m/e 470.4 (M+H)⁺; ¹H NMR [(CD₃)₂SO, 400 MHz] δ 7.91 (broad s, 3H), 7.23 (s, 1H), 7.02 (broad s, 2H), 4.63 (q, J=6.8 Hz, 2H), 4.47-4.37 (m, 2H), 3.97-3.87 (m, 2H), 3.30 (app. dd, J=11.2, 11.2, 5.2 Hz, 2H), 3.25-3.15 (m, 1H), 2.19-2.06 (m, 1H), 2.02-1.84 (m, 2H), 1.61 (app. d, J=12.4, 2H), 1.54 (s, 6H), 1.45-1.39 (m, 1H), 1.39-1.30 (m, 4H).

### Example 177

### Preparation of 4-[2-(4-amino-1,2,5-oxadiazol-3-yl)-1-ethyl-6-({2-[(3R)-1,2,3,4-tetrahydro-3-isoquinolinyl]ethyl}oxy)-1H-imidazo[4,5-c]pyridin-4-yl]-2-methyl-3-butyn-2-ol

The title compound was prepared as a pale yellow solid according to the procedures of Example 168, except substituting ((3R)-2-{[(1,1-dimethylethyl)oxy]carbonyl}-1,2,3,4-tetrahydro-3-isoquinolinyl)acetic acid (1.0 g, 3.43 mmol) for ((3S)-2-{[(1,1-dimethylethyl)oxy]carbonyl}-1,2,3,4-tetrahydro-3-isoquinolinyl)acetic acid: LCMS (ES) m/e 488.6 (M+H)⁺; ¹H NMR [(CD₃)₂SO, 400 MHz] δ 9.29 (broad s, 1H), 9.20 (broad s, 1H), 7.32-7.21 (m, 5H), 7.03 (broad s, 2H), 4.62 (broad q, J=7.2 Hz, 2H), 4.56-4.48 (m, 2H), 4.42-4.35 (m, 2H), 3.74 (app. broad s, 1H), 3.24 (dd, J=16.8, 3.2 Hz, 1H), 2.98 (dd, J=16.8, 10.8 Hz, 1H), 2.38-2.26 (m, 1H), 2.16-2.04 (m, 1H), 1.55 (s, 6H), 1.35 (t, J=7.2 Hz, 3H).

### Example 178

### Preparation of 4-(2-(4-amino-1,2,5-oxadiazol-3-yl)-1-ethyl-6-{[(3S)-1,2,8,4-tetrahydro-3-isoquinolinylmethyl]oxy}-1H-imidazo[4,5-c]pyridin-4-yl)-2-methyl-3-butyn-2-ol

The title compound was prepared as a pale yellow solid according to the procedures of Example 161, except substituting 1,1-dimethylethyl (3S)-3-(hydroxymethyl)-3,4-dihydro-2(1H)-isoquinolinecarboxylate (176 mg, 0.688 mmol) for N-Boc-D-2-furylalanine: LCMS (ES) m/e 474.5 (M+H)⁺; ¹H NMR [(CD₃)₂SO, 400 MHz] δ 9.57 (broad s, 1H), 9.36 (broad s, 1H), 7.34-7.26 (m, 4H), 7.24 (s, 1H), 7.03 (broad s, 2H), 5.75 (s, 1H), 4.72-4.61 (m, 3H), 4.57 (dd, J=11.6, 6.4 Hz, 1H), 4.52-4.34 (m, 2H), 4.03 (app. broad s, 1H), 3.19 (dd, J=17.2, 4.8 Hz, 1H), 3.10 (dd, J=17.2, 10.8 Hz, 1H), 1.55 (s, 6H), 1.35 (t, J=7.2 Hz, 3H).

### Example 179

### Preparation of 4-[6-{[2-(aminomethyl)-4-phenylbutyl]oxy}-2-(4-amino-1,2,5-oxadiazol-3-yl)-1-ethyl-1H-imidazo[4,5-c]pyridin-4-yl]-2-methyl-3-butyn-2-ol

### a) 2-({[(4-methylphenyl)sulfonyl]oxy}methyl)-4-phenylbutyl 4-methylbenzenesulfonate

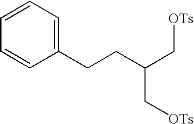

To a solution of 2-(2-phenylethyl)-1,3-propanediol (500 mg, 2.77 mmol), triethylamine (0.966 mL, 6.93 mmol), and DMAP (34 mg, 0.277 mmol) in dry dichloromethane (25 mL) at 0° C. was added tosyl chloride (1.269 g, 6.66 mmol). The resultant mixture was allowed to warm to ambient temperature and stirred overnight. Upon concentration in vacuo, the crude reaction mixture was purified on silica gel (CH₂Cl₂/Hexanes, 1:1→neat CH₂Cl₂) to furnish the title material as a white crystalline solid (900 mg, 73% yield). LCMS (ES) m/e 489.2 (M+H)⁺.

### b) bis(1,1-dimethylethyl) [2-({[2-(4-amino-1,2,5-oxadiazol-3-yl)-4-chloro-1-ethyl-1H-imidazo[4,5-c]pyridin-6-yl]oxy}methyl)-4-phenylbutyl]imidodicarbonate

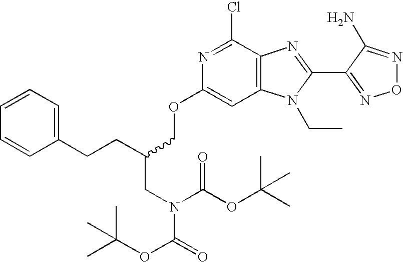

To a solution of the compound of Example 179(a) [441 mg, 0.903 mmol] and the compound of Example 17(b) (169 mg, 0.602 mmol) in DMF (6 mL) at 35° C. was added solid Cs₂CO₃ (491 mg, 1.50 mmol). The resultant mixture was stirred under N₂ at the above temperature for 16 h, at which time solid bis(1,1-dimethylethyl)imidodicarbonate (313 mg, 1.44 mmol) and Cs₂CO₃ (941 mg, 2.88 mmol) were added. The mixture was stirred under N₂ at 60° C. for 3.5 h, cooled to ambient temperature, and then partitioned between EtOAc (60 mL) and water (15 mL). The layers were separated, and the organic layer was washed with water (2×15 mL) and brine (15 mL), dried (MgSO₄), and concentrated in vacuo. Flash chromatography on silica gel (CH₂Cl₂/MeOH, 60:1→50:1) gave the title material as a pale yellow oil, (273 mg, 70% yield). LCMS (ES) m/e 642.6 (M+H)⁺.

### c) 4-[6-{[2-(aminomethyl)-4-phenylbutyl]oxy}-2-(4-amino-1,2,5-oxadiazol-3-yl)-1-ethyl-1H-imidazo[4,5-c]pyridin-4-yl]-2-methyl-3-butyn-2-ol

The title compound was prepared as a pale yellow solid according to the procedures of Example 161, except substituting the compound of Example 179(b) for the compound of example 161(b): LCMS (ES) m/e 490.6 (M+H)⁺; ¹H NMR [(CD₃)₂SO, 400 MHz] δ 7.84 (broad s, 3H), 7.33-7.23 (complex m, 5H), 7.22-7.15 (m, 1H), 7.02 (broad s, 2H), 5.73 (broad s, 1H), 4.73 (q, J=7.2 Hz, 2H), 4.41 (dd, J=11.2, 4.8 Hz, 1H), 4.35 (dd, J=10.8, 5.6 Hz, 1H), 3.10-2.90 (m, 2H), 2.81-2.62 (m, 2H), 2.16 (m, 1H), 1.85-1.72 (m, 2H), 1.54 (s, 6H), 1.36 (t, J=6.8 Hz, 3H).

### Example 180

### Preparation of 4-[6-[(3-amino-1-{[3-(methyloxy)phenyl]methyl}propyl)oxy]-2-(4-amino-1,2,5-oxadiazol-3-yl)-1-ethyl-1H-imidazo[4,5-c]pyridin-4-yl]-2-methyl-3-butyn-2-ol

### a) 4-[3-(methyloxy)phenyl]-3-oxobutanenitrile

To a solution of cyanoacetic acid (1.191 g, 14.0 mmol) and 2-2′-bipyridyl (˜1 mg) in dry THF (84 mL) at −78° C. was added n-BuLi (2.5 M solution in hexanes, 10 mL, 25 mmol), at which time a persistent pink color was observed, indicating that a slight excess of the base was present. The reaction mixture was allowed to warm to 0° C., and additional n-BuLi (1.2 mL) was added to maintain the pink color. The reaction was re-cooled to −78° C., and treated with [3-(methyloxy)phenyl]acetyl chloride (1.1 mL, 7.0 mmol) dropwise via syringe. The reaction mixture was stirred at the above temperature for 45 min and then allowed to warm to ambient temperature over 1 h, at which time 1 N aqueous HCl (35 mL) and diethyl ether (100 mL) were added. The layers were separated, and the organic layer was washed with sat. aqueous NaHCO₃ solution (50 mL) and brine (50 mL), dried (MgSO₄), and concentrated in vacuo to provide the title material as a pale brown oil (1.29 g, 97%). ¹H NMR (CDCl₃, 400 MHz) δ 7.29 (app. t, J=8.0 Hz, 1H), 6.87 (ddd, J=8.4, 2.4, 0.8 Hz, 1H), 6.83-6.78 (m, 1H), 6.75 (app. broad t, J=2.0 Hz, 1H), 3.82 (broad s, 2H), 3.81 (s, 3H), 3.46 (s, 2H).

### b) 1,1-dimethylethyl {3-hydroxy-4-[3-(methyloxy)phenyl]butyl}carbamate

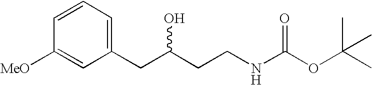

To a solution of the compound of Example 11(a) [1.28 g, 6.76 mmol) in THF (25 mL) at 0° C. was added LAH (1 M solution in THF, 22 mL, 22.0 mmol) dropwise via syringe, and the resultant mixture was stirred at ambient temperature for 16 h. The reaction was re-cooled to 0° C. and treated drop-wise with water (1 mL), followed by 2.5 NNaOH (1.5 mL) and water (3 mL). The solid were filtered off and washed with EtOAc (100 mL). The combined organics were dried (MgSO₄) and concentrated in vacuo.

The resultant oily residue (1.21 g) was dissolved in THF (25 mL) and treated with solid bis(1,1-dimethylethyl)dicarbonate (1.48 g, 6.79 mmol). The mixture was then stirred at ambient temperature for 16 h, concentrated, and purified on silica gel (Hexanes/EtOAc, 2:1→1:1) to yield the title material as a pale yellow oil (558 mg, 28% combined yield). ¹H NMR (CDCl₃, 400 MHz) δ 7.26-7.19 (m, 1H), 6.82-6.74 (complex m, 3H), 4.85 (s, 1H), 3.93-3.82 (m, 1H), 3.80 (s, 3H), 3.20-3.10 (m, 1H), 2.81-2.70 (m, 3H), 1.74-1.65 (m, 1H), 1.59-1.50 (m, 1H), 1.44 (s, 9H).

### c) 4-[6-[(3-amino-1-{[3-(methyloxy)phenyl]methyl}propyl)oxy]-2-(4-amino-1,2,5-oxadiazol-3-yl)-1-ethyl-1H-imidazo[4,5-c]pyridin-4-yl]-2-methyl-3-butyn-2-ol

The title compound was prepared as a yellow solid according to the procedures of Example 161(b)-(d), except substituting the compound of Example 180(b) [133 mg, 0.450 mol] for the compound of Example 161(a): LCMS (ES) m/e 506.4 (M+H)⁺; ¹H NMR [(CD₃)₂SO, 400 MHz] δ 7.70 (broad s, 3H), 7.21 (app. t, J=7.6 Hz, 1H), 7.21 (overlapping s, 1H), 7.02 (broad s, 2H), 6.93-6.88 (m, 2H), 6.79 (dd, J=8.4, 2.4 Hz, 1H), 5.72 (s, 1H), 5.43-5.34 (m, 1H), 4.62 (q, J=7.0 Hz, 2H), 3.73 (s, 3H), 3.06 (dd, J=13.6, 6.4 Hz, 1H), 3.00-2.85 (m, 3H), 2.03-1.84 (complex m, 2H), 1.56 (s, 6H), 1.35 (t, J=6.8 Hz, 3H).

### Example 181

### Preparation of 4-(2-(4-amino-1,2,5-oxadiazol-3-yl)-6-{[3-amino-1-(3-thienylmethyl)propyl]oxy}-1-ethyl-1H-imidazo[4,5-c]pyridin-4-yl)-2-methyl-3-butyn-2-ol

The title compound was prepared as a yellow solid according to the procedures of Example 180, except substituting 3-thienylacetyl chloride (1.13 g, 7.03 mmol) for [3-(methyloxy)phenyl]acetyl chloride: LCMS (ES) m/e 482.2 (M+H)⁺; ¹H NMR [(CD₃)₂SO, 400 MHz] δ 7.77 (broad s, 3H), 7.47 (broad dd, J=4.4, 3.2 Hz, 1H), 7.30 (s, 1H), 7.24 (s, 1H), 7.1 (d, J=4.4 Hz, 1H), 7.02 (broad s, 2H), 5.43-5.34 (m, 1H), 4.62 (q, J=7.0 Hz, 2H), 3.73 (s, 3H), 3.10-2.85 (complex m, 4H), 2.05-1.84 (complex m, 2H), 1.56 (s, 6H), 1.36 (t, J=6.8 Hz, 3H).

### Example 182

### Preparation of 4-[6-[(3-amino-1-{[3,4-bis(methyloxy)phenyl]methyl}propyl)oxy]-2-(4-amino-1,2,5-oxadiazol-3-yl)-1-ethyl-1H-imidazo[4,5-c]pyridin-4-yl]-2-methyl-3-butyn-2-ol

The title compound was prepared as a yellow solid according to the procedures of Example 180, except substituting [3,4-bis(methyloxy)phenyl]acetyl chloride (1.509 g, 7.03 mmol) for [3-(methyloxy)phenyl]acetyl chloride: LCMS (ES) m/e 536.4 (M+H)⁺; ¹H NMR [(CD₃)₂SO, 400 MHz] δ 7.73 (broad s, 3H), 7.21 (s, 1H), 7.02 (broad s, 2H), 6.89 (d, J=2.0 Hz, 1H), 6.86 (d, J=8.0 Hz, 1H), 6.82 (dd, J=8.0 Hz, 2.0 Hz, 1H), 5.72 (s, 1H), 5.42-5.33 (m, 1H), 4.62 (q, J=7.0 Hz, 2H), 3.74 (s, 3H), 3.70 (s, 3H), 3.00 (dd, J=13.6, 5.6 Hz, 1H), 3.00-2.86 (overlapping m, 2H), 2.85 (dd, J=13.6, 6.4 Hz, 1H), 2.05-1.84 (complex m, 2H), 1.56 (s, 6H), 1.35 (t, J=6.8 Hz, 3H).

### Example 183

### Preparation of 4-{2-(4-amino-1,2,5-oxadiazol-3-yl)-6-[(3-aminoethyl)oxy]-1-ethyl-1H-imidazo[4,5-c]pyridin-4-yl}-2-methyl-3-butyn-2-ol

The title compound was prepared as a brown solid according the Example 17, except substituting 1,1-dimethylethyl (2-hydroxyethyl)carbamate (313 mg, 1.94 mmole) for 1,1-dimethylethyl (4-hydroxybutyl)carbamate: LCMS (ES) m/e 372 (M+H)⁺; ¹H NMR ((CD₃)₂NCOD, 400 MHz) δ 7.49 (s, 1H), 4.93 (m, 4H), 3.70 (m, 2H), 1.81 (s, 6H), 1.66 (m, 3H).

### Example 184

### Capsule Composition

An oral dosage form for administering the present invention is produced by filing a standard two piece hard gelatin capsule with the ingredients in the proportions shown in Table I, below.

TABLE I

| INGREDIENTS | AMOUNTS |
|---|---|
| 4,4′-[2-(4-amino-1,2,5-oxadiazol-3-yl)-1-ethyl-1H- | 25 mg |
| imidazo[4,5-c]pyridine-4,6-diyl]bis(2-methyl-3-butyn-2-ol) | |
| (Compound of Example 1) | |
| Lactose | 55 mg |
| Talc | 16 mg |
| Magnesium Stearate | 4 mg |

### Example 185

### Injectable Parenteral Composition

An injectable form for administering the present invention is produced by stirring 1.5% by weight of 4-[2-(4-amino-1,2,5-oxadiazol-3-yl)-6-(3-aminophenyl)-1-ethyl-1H-imidazo[4,5-c]pyridin-4-yl]-2-methyl-3-butyn-2-ol (Compound of Example 2) in 10% by volume propylene glycol in water.

### Example 186

### Tablet Composition

The sucrose, calcium sulfate dihydrate and an Akt inhibitor as shown in Table II below, are mixed and granulated in the proportions shown with a 10% gelatin solution. The wet granules are screened, dried, mixed with the starch, talc and stearic acid; screened and compressed into a tablet.

TABLE II

| INGREDIENTS | AMOUNTS |
|---|---|
| 4-[2-(4-amino-1,2,5-oxadiazol-3-yl)-6-(2-aminophenyl)-1- | 20 mg |
| ethyl-1H-imidazo[4,5-c]pyridin-4-yl]-2-methyl-3-butyn-2-ol | |
| (Compound of Example 3) | |
| calcium sulfate dihydrate | 30 mg |
| sucrose | 4 mg |
| starch | 2 mg |
| talc | 1 mg |
| stearic acid | 0.5 mg |

While the preferred embodiments of the invention are illustrated by the above, it is to be understood that the invention is not limited to the precise instructions herein disclosed and that the right to all modifications coming within the scope of the following claims is reserved.

1. A compound of Formula (I): 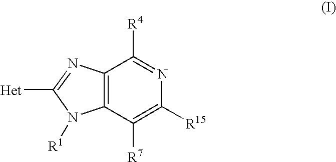
wherein:
Het is selected from the group consisting of: 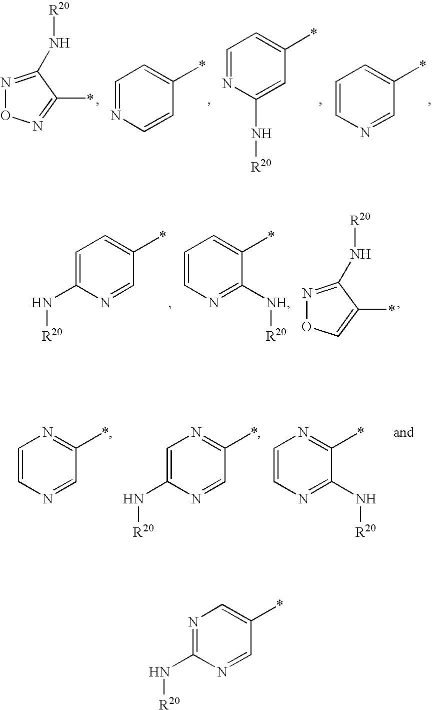
R²⁰ is selected from hydrogen, alkyl, alkyl substituted with one or more substituents selected from the group consisting of: hydroxy, alkoxy, amino, N-acylamino, cyclopropyl and halogen, cycloalkyl, cycloalkyl substituted with one or more substituents selected from the group consisting of: hydroxy, alkoxy, amino, N-acylamino and halogen, cycloalkyl containing from 1 to 4 heteroatoms, cycloalkyl containing from 1 to 4 heteroatoms substituted with one or more substituents selected from the group consisting of: hydroxy, alkoxy, amino, N-acylamino and halogen, C₁-C₁₂aryl and C₁-C₁₂aryl substituted with one or more substituents selected from the group consisting of: hydroxy, alkoxy, amino, N-acylamino and halogen;
R¹ is selected from hydrogen, alkyl, alkyl substituted with one or more substituents selected from the group consisting of: hydroxy, alkoxy, amino, N-acylamino, cyclopropyl and halogen, cycloalkyl, cycloalkyl substituted with one or more substituents selected from the group consisting of: hydroxy, alkoxy, amino, N-acylamino and halogen, cycloalkyl containing from 1 to 4 heteroatoms, cycloalkyl containing from 1 to 4 heteroatoms substituted with one or more substituents selected from the group consisting of: hydroxy, alkoxy, amino, N-acylamino and halogen, C₁-C₁₂aryl and C₁-C₁₂aryl substituted with one or more substituents selected from the group consisting of: hydroxy, alkoxy, amino, N-acylamino and halogen;
R⁴ is selected from hydrogen, halogen, alkyl, substituted alkyl, alkoxy, substituted alkoxy, acetamide, cyano, urea, substituted urea, aryl, substituted aryl, aryloxy, substituted aryloxy, oxo, hydroxy, acyloxy, amino, N-acylamino, substituted N-acylamino, cycloalkyl, substituted cycloalkyl, cycloalkyl containing from 1 to 4 heteroatoms and substituted cycloalkyl containing from 1 to 4 heteroatoms;
R¹⁵ is selected from halogen, alkyl, substituted alkyl, alkoxy, substituted alkoxy, acetamide, cyano, urea, substituted urea, aryl, substituted aryl, aryloxy, substituted aryloxy, oxo, hydroxy, acyloxy, amino, N-acylamino, substituted N-acylamino, cycloalkyl, substituted cycloalkyl, cycloalkyl containing from 1 to 4 heteroatoms, substituted cycloalkyl containing from 1 to 4 heteroatoms, cycloalkyloxy, substituted cycloalkyloxy, cycloalkyloxy containing from 1 to 4 heteroatoms and substituted cycloalkyloxy containing from 1 to 4 heteroatoms; and when R²⁰ is other than hydrogen, R¹⁵ can additionally be hydrogen;
R⁷ is selected from hydrogen, halogen, alkyl, substituted alkyl, alkoxy, substituted alkoxy, acetamide, cyano, urea, substituted urea, aryl, substituted aryl, aryloxy, substituted aryloxy, oxo, hydroxy, acyloxy, amino, N-acylamino, substituted N-acylamino, cycloalkyl, substituted cycloalkyl, cycloalkyl containing from 1 to 4 heteroatoms and substituted cycloalkyl containing from 1 to 4 heteroatoms;
or R¹⁵ and R⁷ taken together represent a 5 to 6 member saturated ring containing up to one heteroatom selected from oxygen and nitrogen, where the ring is optionally substituted with one or more substituents selected from amino, methylamino and dimethylamino.

2. A pharmaceutically acceptable salt, hydrate, solvate or pro-drug of a compound of Formula (I), as described in claim 1.

3. A compound of claim 1 represented by the following Formula (II): 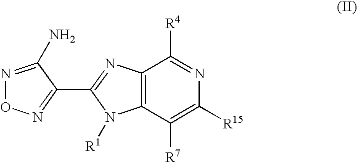
wherein:
R¹ is selected from hydrogen, alkyl, alkyl substituted with one or more substituents selected from the group consisting of: hydroxy, alkoxy, amino, N-acylamino, cyclopropyl and halogen, cycloalkyl, cycloalkyl substituted with one or more substituents selected from the group consisting of: hydroxy, alkoxy, amino, N-acylamino and halogen, cycloalkyl containing from 1 to 4 heteroatoms, cycloalkyl containing from 1 to 4 heteroatoms substituted with one or more substituents selected from the group consisting of: hydroxy, alkoxy, amino, N-acylamino and halogen, C₁-C₁₂aryl and C₁-C₁₂aryl substituted with one or more substituents selected from the group consisting of: hydroxy, alkoxy, amino, N-acylamino and halogen;
R⁴ is selected from hydrogen, halogen, alkyl, substituted alkyl, alkoxy, substituted alkoxy, acetamide, cyano, urea, substituted urea, aryl, substituted aryl, aryloxy, substituted aryloxy, oxo, hydroxy, acyloxy, amino, N-acylamino, substituted N-acylamino, cycloalkyl, substituted cycloalkyl, cycloalkyl containing from 1 to 4 heteroatoms and substituted cycloalkyl containing from 1 to 4 heteroatoms;
R¹⁵ is selected from halogen, alkyl, substituted alkyl, alkoxy, substituted alkoxy, acetamide, cyano, urea, substituted urea, aryl, substituted aryl, aryloxy, substituted aryloxy, oxo, hydroxy, acyloxy, amino, N-acylamino, substituted N-acylamino, cycloalkyl, substituted cycloalkyl, cycloalkyl containing from 1 to 4 heteroatoms, substituted cycloalkyl containing from 1 to 4 heteroatoms, cycloalkyloxy, substituted cycloalkyloxy, cycloalkyloxy containing from 1 to 4 heteroatoms and substituted cycloalkyloxy containing from 1 to 4 heteroatoms;
R⁷ is selected from hydrogen, halogen, alkyl, substituted alkyl, alkoxy, substituted alkoxy, acetamide, cyano, urea, substituted urea, aryl, substituted aryl, aryloxy, substituted aryloxy, oxo, hydroxy, acyloxy, amino, N-acylamino, substituted N-acylamino, cycloalkyl, substituted cycloalkyl, cycloalkyl containing from 1 to 4 heteroatoms and substituted cycloalkyl containing from 1 to 4 heteroatoms;
or R¹⁵ and R⁷ taken together represent a 5 to 6 member saturated ring containing up to one heteroatom selected from oxygen and nitrogen, where the ring is optionally substituted with one or more substituents selected from amino, methylamino and dimethylamino.

4. A pharmaceutically acceptable salt, hydrate, solvate or pro-drug of a compound of Formula (II), as described in claim 3.

5. A compound of claim 1 wherein R⁷ is hydrogen.

6. A pharmaceutically acceptable salt, hydrate, solvate or pro-drug of the compound described in claim 5.

7. A compound of claim 3 wherein R⁷ is hydrogen.

8. A pharmaceutically acceptable salt, hydrate, solvate or pro-drug of the described in claim 7.

9. A compound of claim 1 wherein:
R²⁰ is hydrogen;
R¹ is from: alkyl;
R⁴ is selected from alkyl and alkyl substituted with from one to three substituents selected from the group consisting of: hydroxy, alkoxy, amino, N-acylamino, cyclopropyl and halogen;
R¹⁵ is selected from halogen, alkyl, substituted alkyl, oxo, cycloalkyl, alkoxy, substituted alkoxy, cycloalkyl containing from 1 to 3 heteroatoms, substituted cycloalkyl, substituted cycloalkyl containing from 1 to 3 heteroatoms, cycloalkyloxy, cycloalkyloxy containing from 1 to 3 heteroatoms, substituted cycloalkyloxy, substituted cycloalkyloxy containing from 1 to 3 heteroatoms, C₁-C₁₂aryl, C₁-C₁₂aryloxy, C₁-C₁₂aryloxy substituted with from one to three substituents selected from the group consisting of: alkyl, hydroxy, alkoxy, acyloxy, amino, N-acylamino, substituted N-acylamino, hydroxyalkyl, aminoalkoxy, aminoalkyl, nitro, nitrile, cyano and halogen, and C₁-C₁₂aryl substituted with from one to three substituents selected from the group consisting of: alkyl, hydroxy, alkoxy, acyloxy, amino, N-acylamino, substituted N-acylamino, hydroxyalkyl, aminoalkoxy, aminoalkyl, nitro, nitrile, cyano and halogen, and
R⁷ is hydrogen.

10. A pharmaceutically acceptable salt, hydrate, solvate or pro-drug of a compound of Formula (I), as described in claim 9.

11. A compound of claim 3 wherein:
R¹ is selected from: alkyl, alkyl substituted with from one to three substituents selected from the group consisting of: hydroxy, alkoxy, amino, N-acylamino, cyclopropyl and halogen;
R⁴ is selected from hydrogen, halogen, alkyl, substituted alkyl, alkoxy, substituted alkoxy, cycloalkyl, cycloalkyl containing from 1 to 3 heteroatoms, C₁-C₁₂aryl and C₁-C₁₂aryl substituted with from one to three substituents selected from the group consisting of: alkyl, substituted alkyl, aryloxy, hydroxy, alkoxy, acyloxy, amino, N-acylamino, nitro, cyano and halogen;
R¹⁵ is selected from alkyl, substituted alkyl, oxo, cycloalkyl, alkoxy, substituted alkoxy, cycloalkyl containing from 1 to 3 heteroatoms, substituted cycloalkyl, substituted cycloalkyl containing from 1 to 3 heteroatoms, cycloalkyloxy, cycloalkyloxy containing from 1 to 3 heteroatoms, substituted cycloalkyloxy, substituted cycloalkyloxy containing from 1 to 3 heteroatoms, C₁-C₁₂aryl, C₁-C₁₂aryloxy, C₁-C₁₂aryloxy substituted with from one to three substituents selected from the group consisting of: alkyl, hydroxy, alkoxy, acyloxy, amino, N-acylamino, substituted N-acylamino, hydroxyalkyl, aminoalkoxy, aminoalkyl, nitro, nitrile, cyano and halogen, and C₁-C₁₂aryl substituted with from one to three substituents selected from the group consisting of: alkyl, hydroxy, alkoxy, acyloxy, amino, N-acylamino, substituted N-acylamino, hydroxyalkyl, aminoalkoxy, aminoalkyl, nitro, nitrile, cyano and halogen, and
R⁷ is hydrogen.

12. A pharmaceutically acceptable salt, hydrate, solvate or pro-drug of a compound of Formula (II), as described in claim 11.

13. A compound of claim 1 selected from:
4,4′-[2-(4-amino-1,2,5-oxadiazol-3-yl)-1-ethyl-1H-imidazo[4,5-c]pyridine-4,6-diyl]bis(2-methyl-3-butyn-2-ol);
4-[2-(4-amino-1,2,5-oxadiazol-3-yl)-6-(3-aminophenyl)-1-ethyl-1H-imidazo[4,5-c]pyridin-4-yl]-2-methyl-3-butyn-2-ol;
4-[2-(4-amino-1,2,5-oxadiazol-3-yl)-6-(2-aminophenyl)-1-ethyl-1H-imidazo[4,5-c]pyridin-4-yl]-2-methyl-3-butyn-2-ol;
4-[6-[3-(aminomethyl)phenyl]-2-(4-amino-1,2,5-oxadiazol-3-yl)-1-ethyl-1H-imidazo[4,5-c]pyridin-4-yl]-2-methyl-3-butyn-2-ol;
2-[2-(4-amino-1,2,5-oxadiazol-3-yl)-1-ethyl-4-(3-hydroxy-3-methyl-1-butyn-1-yl)-1H-imidazo[4,5-c]pyridin-6-yl]benzonitrile;
4-{2-(4-amino-1,2,5-oxadiazol-3-yl)-1-ethyl-6-[2-(hydroxymethyl)phenyl]-1H-imidazo[4,5-c]pyridin-4-yl}-2-methyl-3-butyn-2-ol;
N-{4-[2-(4-amino-1,2,5-oxadiazol-3-yl)-1-ethyl-4-(3-hydroxy-3-methyl-1-butyn-1-yl)-1H-imidazo[4,5-c]pyridin-6-yl]phenyl}acetamide;
4-[2-(4-amino-1,2,5-oxadiazol-3-yl)-1-ethyl-6-(1H-indol-5-yl)-1H-imidazo[4,5-c]pyridin-4-yl]-2-methyl-3-butyn-2-ol;
N-{3-[2-(4-amino-1,2,5-oxadiazol-3-yl)-1-ethyl-4-(3-hydroxy-3-methyl-1-butyn-1-yl)-1H-imidazo[4,5-c]pyridin-6-yl]phenyl}acetamide;
4-[6-[3-(aminomethyl)phenyl]-1-ethyl-4-(1H-pyrrol-3-yl)-1H-imidazo[4,5-c]pyridin-2-yl]-1,2,5-oxadiazol-3-amine;
N¹-{3-[2-(4-amino-1,2,5-oxadiazol-3-yl)-1-ethyl-4-(3-hydroxy-3-methyl-1-butyn-1-yl)-1H-imidazo[4,5-c]pyridin-6-yl]phenyl}glycinamide;
4-[2-(4-amino-1,2,5-oxadiazol-3-yl)-6-(4-aminophenyl)-1-ethyl-1H-imidazo[4,5-c]pyridin-4-yl]-2-methyl-3-butyn-2-ol;
3-[2-(4-amino-1,2,5-oxadiazol-3-yl)-1-ethyl-4-(3-hydroxy-3-methyl-1-butyn-1-yl)-1H-imidazo[4,5-c]pyridin-6-yl]phenol;
4-(2-(4-amino-1,2,5-oxadiazol-3-yl)-6-{3-[(3-aminopropyl)oxy]phenyl}-1-ethyl-1H-imidazo[4,5-c]pyridin-4-yl)-2-methyl-3-butyn-2-ol;
4-[6-{3-[(2-aminoethyl)oxy]phenyl}-2-(4-amino-1,2,5-oxadiazol-3-yl)-1-ethyl-1H-imidazo[4,5-c]pyridin-4-yl]-2-methyl-3-butyn-2-ol;
2-[2-(4-amino-1,2,5-oxadiazol-3-yl)-1-ethyl-4-(3-hydroxy-3-methyl-1-butyn-1-yl)-1H-imidazo[4,5-c]pyridin-6-yl]phenol;
4-[6-[(4-aminobutyl)oxy]-2-(4-amino-1,2,5-oxadiazol-3-yl)-1-ethyl-1H-imidazo[4,5-c]pyridin-4-yl]-2-methyl-3-butyn-2-ol;
4-{2-(4-amino-1,2,5-oxadiazol-3-yl)-6-[(3-aminopropyl)oxy]-1-ethyl-1H-imidazo[4,5-c]pyridin-4-yl}-2-methyl-3-butyn-2-ol;
4-{2-(4-amino-1,2,5-oxadiazol-3-yl)-6-[(5-aminopentyl)oxy]-1-ethyl-1H-imidazo[4,5-c]pyridin-4-yl}-2-methyl-3-butyn-2-ol;
4-(2-(4-amino-1,2,5-oxadiazol-3-yl)-1-ethyl-6-{[2-(4-piperidinyl)ethyl]oxy}-1H-imidazo[4,5-c]pyridin-4-yl)-2-methyl-3-butyn-2-ol;
4-(2-(4-amino-1,2,5-oxadiazol-3-yl)-1-ethyl-6-{[2-(3-pyrrolidinyl)ethyl]oxy}-1H-imidazo[4,5-c]pyridin-4-yl)-2-methyl-3-butyn-2-ol;
4-[2-(4-amino-1,2,5-oxadiazol-3-yl)-1-ethyl-6-(methyloxy)-1H-imidazo[4,5-c]pyridin-4-yl]-2-methyl-3-butyn-2-ol;
4-{2-(4-amino-1,2,5-oxadiazol-3-yl)-1-ethyl-6-[(2-morpholinylmethyl)oxy]-1H-imidazo[4,5-c]pyridin-4-yl}-2-methyl-3-butyn-2-ol;
4-[2-(4-amino-1,2,5-oxadiazol-3-yl)-1-ethyl-6-(3-pyrrolidinyloxy)-1H-imidazo[4,5-c]pyridin-4-yl]-2-methyl-3-butyn-2-ol;
4-{2-(4-amino-1,2,5-oxadiazol-3-yl)-1-ethyl-6-[(3S)-3-pyrrolidinyloxy]-1H-imidazo[4,5-c]pyridin-4-yl}-2-methyl-3-butyn-2-ol;
4-{2-(4-amino-1,2,5-oxadiazol-3-yl)-1-ethyl-6-[(3R)-3-pyrrolidinyloxy]-1H-imidazo[4,5-c]pyridin-4-yl}-2-methyl-3-butyn-2-ol;
4-(2-(4-amino-1,2,5-oxadiazol-3-yl)-6-{[(2R)-2-amino-3-(3-thienyl)propyl]oxy}-1-ethyl-1H-imidazo[4,5-c]pyridin-4-yl)-2-methyl-3-butyn-2-ol;
4-[6-{[(2S)-2-amino-3-(1H-indol-3-yl)propyl]oxy}-2-(4-amino-1,2,5-oxadiazol-3-yl)-1-ethyl-1H-imidazo[4,5-c]pyridin-4-yl]-2-methyl-3-butyn-2-ol;
4-[6-{[(1R,2S)-2-aminocyclopentyl]oxy}-2-(4-amino-1,2,5-oxadiazol-3-yl)-1-ethyl-1H-imidazo[4,5-c]pyridin-4-yl]-2-methyl-3-butyn-2-ol;
4-(2-(4-amino-1,2,5-oxadiazol-3-yl)-1-ethyl-6-{[2-(methylamino)ethyl]oxy}-1H-imidazo[4,5-c]pyridin-4-yl)-2-methyl-3-butyn-2-ol;
4-[6-{[(1S,2R)-2-aminocyclopentyl]oxy}-2-(4-amino-1,2,5-oxadiazol-3-yl)-1-ethyl-1H-imidazo[4,5-c]pyridin-4-yl]-2-methyl-3-butyn-2-ol;
4-{2-(4-amino-1,2,5-oxadiazol-3-yl)-1-ethyl-6-[(phenylmethyl)oxy]-1H-imidazo[4,5-c]pyridin-4-yl}-2-methyl-3-butyn-2-ol;
4-{2-(4-amino-1,2,5-oxadiazol-3-yl)-1-ethyl-6-[(4-piperidinylmethyl)oxy]-1H-imidazo[4,5-c]pyridine-4-yl}-2-methyl-3-butyn-2-ol;
4-(2-(4-amino-1,2,5-oxadiazol-3-yl)-1-ethyl-6-{[2-(3-piperidinyl)ethyl]oxy}-1H-imidazo[4,5-c]pyridin-4-yl)-2-methyl-3-butyn-2-ol;
4-{2-(4-amino-1,2,5-oxadiazol-3-yl)-1-ethyl-6-[(3-piperidinylmethyl)oxy]-1H-imidazo[4,5-c]pyridin-4-yl}-2-methyl-3-butyn-2-ol;
4-[6-{[(2R)-2-amino-3-(1H-indol-3-yl)propyl]oxy}-2-(4-amino-1,2,5-oxadiazol-3-yl)-1-ethyl-1H-imidazo[4,5-c]pyridin-4-yl]-2-methyl-3-butyn-2-ol;
4-(2-(4-amino-1,2,5-oxadiazol-3-yl)-1-ethyl-6-{[(2R)-2-pyrrolidinylmethyl]oxy}-1H-imidazo[4,5-c]pyridin-4-yl)-2-methyl-3-butyn-2-ol;
4-(2-(4-amino-1,2,5-oxadiazol-3-yl)-1-ethyl-6-{[(2S)-2-pyrrolidinylmethyl]oxy}-1H-imidazo[4,5-c]pyridin-4-yl)-2-methyl-3-butyn-2-ol;
4-{2-(4-amino-1,2,5-oxadiazol-3-yl)-1-ethyl-6-[(1H-indol-3-ylmethyl)oxy]-1H-imidazo[4,5-c]pyridin-4-yl}-2-methyl-3-butyn-2-ol;
4-[6-[(4-amino-2-methylbutyl)oxy]-2-(4-amino-1,2,5-oxadiazol-3-yl)-1-ethyl-1H-imidazo[4,5-c]pyridin-4-yl]-2-methyl-3-butyn-2-ol;
4-(2-(4-amino-1,2,5-oxadiazol-3-yl)-6-{[(2S)-2-amino-2-phenylethyl]oxy}-1-ethyl-1H-imidazo[4,5-c]pyridin-4-yl)-2-methyl-3-butyn-2-ol;
4-(2-(4-amino-1,2,5-oxadiazol-3-yl)-6-{[(2R)-2-amino-2-phenylethyl]oxy}-1-ethyl-1H-imidazo[4,5-c]pyridin-4-yl)-2-methyl-3-butyn-2-ol;
4-(2-(4-amino-1,2,5-oxadiazol-3-yl)-6-{([(2R)-2-amino-3-phenylpropyl]oxy}-1-ethyl-1H-imidazo[4,5-c]pyridin-4-yl)-2-methyl-3-butyn-2-ol;
4-(2-(4-amino-1,2,5-oxadiazol-3-yl)-6-{[(2S)-2-amino-3-phenylpropyl]oxy}-1-ethyl-1H-imidazo[4,5-c]pyridin-4-yl)-2-methyl-3-butyn-2-ol;
4-[6-{[(2S)-2-amino-3-methylbutyl]oxy}-2-(4-amino-1,2,5-oxadiazol-3-yl)-1-ethyl-1H-imidazo[4,5-c]pyridin-4-yl]-2-methyl-3-butyn-2-ol;
4-[6-[(2-aminoethyl)oxy]-2-(4-amino-1,2,5-oxadiazol-3-yl)-1-ethyl-1H-imidazo[4,5-c]pyridin-4-yl]-3-butyn-2-ol;
3-[6-[(2-aminoethyl)oxy]-2-(4-amino-1,2,5-oxadiazol-3-yl)-1-ethyl-1H-imidazo[4,5-c]pyridin-4-yl]-2-propyn-1-ol;
4-(2-(4-amino-1,2,5-oxadiazol-3-yl)-6-{[(2R)-2-amino-3-phenylpropyl]oxy}-1-ethyl-1H-imidazo[4,5-c]pyridin-4-yl)-2-methyl-3-butyn-2-ol;
4-(2-(4-amino-1,2,5-oxadiazol-3-yl)-6-{[(2S)-2-azetidinylmethyl]oxy}-1-ethyl-1H-imidazo[4,5-c]pyridin-4-yl)-2-methyl-3-butyn-2-ol;
4-[6-{[(1R,2S)-2-aminocyclohexyl]oxy}-2-(4-amino-1,2,5-oxadiazol-3-yl)-1-ethyl-1H-imidazo[4,5-c]pyridin-4-yl]-2-methyl-3-butyn-2-ol;
4-[6-{[(1S,2R)-2-aminocyclohexyl]oxy}-2-(4-amino-1,2,5-oxadiazol-3-yl)-1-ethyl-1H-imidazo[4,5-c]pyridin-4-yl]-2-methyl-3-butyn-2-ol;
(racemic)4-[6-{[(1S,2S)-2-aminocyclohexyl]oxy}-2-(4-amino-1,2,5-oxadiazol-3-yl)-1-ethyl-1H-imidazo[4,5-c]pyridin-4-yl]-2-methyl-3-butyn-2-ol;
4-(2-(4-amino-1,2,5-oxadiazol-3-yl)-1-ethyl-6-{[2-(2-morpholinyl)ethyl]oxy}-1H-imidazo[4,5-c]pyridin-4-yl)-2-methyl-3-butyn-2-ol;
4-[2-(4-amino-1,2,5-oxadiazol-3-yl)-1-ethyl-6-({3-[(2S)-2-pyrrolidinyl]propyl}oxy)-1H-imidazo[4,5-c]pyridin-4-yl]-2-methyl-3-butyn-2-ol;
2-(4-amino-1,2,5-oxadiazol-3-yl)-1-ethyl-4-(3-hydroxy-3-methyl-1-butyn-1-yl)-1,5-dihydro-6H-imidazo[4,5-c]pyridin-6-one;
4-(2-(4-amino-1,2,5-oxadiazol-3-yl)-6-{[(2S)-2-aminopropyl]oxy}-1-ethyl-1H-imidazo[4,5-c]pyridin-4-yl)-2-methyl-3-butyn-2-ol;
4-[6-{[(2R)-2-amino-3-methylbutyl]oxy}-2-(4-amino-1,2,5-oxadiazol-3-yl)-1-ethyl-1H-imidazo[4,5-c]pyridin-4-yl]-2-methyl-3-butyn-2-ol;
4-[6-{[(2R)-2-amino-4-methylpentyl]oxy}-2-(4-amino-1,2,5-oxadiazol-3-yl)-1-ethyl-1H-imidazo[4,5-c]pyridin-4-yl]-2-methyl-3-butyn-2-ol;
4-[6-{[(2S)-2-amino-4-methylpentyl]oxy}-2-(4-amino-1,2,5-oxadiazol-3-yl)-1-ethyl-1H-imidazo[4,5-c]pyridin-4-yl]-2-methyl-3-butyn-2-ol;
4-(2-(4-amino-1,2,5-oxadiazol-3-yl)-6-{[(2R)-2-aminopropyl]oxy}-1-ethyl-1H-imidazo[4,5-c]pyridin-4-yl)-2-methyl-3-butyn-2-ol;
4-[6-{[(2S)-2-amino-3-cyclohexylpropyl]oxy}-2-(4-amino-1,2,5-oxadiazol-3-yl)-1-ethyl-1H-imidazo[4,5-c]pyridin-4-yl]-2-methyl-3-butyn-2-ol;
4-(2-(4-amino-1,2,5-oxadiazol-3-yl)-6-{[(2S)-2-amino-4-phenylbutyl]oxy}-1-ethyl-1H-imidazo[4,5-c]pyridin-4-yl)-2-methyl-3-butyn-2-ol;
4-[6-[(2-aminoethyl)oxy]-1-ethyl-4-(3-furanyl)-1H-imidazo[4,5-c]pyridin-2-yl]-1,2,5-oxadiazol-3-amine;
4-[6-{[(2S)-2-amino-3-(1H-imidazol-4-yl)propyl]oxy}-2-(4-amino-1,2,5-oxadiazol-3-yl)-1-ethyl-1H-imidazo[4,5-c]pyridin-4-yl]-2-methyl-3-butyn-2-ol;
(5S)-5-({[2-(4-amino-1,2,5-oxadiazol-3-yl)-1-ethyl-4-(3-hydroxy-3-methyl-1-butyn-1-yl)-1H-imidazo[4,5-c]pyridin-6-yl]oxy}methyl)-2-pyrrolidinone;
(5R)-5-({[2-(4-amino-1,2,5-oxadiazol-3-yl)-1-ethyl-4-(3-hydroxy-3-methyl-1-butyn-1-yl)-1H-imidazo[4,5-c]pyridin-6-yl]oxy}methyl)-2-pyrrolidinone;
4-(2-(4-amino-1,2,5-oxadiazol-3-yl)-1-ethyl-6-{[2-(1-pyrrolidinyl)ethyl]oxy}-1H-imidazo[4,5-c]pyridin-4-yl)-2-methyl-3-butyn-2-ol;
4-(2-(4-amino-1,2,5-oxadiazol-3-yl)-1-ethyl-6-{[2-({[4-(methyloxy)phenyl]methyl}amino)ethyl]oxy}-1H-imidazo[4,5-c]pyridin-4-yl)-2-methyl-3-butyn-2-ol;
4-(2-(4-amino-1,2,5-oxadiazol-3-yl)-1-ethyl-6-{[2-({[4-(trifluoromethyl)phenyl]methyl}amino)ethyl]oxy}-1H-imidazo[4,5-c]pyridin-4-yl)-2-methyl-3-butyn-2-ol;
4-[2-(4-amino-1,2,5-oxadiazol-3-yl)-1-ethyl-6-(4-piperidinyloxy)-1H-imidazo[4,5-c]pyridin-4-yl]-2-methyl-3-butyn-2-ol;
4-(2-(4-amino-1,2,5-oxadiazol-3-yl)-1-ethyl-6-{[2-(4-morpholinyl)ethyl]oxy}-1H-imidazo[4,5-c]pyridin-4-yl)-2-methyl-3-butyn-2-ol;
4-(2-(4-amino-1,2,5-oxadiazol-3-yl)-1-ethyl-6-{[2-(phenylamino)ethyl]oxy}-1H-imidazo[4,5-c]pyridin-4-yl)-2-methyl-3-butyn-2-ol;
4-[6-{[(2S)-2-amino-3-(1-methyl-1H-indol-3-yl)propyl]oxy}-2-(4-amino-1,2,5-oxadiazol-3-yl)-1-ethyl-1H-imidazo[4,5-c]pyridin-4-yl]-2-methyl-3-butyn-2-ol;
4-[2-(4-amino-1,2,5-oxadiazol-3-yl)-6-({(2S)-2-amino-3-[(phenylmethyl)thio]propyl}oxy)-1-ethyl-1H-imidazo[4,5-c]pyridin-4-yl]-2-methyl-3-butyn-2-ol;
4-(2-(4-amino-1,2,5-oxadiazol-3-yl)-6-{[(2R)-2-amino-3-(3-pyridinyl)propyl]oxy}-1-ethyl-1H-imidazo[4,5-c]pyridin-4-yl)-2-methyl-3-butyn-2-ol;
4-(2-(4-amino-1,2,5-oxadiazol-3-yl)-6-{[(2R)-2-amino-4-phenylbutyl]oxy}-1-ethyl-1H-imidazo[4,5-c]pyridin-4-yl)-2-methyl-3-butyn-2-ol;
4-{2-(4-amino-1,2,5-oxadiazol-3-yl)-6-[(2-amino-1-phenylethyl)oxy]-1-ethyl-1H-imidazo[4,5-c]pyridin-4-yl}-2-methyl-3-butyn-2-ol;
4-[6-(aminomethyl)-2-(4-amino-1,2,5-oxadiazol-3-yl)-1-ethyl-1H-imidazo[4,5-c]pyridin-4-yl]-2-methyl-3-butyn-2-ol;
4-{2-(4-amino-1,2,5-oxadiazol-3-yl)-1-ethyl-6-[(methylamino)methyl]-1H-imidazo[4,5-c]pyridin-4-yl}-2-methyl-3-butyn-2-ol;
4-(2-(4-amino-1,2,5-oxadiazol-3-yl)-1-ethyl-6-{[(phenylmethyl)amino]methyl}-1H-imidazo[4,5-c]pyridin-4-yl)-2-methyl-3-butyn-2-ol;
4-[2-(4-amino-1,2,5-oxadiazol-3-yl)-6-(3-aminopropyl)-1-ethyl-1H-imidazo[4,5-c]pyridin-4-yl]-2-methyl-3-butyn-2-ol;
4-[6-(2-aminoethyl)-2-(4-amino-1,2,5-oxadiazol-3-yl)-1-ethyl-1H-imidazo[4,5-c]pyridin-4-yl]-2-methyl-3-butyn-2-ol;
4-[2-(4-amino-1,2,5-oxadiazol-3-yl)-1-ethyl-6-(4-morpholinylmethyl)-1H-imidazo[4,5-c]pyridin-4-yl]-2-methyl-3-butyn-2-ol;
4-(2-(4-amino-1,2,5-oxadiazol-3-yl)-1-ethyl-6-{[methyl(phenylmethyl)amino]methyl}-1H-imidazo[4,5-c]pyridin-4-yl)-2-methyl-3-butyn-2-ol;
4-(2-(4-amino-1,2,5-oxadiazol-3-yl)-1-ethyl-6-{[methyl(2-phenylethyl)amino]methyl}-1H-imidazo[4,5-c]pyridin-4-yl)-2-methyl-3-butyn-2-ol;
4-(2-(4-amino-1,2,5-oxadiazol-3-yl)-6-{[(1R)-2-amino-1-phenylethyl]oxy}-1-ethyl-1H-imidazo[4,5-c]pyridin-4-yl)-2-methyl-3-butyn-2-ol;
4-(2-(4-amino-1,2,5-oxadiazol-3-yl)-1-ethyl-6-{[2-(methylamino)-1-phenylethyl]oxy}-1H-imidazo[4,5-c]pyridin-4-yl)-2-methyl-3-butyn-2-ol;
4-[6-[(2-amino-1-cyclohexylethyl)oxy]-2-(4-amino-1,2,5-oxadiazol-3-yl)-1-ethyl-1H-imidazo[4,5-c]pyridin-4-yl]-2-methyl-3-butyn-2-ol;
4-(2-(4-amino-1,2,5-oxadiazol-3-yl)-6-{[2-amino-1-(tetrahydro-2H-pyran-4-yl)ethyl]oxy}-1-ethyl-1H-imidazo[4,5-c]pyridin-4-yl)-2-methyl-3-butyn-2-ol;
4-(2-(4-amino-1,2,5-oxadiazol-3-yl)-6-{[2-amino-1-(3-pyridinyl)ethyl]oxy}-1-ethyl-1H-imidazo[4,5-c]pyridin-4-yl)-2-methyl-3-butyn-2-ol;
4-[6-[(2-amino-1-cyclopropylethyl)oxy]-2-(4-amino-1,2,5-oxadiazol-3-yl)-1-ethyl-1H-imidazo[4,5-c]pyridin-4-yl]-2-methyl-3-butyn-2-ol;
(rac)-4-[6-{[2-amino-1-(1,3-benzodioxol-4-yl)ethyl]oxy}-2-(4-amino-1,2,5-oxadiazol-3-yl)-1-ethyl-1H-imidazo[4,5-c]pyridin-4-yl]-2-methyl-3-butyn-2-ol;
(ent-1)-4-[6-{[2-amino-1-(1,3-benzodioxol-4-yl)ethyl]oxy}-2-(4-amino-1,2,5-oxadiazol-3-yl)-1-ethyl-1H-imidazo[4,5-c]pyridin-4-yl]-2-methyl-3-butyn-2-ol;
(ent-2)-4-[6-{[2-amino-1-(1,3-benzodioxol-4-yl)ethyl]oxy}-2-(4-amino-1,2,5-oxadiazol-3-yl)-1-ethyl-1H-imidazo[4,5-c]pyridin-4-yl]-2-methyl-3-butyn-2-ol;
4-(2-(4-amino-1,2,5-oxadiazol-3-yl)-6-{[2-(dimethylamino)-1-phenylethyl]oxy}-1-ethyl-1H-imidazo[4,5-c]pyridin-4-yl)-2-methyl-3-butyn-2-ol;
4-[6-{[(cis)-1-amino-2,3-dihydro-1H-inden-2-yl]oxy}-2-(4-amino-1,2,5-oxadiazol-3-yl)-1-ethyl-1H-imidazo[4,5-c]pyridin-4-yl]-2-methyl-3-butyn-2-ol;
4-(2-(4-amino-1,2,5-oxadiazol-3-yl)-1-ethyl-6-{[(S)-(2R)-2-morpholinyl(phenyl)methyl]oxy}-1H-imidazo[4,5-c]pyridin-4-yl)-2-methyl-3-butyn-2-ol;
4-(2-(4-amino-1,2,5-oxadiazol-3-yl)-1-ethyl-6-{[(R)-(2S)-2-morpholinyl(phenyl)methyl]oxy}-1H-imidazo[4,5-c]pyridin-4-yl)-2-methyl-3-butyn-2-ol;
4-[2-(4-amino-1,2,5-oxadiazol-3-yl)-1-ethyl-6-(1-pyrrolidinylmethyl)-1H-imidazo[4,5-c]pyridin-4-yl]-2-methyl-3-butyn-2-ol;
4-{2-(4-amino-1,2,5-oxadiazol-3-yl)-6-[(dimethylamino)methyl]-1-ethyl-1H-imidazo[4,5-c]pyridin-4-yl}-2-methyl-3-butyn-2-ol;
4-[2-(4-amino-1,2,5-oxadiazol-3-yl)-1-ethyl-6-(1-piperidinylmethyl)-1H-imidazo[4,5-c]pyridin-4-yl]-2-methyl-3-butyn-2-ol;
N-{[2-(4-amino-1,2,5-oxadiazol-3-yl)-1-ethyl-4-(3-hydroxy-3-methyl-1-butyn-1-yl)-1H-imidazo[4,5-c]pyridin-6-yl]methyl}-N-methylacetamide;
4-(2-(4-amino-1,2,5-oxadiazol-3-yl)-6-{[(2R)-2-amino-3-(4-pyridinyl)propyl]oxy}-1-ethyl-1H-imidazo[4,5-c]pyridin-4-yl)-2-methyl-3-butyn-2-ol;
4-(2-(4-amino-1,2,5-oxadiazol-3-yl)-6-{[(1S)-2-amino-1-phenylethyl]oxy}-1-ethyl-1H-imidazo[4,5-c]pyridin-4-yl)-2-methyl-3-butyn-2-ol;
4-[6-[(2-amino-1-methylethyl)oxy]-2-(4-amino-1,2,5-oxadiazol-3-yl)-1-ethyl-1H-imidazo[4,5-c]pyridin-4-yl]-2-methyl-3-butyn-2-ol;
4-(2-(4-amino-1,2,5-oxadiazol-3-yl)-6-{[2-amino-1-(phenylmethyl)ethyl]oxy}-1-ethyl-1H-imidazo[4,5-c]pyridin-4-yl)-2-methyl-3-butyn-2-ol;
4-{2-(4-amino-1,2,5-oxadiazol-3-yl)-6-[(3-amino-1-phenylpropyl)oxy]-1-ethyl-1H-imidazo[4,5-c]pyridin-4-yl}-2-methyl-3-butyn-2-ol;
4-(2-(4-amino-1,2,5-oxadiazol-3-yl)-6-{[(1S)-3-amino-1-phenylpropyl]oxy}-1-ethyl-1H-imidazo[4,5-c]pyridin-4-yl)-2-methyl-3-butyn-2-ol;
4-(2-(4-amino-1,2,5-oxadiazol-3-yl)-6-{[(1R)-3-amino-1-phenylpropyl]oxy}-1-ethyl-1H-imidazo[4,5-c]pyridin-4-yl)-2-methyl-3-butyn-2-ol;
4-[6-[(3-amino-1-cyclohexylpropyl)oxy]-2-(4-amino-1,2,5-oxadiazol-3-yl)-1-ethyl-1H-imidazo[4,5-c]pyridin-4-yl]-2-methyl-3-butyn-2-ol;
4-(2-(4-amino-1,2,5-oxadiazol-3-yl)-6-{[2-amino-1-(4-pyridinyl)ethyl]oxy}-1-ethyl-1H-imidazo[4,5-c]pyridin-4-yl)-2-methyl-3-butyn-2-ol;
4-(2-(4-amino-1,2,5-oxadiazol-3-yl)-6-{[2-amino-1-(2-pyridinyl)ethyl]oxy}-1-ethyl-1H-imidazo[4,5-c]pyridin-4-yl)-2-methyl-3-butyn-2-ol;
4-[6-{[1-(aminomethyl)-3-phenylpropyl]oxy}-2-(4-amino-1,2,5-oxadiazol-3-yl)-1-ethyl-1H-imidazo[4,5-c]pyridin-4-yl]-2-methyl-3-butyn-2-ol;
4-{2-(4-amino-1,2,5-oxadiazol-3-yl)-6-[(4-amino-1-phenylbutyl)oxy]-1-ethyl-1H-imidazo[4,5-c]pyridin-4-yl}-2-methyl-3-butyn-2-ol;
(rac)-4-(2-(4-amino-1,2,5-oxadiazol-3-yl)-6-{[(1R,2S)-1-amino-1,2,3,4-tetrahydro-2-naphthalenyl]oxy}-1-ethyl-1H-imidazo[4,5-c]pyridin-4-yl)-2-methyl-3-butyn-2-ol;
4-[2-(4-amino-1,2,5-oxadiazol-3-yl)-1-ethyl-6-({(R)-phenyl[(2S)-2-pyrrolidinyl]methyl}oxy)-1H-imidazo[4,5-c]pyridin-4-yl]-2-methyl-3-butyn-2-ol;
4-[2-(4-amino-1,2,5-oxadiazol-3-yl)-1-ethyl-6-({(S)-phenyl[(2R)-2-pyrrolidinyl]methyl}oxy)-1H-imidazo[4,5-c]pyridin-4-yl]-2-methyl-3-butyn-2-ol;
4-(2-(4-amino-1,2,5-oxadiazol-3-yl)-6-{[2-amino-1-(4-piperidinyl)ethyl]oxy}-1-ethyl-1H-imidazo[4,5-c]pyridin-4-yl)-2-methyl-3-butyn-2-ol;
4-[6-{[2-amino-1-(1-methyl-4-piperidinyl)ethyl]oxy}-2-(4-amino-1,2,5-oxadiazol-3-yl)-1-ethyl-1H-imidazo[4,5-c]pyridin-4-yl]-2-methyl-3-butyn-2-ol;
4-(2-(4-amino-1,2,5-oxadiazol-3-yl)-6-{[3-amino-1-(4-piperidinyl)propyl]oxy}-1-ethyl-1H-imidazo[4,5-c]pyridin-4-yl)-2-methyl-3-butyn-2-ol;
4-[6-{[3-amino-1-(1-methyl-4-piperidinyl)propyl]oxy}-2-(4-amino-1,2,5-oxadiazol-3-yl)-1-ethyl-1H-imidazo[4,5-c]pyridin-4-yl]-2-methyl-3-butyn-2-ol;
4-[6-{[3-amino-1-(1-methyl-4-piperidinyl)propyl]oxy}-2-(4-amino-1,2,5-oxadiazol-3-yl)-1-ethyl-1H-imidazo[4,5-c]pyridin-4-yl]-2-methyl-3-butyn-2-ol;
(ent-2)-4-[6-[(2-amino-1-cyclohexylethyl)oxy]-2-(4-amino-1,2,5-oxadiazol-3-yl)-1-ethyl-1H-imidazo[4,5-c]pyridin-4-yl]-2-methyl-3-butyn-2-ol;
(ent-1)-4-[6-[(2-amino-1-cyclohexylethyl)oxy]-2-(4-amino-1,2,5-oxadiazol-3-yl)-1-ethyl-1H-imidazo[4,5-c]pyridin-4-yl]-2-methyl-3-butyn-2-ol;
1-[6-[(2-aminoethyl)oxy]-2-(4-amino-1,2,5-oxadiazol-3-yl)-1-ethyl-1H-imidazo[4,5-c]pyridin-4-yl]-3-methyl-1-pentyn-3-ol;
4-(2-(4-amino-1,2,5-oxadiazol-3-yl)-6-{[(1R,2S)-2-amino-1-phenylpropyl]oxy}-1-ethyl-1H-imidazo[4,5-c]pyridin-4-yl)-2-methyl-3-butyn-2-ol;
4-(2-(4-amino-1,2,5-oxadiazol-3-yl)-1-ethyl-6-{[3-(methylamino)-1-phenylpropyl]oxy}-1H-imidazo[4,5-c]pyridin-4-yl)-2-methyl-3-butyn-2-ol;
4-(2-(4-amino-1,2,5-oxadiazol-3-yl)-6-{[3-(dimethylamino)-1-phenylpropyl]oxy}-1-ethyl-1H-imidazo[4,5-c]pyridin-4-yl)-2-methyl-3-butyn-2-ol;
4-[6-{[3-amino-1-(4-chlorophenyl)propyl]oxy}-2-(4-amino-1,2,5-oxadiazol-3-yl)-1-ethyl-1H-imidazo[4,5-c]pyridin-4-yl]-2-methyl-3-butyn-2-ol;
4-[6-{[3-amino-1-(3-chlorophenyl)propyl]oxy}-2-(4-amino-1,2,5-oxadiazol-3-yl)-1-ethyl-1H-imidazo[4,5-c]pyridin-4-yl]-2-methyl-3-butyn-2-ol;
4-[6-{[3-amino-1-(2-chlorophenyl)propyl]oxy}-2-(4-amino-1,2,5-oxadiazol-3-yl)-1-ethyl-1H-imidazo[4,5-c]pyridin-4-yl]-2-methyl-3-butyn-2-ol;
4-[6-({2-amino-1-[3-(methyloxy)phenyl]ethyl}oxy)-2-(4-amino-1,2,5-oxadiazol-3-yl)-1-ethyl-1H-imidazo[4,5-c]pyridin-4-yl]-2-methyl-3-butyn-2-ol;
4-[6-{[(1S,2S)-2-amino-3-(methyloxy)-1-phenylpropyl]oxy}-2-(4-amino-1,2,5-oxadiazol-3-yl)-1-ethyl-1H-imidazo[4,5-c]pyridin-4-yl]-2-methyl-3-butyn-2-ol;
4-[6-({3-amino-1-[2-fluoro-3-(methyloxy)phenyl]propyl}oxy)-2-(4-amino-1,2,5-oxadiazol-3-yl)-1-ethyl-1H-imidazo[4,5-c]pyridin-4-yl]-2-methyl-3-butyn-2-ol;
4-[6-({3-amino-1-[3-(methyloxy)phenyl]propyl}oxy)-2-(4-amino-1,2,5-oxadiazol-3-yl)-1-ethyl-1H-imidazo[4,5-c]pyridin-4-yl]-2-methyl-3-butyn-2-ol;
4-[6-({2-amino-1-[2-fluoro-3-(methyloxy)phenyl]ethyl}oxy)-2-(4-amino-1,2,5-oxadiazol-3-yl)-1-ethyl-1H-imidazo[4,5-c]pyridin-4-yl]-2-methyl-3-butyn-2-ol;
4-[6-{[3-amino-1-(2-fluorophenyl)propyl]oxy}-2-(4-amino-1,2,5-oxadiazol-3-yl)-1-ethyl-1H-imidazo[4,5-c]pyridin-4-yl]-2-methyl-3-butyn-2-ol;
4-[6-{[(2R)-2-amino-3-(4-fluorophenyl)propyl]oxy}-2-(4-amino-1,2,5-oxadiazol-3-yl)-1-ethyl-1H-imidazo[4,5-c]pyridin-4-yl]-2-methyl-3-butyn-2-ol;
4-[6-{[(2R)-2-amino-3-(2-fluorophenyl)propyl]oxy}-2-(4-amino-1,2,5-oxadiazol-3-yl)-1-ethyl-1H-imidazo[4,5-c]pyridin-4-yl]-2-methyl-3-butyn-2-ol;
4-[2-(4-amino-1,2,5-oxadiazol-3-yl)-6-({(2R)-2-amino-3-[2-(trifluoromethyl)phenyl]propyl}oxy)-1-ethyl-1H-imidazo[4,5-c]pyridin-4-yl]-2-methyl-3-butyn-2-ol;
4-[6-{[(2R)-2-amino-3-(4-chlorophenyl)propyl]oxy}-2-(4-amino-1,2,5-oxadiazol-3-yl)-1-ethyl-1H-imidazo[4,5-c]pyridin-4-yl]-2-methyl-3-butyn-2-ol;
4-[6-{[(2R)-2-amino-3-(3-chlorophenyl)propyl]oxy}-2-(4-amino-1,2,5-oxadiazol-3-yl)-1-ethyl-1H-imidazo[4,5-c]pyridin-4-yl]-2-methyl-3-butyn-2-ol;
4-[6-{[(2R)-2-amino-3-(2-chlorophenyl)propyl]oxy}-2-(4-amino-1,2,5-oxadiazol-3-yl)-1-ethyl-1H-imidazo[4,5-c]pyridin-4-yl]-2-methyl-3-butyn-2-ol;
4-[2-(4-amino-1,2,5-oxadiazol-3-yl)-6-({(2R)-2-amino-3-[3-(trifluoromethyl)phenyl]propyl}oxy)-1-ethyl-1H-imidazo[4,5-c]pyridin-4-yl]-2-methyl-3-butyn-2-ol;
4-[6-{[(2R)-2-amino-3-(3-fluorophenyl)propyl]oxy}-2-(4-amino-1,2,5-oxadiazol-3-yl)-1-ethyl-1H-imidazo[4,5-c]pyridin-4-yl]-2-methyl-3-butyn-2-ol;
4-[2-(4-amino-1,2,5-oxadiazol-3-yl)-6-({(2R)-2-amino-3-[4-(trifluoromethyl)phenyl]propyl}oxy)-1-ethyl-1H-imidazo[4,5-c]pyridin-4-yl]-2-methyl-3-butyn-2-ol;
4-[6-{[(2R)-2-amino-3-(1-benzothien-2-yl)propyl]oxy}-2-(4-amino-1,2,5-oxadiazol-3-yl)-1-ethyl-1H-imidazo[4,5-c]pyridin-4-yl]-2-methyl-3-butyn-2-ol;
4-[6-{[(2R)-2-amino-3-cyclohexylpropyl]oxy}-2-(4-amino-1,2,5-oxadiazol-3-yl)-1-ethyl-1H-imidazo[4,5-c]pyridin-4-yl]-2-methyl-3-butyn-2-ol;
4-{2-(4-amino-1,2,5-oxadiazol-3-yl)-6-[(3-amino-3-phenylpropyl)oxy]-1-ethyl-1H-imidazo[4,5-c]pyridin-4-yl}-2-methyl-3-butyn-2-ol;
4-{2-(4-amino-1,2,5-oxadiazol-3-yl)-6-[(3-amino-2-phenylpropyl)oxy]-1-ethyl-1H-imidazo[4,5-c]pyridin-4-yl}-2-methyl-3-butyn-2-ol;
4-[6-{[2-amino-1-(3-chlorophenyl)ethyl]oxy}-2-(4-amino-1,2,5-oxadiazol-3-yl)-1-ethyl-1H-imidazo[4,5-c]pyridin-4-yl]-2-methyl-3-butyn-2-ol;
4-[6-{[2-amino-1-(2-chlorophenyl)ethyl]oxy}-2-(4-amino-1,2,5-oxadiazol-3-yl)-1-ethyl-1H-imidazo[4,5-c]pyridin-4-yl]-2-methyl-3-butyn-2-ol;
4-[6-{[2-amino-1-(4-chlorophenyl)ethyl]oxy}-2-(4-amino-1,2,5-oxadiazol-3-yl)-1-ethyl-1H-imidazo[4,5-c]pyridin-4-yl]-2-methyl-3-butyn-2-ol;
4-[6-{[3-amino-1-(3-fluorophenyl)propyl]oxy}-2-(4-amino-1,2,5-oxadiazol-3-yl)-1-ethyl-1H-imidazo[4,5-c]pyridin-4-yl]-2-methyl-3-butyn-2-ol;
4-[6-{[3-amino-1-(4-fluorophenyl)propyl]oxy}-2-(4-amino-1,2,5-oxadiazol-3-yl)-1-ethyl-1H-imidazo[4,5-c]pyridin-4-yl]-2-methyl-3-butyn-2-ol;
4-[6-{[2-amino-1-(2-fluorophenyl)ethyl]oxy}-2-(4-amino-1,2,5-oxadiazol-3-yl)-1-ethyl-1H-imidazo[4,5-c]pyridin-4-yl]-2-methyl-3-butyn-2-ol;
4-[6-{[2-amino-1-(3-fluorophenyl)ethyl]oxy}-2-(4-amino-1,2,5-oxadiazol-3-yl)-1-ethyl-1H-imidazo[4,5-c]pyridin-4-yl]-2-methyl-3-butyn-2-ol;
4-[6-{[2-amino-1-(4-fluorophenyl)ethyl]oxy}-2-(4-amino-1,2,5-oxadiazol-3-yl)-1-ethyl-1H-imidazo[4,5-c]pyridin-4-yl]-2-methyl-3-butyn-2-ol;
4-[6-({3-amino-1-[4-fluoro-3-(methyloxy)phenyl]propyl}oxy)-2-(4-amino-1,2,5-oxadiazol-3-yl)-1-ethyl-1H-imidazo[4,5-c]pyridin-4-yl]-2-methyl-3-butyn-2-ol;
4-[6-({2-amino-1-[4-fluoro-3-(methyloxy)phenyl]ethyl}oxy)-2-(4-amino-1,2,5-oxadiazol-3-yl)-1-ethyl-1H-imidazo[4,5-c]pyridin-4-yl]-2-methyl-3-butyn-2-ol;
4-[6-{[(2R)-2-Amino-3-(2-furanyl)propyl]oxy}-2-(4-amino-1,2,5-oxadiazol-3-yl)-1-ethyl-1H-imidazo[4,5-c]pyridin-4-yl]-2-methyl-3-butyn-2-ol;
3-(3-Amino-1-{[2-(4-amino-1,2,5-oxadiazol-3-yl)-1-ethyl-4-(3-hydroxy-3-methyl-1-butyn-1-yl)-1H-imidazo[4,5-c]pyridin-6-yl]oxy}propyl)phenol;
4-((2R)-2-Amino-3-{[2-(4-amino-1,2,5-oxadiazol-3-yl)-1-ethyl-4-(3-hydroxy-3-methyl-1-butyn-1-yl)-1H-imidazo[4,5-c]pyridin-6-yl]oxy}propyl)phenol;
4-((2S)-2-Amino-3-{[2-(4-amino-1,2,5-oxadiazol-3-yl)-1-ethyl-4-(3-hydroxy-3-methyl-1-butyn-1-yl)-1H-imidazo[4,5-c]pyridin-6-yl]oxy}propyl)phenol;
4-(2-(4-Amino-1,2,5-oxadiazol-3-yl)-1-ethyl-6-{[(3R)-1,2,3,4-tetrahydro-3-isoquinolinylmethyl]-oxy}-1H-imidazo[4,5-c]pyridin-4-yl)-2-methyl-3-butyn-2-ol;
4-[6-({(2R)-2-Amino-3-[3-(methyloxy)phenyl]propyl}oxy)-2-(4-amino-1,2,5-oxadiazol-3-yl)-1-ethyl-1H-imidazo[4,5-c]pyridin-4-yl]-2-methyl-3-butyn-2-ol;
3-(3-Amino-1-{[2-(4-amino-1,2,5-oxadiazol-3-yl)-1-ethyl-4-(3-hydroxy-3-methyl-1-butyn-1-yl)-1H-imidazo[4,5-c]pyridin-6-yl]oxy}propyl)phenol;
4-[2-(4-amino-1,2,5-oxadiazol-3-yl)-1-ethyl-6-({2-[(3R)-1,2,3,4-tetrahydro-3-isoquinolinyl]ethyl}oxy)-1H-imidazo[4,5-c]pyridin-4-yl]-2-methyl-3-butyn-2-ol;
4-(2-(4-amino-1,2,5-oxadiazol-3-yl)-6-{[(2S)-2-amino-3-(3-pyridinyl)propyl]oxy}-1-ethyl-1H-imidazo[4,5-c]pyridin-4-yl)-2-methyl-3-butyn-2-ol;
4-(2-(4-amino-1,2,5-oxadiazol-3-yl)-6-{[(2S)-2-amino-3-(4-pyridinyl)propyl]oxy}-1-ethyl-1H-imidazo[4,5-c]pyridin-4-yl)-2-methyl-3-butyn-2-ol;
4-[6-({(2R)-2-amino-3-[4-(methyloxy)phenyl]propyl}oxy)-2-(4-amino-1,2,5-oxadiazol-3-yl)-1-ethyl-1H-imidazo[4,5-c]pyridin-4-yl]-2-methyl-3-butyn-2-ol;
4-[6-{[(2S)-2-amino-3-(2-furanyl)propyl]oxy}-2-(4-amino-1,2,5-oxadiazol-3-yl)-1-ethyl-1H-imidazo[4,5-c]pyridin-4-yl]-2-methyl-3-butyn-2-ol;
4-[6-({(2R)-2-amino-3-[2-(methyloxy)phenyl]propyl}oxy)-2-(4-amino-1,2,5-oxadiazol-3-yl)-1-ethyl-1H-imidazo[4,5-c]pyridin-4-yl]-2-methyl-3-butyn-2-ol;
4-[6-[(3-amino-3-cyclohexylpropyl)oxy]-2-(4-amino-1,2,5-oxadiazol-3-yl)-1-ethyl-1H-imidazo[4,5-c]pyridin-4-yl]-2-methyl-3-butyn-2-ol;
4-(2-(4-amino-1,2,5-oxadiazol-3-yl)-6-{[3-amino-3-(tetrahydro-2H-pyran-4-yl)propyl]oxy}-1-ethyl-1H-imidazo[4,5-c]pyridin-4-yl)-2-methyl-3-butyn-2-ol;
4-(2-(4-amino-1,2,5-oxadiazol-3-yl)-6-{[3-amino-3-(tetrahydro-2H-pyran-4-yl)propyl]oxy}-1-ethyl-1H-imidazo[4,5-c]pyridin-4-yl)-2-methyl-3-butyn-2-ol;
4-[2-(4-amino-1,2,5-oxadiazol-3-yl)-1-ethyl-6-({2-[(3R)-1,2,3,4-tetrahydro-3-isoquinolinyl]ethyl}oxy)-1H-imidazo[4,5-c]pyridin-4-yl]-2-methyl-3-butyn-2-ol;
4-(2-(4-amino-1,2,5-oxadiazol-3-yl)-1-ethyl-6-{[(3S)-1,2,3,4-tetrahydro-3-isoquinolinylmethyl]oxy}-1H-imidazo[4,5-c]pyridin-4-yl)-2-methyl-3-butyn-2-ol;
4-[6-{[2-(aminomethyl)-4-phenylbutyl]oxy}-2-(4-amino-1,2,5-oxadiazol-3-yl)-1-ethyl-1H-imidazo[4,5-c]pyridin-4-yl]-2-methyl-3-butyn-2-ol;
4-[6-[(3-amino-1-{[3-(methyloxy)phenyl]methyl}propyl)oxy]-2-(4-amino-1,2,5-oxadiazol-3-yl)-1-ethyl-1H-imidazo[4,5-c]pyridin-4-yl]-2-methyl-3-butyn-2-ol;
4-(2-(4-amino-1,2,5-oxadiazol-3-yl)-6-{[3-amino-1-(3-thienylmethyl)propyl]oxy}-1-ethyl-1H-imidazo[4,5-c]pyridin-4-yl)-2-methyl-3-butyn-2-ol;
4-[6-[(3-amino-1-{[3,4-bis(methyloxy)phenyl]methyl}propyl)oxy]-2-(4-amino-1,2,5-oxadiazol-3-yl)-1-ethyl-1H-imidazo[4,5-c]pyridin-4-yl]-2-methyl-3-butyn-2-ol; and
4-{2-(4-amino-1,2,5-oxadiazol-3-yl)-6-[(3-aminoethyl)oxy]-1-ethyl-1H-imidazo[4,5-c]pyridin-4-yl}-2-methyl-3-butyn-2-ol.

14. A pharmaceutically acceptable salt, hydrate, solvate or pro-drug of a compound of claim 13.

15. A compound of claim 1 selected from:
4-(2-(4-amino-1,2,5-oxadiazol-3-yl)-6-{[(2R)-2-amino-3-phenylpropyl]oxy}-1-ethyl-1H-imidazo[4,5-c]pyridin-4-yl)-2-methyl-3-butyn-2-ol;
4-{2-(4-amino-1,2,5-oxadiazol-3-yl)-6-[(2-amino-1-phenylethyl)oxy]-1-ethyl-1H-imidazo[4,5-c]pyridin-4-yl}-2-methyl-3-butyn-2-ol;
4-(2-(4-amino-1,2,5-oxadiazol-3-yl)-6-{[(1R)-2-amino-1-phenylethyl]oxy}-1-ethyl-1H-imidazo[4,5-c]pyridin-4-yl)-2-methyl-3-butyn-2-ol;
(rac)-4-[6-{[2-amino-1-(1,3-benzodioxol-4-yl)ethyl]oxy}-2-(4-amino-1,2,5-oxadiazol-3-yl)-1-ethyl-1H-imidazo[4,5-c]pyridin-4-yl]-2-methyl-3-butyn-2-ol;
(ent-1)-4-[6-{[2-amino-1-(1,3-benzodioxol-4-yl)ethyl]oxy}-2-(4-amino-1,2,5-oxadiazol-3-yl)-1-ethyl-1H-imidazo[4,5-c]pyridin-4-yl]-2-methyl-3-butyn-2-ol;
(ent-2)-4-[6-{[2-amino-1-(1,3-benzodioxol-4-yl)ethyl]oxy}-2-(4-amino-1,2,5-oxadiazol-3-yl)-1-ethyl-1H-imidazo[4,5-c]pyridin-4-yl]-2-methyl-3-butyn-2-ol;
4-(2-(4-amino-1,2,5-oxadiazol-3-yl)-6-{[2-amino-1-(phenylmethyl)ethyl]oxy}-1-ethyl-1H-imidazo[4,5-c]pyridin-4-yl)-2-methyl-3-butyn-2-ol;
4-(2-(4-amino-1,2,5-oxadiazol-3-yl)-6-{[(1S)-3-amino-1-phenylpropyl]oxy}-1-ethyl-1H-imidazo[4,5-c]pyridin-4-yl)-2-methyl-3-butyn-2-ol;
4-[2-(4-amino-1,2,5-oxadiazol-3-yl)-1-ethyl-6-({(S)-phenyl[(2R)-2-pyrrolidinyl]methyl}oxy)-1H-imidazo[4,5-c]pyridin-4-yl]-2-methyl-3-butyn-2-ol;
4-[6-({2-amino-1-[3-(methyloxy)phenyl]ethyl}oxy)-2-(4-amino-1,2,5-oxadiazol-3-yl)-1-ethyl-1H-imidazo[4,5-c]pyridin-4-yl]-2-methyl-3-butyn-2-ol;
4-[6-{[(2R)-2-amino-3-(2-chlorophenyl)propyl]oxy}-2-(4-amino-1,2,5-oxadiazol-3-yl)-1-ethyl-1H-imidazo[4,5-c]pyridin-4-yl]-2-methyl-3-butyn-2-ol;
4-[6-{[2-amino-1-(3-chlorophenyl)ethyl]oxy}-2-(4-amino-1,2,5-oxadiazol-3-yl)-1-ethyl-1H-imidazo[4,5-c]pyridin-4-yl]-2-methyl-3-butyn-2-ol;
4-[6-{[2-amino-1-(2-chlorophenyl)ethyl]oxy}-2-(4-amino-1,2,5-oxadiazol-3-yl)-1-ethyl-1H-imidazo[4,5-c]pyridin-4-yl]-2-methyl-3-butyn-2-ol;
(ent-2)-4-[6-[(2-amino-1-cyclohexylethyl)oxy]-2-(4-amino-1,2,5-oxadiazol-3-yl)-1-ethyl-1H-imidazo[4,5-c]pyridin-4-yl]-2-methyl-3-butyn-2-ol; and
(ent-1)-4-[6-[(2-amino-1-cyclohexylethyl)oxy]-2-(4-amino-1,2,5-oxadiazol-3-yl)-1-ethyl-1H-imidazo[4,5-c]pyridin-4-yl]-2-methyl-3-butyn-2-ol.

16. A pharmaceutically acceptable salt, hydrate, solvate or pro-drug of a compound of claim 15.

17. A pharmaceutical composition comprising a compound according to claim 1, and/or a pharmaceutically acceptable salt, hydrate, solvate or pro-drug thereof and a pharmaceutically acceptable carrier.

18. A process for preparing a pharmaceutical composition containing a pharmaceutically acceptable carrier or diluent and an effective amount of a compound of Formula (I) as described in claim 1 and/or a pharmaceutically acceptable salt, hydrate, solvate or pro-drug thereof, which process comprises bringing the compound of Formula (I) and/or a pharmaceutically acceptable salt, hydrate, solvate or pro-drug thereof into association with a pharmaceutically acceptable carrier or diluent.

19. A method of treating or lessening the severity of a disease or condition selected from cancer and arthritis in a mammal in need thereof, which comprises administering to such mammal a therapeutically effective amount of a compound of Formula I, as described in claim 1 and/or a pharmaceutically acceptable salt, hydrate, solvate or pro-drug thereof.

20. The method of claim 19 wherein the mammal is a human.

21. A method of treating or lessening the severity of a disease or condition selected from cancer and arthritis in a mammal in need thereof, which comprises administering to such mammal a therapeutically effective amount of a compound of Formula II, as described in claim 3 and/or a pharmaceutically acceptable salt, hydrate, solvate or pro-drug thereof.

22. The method of claim 21 wherein the mammal is a human.

23. The method according to claim 19 wherein said cancer is selected from brain (gliomas), glioblastomas, Bannayan-Zonana syndrome, Cowden disease, Lhermitte-Duclos disease, breast, colon, head and neck, kidney, lung, liver, melanoma, ovarian, pancreatic, prostate, sarcoma and thyroid.

24. The method according to claim 21 wherein said cancer is selected from brain (gliomas), glioblastomas, Bannayan-Zonana syndrome, Cowden disease, Lhermitte-Duclos disease, breast, colon, head and neck, kidney, lung, liver, melanoma, ovarian, pancreatic, prostate, sarcoma and thyroid.

25. (canceled)

26. The method of inhibiting Akt activity in a mammal in need thereof, which comprises administering to such mammal a therapeutically effective amount of a compound of Formula I, as described in claim 1 and/or a pharmaceutically acceptable salt, hydrate, solvate or pro-drug thereof.

27. The method of claim 26 wherein the mammal is a human.

28. A method of treating cancer in a mammal in need thereof, which comprises: administering to such mammal a therapeutically effective amount of
a) a compound of Formula (I), as described in claim 1 and/or a pharmaceutically acceptable salt, hydrate, solvate or pro-drug thereof; and
b) at least one anti-neoplastic agent.

29. The method claim 28, wherein the at least one anti-neoplastic agent is selected from the group consisting essentially of anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors; non-receptor tyrosine kinase angiogenesis inhibitors; immunotherapeutic agents; proapoptotic agents; and cell cycle signaling inhibitors.

30. The method of claim 28, wherein the at least one anti-neoplastic agent is an anti-microtubule agent selected from diterpenoids and vinca alkaloids.

31. The method of claim 28, wherein the at least one anti-neoplastic agent is a diterpenoid.

32. The method of claim 28, wherein the at least one anti-neoplastic agent is a vinca alkaloid.

33. The method of claim 28, wherein the at least one anti-neoplastic agent is a platinum coordination complex.

34. The method of claim 28, wherein the at least one anti-neoplastic agent is paclitaxel, carboplatin, or vinorelbine.

35. The method of claim 28, wherein the at least one anti-neoplastic agent is paclitaxel.

36. The method of claim 28, wherein the at least one anti-neoplastic agent is carboplatin.

37. The method of claim 28, wherein the at least one anti-neoplastic agent is vinorelbine.

38. The method of claim 28, wherein the at least one anti-neoplatic agent is a signal transduction pathway inhibitor.

39. The method of claim 38, wherein the signal transduction pathway inhibitor is an inhibitor of a growth factor receptor kinase selected from the group consisting of VEGFR2, TIE2, PDGFR, BTK, IGFR-1, TrkA, TrkB, TrkC, and c-fms.

40. The method of claim 38, wherein the signal transduction pathway inhibitor is an inhibitor of a serine/threonine kinase selected from the group consisting of rafk, akt, and PKC-zeta.

41. The method of claim 38, wherein the signal transduction pathway inhibitor is an inhibitor of a serine/threonine kinase selected from the src family of kinases.

42. The method of claim 41, wherein the signal transduction pathway inhibitor is an inhibitor of c-src.

43. The method of claim 38, wherein the signal transduction pathway inhibitor is an inhibitor of Ras oncogene selected from inhibitors of farnesyl transferase and geranylgeranyl transferase.

44. The method of claim 38, wherein the signal transduction pathway inhibitor is an inhibitor of a serine/threonine kinase selected from the group consisting of PI3K.

45. The method of claim 28, wherein the at least one anti-neoplastic agent is a cell cycle signaling inhibitor.

46. The method of claim 45, wherein the cell cycle signaling inhibitor is selected from inhibitors of the group CDK2, CDK4, and CDK6.

47-48. (canceled)
